SEQUENCE LISTING (1 sequence)

SEQ ID NO: 1 - protein, 16 aa, Artificial Sequence
  Description of Artificial Sequence Synthetic peptide
AFFAQLQLDEETGEFL

(19) United States
(12) Patent Application Publication
(10) Pub. No.: US 2023/0127304 A1
(43) Pub. Date: Apr. 27, 2023

(54) NEW INHIBITORS FOR THE KEAP1-NRF2 PROTEIN-PROTEIN INTERACTION

(71) Applicants: PRESIDENT AND FELLOWS OF HARVARD COLLEGE, Cambridge, MA (US); DANA-FARBER CANCER INSTITUTE, INC., Boston, MA (US)

(72) Inventors: Gerhard WAGNER, Cambridge, MA (US); Christoph GORGULLA, Cambridge, MA (US); Zi-Fu WANG, Cambridge, MA (US); Haribabu ARTHANARI, Cambridge, MA (US); Andras Pal BOESZOERMENYI, Boston, MA (US)

(73) Assignees: PRESIDENT AND FELLOWS OF HARVARD COLLEGE, Cambridge, MA (US); DANA-FARBER CANCER INSTITUTE, INC., Boston, MA (US)

(21) Appl. No.: 17/908,422

(22) PCT Filed: Mar. 2, 2021
(86) PCT No.: PCT/US2021/020389

§ 371 Date: Aug. 31, 2022

Related U.S. Application Data
(60) Provisional application No. 62/984,010, filed on Mar. 2, 2020.

(51) Int. Cl. A61K 31/655; A61K 31/341; A61K 31/196; A61K 31/53; A61K 31/55; A61K 31/454; A61K 31/421; A61K 31/635; A61K 31/4245; A61K 31/50; A61K 31/437; A61K 31/4709; A61K 31/4155; A61K 31/351; A61K 31/422; A61K 31/18; A61K 31/4192; A61K 31/433; A61K 31/4375; A61K 31/5513; A61K 31/47; A61K 31/4985; A61K 31/473; A61K 31/5025; A61K 31/506; A61K 31/366; A61K 31/44; A61K 31/4439; A61K 31/4725; A61K 31/403; A61K 31/4184; A61K 31/5377; A61K 31/538; A61K 31/41; A61K 31/498; A61K 31/502

(52) U.S. Cl. CPC A61K 31/655; A61K 31/341; A61K 31/196; A61K 31/53; A61K 31/55; A61K 31/454; A61K 31/421; A61K 31/635; A61K 31/4245; A61K 31/50; A61K 31/437; A61K 31/4709; A61K 31/4155; A61K 31/351; A61K 31/422; A61K 31/18; A61K 31/4192; A61K 31/433; A61K 31/4375; A61K 31/5513; A61K 31/47; A61K 31/4985; A61K 31/473; A61K 31/5025; A61K 31/506; A61K 31/366; A61K 31/44; A61K 31/4439; A61K 31/4725; A61K 31/403; A61K 31/4184; A61K 31/5377; A61K 31/538; A61K 31/41; A61K 31/498; A61K 31/502

(57) ABSTRACT

Described herein are compounds, compositions and methods useful for inhibiting Kelch-like ECH-associated protein 1 (KEAP1). The compounds, compositions and methods described herein are useful for treating diseases, disorders or conditions associated with KEAP1.

### CROSS REFERENCE TO RELATED APPLICATIONS

This application claims benefit under 35 U.S.C. § 119(e) of the U.S. Provisional Application No. 62/984,010, filed Mar. 2, 2020, the content of which is incorporated herein by reference in its entirety.

### GOVERNMENT SUPPORT

This invention was made with government support under CA200913 awarded by National Institutes of Health. The government has certain rights in the invention.

### TECHNICAL FIELD

The present disclosure relates generally to compounds, compositions and methods useful for inhibiting Kelch-like ECH-associated protein 1 (KEAP1). For example, compounds, compositions and methods useful for inhibiting Nuclear factor erythroid-derived 2-related factor 2 (NRF2)-KEAP1 interaction.

### BACKGROUND

Nuclear factor erythroid-derived 2-related factor 2 (NRF2) is a master regulator of cellular resistance to oxidative stress and cellular repair (Yonchuk, J. G. et al., J Pharmacol. Exp. Ther. 363, 114-125 (2017)). Under unstressed conditions, NRF2 is sequestered by Kelch-like ECH-associated protein 1 (KEAP1), an E3 ubiquitin ligase substrate adaptor, and targeted for degradation (Pallesen, J. S., Tran, K. T. & Bach, A. J. Med. Chem. 61, 8088-8103 (2018)). However, upon oxidative stress, reactive oxidants dissociate NRF2 from KEAP1 and NRF2 translocates to the nucleus to activate its transcriptional program of approximately 250 genes (Davies, T. G. et al., J Med. Chem. 59, 3991-4006 (2016)). The NRF2-KEAP1 pathway is critical in protecting the cell under oxidative stress and inflammation and is implicated in a number of diseases (Cuadrado, A. et al., Nat. Rev. Drug Discov. 18, 295-317 (2019)). There are ten drugs targeting KEAP1 that are in clinical trials and nine more that are at the preclinical stage (Cuadrado, A. et al., Nat. Rev. Drug Discov. 18, 295-317 (2019)). However, challenges regarding target specificity, pharmacodynamic properties, efficacy and safety remain. Accordingly, there remains a need in the art for compounds, compositions and methods inhibiting KEAP1 for use in treating KEAP1 related diseases or conditions. The present disclosure addresses some of these needs.

### SUMMARY OF THE INVENTION

In one aspect, the disclosure provides compounds of Formula (I):

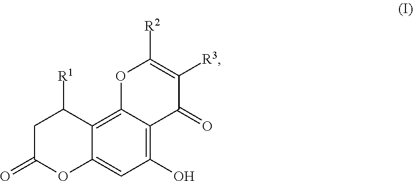

wherein:
   R¹ is substituted or unsubstituted aryl, substituted or unsubstituted heteroaryl, or substituted or unsubstituted heterocyclyl;
   R² is substituted or unsubstituted aryl, substituted or unsubstituted heteroaryl, or substituted or unsubstituted heterocyclyl;
   R³ is hydrogen, —OR^{A}, —SR^{A}, or —N(R^{A})₂; and
   each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl,
or a pharmaceutically acceptable salt, solvate, hydrate, polymorph, co-crystal, tautomer, stereoisomer, isotopically labeled derivative, or prodrug thereof.

In one aspect, the disclosure provides compounds of Formula (II):

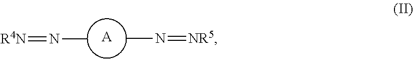

wherein:
   A is substituted or unsubstituted arylene, substituted or unsubstituted biarylene, or substituted or unsubstituted heteroarylene;
   R⁴ is substituted or unsubstituted aryl, substituted or unsubstituted heteroaryl, or substituted or unsubstituted heterocyclyl; and
   R⁵ is substituted or unsubstituted aryl, substituted or unsubstituted heteroaryl, or substituted or unsubstituted heterocyclyl;
   or a pharmaceutically acceptable salt, solvate, hydrate, polymorph, co-crystal, tautomer, stereoisomer, isotopically labeled derivative, or prodrug thereof.

In one aspect, the disclosure provides compounds of Formula (III):

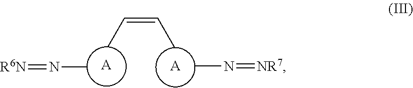

wherein:
   each A is independently substituted or unsubstituted aryl, or substituted or unsubstituted heteroaryl;
   R⁶ is substituted or unsubstituted aryl, substituted or unsubstituted heteroaryl, or substituted or unsubstituted heterocyclyl; and
   R⁷ is substituted or unsubstituted aryl, substituted or unsubstituted heteroaryl, or substituted or unsubstituted heterocyclyl;
      or a pharmaceutically acceptable salt, solvate, hydrate, polymorph, co-crystal, tautomer, stereoisomer, isotopically labeled derivative, or prodrug thereof.

In another aspect, the disclosure provides a compound selected from Group A, where the Group A comprises the following compounds:

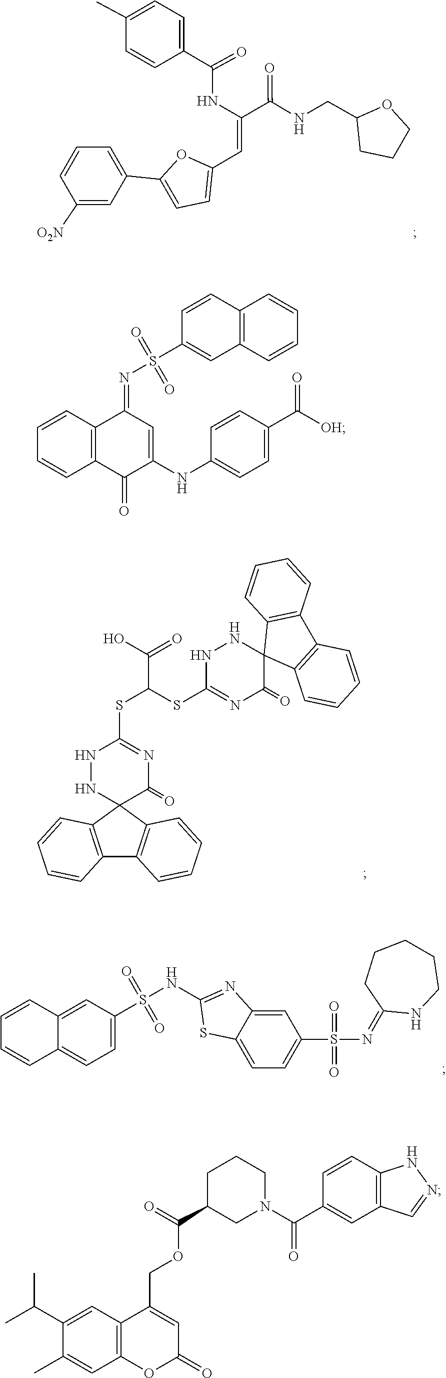 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1 ; Cc1cc2oc(=O)cc(COC(=O)[C@H]3CCCN(C(=O)c4ccc5[nH]ncc5c4)C3)c2cc1C(C)C]

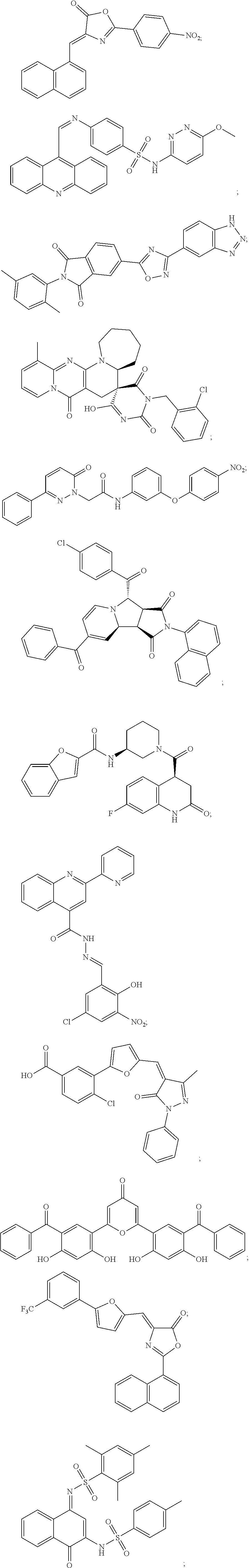

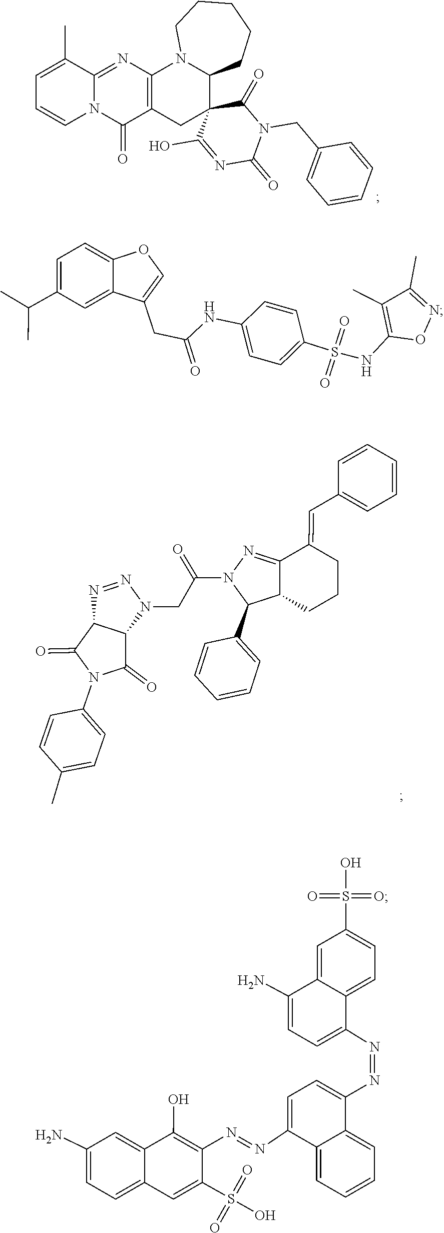 [SMILES: Cc1cccn2c(=O)c3c(nc12)N1CCCCC[C@H]1[C@]1(C3)C(=O)N(Cc2ccccc2)C(=O)N=C1O ; Cc1noc(NS(=O)(=O)c2ccc(NC(=O)Cc3coc4ccc(C(C)C)cc34)cc2)c1C ; Cc1ccc(N2C(=O)[C@@H]3[C@@H](N=NN3CC(=O)N3N=C4/C(=C/c5ccccc5)CCC[C@@H]4[C@H]3c3ccccc3)C2=O)cc1 ; Nc1ccc2cc(S(=O)(=O)O)c(/N=N/c3ccc(/N=N\c4ccc(N)c5cc(S(=O)(=O)O)ccc45)c4ccccc34)c(O)c2c1]

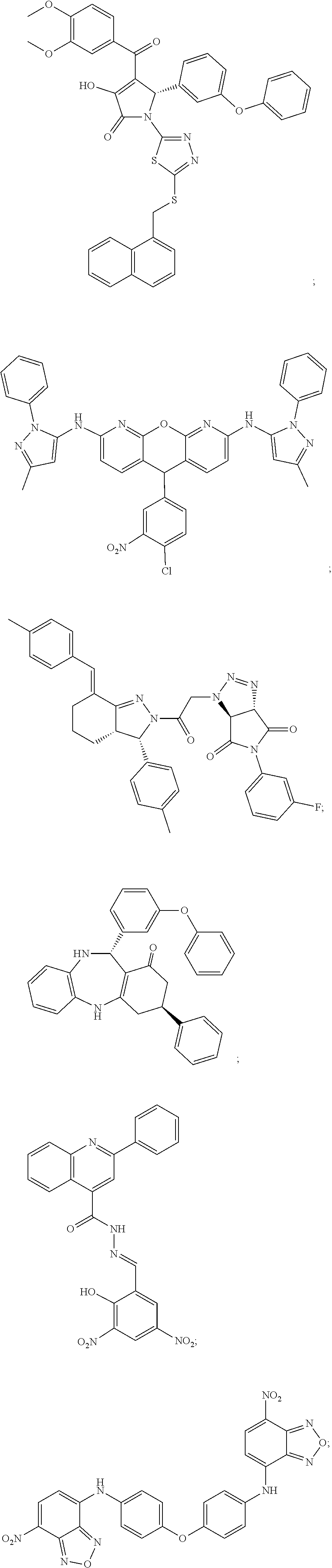

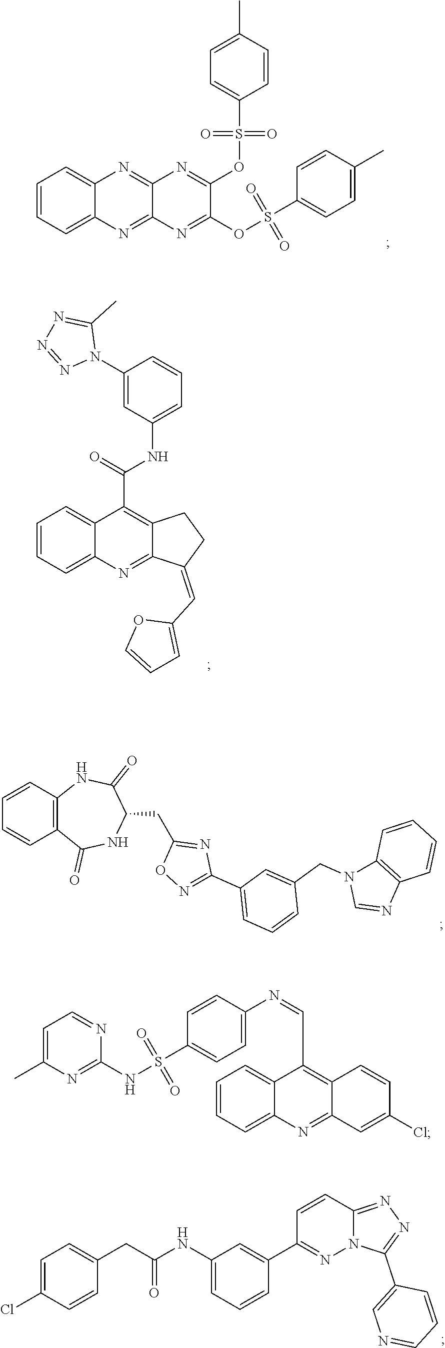

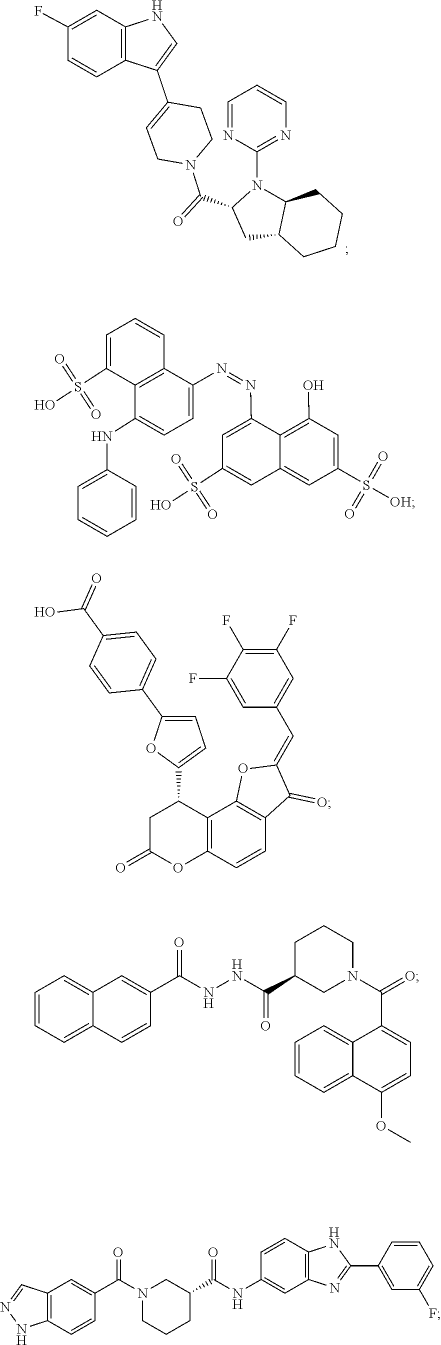

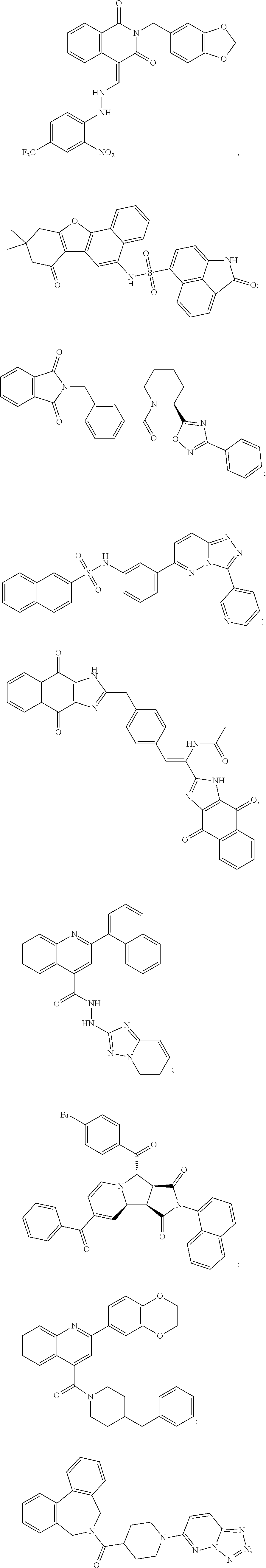

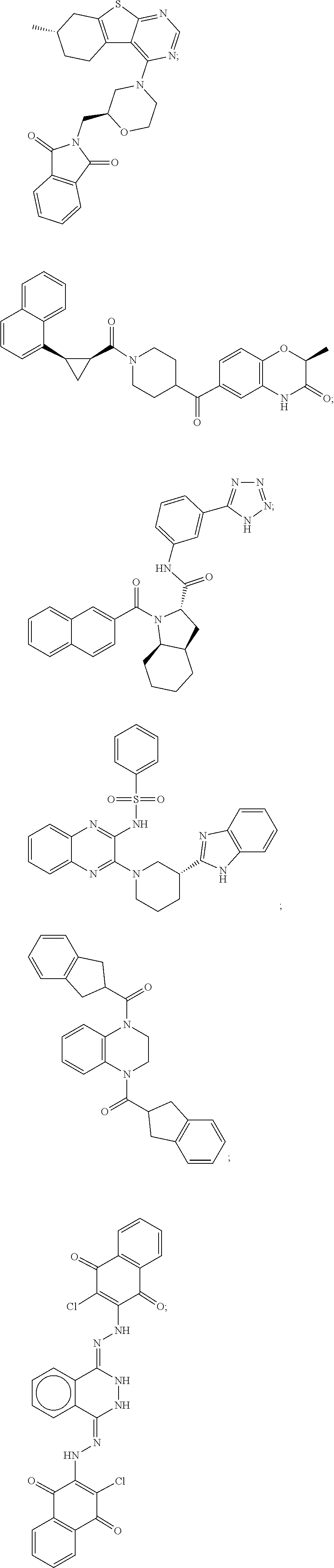

or a pharmaceutically acceptable salt, solvate, hydrate, polymorph, co-crystal, tautomer, stereoisomer, isotopically labeled derivative, or prodrug thereof

It is noted that any reference to a compound of the disclosure includes a pharmaceutically acceptable salt, solvate, hydrate, polymorph, co-crystal, tautomer, stereoisomer, isotopically labeled derivative, or prodrug thereof.

The compounds described herein can be formulated into compositions or formulations. Accordingly, in another aspect provided herein is pharmaceutical composition comprising a compound described herein and a pharmaceutically acceptable carrier or excipient.

The compounds of the disclosure can inhibit KEAP1. Accordingly, another aspect of the disclosure provides a method of inhibiting KEAP1. The method comprising contacting KEAP1 with a compound of the disclosure.

Still another aspect of the disclosure provides a method of inhibiting KEAP1-Nrf2 interaction. The method comprising contacting KEAP1 with a compound of the disclosure.

Still yet another aspect of the disclosure provides a method of activating Nrf2 The method comprising contacting KEAP1 with a compound of the disclosure.

Compounds, composition or methods of inhibiting KEAP1 can be useful for treating, preventing, or ameliorating a disease, disorder or condition associated with dysfunction of KEAP1 Nrf2 axis in a subject. Accordingly, another aspect of the disclosure provides a method of treating a disease, disorder or condition associated with dysfunction of the KEAP1-Nrf2 axis. The method comprising administering to a subject in need thereof a therapeutically effective amount of a compound of the disclosure.

By a disease, disorder or condition associated with dysfunction of the KEAP1-Nrf2 axis is meant a disease or disorder whose pathology involves a KEAP1-Nrf2 interaction. Accordingly, yet another aspect of the disclosure provides a method of treating a disease, disorder or condition associated with KEAP1-Nrf2 interaction. The method comprising administering to a subject in need thereof a therapeutically effective amount of a compound of the disclosure.

In some embodiments, the disease, disorder or condition that can be treated with a compound, composition or method of the disclosure can be selected from the group consisting abdominal aortic aneurysm, acute kidney injury, adult brain glioblastoma, advanced solid tumors lymphoid malignancies, aging, alcohol sensitivity, allergic, Alport syndrome, Alzheimer's disease, asthma, atopic asthmatics, autism spectrum disorder, autosomal dominant polycystic kidney, Barrett esophagus, low-grade dysplasia, brain ischemia, breast cancer or breast neoplasm, cardiovascular risk, cataract surgery, cholelithiasis, cholestasis, chronic hepatitis c, chronic kidney disease, chronic lymphocytic leukemia, chronic renal insufficiency, chronic schizophrenia, chronic subclinical inflammation, CKD associated with type 1 diabetes, cognition, colon cancer, COPD, corneal endothelial cell loss, crohn's disease, cutaneous t cell lymphoma, diabetes mellitus, diabetic nephropathy, diarrhea, endometriosis, environmental carcinogenesis, focal segmental glomerulosclerosis, Friedreich's ataxia, healthy, Helicobacter pylori infection, hepatic impairment, healthy, huntington disease, IgA nephropathy, inflammation and pain following ocular surgery, insulin resistance, liver disease, lung cancer, major depression, melanoma, metabolic syndrome x, mild cognitive impairment, mitochondrial myopathy, multiple sclerosis, neoplasms, nonalcoholic fatty liver or nonalcoholic steatohepatitis, noninsulin-dependent, nonischemic cardiomyopathy, obstructive sleep apnea, ocular inflammation, ocular pain, polymorphism, prediabetes, primary biliary cirrhosis, primary focal segmental glomerulosclerosis (FSGS), prostate cancer, psoriasis, psychosis, pulmonary arterial hypertension (pah), pulmonary hypertension, redox status, rheumatoid arthritis, rhinitis, schistosomiasis, schizophrenia, small lymphocytic lymphoma, subarachnoid haemorrhage, and type 2 (type 2 diabetes).

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1H show docking poses (FIGS. 1A and 1B) and experimental verification (FIGS. 1C-1H) of two exemplary compounds (iKeap1 and iKeap2). The docking poses (FIGS. 1A and 1Bb) were obtained from stage-2 of the virtual screening. SPR steady-state binding curves are shown for iKeap1 (FIG. 1C) and iKeap2 (FIG. 1D), showing clear binding with nanomolar K_{d}. Shown is one representative data from three independent experiments (n=3) with similar results. Ligand-detected NMR experiments, CPMG-R2 and STD-NMR (FIGS. 1E and 1F) confirm the binding of the two compounds. The two hits were also functional in the FP assay (FIGS. 1G and 1H) confirming that the compounds displace the peptide. The FP data shown here is from three technical replicates and the curve was fitted to the average value of the three the technical replicates. The mean and the standard deviation for the individual data points are shown. The FP was repeated independently twice with similar results and one representative result is shown here.

FIGS. 2A and 2B show binding of the NRF2 peptide to KEAP1 as assayed by FP (FIG. 2A) and BLI (FIG. 2B). In FIG. 2A, for the FP assay a TAMRA-tagged NRF2 peptide and for the BLI assay a Biotin-tagged NRF2 peptide were used. The FP assay was performed with three technical replicates per point. The mean and standard deviation are shown for each titration point, along with the fitted curve. Two independent experiments (n=2) were performed, each with similar results and one representative result is shown here. In FIG. 2B, for the BLI assay a biotin-tagged NRF2 peptide was used. The BLI experiment was repeated independently twice (n=2) with similar results and one representative result is shown here.

FIGS. 3A-3D show comparison of exemplary compound (iKeap1) with a previously identified displacer C17. FIG. 3A shows crystal structure (PDB ID: 5FNQ⁹) of KEAP1 with its ligand removed, the structure used for the primary virtual screening procedure. FIG. 3B shows structure of KEAP1 (PDB code 4IQK) with ligand C17 (Table 2), which is also shown in FIG. 3D. FIG. 3C shows iKeap1, the best binder as accessed by array of experimental validations, is similar to compound C17 previously identified by experimental methods (FIG. 3D). Though iKeap1 and C17 look similar they differ in a number of aspects in their core scaffold (therefore analogues of the two compounds cover distinct chemical spaces, assuming the analogues retain the core scaffold of the parent compound). This similarity, as well as the fact that the predicted docking positions (FIG. 1A) of both ligands (FIG. 3B) are nearly identical, is an additional evidence that iKeap1 is binding at the predicted site.

FIGS. 4A-4H shows the difference between binders and displacers for two exemplary compounds, iKeap8 and. SPR confirms that both iKeap8 and iKeap9 bind KEAP1 (FIGS. 4A and 4B) with similar K_{d} values. Shown are representative results from the SPR assay for iKeap8 and iKeap9. For each compound, three independent SPR experiments were performed, each with similar results and one representative result is shown here. Ligand-detected NMR experiments shows that both iKeap8 and iKeap9 bind to KEAP1 (FIGS. 4C and 4D). However, FP (FIGS. 4E and 4F) and BLI (FIGS. 4G and 4H) assays show that iKeap8 is able to displace the NRF2 peptide while iKeap9 is not able to effectively displace the NRF2 peptide. The fluorescence polarization (FP) assay was performed with three technical replicates per concentration measured. The mean and standard deviation are shown for each titration point, along with the fitted curve.

FIGS. 5A-5F shows two more exemplary displacers, iKeap7 and iKeap22, both of which were confirmed as binders by SPR (top panels). Ligand-detected NMR experiments shows that both iKeap7 and iKeap22 bind to KEAP1 (FIGS. 4C and 4D). iKeap7 is confirmed to be a displacer of the NRF2 peptide by both FP (bottom left panel) and BLI (not shown). Since the FP experiments on iKeap22 were affected by autofluorescence, BLI (bottom right panel) was needed to confirm that this compounds is a displacer. The FP assay was performed with three technical replicates per concentration measured. The mean and standard deviation are shown for each titration point, along with the fitted curve. Two independent BLI experiment were performed with similar results and one representative result shown here.

FIG. 6 shows the docking pose of one of the hit compounds (iKeap9, ball-and-stick representation) bound to KEAP1, together with the NRF2 peptide (PDB ID: 4IFL; peptide in violet). iKeap9 is a tight binder (180 nM by steady-state SPR) but cannot displace NRF2. The left figure shows the top view, while the right figure shows the side view of the cross-section of KEAP1 along the central plane. The violet box in right figure indicates the docking region (where the ligands were allowed to bind) which was used in the virtual screening. The site of interest includes a part of the deep pocket/tunnel of the β-barrel-shaped KEAP1, since it can allow ligands to bind more tightly by insertion into the channel than on a shallow surface. However, the deep tunnel is largely non-overlapping with the peptide binding site (which binds to the entrance site of the tunnel). Thus, binding molecules might only partially interfere with the peptide binding, which might reduce or eliminate the ability of small molecule binders to displace the peptide. The ability of a small molecule to displace the peptide is hard to predict, and was not attempted in this study. In some cases, small molecules can also act as molecular glues and strengthen the interaction between NRF2 and KEAP1.

FIGS. 7A-7D are ¹H-¹³C HMQC experiments showing the binding of the NRF2 peptide (FIGS. 7A and 7C), iKeap1 (FIG. 7B) and iKeap2 (FIG. 7D) to KEAP1 as monitored by chemical shift perturbation to the methyl resonances of Ile, Leu and Val of KEAP1. Upon addition of iKeap1 and iKeap2 we see selective and specific changes to a subset of resonances and these correlate to the changes we observe when we add the NRF2 peptide. The rest of the resonances are largely unaffected. The indicates that the protein is folded and does not aggregate after the addition of the compounds.

FIGS. 8A-8D are ¹H-¹³C HMQC experiments showing the binding of the NRF2 peptide (FIGS. 8A and 8C), iKeap8 (FIG. 8B) and iKeap9 (FIG. 8D) to KEAP1 as monitored by chemical shift perturbation to the methyl resonances of Ile, Leu and Val of KEAP1. Upon addition of iKeap8 and iKeap9 we see selective and specific changes to a subset of resonances and these correlate to the changes we observe when we add the NRF2 peptide. The rest of the resonances are largely unaffected. The indicates that the protein is folded and does not aggregate after the addition of the compounds.

FIGS. 9A-9D are ¹H-¹³C HMQC experiments showing the binding of the NRF2 peptide (FIGS. 9A and 9C), iKeap7 (FIG. 9B) and iKeap22 (FIG. 9D) to KEAP1 as monitored by chemical shift perturbation to the methyl resonances of Ile, Leu and Val of KEAP1. Upon addition of iKeap7 and iKeap22 we see selective and specific changes to a subset of resonances and these correlate to the changes we observe when we add the NRF2 peptide. The rest of the resonances are largely unaffected. The indicates that the protein is folded and does not aggregate after the addition of the compounds.

FIG. 10 shows NMR solubility assay of iKeap1. Determination of the solubility of iKeap1 via an NMR solubility assay as described in [LCG+13]. As can be seen in the figure, the NMR intensity of iKeap1 remains linear over the range of concentrations measured here, indicating that iKeap1 does not aggregate at these concentrations.

FIG. 11 shows NQO1 assay results.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

Various aspects described herein are based on inventors' discovery inter alia of compounds that inhibit KEAP1. For example, the inventors have discovered compounds that can disrupt or inhibit KEAP1-Nrf2 interactions.

Accordingly, in one aspect provided herein is a compound of Formula (I).

In some embodiments of any one of the aspects, R¹ in compounds of Formula (I) is phenyl, pyridinyl, pyrimidinyl, furanyl, quinolinyl, quinolonyl, naphthyl, anthracenyl or chromenyl, each of which can be optionally substituted. For example, R¹ is phenyl, quinolinyl, quinolonyl or chromenyl, each of which can be optionally substituted. In some embodiments, R¹ is phenyl or phenyl substituted with an alkoxy group. In some embodiments, R¹ is phenyl, 4-[2-(2,3-Dihydro-1-benzofuran-5-yl)ethoxy]-phenyl, 3-benzyloxy-phenyl, quinolinyl, quinolonyl, 7-methyl-2-oxo-1,2-dihydro-3-quinolinyl, 4H-chromenyl, or 7-hydroxy-4-oxo-4H-chromenyl. Preferably, R¹ is 4-[2-(2,3-Dihydro-1-benzofuran-5-yl)ethoxy]-phenyl, 3-benzyloxy-phenyl, 7-methyl-2-oxo-1,2-dihydro-3-quinolinyl, or 7-hydroxy-4-oxo-4H-chromenyl.

In some embodiments of any one of the aspects, R² in compounds of Formula (I) is phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, or indanyl. For example, R¹ is phenyl, pyridinyl, pyrimidinyl, furanyl, naphthyl or anthracenyl, each of which can be optionally substituted. In some embodiments, R² is phenyl, which can be optionally substituted. In some embodiments, R² is 4-hydroxyphenyl, which can be optionally further substituted. In some preferred embodiments, R² is 4-hydroxyphenyl or 3,4-dihydroxyphenyl.

In some embodiments of any one of the aspects, R³ in compounds of Formula (I) is H or OR^{A}, where R^{A} is H or C₁-C₆alkyl. For example, R³ is H, OH, methoxy, ethoxy, propoxy, isopropoxy or butoxy. In some preferred embodiments, R³ in compounds of Formula (I) is H or OH.

In some embodiments of any one of the aspects, R^{A} in compounds of Formula (I) is H, substituted or unsubstituted alkyl. For example, R^{A} is H or C₁-C₆alkyl. In some embodiments, R^{A} is H, methyl, ethyl, propyl, isopropyl, butyl or pentyl. Preferably R^{A} is H.

Exemplary compounds of Formula (I) include, but are not limited to, the following:

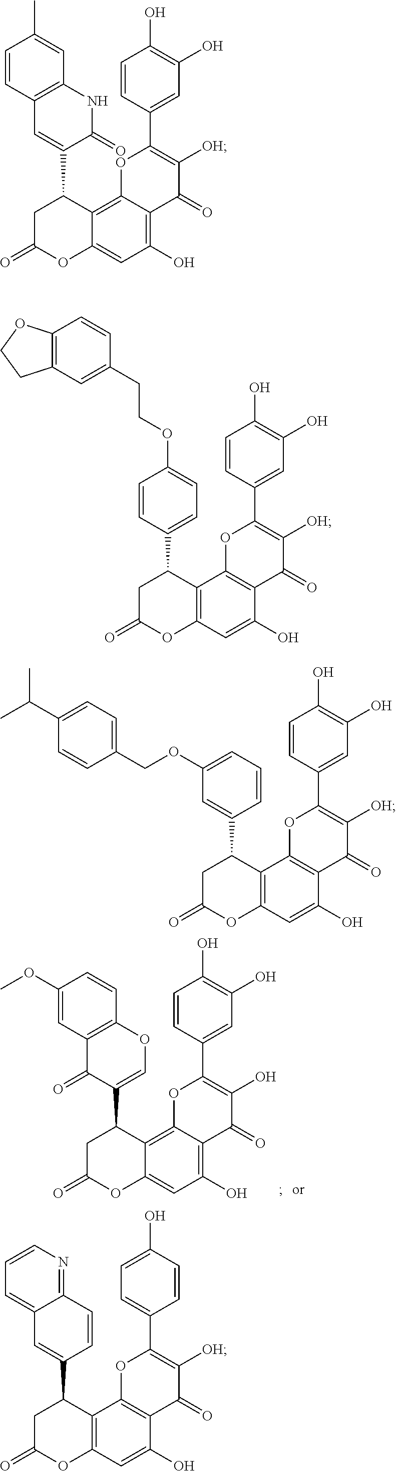

or a pharmaceutically acceptable salt, solvate, hydrate, polymorph, co-crystal, tautomer, stereoisomer, isotopically labeled derivative, or prodrug thereof.

In one aspect, provided herein are compounds of Formula (II).

In some embodiments, of any one of the aspects, A in compounds of Formula (II) is benzene, pyridine, pyrimidine, furan, naphthalene, quinolone, quinolone, or anthracene, each of which can be optionally substituted. For example, A can be benzene, pyridine, naphthalene or anthracene, each of which can be optionally substituted. In some embodiments, A is benzene, methylbenzene, methoxybenzene, aminodimethylbenzene, naphthalene, or 3-sulfonyl-1-hydroxy-6-aminonaphthalene.

In some embodiments, of any one of the aspects, R⁴ in compounds of Formula (II) is phenyl, pyridinyl, pyrimidinyl, furanyl, quinolinyl, quinolonyl, naphthyl, anthracenyl or chromenyl, each of which can be optionally substituted. For example, R⁴ is phenyl, naphthyl, quinolinyl, quinolonyl or chromenyl, each of which can be optionally substituted. In some embodiments, R⁴ is phenyl or naphthyl, each of which can be optionally substituted. For example, R⁴ is phenyl, 4-Sulfonylphenyl, 4-sulfonyl-2-methylphenyl, 2-methylphenyl, 3-carboxy, 5-sulfonyl-4-hydroxyphenyl, phenylazophenyl, (4-methylphenyl)azophenyl, 3-(4-methylphenyl)azophenayl, (4-methylphenyl)azo-2-amino-3,6-dimethylphenyl, 5-(4-methylphenyl)azo-2-amino-3,6-dimethylphenyl, phenylazo-2-methylphenyl, (3-carboxy, 5-sulfonyl-4-hydroxyphenyl)azophenyl, nitrophenyl, 4-nitophenyl, (3-carboxy, 5-sulfonyl-4-hydroxyphenyl)azo-methylphenyl, 4-(3-carboxy, 5-sulfonyl-4-hydroxyphenyl)azo-methylphenyl, (3-carboxy, 5-sulfonyl-4-hydroxyphenyl)azo-3-methylphenyl, 4-(3-carboxy, 5-sulfonyl-4-hydroxyphenyl)azo-3-methylphenyl, phenylazonaphthyl, nitrophenylazonaphthyl, (4-nitrophenyl)azonaphthyl, 5-(nitrophenyl)azo-3-sulfonyl-1-hydroxy-6-aminonaphthyl, 5-(4-nitrophenyl)azo-3-sulfonyl-1-hydroxy-6-aminonaphthyl, hydroxysulfonylnaphthyl, aminosulfonyl, aminohydroxysulfonylnaphthyl, 1-sulfonyl-4-hydroxynaphthyl, 3-sulfonyl-1-hydroxynaphthyl, 1-hydroxysulfonylnaphthyl, 1-hydroxy-3-sulfonylnaphthyl, 1-hydroxy-5-sulfonylnaphthyl, 1-hydroxy-3-sulfonyl-7-aminonaphthyl, 1-aminosulfonylnaphthyl, 1-amino-3-sulfonylnaphthyl, 1-amino-7-sulfonylnaphthyl, 4-amino-6-sulfonylnaphthyl, (methylphenyl)aminosulfonylnaphthyl, 4-(methylphenyl)aminosulfonylnaphthyl, (methylphenyl)amino-5-sulfonylnaphthyl, 4-(methylphenyl)amino-5-sulfonylnaphthyl, 1-(methylphenyl)amino-8-sulfonylnaphthyl, (4-methylphenyl)aminosulfonylnaphthyl, 4-(4-methylphenyl)aminosulfonylnaphthyl, (4-methylphenyl)amino-5-sulfonylnaphthyl, 4-(4-methylphenyl)amino-5-sulfonylnaphthyl, or 1-(4-methylphenyl)amino-8-sulfonylnaphthyl, each of which can be optionally further substituted.

In some embodiments, of any one of the aspects, R⁵ in compounds of Formula (II) is phenyl, pyridinyl, pyrimidinyl, furanyl, quinolinyl, quinolonyl, naphthyl, anthracenyl or chromenyl, each of which can be optionally substituted. For example, R⁵ is phenyl, naphthyl, quinolinyl, quinolonyl or chromenyl, each of which can be optionally substituted. In some embodiments, R⁵ is phenyl or naphthyl, each of which can be optionally substituted. For example, R⁵ is phenyl, 4-Sulfonylphenyl, 4-sulfonyl-2-methylphenyl, 2-methylphenyl, 3-carboxy, 5-sulfonyl-4-hydroxyphenyl, phenylazophenyl, (4-methylphenyl)azophenyl, 3-(4-methylphenyl)azophenayl, (4-methylphenyl)azo-2-amino-3,6-dimethylphenyl, 5-(4-methylphenyl)azo-2-amino-3,6-dimethylphenyl, phenylazo-2-methylphenyl, (3-carboxy, 5-sulfonyl-4-hydroxyphenyl)azophenyl, nitrophenyl, 4-nitophenyl, (3-carboxy, 5-sulfonyl-4-hydroxyphenyl)azo-methylphenyl, 4-(3-carboxy, 5-sulfonyl-4-hydroxyphenyl)azo-methylphenyl, (3-carboxy, 5-sulfonyl-4-hydroxyphenyl)azo-3-methylphenyl, 4-(3-carboxy, 5-sulfonyl-4-hydroxyphenyl)azo-3-methylphenyl, phenylazonaphthyl, nitrophenylazonaphthyl, (4-nitrophenyl)azonaphthyl, 5-(nitrophenyl)azo-3-sulfonyl-1-hydroxy-6-aminonaphthyl, 5-(4-nitrophenyl)azo-3-sulfonyl-1-hydroxy-6-aminonaphthyl, hydroxysulfonylnaphthyl, aminosulfonyl, aminohydroxysulfonylnaphthyl, 1-sulfonyl-4-hydroxynaphthyl, 3-sulfonyl-1-hydroxynaphthyl, 1-hydroxysulfonylnaphthyl, 1-hydroxy-3-sulfonylnaphthyl, 1-hydroxy-5-sulfonylnaphthyl, 1-hydroxy-3-sulfonyl-7-aminonaphthyl, 1-aminosulfonylnaphthyl, 1-amino-3-sulfonylnaphthyl, 1-amino-7-sulfonylnaphthyl, 4-amino-6-sulfonylnaphthyl, (methylphenyl)aminosulfonylnaphthyl, 4-(methylphenyl)aminosulfonylnaphthyl, (methylphenyl)amino-5-sulfonylnaphthyl, 4-(methylphenyl)amino-5-sulfonylnaphthyl, 1-(methylphenyl)amino-8-sulfonylnaphthyl, (4-methylphenyl)aminosulfonylnaphthyl, 4-(4-methylphenyl)aminosulfonylnaphthyl, (4-methylphenyl)amino-5-sulfonylnaphthyl, 4-(4-methylphenyl)amino-5-sulfonylnaphthyl, or 1-(4-methylphenyl)amino-8-sulfonylnaphthyl, each of which can be optionally further substituted.

In compounds of Formula (II), R⁴ and R⁵ can be same or different. In some embodiments, R⁴ and R⁵ are same. In some other embodiments, R⁴ and R⁵ are different.

In compounds of Formula (II), R⁴ and R⁵ can be same or different. In some embodiments, R⁴ and R⁵ are same. In some other embodiments, R⁴ and R⁵ are different.

Exemplary compounds of Formula (II) include, but are not limited to, the following:

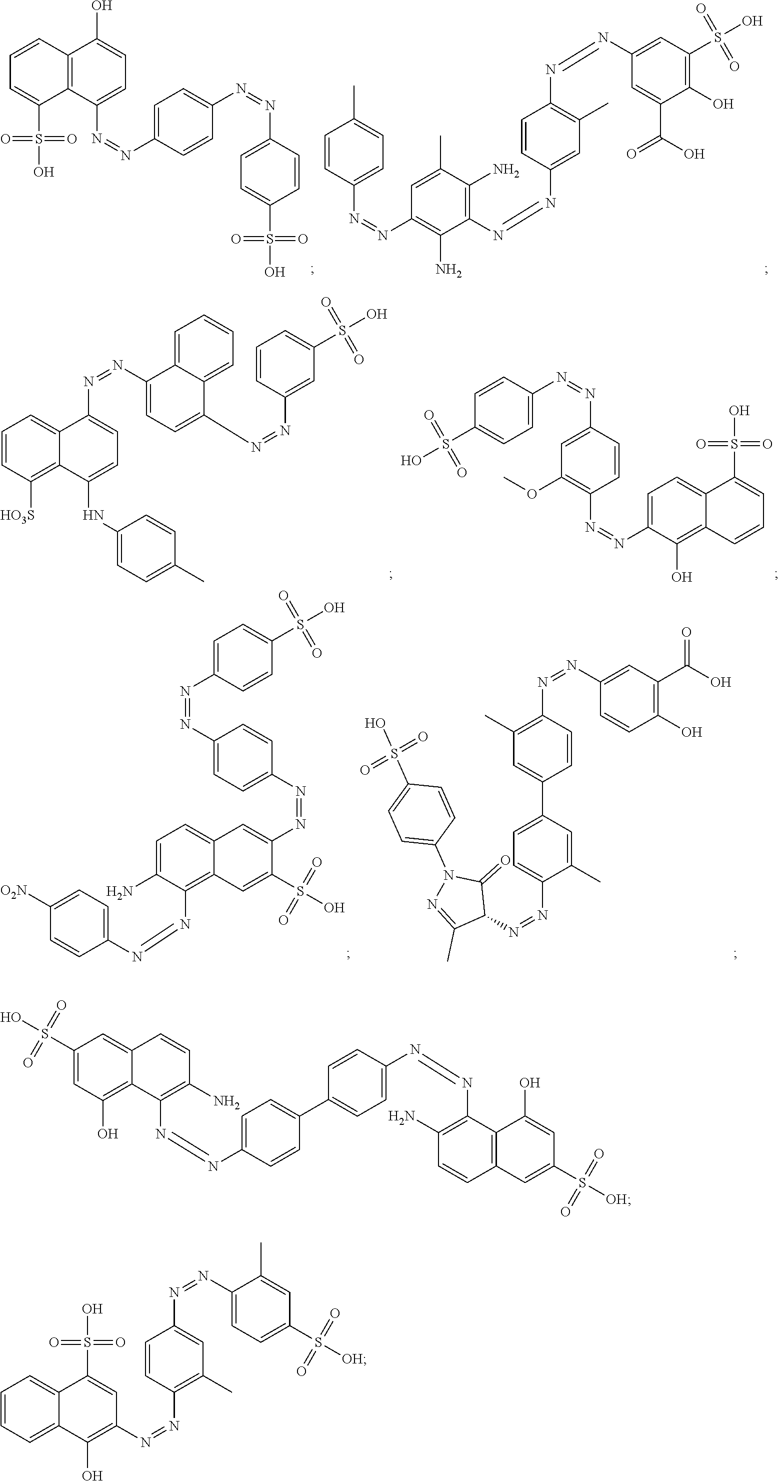

or a pharmaceutically acceptable salt, solvate, hydrate, polymorph, co-crystal, tautomer, stereoisomer, isotopically labeled derivative, or prodrug thereof.

In one aspect, provided herein are compounds of Formula (III).

In some embodiments, of any one of the aspects, each A in compounds of Formula (III) is phenyl, pyridinyl, pyrimidinyl, furanyl, quinolinyl, quinolonyl, naphthyl, anthracenyl or chromenyl, each of which can be optionally substituted. For example, each A can be phenyl, which can be optionally substituted. In some embodiments, each is independently phenyl, sulfonylphenyl, or 2-sulfonylphenyl, each of which can be optionally further substituted.

In some embodiments, of any one of the aspects, R⁶ in compounds of Formula (II) is phenyl, pyridinyl, pyrimidinyl, furanyl, quinolinyl, quinolonyl, naphthyl, anthracenyl or chromenyl, each of which can be optionally substituted. For example, R⁶ is phenyl, naphthyl, quinolinyl, quinolonyl or chromenyl, each of which can be optionally substituted. In some embodiments, R⁶ is phenyl or naphthyl, each of which can be optionally substituted. For example, R⁶ is aminonaphthyl, 1-aminonaphthyl, 4-aminonaphthyl, 1-hydroxynaphthyl, 4-hydroxynaphthyl, phenyl, 2-hydroxy-1-carboxyphenyl, 3-hydroxy-4-carboxyphenyl, 4-Sulfonylphenyl, 4-sulfonyl-2-methylphenyl, 2-methylphenyl, 3-carboxy, 5-sulfonyl-4-hydroxyphenyl, phenylazophenyl, (4-methylphenyl)azophenyl, 3-(4-methylphenyl)azophenayl, (4-methylphenyl)azo-2-amino-3,6-dimethylphenyl, 5-(4-methylphenyl)azo-2-amino-3,6-dimethylphenyl, phenylazo-2-methylphenyl, (3-carboxy, 5-sulfonyl-4-hydroxyphenyl)azophenyl, nitrophenyl, 4-nitophenyl, (3-carboxy, 5-sulfonyl-4-hydroxyphenyl)azo-methylphenyl, 4-(3-carboxy, 5-sulfonyl-4-hydroxyphenyl)azo-methylphenyl, (3-carboxy, 5-sulfonyl-4-hydroxyphenyl)azo-3-methylphenyl, 4-(3-carboxy, 5-sulfonyl-4-hydroxyphenyl)azo-3-methylphenyl, phenylazonaphthyl, nitrophenylazonaphthyl, (4-nitrophenyl)azonaphthyl, 5-(nitrophenyl)azo-3-sulfonyl-1-hydroxy-6-aminonaphthyl, 5-(4-nitrophenyl)azo-3-sulfonyl-1-hydroxy-6-aminonaphthyl, hydroxysulfonylnaphthyl, aminosulfonyl, aminohydroxysulfonylnaphthyl, 1-sulfonyl-4-hydroxynaphthyl, 3-sulfonyl-1-hydroxynaphthyl, 1-hydroxysulfonylnaphthyl, 1-hydroxy-3-sulfonylnaphthyl, 1-hydroxy-5-sulfonylnaphthyl, 1-hydroxy-3-sulfonyl-7-aminonaphthyl, 1-aminosulfonylnaphthyl, 1-amino-3-sulfonylnaphthyl, 1-amino-7-sulfonylnaphthyl, 4-amino-6-sulfonylnaphthyl, (methylphenyl)aminosulfonylnaphthyl, 4-(methylphenyl)aminosulfonylnaphthyl, (methylphenyl)amino-5-sulfonylnaphthyl, 4-(methylphenyl)amino-5-sulfonylnaphthyl, 1-(methylphenyl)amino-8-sulfonylnaphthyl, (4-methylphenyl)aminosulfonylnaphthyl, 4-(4-methylphenyl)aminosulfonylnaphthyl, (4-methylphenyl)amino-5-sulfonylnaphthyl, 4-(4-methylphenyl)amino-5-sulfonylnaphthyl, or 1-(4-methylphenyl)amino-8-sulfonylnaphthyl, each of which can be optionally further substituted. Preferably, R⁶ is aminonaphthyl, 1-aminonaphthyl, 4-aminonaphthyl, 1-hydroxynaphthyl, 4-hydroxynaphthyl, phenyl, 2-hydroxy-1-carboxyphenyl, or 3-hydroxy-4-carboxyphenyl.

In some embodiments, of any one of the aspects, R⁷ in compounds of Formula (III) is phenyl, pyridinyl, pyrimidinyl, furanyl, quinolinyl, quinolonyl, naphthyl, anthracenyl or chromenyl, each of which can be optionally substituted. For example, R⁷ is phenyl, naphthyl, quinolinyl, quinolonyl or chromenyl, each of which can be optionally substituted. In some embodiments, R⁷ is phenyl or naphthyl, each of which can be optionally substituted. For example, R⁷ is aminonaphthyl, 1-aminonaphthyl, 4-aminonaphthyl, 1-hydroxynaphthyl, 4-hydroxynaphthyl, phenyl, 2-hydroxy-1-carboxyphenyl, 3-hydroxy-4-carboxyphenyl, 4-Sulfonylphenyl, 4-sulfonyl-2-methylphenyl, 2-methylphenyl, 3-carboxy, 5-sulfonyl-4-hydroxyphenyl, phenylazophenyl, (4-methylphenyl)azophenyl, 3-(4-methylphenyl)azophenayl, (4-methylphenyl)azo-2-amino-3,6-dimethylphenyl, 5-(4-methylphenyl)azo-2-amino-3,6-dimethylphenyl, phenylazo-2-methylphenyl, (3-carboxy, 5-sulfonyl-4-hydroxyphenyl)azophenyl, nitrophenyl, 4-nitophenyl, (3-carboxy, 5-sulfonyl-4-hydroxyphenyl)azo-methylphenyl, 4-(3-carboxy, 5-sulfonyl-4-hydroxyphenyl)azo-methylphenyl, (3-carboxy, 5-sulfonyl-4-hydroxyphenyl)azo-3-methylphenyl, 4-(3-carboxy, 5-sulfonyl-4-hydroxyphenyl)azo-3-methylphenyl, phenylazonaphthyl, nitrophenylazonaphthyl, (4-nitrophenyl)azonaphthyl, 5-(nitrophenyl)azo-3-sulfonyl-1-hydroxy-6-aminonaphthyl, 5-(4-nitrophenyl)azo-3-sulfonyl-1-hydroxy-6-aminonaphthyl, hydroxysulfonylnaphthyl, aminosulfonyl, aminohydroxysulfonylnaphthyl, 1-sulfonyl-4-hydroxynaphthyl, 3-sulfonyl-1-hydroxynaphthyl, 1-hydroxysulfonylnaphthyl, 1-hydroxy-3-sulfonylnaphthyl, 1-hydroxy-5-sulfonylnaphthyl, 1-hydroxy-3-sulfonyl-7-aminonaphthyl, 1-aminosulfonylnaphthyl, 1-amino-3-sulfonylnaphthyl, 1-amino-7-sulfonylnaphthyl, 4-amino-6-sulfonylnaphthyl, (methylphenyl)aminosulfonylnaphthyl, 4-(methylphenyl)aminosulfonylnaphthyl, (methylphenyl)amino-5-sulfonylnaphthyl, 4-(methylphenyl)amino-5-sulfonylnaphthyl, 1-(methylphenyl)amino-8-sulfonylnaphthyl, (4-methylphenyl)aminosulfonylnaphthyl, 4-(4-methylphenyl)aminosulfonylnaphthyl, (4-methylphenyl)amino-5-sulfonylnaphthyl, 4-(4-methylphenyl)amino-5-sulfonylnaphthyl, or 1-(4-methylphenyl)amino-8-sulfonylnaphthyl, each of which can be optionally further substituted. Preferably, R⁷ is aminonaphthyl, 1-aminonaphthyl, 4-aminonaphthyl, 1-hydroxynaphthyl, 4-hydroxynaphthyl, phenyl, 2-hydroxy-1-carboxyphenyl, or 3-hydroxy-4-carboxyphenyl.

In compounds of Formula (III), R⁶ and R⁶ can be same or different. In some embodiments, R⁶ and R⁷ are same. In some other embodiments, R⁶ and R⁷ are different

Exemplary compounds of Formula (III) include, but are not limited to, the following:

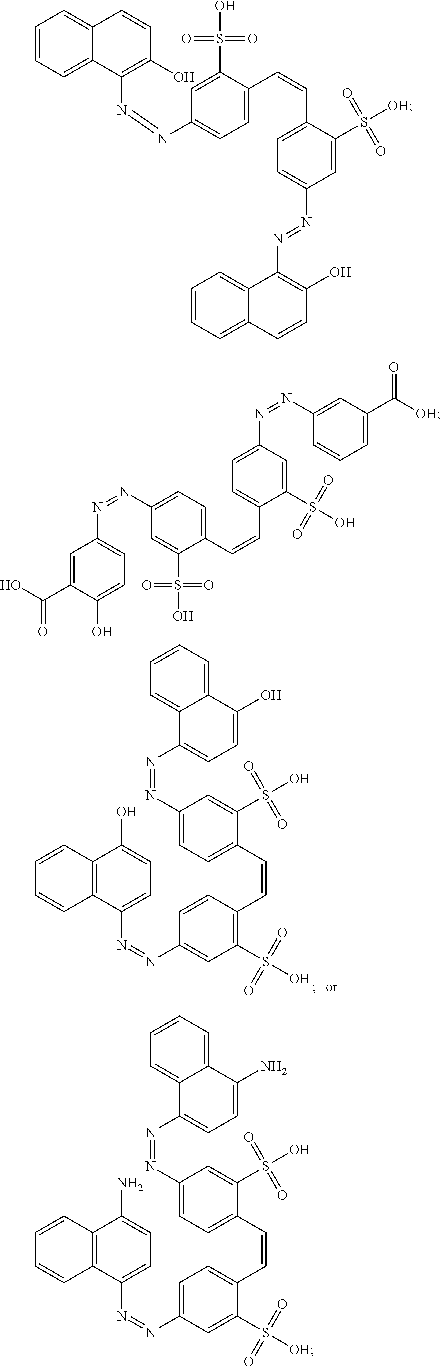

or a pharmaceutically acceptable salt, solvate, hydrate, polymorph, co-crystal, tautomer, stereoisomer, isotopically labeled derivative, or prodrug thereof.

In Table 1, some exemplary compounds of the disclosure are described by their Simplified Molecular Input Line Entry System (SMILES). SMILES allows rigorous structure specification by use a compact use of natural grammar as described in detail by D. Weiniger “SMILES, a Chemical Language and Information System. 1. Introduction to Methodology and Encoding Rules” J. Chem. Inf. Comput. Sci., Vol. 28, No. 1, 1988, pages 31-36; the entirety of which are incorporated herein by reference

TABLE 1
Some exemplary compounds of the disclosure

| Compound | SMILES |
|---|---|
| iKeap28 | CC1═CC═C(C(═O)NC(═CC2═CC═C(C3═CC═CC([N+](═O) |
| | [O−])═C3)O2)C(═O)NCC2CCCO2)C═C1 |
| iKeap2 | O═C(O)c1ccc(NC2═C/C(═N\S(═O)(═O)c3ccc4ccccc4c3)c3ccccc3C2═O)cc1 |
| iKeap24 | O═C(O)C(SC1═NC(═O)C2(NN1)c1ccccc1- |
| | c1ccccc12)SC1═NC(═O)C2(NN1)c1ccccc1-c1ccccc12 |
| iKeap9 | O═S(═O)(N═C1CCCCCN1)C1═CC═C2SC(NS(═O)(═O) |
| | C3═CC═C4C═CC═CC4═C3)═NC2═C1 |
| iKeap20 | Cc1cc2oc(═O)cc(COC(═O)[C@H]3CCCN(C(═O)c4ccc5[nH]ncc5c4)C3) |
| | c2cc1C(C)C |
| iKeap4 | O═C1OC(c2ccc([N+](═O)[O−])cc2)═N/C1═C\c1cccc2ccccc12 |
| iKeap29 | COc1ccc(NS(═O)(═O)c2ccc(/N═C\c3c4ccccc4nc4ccccc43)cc2)nn1 |
| iKeap27 | Cc1ccc(C)c(N2C(═O)c3ccc(-c4nc(-c5ccc6[nH]nnc6c5)no4)cc3C2═O)c1 |
| iKeap75 | Cc1cccn2c(═O)c3c(nc12)N1CCCCC[C@H]1[C@]1(C3)C(═O)N(Cc2ccccc2Cl) |
| | C(═O)N═C1O |
| iKeap51 | O═C(Cn1nc(-c2ccccc2)ccc1═O)Nc1cccc(Oc2ccc([N+](═O)[O−])cc2)c1 |
| iKeap12 | O═C(c1ccccc1)C1═C[C@@H]2[C@@H]3C(═O)N(c4cccc5ccccc54)C(═O) |
| | [C@@H]3[C@@H](C(═O)3ccc(Cl)cc3)N2C═C1 |
| iKeap18 | O═S(═O)(O)c1ccc(/N═N\c2ccc(/N═N\c3ccc(O)c4cccc(S(═O)(═O)O)c34) |
| | cc2)cc1 |
| iKeap26 | O═C1C[C@H](C(═O)N2CCC[C@H](NC(═O)c3cc4ccccc4o3)C2)c2ccc(F) |
| | cc2N1 |
| iKeap36 | O═C(NN═CC1═C(O)C([N+](═O) |
| | [O−])═CC(Cl)═C1)C1═CC(C2═CC═CC═N2)═NC2═CC═CC═C12 |
| iKeap52 | Cc1ccc2cc([C@H]3CC(═O)Oc4cc(O)c5c(═O)c(O)c(-c6ccc(O)c(O)c6) |
| | oc5c43)c(═O)[nH]c2c1 |
| iKeap7 | CC1═NN(c2ccccc2)C(═O)/C1═C\c1ccc(-c2cc(C(═O)O)ccc2Cl)o1 |
| iKeap31 | O═C1C[C@H](c2ccc(OCCc3ccc4c(c3)CCO4)cc2)c2c(cc(O)c3c(═O)c(O) |
| | c(-c4ccc(O)c(O)c4)oc32)O1 |
| iKeap16 | O═C(c1ccccc1)c1cc(-c2cc(═O)cc(-c3cc(C(═O)c4ccccc4)c(O)cc3O) |
| | o2)c(O)cc1O |
| iKeap34 | CC(C)c1ccc(COc2cccc([C@H]3CC(═O)Oc4cc(O)c5c(═O)c(O) |
| | c(-c6ccc(O)c(O)c6)oc5c43)c2)cc1 |
| iKeap13 | O═C1OC(c2cccc3ccccc32)═N/C1═C\c1ccc(-c2cccc(C(F)(F)F)c2)o1 |
| iKeap62 | COc1ccc2occ([C@@H]3CC(═O)Oc4cc(O)c5c(═O)cc(-c6ccc(O)c(O)c6) |
| | oc5c43)c(═O)c2c1 |
| iKeap69 | CC1═CC═C(S(═O)(═O)NC2═CC(═NS(═O)(═O)C3═C(C)C═C(C) |
| | C═C3C)C3═CC═CC═C3C2═O)C═C1 |
| iKeap39 | Cc1cccn2c(═O)c3c(nc12)N1CCCCC[C@H]1[C@]1(C3)C(═O)N |
| | (Cc2ccccc2)C(═O)N═C1O |
| iKeap68 | CC1═NOC(NS(═O)(═O)C2═CC═C(NC(═O)CC3═COC4═CC═C(C(C)C) |
| | C═C34)C═C2)═C1C |
| iKeap40 | Cc1ccc(N2C(═O)[C@@H]3N═NN(CC(═O)N4N═C5/C(═C/c6ccccc6) |
| | CCC[C@@H]5[C@H]4c4ccccc4)[C@@H]3C2═O)cc1 |
| iKeap5 | Nc1ccc2cc(S(═O)(═O)O)c(/N═N/c3ccc(/N═N\c4ccc(N)c5cc(S(═O)(═O)O) |
| | ccc45)c4ccccc34)c(O)c2c1 |
| iKeap30 | Cc1ccc(/N═N\c2cc(C)c(N)c(/N═N\c3ccc(/N═N\c4cc(C(═O)O)c(O)c(S(═O) |
| | (═O)O)c4)c(C)c3)c2N)cc1 |
| iKeap56 | COc1ccc(C(═O)C2═C(O)C(═O)N(c3nnc(SCc4cccc5ccccc54)s3) |
| | [C@H]2c2cccc(Oc3ccccc3)c2)cc1OC |
| iKeap35 | CC1═NN(C2═CC═CC═C2)C(NC2═CC═C3C(═N2)OC2═NC |
| | (NC4═CC(C)═NN4C4═CC═CC═C4)═CC═C2C3C2═CC═C(Cl) |
| | C([N+](═O)[O−])═C2)═C1 |
| iKeap57 | Cc1ccc(/C═C2\CCC[C@@H]3C2═NN(C(═O)CN2N═N[C@@H]4C(═O) |
| | N(c5cccc(F)c5)C(═O)[C@H]42)C@@H]3c2ccc(C)cc2)cc1 |
| iKeap6 | Cc1ccc(Nc2ccc(/N═N\c3ccc(/N═N\c4cccc(S(═O)(═O)O)c4)c4ccccc34) |
| | c3cccc(S(═O)(═O)O)c23)cc1 |
| iKeap11 | COc1cc(/N═N\c2ccc(S(═O)(═O)O)cc2)ccc1/N═N\c1ccc2c(cccc2S(═O)(═O) |
| | O)c1O |
| iKeap77 | O═C1C[C@@H](c2ccccc2)CC2═C1[C@@H](c1cccc(Oc3ccccc3)c1) |
| | Nc1ccccc1N2 |
| iKeap38 | O═S(═O)(O)c1cc(/N═N/c2c(O)ccc3ccccc32)ccc1/C═C\c1ccc(/N═N\c2c(O) |
| | ccc3ccccc32)cc1S(═O)(═O)O |
| iKeap23 | O═C(O)c1cc(/N═N\c2ccc(/C═C\c3ccc(/N═N\c4ccc(O)c(C(═O)O)c4) |
| | cc3S(═O)(═O)O)c(S(═O)(═O)O)c2)ccc1O |
| iKeap54 | O═C(N/N═C/c1cc([N+](═O)[O−])cc([N+](═O)[O−])c1O)c1cc(-c2ccccc2) |
| | nc2ccccc21 |
| iKeap14 | O═[N+]([O−])c1ccc(Nc2ccc(Oc3ccc(Nc4ccc([N+](═O) |
| | [O−])c5nonc54)cc3)cc2)c2nonc21 |
| iKeap1 | Cc1ccc(S(═O)(═O)Oc2nc3nc4ccccc4nc3nc2OS(═O)(═O)c2ccc(C)cc2)cc1 |
| iKeap46 | Cc1nnnn1-c1cccc(NC(═O)c2c3c(nc4ccccc24)/C(═C\c2ccco2)CC3)c1 |
| iKeap60 | O═C1N[C@@H](Cc2nc(-c3cccc(Cn4cnc5ccccc54)c3)no2)C(═O)Nc2ccccc21 |
| iKeap50 | Cc1ccnc(NS(═O)(═O)c2ccc(/N═C\c3c4ccccc4nc4cc(Cl)ccc34)cc2)n1 |
| iKeap32 | O═C(Cc1ccc(Cl)cc1)Nc1cccc(-c2ccc3nnc(-c4cccnc4)n3n2)c1 |
| iKeap67 | O═C([C@H]1C[C@H]2CCCC[C@@H]2N1c1ncccn1)N1CC═C |
| | (c2c[nH]c3cc(F)ccc23)CC1 |
| iKeap55 | Nc1ccc2c(O)c(/N═N\c3ccc(/N═N\c4ccc(S(═O)(═O)O)cc4)cc3)c(S(═O) |
| | (═O)O)cc2c1/N═N\c1ccc([N+](═O)[O−])cc1 |
| iKeap3 | Cc1cc(/N═N\c2ccc(S(═O)(═O)O)cc2C)ccc1/N═N\c1cc(S(═O)(═O)O) |
| | c2ccccc2c1O |
| iKeap25 | O═S(═O)(O)c1cc(O)c2c(c1)cc(S(═O)(═O)O)cc2/N═N\c1ccc(Nc2ccccc2) |
| | c2c1cccc2S(═O)(═O)O |
| iKeap59 | O═S(═O)(O)c1cc(/N═N\c2ccc(O)c3ccccc23)ccc1/C═C\c1ccc(/N═N\ |
| | c2ccc(O)c3ccccc23)cc1S(═O)(═O)O |
| iKeap58 | CC1═NN(c2ccc(S(═O)(═O)O)cc2)C(═O)[C@@H]1/N═N\c1ccc |
| | (-c2ccc(/N═N\c3ccc(O)c(C(═O)O)c3)c(C)c2)cc1C |
| iKeap10 | Nc1ccc2cc(S(═O)(═O)O)cc(O)c2c1/N═N\c1ccc |
| | (-c2ccc(/N═N\c3c(N)ccc4cc(S(═O)(═O)O)cc(O)c43)cc2)cc1 |
| iKeap71 | O═C(O)c1ccc(-c2ccc([C@H]3CC(═O)Oc4ccc5c(c43)O/C |
| | (═C\c3cc(F)c(F)c(F)c3)C5═O)o2)cc1 |
| iKeap45 | Nc1ccc(/N═N\c2ccc(/C═C\c3ccc(/N═N\c4ccc(N)c5ccccc45)cc3S(═O)(═O)O) |
| | c(S(═O)(═O)O)c2)c2ccccc12 |
| iKeap70 | COc1ccc(C(═O)N2CCC[C@H](C(═O)NNC(═O)c3ccc4ccccc4c3)C2)c2ccccc12 |
| iKeap64 | O═C(Nc1ccc2[nH]c(-c3cccc(F)c3)nc2c1)[C@@H]1CCCN(C(═O) |
| | c2ccc3[nH]ncc3c2)C1 |
| iKeap43 | O═C1c2ccccc2/C(═C\NNc2ccc(C(F)(F)F)cc2[N+](═O) |
| | [O−])C(═O)N1Cc1ccc2c(c1)OCO2 |
| iKeap65 | CC1(C)Cc2oc3c(cc(NS(═O)(═O)c4ccc5c6c(cccc64)C(═O)N5)c4ccccc34) |
| | c2C(═O)C1 |
| iKeap44 | O═C1c2ccccc2C(═O)N1Cc1cccc(C(═O)N2CCCC[C@H]2c2nc |
| | (-c3ccccc3)no2)c1 |
| iKeap66 | O═S(═O)(Nc1cccc(-c2ccc3nnc(-c4cccnc4)n3n2)c1)c1ccc2ccccc2c1 |
| iKeap48 | CC(═O)N/C(═C\c1ccc(Cc2nc3c([nH]2)C(═O)c2ccccc2C3═O)cc1) |
| | c1nc2c([nH]1)C(═O)c1ccccc1C2═O |
| iKeap21 | O═C(NNc1nc2ccccn2n1)c1cc(-c2cccc3ccccc23)nc2ccccc12 |
| iKeap15 | O═C(c1ccccc1)C1═C[C@@H]2[C@@H]3C(═O)N(c4cccc5ccccc54)C(═O) |
| | [C@@H]3[C@@H](C(═O)c3ccc(Br)cc3)N2C═C1 |
| iKeap41 | O═C(c1cc(-c2ccc3c(c2)OCCO3)nc2ccccc21)N1CCC(Cc2ccccc2)CC1 |
| iKeap76 | O═C(C1CCN(c2ccc3nnnn3n2)CC1)N1Cc2ccccc2-c2ccccc2C1 |
| iKeap22 | O═c1cc(-c2ccc(O)cc2)oc2c1c(O)cc1c2[C@H](c2ccc3ncccc3c2)CC(═O)O1 |
| iKeap73 | C[C@H]1CCc2c(sc3ncnc(N4CCO[C@@H](CN5C(═O)c6ccccc6C5═O)C4) |
| | c23)C1 |
| iKeap74 | C[C@@H]1Oc2ccc(C(═O)C3CCN(C(═O)[C@H]4C[C@H]4c4cccc5ccccc45) |
| | CC3)cc2NC1═O |
| iKeap8 | O═C(Nc1cccc(-c2nnn/nH]2)c1)[C@@H]1C[C@H]2CCCC[C@H]2N1C |
| | (═O)c1ccc2ccccc2c1 |
| iKeap33 | O═S(═O)(Nc1nc2ccccc2nc1N1CCC[C@@H](c2nc3ccccc3[nH]2)C1)c1ccccc1 |
| iKeap61 | O═C(C1Cc2ccccc2C1)N1CCN(C(═O)C2Cc3ccccc3C2)c2ccccc21 |
| iKeap49 | O═C1c2ccccc2C(═O)C(N/N═c2/[nH][nH]/c(═N\NC3═C(Cl)C(═O) |
| | c4ccccc4C3═O)c3ccccc32)═C1C1 |
| iKeap19 | CC(═O)N5CCCC4═CC(NC(═O)c3cccc(NC(═O)C2Cc1ccccc1O2)c3)═CCC45 |
| iKeap47 | O═C(OCc2cc([N+](═O)O)cc1COCOc12)c6c5CCC/C(═C/c4ccc3OCOc3c4) |
| | c5nc7ccccc67 |
| iKeap72 | O═c3[nH]c2ccc(c1ccccc1)cc2c3═NNc6nc(c4ccccn4)nc5CCCc56 |

The compounds of the disclosure can inhibit KEAP1. Accordingly, another aspect of the disclosure provides a method of inhibiting KEAP1. Accordingly, in one aspect, provided herein is method for inhibiting KEAP1. Generally, the method comprises contacting KEAP1 with a compound described herein. By inhibiting KEAP1 is meant inhibiting a biological activity of KEAP1. For example, inhibiting its interaction with Nrf2.

The KEAP1 can be inside a cell whom contacted with a compound of the disclosure. For example, a compound described herein can be administered to a cell expressing KEAP1. It is noted that administering the compound to the cell can be in vitro or in-vivo. Methods for administering a compound to a cell are well known and available to one of skill in the art. As used herein, administering the compound to the cell means contacting the cell with the compound so that the compound is taken up by the cell. Generally, the cell can be contacted with the compound in a cell culture e.g., in vitro or ex vivo, or the compound can be administrated to a subject, e.g., in vivo. The term “contacting” or “contact” as used herein in connection with contacting a cell includes subjecting the cells to an appropriate culture media, which comprises a compound described herein. Where the cell is in vivo, “contacting” or “contact” includes administering the compound, e.g., in a pharmaceutical composition to a subject via an appropriate administration route such that the compound contacts the cell in vivo.

For example, when the cell is in vitro, said administering to the cell can include subjecting the cell to an appropriate culture media which comprises the indicated compound. Where the cell is in vivo, said administering to the cell includes administering the compound to a subject via an appropriate administration route such that the compound is administered to the cell in vivo.

The compounds of the disclosure can disrupt or inhibit binding of Nrf2 to KEAP1, i.e., inhibit the KEAP1-NRF2 interaction. Accordingly, in another aspect provided herein is a method for disrupting, preventing or inhibiting an interaction between KEAP1 and Nrf2. The method comprises contacting KEAP1 with a compound described herein.

The compounds of the disclosure can inhibit the KEAP1-NRF2 interaction by more than about 50%, 55%, 60%, 65%, 70%, 75%, 80%, 85% 90%, 95%, 97%, or 99% of the positive control. In some embodiments, the compounds of the disclosure can inhibit the KEAP1-NRF2 interaction by more than about 75%, 80%, 85%, 90%, 95%, 97%, or 99% of the positive control. For example, the compounds disclosed herein can inhibit the KEAP1-NRF2 interaction by more than about 85%, 86%, 87%, 88%, 89%, 90%, 91%, 92%, 93%, 94%, 95%, 96%, 97%, 98%, or 99% of the positive control. Furthermore, the compounds disclosed herein can inhibit the KEAP1-NRF2 interaction with an IC₅₀ of less than about 3 mM, or less than about 2 mM, or less than about 1 mM, or less than about 0.5 mM, or less than about 04 mM, or less than about 0.3 mM, or less than about 0.2 mM, or less than about 0.1 mM, or less than about 90 nM, or less than about 80 nM, or less than about 70 nM, or less than about 60 nM, or less than about 50 nM, or less than about 40 nM, or less than about 30 nM, or less than about 20 nM, or less than about 10 nM, or less than about 5 nM.

Disrupting the interactions between KEAP1 and Nrf2 can activate the Nrf2. Accordingly, in yet another aspect, provided herein is a method for activating Nrf2. The method comprises contacting KEAP1 with a compound described herein.

Certain embodiments provided herein relate to methods of inhibiting KEAP1, which can be useful for treating, preventing, or ameliorating a disease, disorder or condition associated with KEAP1 in a subject. For example, a disease, disorder or condition associated with dysfunction of the Nuclear factor erythroid 2-related factor 2 (Nrf2)/Kelch-like ECH-associated protein 1 (KEAP1) axis such as a disease, disorder or condition associated with Nrf2-KEAP1 interaction. Accordingly, in some embodiments of any one of the aspects, the subject has a disease, disorder or condition associated with dysfunction of the KEAP1-Nrf2 axis. For example, a subject diagnosed with a disease, disorder or condition associated with dysfunction of the KEAP1-Nrf2 axis.

Accordingly, in another aspect, provided herein is a method for treating a disease, disorder or condition associated with KEAP1. Generally, the method comprises administering a compound described herein to a subject in need thereof.

In some embodiments of any one of the aspects described herein, the disease, disorder or condition associated with KEAP1 is a disease, disorder or condition associated with dysfunction of the Nrf2-KEAP1 axis.

in some embodiments of any one of the aspects described herein, the disease, disorder or condition associated with KEAP1 is a disease, disorder or condition associated with Nrf2-KEAP1 interaction.

Exemplary Diseases Associated with Dysfunction of KEAP1-Nrf2 Axis

An example of a disease, disorder or condition associated with dysfunction of KEAP1-Nrf2 axis is oxidative stress or a disease, disorder or condition associated with oxidative stress. Some exemplary diseases, disorders or conditions associated with oxidative stress include but are not limited to, metabolic diseases, inflammatory diseases, autoimmune diseases, lung diseases, cardiovascular diseases, liver diseases, kidney diseases, ophthalmological diseases, gastrointestinal tract diseases, neurological diseases, neurodegenerative diseases, and cancers.

In some embodiments of any one of the aspects described herein, the disease, disorder or condition associated with KEAP1 is a metabolic disease. Exemplary metabolic diseases associated with KEAP1 include, but are not limited to, metabolic syndrome, type 2 diabetes, diabetic nephropathy, diabetic cardiomyopathy and insulin resistance.

In some embodiments of any one of the aspects described herein, the disease, disorder or condition associated with KEAP1 is a liver disease. Exemplary diseases, disorder or conditions of the liver associated with KEAP1 include, but are not limited to, hepatic fibrosis, autosomal dominant polycystic liver disease, hepatic steatosis, non-alcoholic steatohepatitis (NASH), and non-alcoholic fatty liver disease (NAFLD).

In some embodiments of any one of the aspects described herein, the disease, disorder or condition associated with KEAP1 is an inflammatory disease. Exemplary inflammatory diseases, disorder or conditions associated with KEAP1 include, but are not limited to, asthma, chronic obstructive pulmonary disease (COPD), cystic fibrosis, and airway hyperresponsiveness.

In some embodiments of any one of the aspects described herein, the disease, disorder or condition associated with KEAP1 is an autoimmune disease. Exemplary autoimmune diseases, disorder or conditions associated with KEAP1 include, but are not limited to, multiple sclerosis, psoriasis, connective tissue disease, and pulmonary arterial hypertension associated with connective tissue disease.

In some embodiments of any one of the aspects described herein, the disease, disorder or condition associated with KEAP1 is a kidney disease. Exemplary kidney diseases, disorder or conditions associated with KEAP1 include, but are not limited to, renal fibrosis, Alport syndrome, autosomal dominant polycystic kidney disease, chronic kidney disease, IgA nephropathy, type 1 diabetes, type 2 diabetes mellitus, and focal segmental glomerulosclerosis, and nephropathy.

In some embodiments of any one of the aspects described herein, the disease, disorder or condition associated with KEAP1 is a lung disease. Exemplary lung diseases, disorder or conditions associated with KEAP1 include, but are not limited to, pulmonary arterial hypertension, pulmonary hypertension-interstitial lung disease, pulmonary fibrosis, cystic fibrosis, emphysema, chronic obstructive pulmonary disease (COPD), and chronic bronchitis.

In some embodiments of any one of the aspects described herein, the disease, disorder or condition associated with KEAP1 is a cardiovascular disease. Exemplary cardiovascular diseases, disorder or conditions associated with KEAP1 include, but are not limited to, atherosclerosis, heart failure, myocardial infarction, reperfusion injury, and stroke.

In some embodiments of any one of the aspects described herein, the disease, disorder or condition associated with KEAP1 is a neurological or neurodegenerative disease. Exemplary neurological or neurodegenerative diseases, disorder or conditions associated with KEAP1 include, but are not limited to, Friedreich's ataxia, subarachnoid hemorrhage, amyotrophic lateral sclerosis, Parkinson's disease, Parkinson's disease with dementia with Lewy body, Huntington's Disease, Batten Disease, multiple system atrophy (MSA), progressive supranuclear palsy (PSA), corticobasal degeneration (CBD), frontotemporal lobe degeneration, Alzheimer's disease, Fragile X syndrome, chronic fatigue syndrome, cerebral ischemia, neuronal cell death, Creutzfeldt-Jakob disease, Lewy body disease, Pick's disease, or neurofibromatosis.

In some embodiments of any one of the aspects described herein, the disease, disorder or condition associated with KEAP1 is an ophthalmological disease. Exemplary ophthalmological diseases, disorder or conditions associated with KEAP1 include, but are not limited to, is dry eye macular degeneration, retinovascular disease, or retinopathy.

In some embodiments of any one of the aspects described herein, the disease, disorder or condition associated with KEAP1 is cancer. It is noted that a cancer associated with KEAP1 can breast cancer, liver cancer, lung cancer, breast cancer, prostate cancer, colon cancer, neuroblastoma, or leukemia.

The disease, disorder or condition associated with dysfunction of the KEAP1-Nrf2 axis can be selected from the group consisting of Alport syndrome, amyotrophic lateral sclerosis, autosomal dominant polycystic kidney disease, bone disease, blood disease, chronic kidney disease, chronic obstructive pulmonary disease, connective tissue disease, dry eye macular degeneration, estrogen receptor-positive breast cancer, eye disease, focal segmental glomerulosclerosis, Friedreich ataxia, immunoglobulin A nephropathy, interstitial lung disease, lung diseases, multiple sclerosis, kidney disease, neurodegenerative disease, primary focal segmental glomerulosclerosis, psoriasis, pulmonary arterial hypertension, retinovascular disease, subarachnoid hemorrhage, type 1 diabetes, and type 2 diabetes mellitus. In various embodiments, the preclinical disease or disorder is selected from the group consisting of, autoimmune diseases (e.g., rheumatoid arthritis, Sjogren syndrome, STING-dependent interferonopathies, systemic lupus erythematous, vitiligo); respiratory diseases (e.g., chronic obstructive pulmonary disease, chronic sarcoidosis, emphysema, hypersensitivity pneumonitis, idiopathic pulmonary fibrosis, pulmonary fibrosis); gastrointestinal diseases (e.g., hemochromatosis, hepatic fibrosis, primary biliary cholangitis and cirrhosis); metabolic diseases (e.g., insulin resistance, glomerulonephritis, nonalcoholic steatohepatitis, type 2 diabetes mellitus, vascular dysfunction); cardiovascular diseases (e.g., atherosclerosis, diabetic vascular disease, hypertension, myocardial ischemia-reperfusion injury, heart failure); neurodegenerative diseases (e.g., Alzheimer disease, Fluntington disease, Friedrich ataxia, Parkinson disease); skin diseases (e.g., chronic/diabetic wound healing); and cancer.

### Routes of Administration

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, administration will generally be local rather than systemic.

In some embodiments, a compound of the disclosure is orally administered. Without limitations, oral administration can be in the form of solutions, suspensions, tablets, pills, capsules, sustained-release formulations, oral rinses, powders and the like.

In some embodiments, a compound of the disclosure is compound is administered in a local rather than systemic manner, for example, via topical application of the compound directly on to skin, or intravenously, or subcutaneously, often in a depot preparation or sustained release formulation. In specific embodiments, long acting formulations are administered by implantation (for example subcutaneously or intramuscularly) or by intramuscular injection. In yet other embodiments, the compound as described herein is provided in the form of a rapid release formulation, in the form of an extended release formulation, or in the form of an intermediate release formulation. In yet other embodiments, the compound described herein is administered topically (e.g., as a patch, an ointment, or in combination with a wound dressing, or as a wash or a spray). In alternative embodiments, a formulation is administered systemically (e.g., by injection, or as a pill).

The phrase “therapeutically-effective amount” as used herein means that amount of a compound, material, or composition comprising a compound described herein which is effective for producing some desired therapeutic effect in at least a sub-population of cells, e.g., inhibit KEAP1 in a subject at a reasonable benefit/risk ratio applicable to any medical treatment. Thus, “therapeutically effective amount” means that amount which, when administered to a subject for treating a disease, is sufficient to affect such treatment for the disease.

Depending on the route of administration, effective doses can be calculated according to the body weight, body surface area, or organ size of the subject to be treated. Optimization of the appropriate dosages can readily be made by one skilled in the art in light of pharmacokinetic data observed in human clinical trials. Alternatively, or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay. The effective plasma concentration for a compound as disclosed herein can be about 0.01 μM to about 10 μM, about 0.2 μM to about 5 μM, or about 0.8 to about 3 μM in a subject, such as a rat, dog, or human.

Generally, the compositions are administered so that a compound of the disclosure herein is used or given at a dose from 1 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

The compounds described herein can be administered at once, or can be divided into a number of smaller doses to be administered at intervals of time. It is understood that the precise dosage and duration of treatment will be a function of the location of where the composition is parenterally administered, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The compound can be administered as a single bolus or multiple boluses, as a continuous infusion, or a combination thereof. For example, the compound can be administered as a single bolus initially, and then administered as a continuous infusion following the bolus. The rate of the infusion can be any rate sufficient to maintain effective concentration, for example, to maintain effective plasma concentration. Some contemplated infusion rates include from 1 μg/kg/min to 100 mg/kg/min, or from 1 μg/kg/hr to 1000 mg/kg/hr. Rates of infusion can include 0.2 to 1.5 mg/kg/min, or more specifically 0.25 to 1 mg/kg/min, or even more specifically 0.25 to 0.5 mg/kg/min. It will be appreciated that the rate of infusion can be determined based upon the dose necessary to maintain effective plasma concentration and the rate of elimination of the compound, such that the compound is administered via infusion at a rate sufficient to safely maintain a sufficient effective plasma concentration of compound in the bloodstream.

### Pharmaceutical Compositions/Formulations

For administration to a subject, the compounds describe herein can be provided in a pharmaceutically acceptable compositions. These pharmaceutically acceptable compositions comprise a compound described herein, formulated together with one or more pharmaceutically acceptable carriers (additives) and/or diluents. As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-Cu alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Examples of solid carriers include starch, sugar, bentonite, silica, and other commonly used carriers. Further non-limiting examples of carriers and diluents which can be used in the formulations comprising a compound described herein as disclosed herein of the present invention include saline, syrup, dextrose, and water.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

The phrase “therapeutically-effective amount” as used herein means that amount of a compound, material, or composition which is effective for producing some desired therapeutic effect in at least a sub-population of cells in an animal at a reasonable benefit/risk ratio applicable to any medical treatment. According, a “therapeutically effective amount” refers to an amount effective, at dosage and periods of time necessary, to achieve a desired therapeutic result. A therapeutic result can be, e.g., lessening of symptoms, prolonged survival, improved mobility, and the like. A therapeutic result need not be a “cure.”

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The compounds can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J Pharm. Sci. Tech. 1997 51:166-171. Methods to make invention formulations include the step of bringing into association or contacting an ActRIIB compound with one or more excipients or carriers. In general, the formulations are prepared by uniformly and intimately bringing into association one or more compounds with liquid excipients or finely divided solid excipients or both, and then, if appropriate, shaping the product.

The preparative procedure may include the sterilization of the pharmaceutical preparations. The compounds may be mixed with auxiliary agents such as lubricants, preservatives, stabilizers, salts for influencing osmotic pressure, etc., which do not react deleteriously with the compounds.

Examples of injectable form include solutions, suspensions and emulsions. Injectable forms also include sterile powders for extemporaneous preparation of injectable solutions, suspensions or emulsions. The compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. A suitable carrier is 5% dextrose in saline. Frequently, it is desirable to include additives in the carrier such as buffers and preservatives or other substances to enhance isotonicity and chemical stability.

In some embodiments, compounds can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185; content of each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like. The compounds can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds may be incorporated with excipients and used in the form of tablets, capsules, elixirs, suspensions, syrups, and the like. Such compositions and preparations should contain at least 0.1% of compound. The percentage of the agent in these compositions may, of course, be varied and may conveniently be between about 2% to about 60% of the weight of the unit. The amount of compound in such therapeutically useful compositions is such that a suitable dosage will be obtained. Preferred compositions according to the present invention are prepared so that an oral dosage unit contains between about 100 and 2000 mg of compound. Examples of bases useful for formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives. The compositions can be given as a bolus dose, to maximize the circulating levels for the greatest length of time after the dose. Continuous infusion may also be used after the bolus dose.

The compounds can also be administrated directly to the airways in the form of an aerosol. For administration by inhalation, the compounds in solution or suspension can be delivered in the form of an aerosol spray from pressured container or dispenser which contains a suitable propellant, e.g., a gas such as carbon dioxide, or hydrocarbon propellant like propane, butane or isobutene. The compounds can also be administrated in a no-pressurized form such as in an atomizer or nebulizer.

In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Representative intranasal formulations are described in, for example, U.S. Pat. Nos. 4,476,116, 5,116,817 and 6,391,452. Formulations that include a compound described herein are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions

The compounds can also be administered parenterally. Solutions or suspensions of these compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of compound calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

For oral or enteral formulations as disclosed herein for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982.

Also provided herein is a tablet formulation comprising a compound described herein with an enteric polymer casing. An example of such a preparation can be found in WO2005/021002. The active material in the core can be present in a micronised or solubilised form. In addition to active materials the core can contain additives conventional to the art of compressed tablets. Appropriate additives in such a tablet can comprise diluents such as anhydrous lactose, lactose monohydrate, calcium carbonate, magnesium carbonate, dicalcium phosphate or mixtures thereof; binders such as microcrystalline cellulose, hydroxypropylmethylcellulose, hydroxypropyl-cellulose, polyvinylpyrrolidone, pre-gelatinised starch or gum acacia or mixtures thereof, disintegrants such as microcrystalline cellulose (fulfilling both binder and disintegrant functions) cross-linked polyvinylpyrrolidone, sodium starch glycollate, croscarmellose sodium or mixtures thereof, lubricants, such as magnesium stearate or stearic acid, glidants or flow aids, such as colloidal silica, talc or starch, and stabilisers such as desiccating amorphous silica, colouring agents, flavours etc. Preferably the tablet comprises lactose as diluent. When a binder is present, it is preferably hydroxypropylmethyl cellulose. Preferably, the tablet comprises magnesium stearate as lubricant. Preferably the tablet comprises croscarmellose sodium as disintegrant. Preferably, the tablet comprises microcrystalline cellulose.

The diluent can be present in a range of 10-80% by weight of the core. The lubricant can be present in a range of 0.25-2% by weight of the core. The disintegrant can be present in a range of 1-10% by weight of the core. Microcrystalline cellulose, if present, can be present in a range of 10-80% by weight of the core.

The active ingredient, e.g., a compound described herein preferably comprises between 10 and 50% of the weight of the core, more preferably between 15 and 35% of the weight of the core (calculated as free base equivalent). The core can contain any therapeutically suitable dosage level of the active ingredient, but preferably contains up to 150 mg of the active ingredient. Particularly preferably, the core contains 20, 30, 40, 50, 60, 80 or 100 mg of the active ingredient. The active ingredient can be present as is or as any pharmaceutically acceptable salt. If the active ingredient is present as a salt, the weight is adjusted such that the tablet contains the desired amount of active ingredient, calculated as free base or free acid of the salt.

The core can be made from a compacted mixture of its components. The components can be directly compressed, or can be granulated before compression. Such granules can be formed by a conventional granulating process as known in the art. In an alternative embodiment, the granules can be individually coated with an enteric casing, and then enclosed in a standard capsule casing.

The core is surrounded by a casing which comprises an enteric polymer. Examples of enteric polymers are cellulose acetate phthalate, cellulose acetate succinate, methylcellulose phthalate, ethylhydroxycellulose phthalate, polyvinylacetate phthalate, polyvinylbutyrate acetate, vinyl acetate-maleic anhydride copolymer, styrene-maleic mono-ester copolymer, methyl acrylate-methacrylic acid copolymer or methacrylate-methacrylic acid-octyl acrylate copolymer. These can be used either alone or in combination, or together with other polymers than those mentioned above. The casing can also include insoluble substances which are neither decomposed nor solubilised in living bodies, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin or 1, 2-, 3, 4-diepoxybutane. The casing can also include starch and/or dextrin.

In some embodiments, an enteric coating materials are the commercially available Eudragit® enteric polymers such as Eudragit® L, Eudragit® S and Eudragit® NE used alone or with a plasticiser. Such coatings are normally applied using a liquid medium, and the nature of the plasticiser depends upon whether the medium is aqueous or non-aqueous. Plasticisers for use with aqueous medium include propylene glycol, triethyl citrate, acetyl triethyl citrate or Citroflex® or Citroflex® A2. Non-aqueous plasticisers include these, and also diethyl and dibutyl phthalate and dibutyl sebacate. A preferred plasticiser is Triethyl citrate. The quantity of plasticiser included will be apparent to those skilled in the art.

The casing can also include an anti-tack agent such as talc, silica or glyceryl monostearate. Preferably the anti-tack agent is glyceryl monostearate. Typically, the casing can include around 5-25 wt % Plasticizers and up to around 50 wt % of anti-tack agent, preferably 1-10 wt % of anti-tack agent.

If desired, a surfactant can be included to aid with forming an aqueous suspension of the polymer. Many examples of possible surfactants are known to the person skilled in the art. Preferred examples of surfactants are polysorbate 80, polysorbate 20, or sodium lauryl sulphate. If present, a surfactant can form 0.1-10% of the casing, preferably 0.2-5% and particularly preferably 0.5-2%.

A seal coat can also be included between the core and the enteric coating. A seal coat is a coating material which can be used to protect the enteric casing from possible chemical attack by any alkaline ingredients in the core. The seal coat can also provide a smoother surface, thereby allowing easier attachment of the enteric casing. A person skilled in the art would be aware of suitable coatings. Preferably the seal coat is made of an Opadry coating, and particularly preferably it is Opadry White OY-S-28876. Other enteric-coated preparations of this sort can be prepared by one skilled in the art, using these materials or their equivalents.

For intravenous injections or drips or infusions, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspension, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

Some exemplary embodiments of various aspects can be described by the following numbered embodiments.

Embodiment 1: A method of treating a disease associated with dysfunction of the Nuclear factor erythroid 2-related factor 2 (Nrf2)/Kelch-like ECH-associated protein 1 (KEAP1) axis in a subject in need thereof, the method comprising administering to the subject a compound of Formula (I):

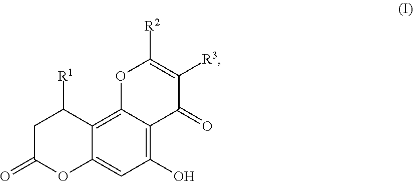

or a pharmaceutically acceptable salt, solvate, hydrate, polymorph, co-crystal, tautomer, stereoisomer, isotopically labeled derivative, or prodrug thereof, wherein:
R¹ is substituted or unsubstituted aryl, substituted or unsubstituted heteroaryl, or substituted or unsubstituted heterocyclyl;
R² is substituted or unsubstituted aryl, substituted or unsubstituted heteroaryl, or substituted or unsubstituted heterocyclyl; and
R³ is hydrogen, —OR^{A}, —SR^{A}, or —N(R^{A})₂;
each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl; or a compound of Formula (II):

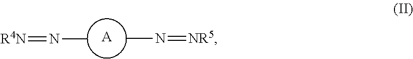

or a pharmaceutically acceptable salt, solvate, hydrate, polymorph, co-crystal, tautomer, stereoisomer, isotopically labeled derivative, or prodrug thereof, wherein:
A is substituted or unsubstituted arylene, substituted or unsubstituted biarylene, or substituted or unsubstituted heteroarylene;
R⁴ is substituted or unsubstituted aryl, substituted or unsubstituted heteroaryl, or substituted or unsubstituted heterocyclyl; and
R⁵ is substituted or unsubstituted aryl, substituted or unsubstituted heteroaryl, or substituted or unsubstituted heterocyclyl; or a compound of Formula (III):

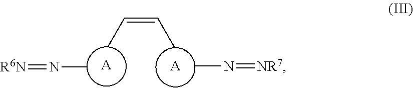

or a pharmaceutically acceptable salt, solvate, hydrate, polymorph, co-crystal, tautomer, stereoisomer, isotopically labeled derivative, or prodrug thereof, wherein:
each A is independently substituted or unsubstituted aryl, or substituted or unsubstituted heteroaryl;
R⁶ is substituted or unsubstituted aryl, substituted or unsubstituted heteroaryl, or substituted or unsubstituted heterocyclyl; and
R⁷ is substituted or unsubstituted aryl, substituted or unsubstituted heteroaryl, or substituted or unsubstituted heterocyclyl; or
   a compound selected from the following:

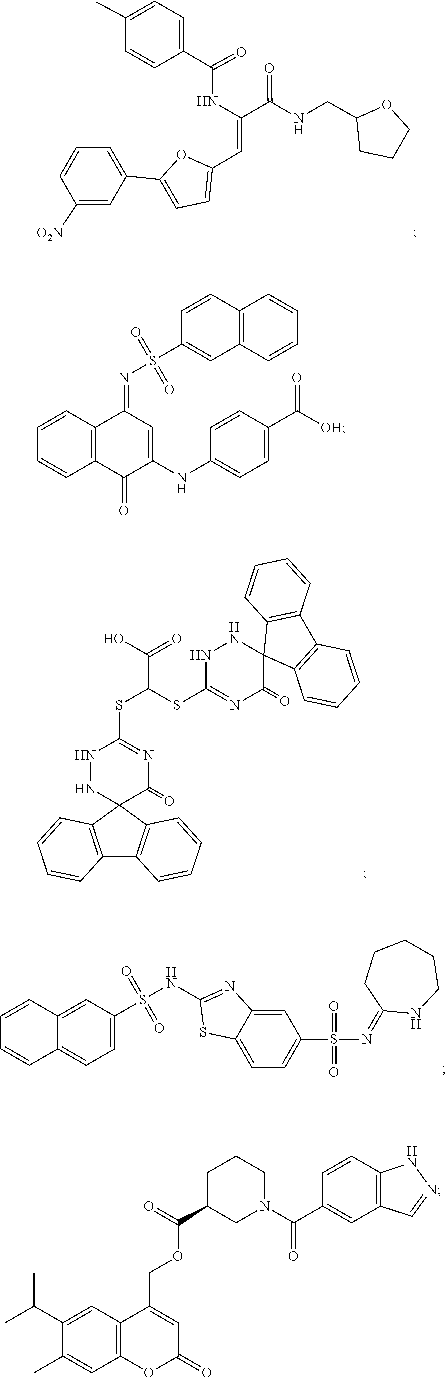 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1 ; Cc1cc2oc(=O)cc(COC(=O)[C@H]3CCCN(C(=O)c4ccc5[nH]ncc5c4)C3)c2cc1C(C)C]

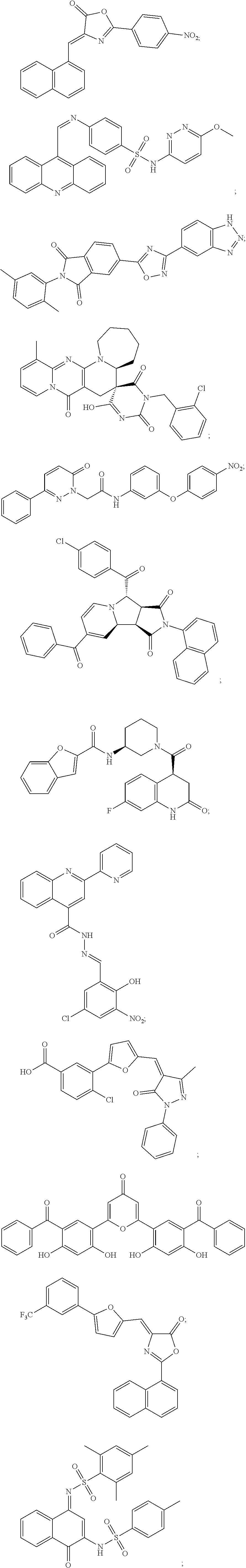

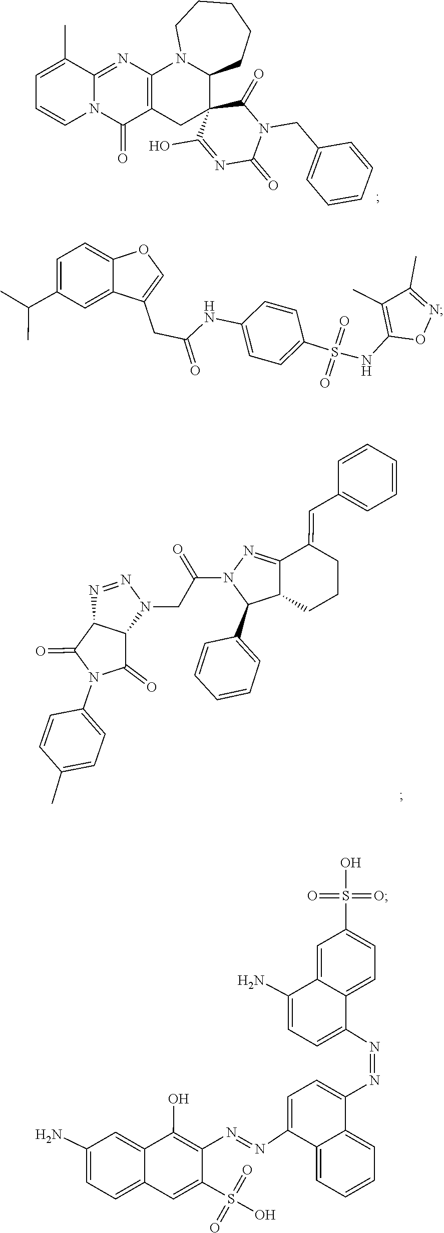 [SMILES: Cc1cccn2c(=O)c3c(nc12)N1CCCCC[C@H]1[C@]1(C3)C(=O)N(Cc2ccccc2)C(=O)N=C1O ; Cc1noc(NS(=O)(=O)c2ccc(NC(=O)Cc3coc4ccc(C(C)C)cc34)cc2)c1C ; Cc1ccc(N2C(=O)[C@@H]3[C@@H](N=NN3CC(=O)N3N=C4/C(=C/c5ccccc5)CCC[C@@H]4[C@H]3c3ccccc3)C2=O)cc1 ; Nc1ccc2cc(S(=O)(=O)O)c(/N=N/c3ccc(/N=N\c4ccc(N)c5cc(S(=O)(=O)O)ccc45)c4ccccc34)c(O)c2c1]

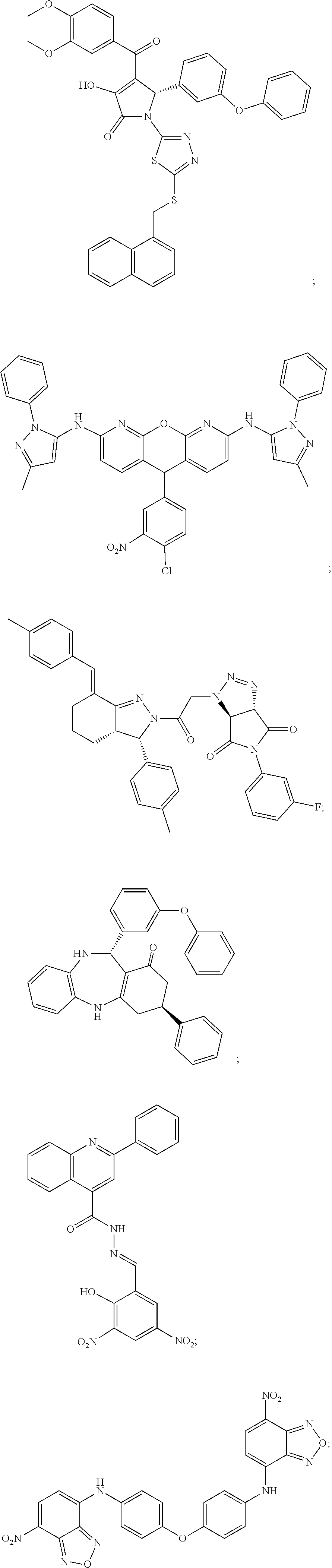

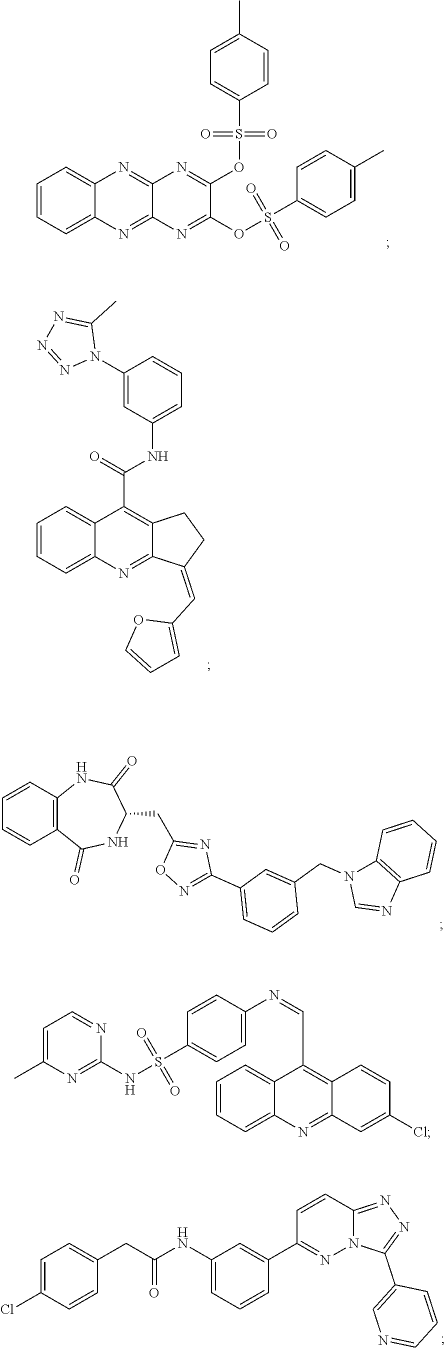

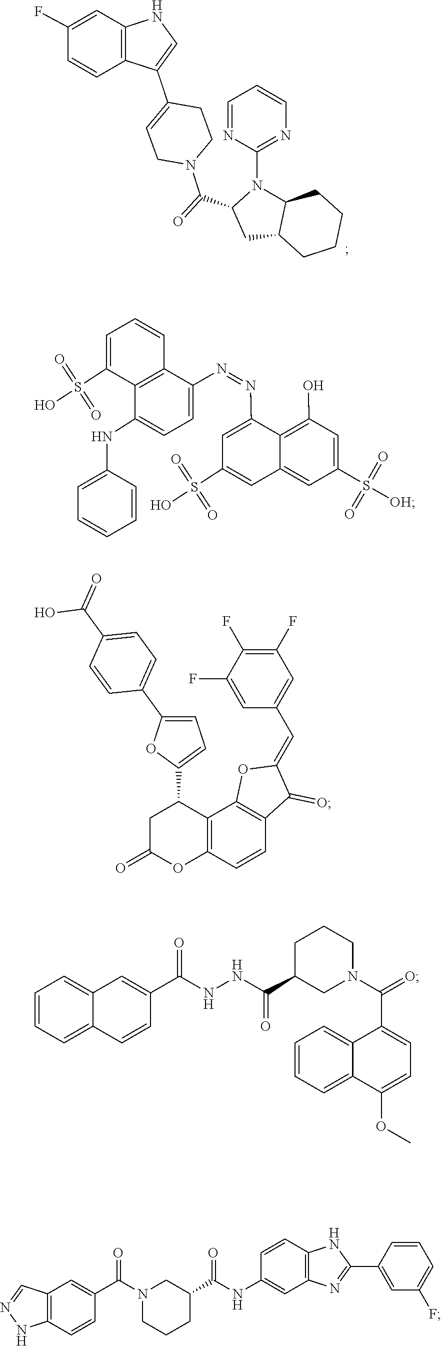

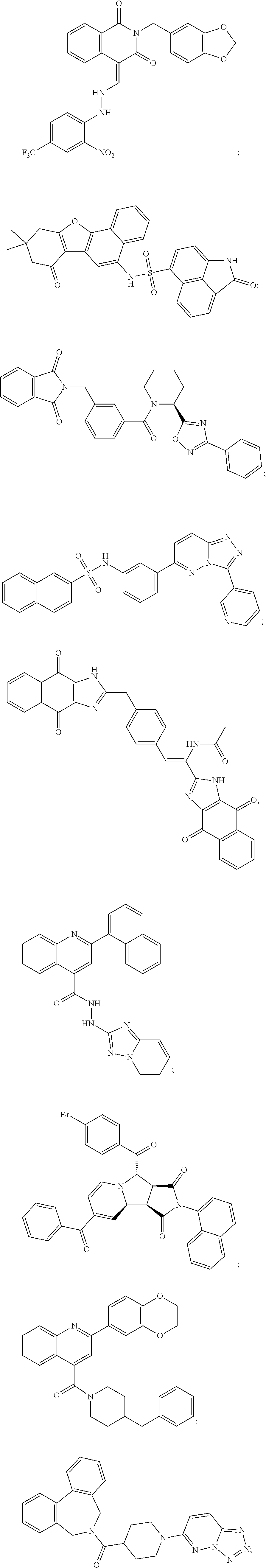

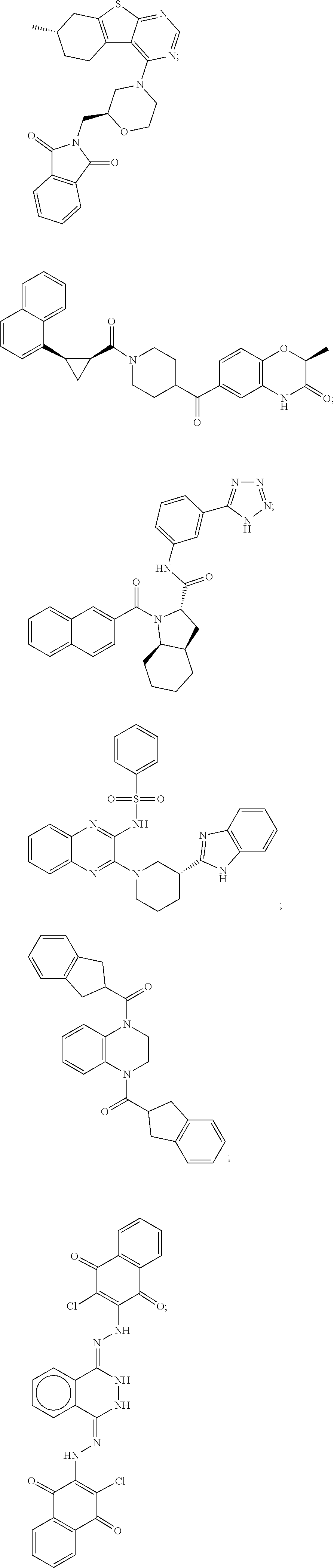

or a pharmaceutically acceptable salt, solvate, hydrate, polymorph, co-crystal, tautomer, stereoisomer, isotopically labeled derivative, or prodrug thereof.

Embodiment 2: A method of treating a disease associated with Nrf2-KEAP1 interaction, the method comprising administering to a subject in need thereof a therapeutically effective amount of a compound of Formula (I):

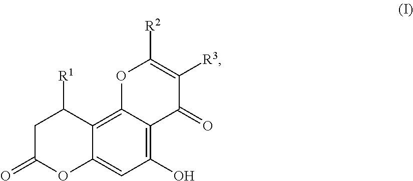

or a pharmaceutically acceptable salt, solvate, hydrate, polymorph, co-crystal, tautomer, stereoisomer, isotopically labeled derivative, or prodrug thereof, wherein:
R¹ is substituted or unsubstituted aryl, substituted or unsubstituted heteroaryl, or substituted or unsubstituted heterocyclyl;
R² is substituted or unsubstituted aryl, substituted or unsubstituted heteroaryl, or substituted or unsubstituted heterocyclyl; and
R³ is hydrogen, —OR^{A}, —SR^{A}, or —N(R^{A})₂;
each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl; or
   a compound of Formula (II):

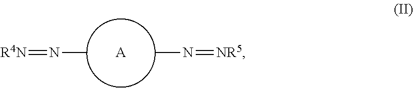

or a pharmaceutically acceptable salt, solvate, hydrate, polymorph, co-crystal, tautomer, stereoisomer, isotopically labeled derivative, or prodrug thereof, wherein:
A is substituted or unsubstituted aryl, substituted or unsubstituted biaryl, or substituted or unsubstituted heteroaryl;
R⁴ is substituted or unsubstituted aryl, substituted or unsubstituted heteroaryl, or substituted or unsubstituted heterocyclyl; and
R⁵ is substituted or unsubstituted aryl, substituted or unsubstituted heteroaryl, or substituted or unsubstituted heterocyclyl; or a compound of Formula (III):

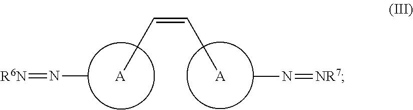

or a pharmaceutically acceptable salt, solvate, hydrate, polymorph, co-crystal, tautomer, stereoisomer, isotopically labeled derivative, or prodrug thereof, wherein:
each A is independently substituted or unsubstituted aryl, or substituted or unsubstituted heteroaryl;
R⁶ is substituted or unsubstituted aryl, substituted or unsubstituted heteroaryl, or substituted or unsubstituted heterocyclyl; and
R⁷ is substituted or unsubstituted aryl, substituted or unsubstituted heteroaryl, or substituted or unsubstituted heterocyclyl; or
   a compound selected from the following:

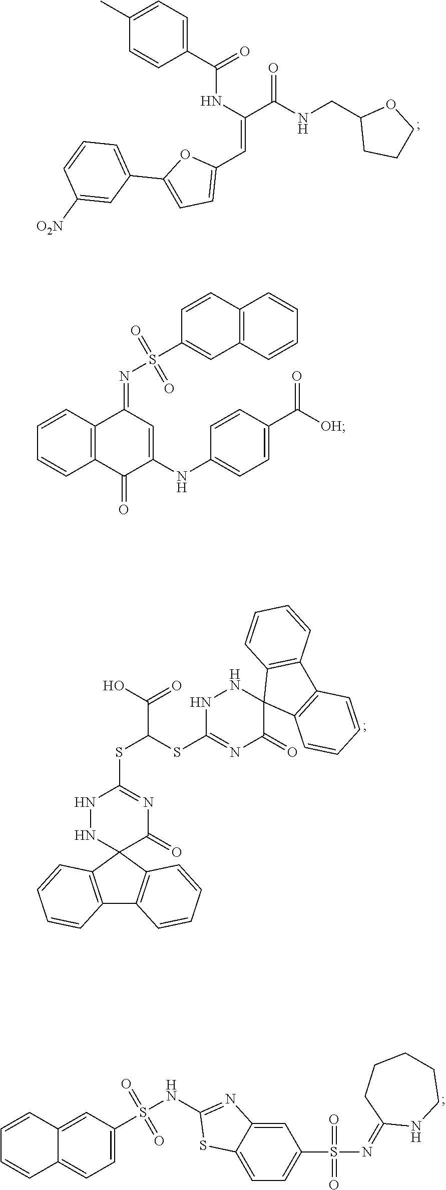 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1]

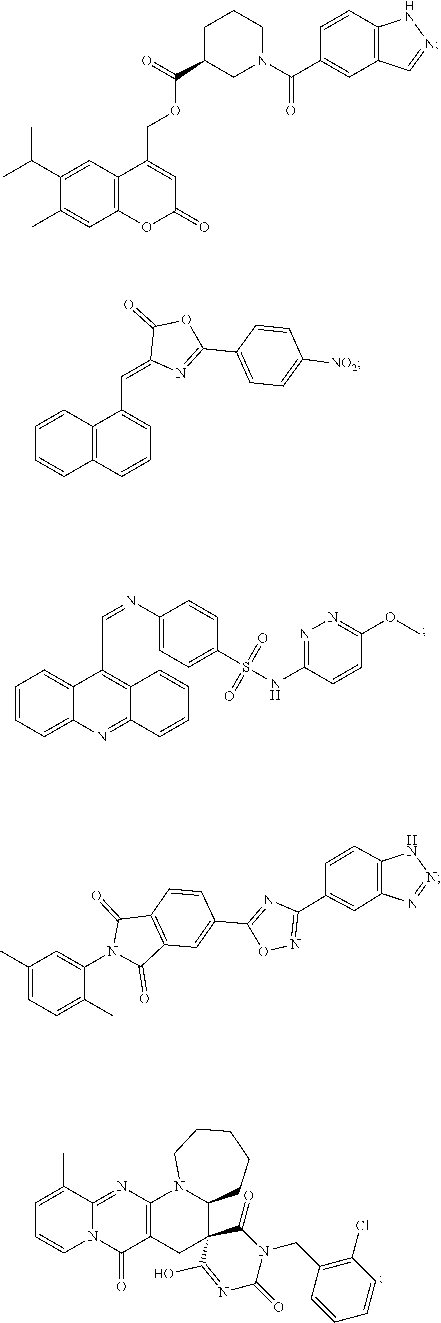

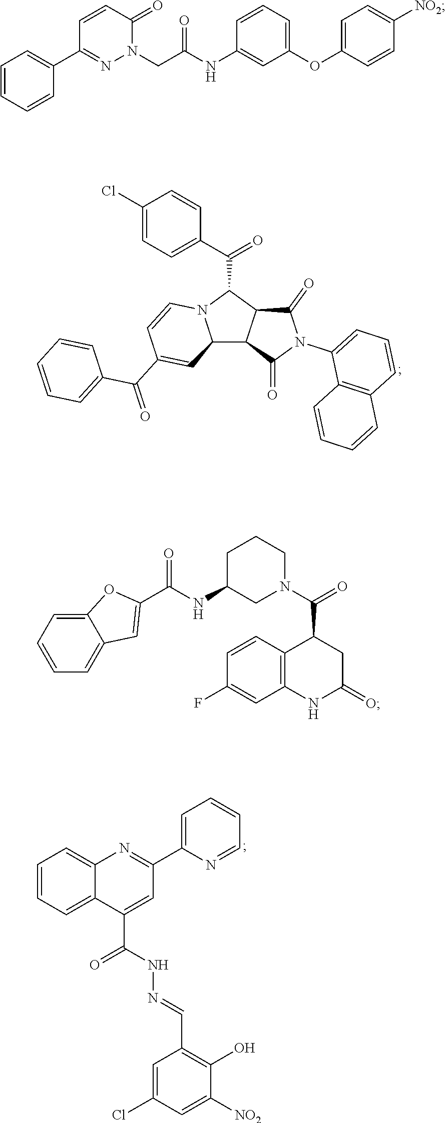 [SMILES: O=C(Cn1nc(-c2ccccc2)ccc1=O)Nc1cccc(Oc2ccc([N+](=O)[O-])cc2)c1 ; O=C(C1=C[C@@H]2[C@@H]3C(=O)N(c4cccc5ccccc45)C(=O)[C@@H]3[C@@H](C(=O)c3ccc(Cl)cc3)N2C=C1)c1ccccc1 ; O=C1C[C@H](C(=O)N2CCC[C@H](NC(=O)c3cc4ccccc4o3)C2)c2ccc(F)cc2N1 ; O=C(N/N=C/c1cc(Cl)cc([N+](=O)[O-])c1O)c1cc(-c2ccccn2)nc2ccccc12]

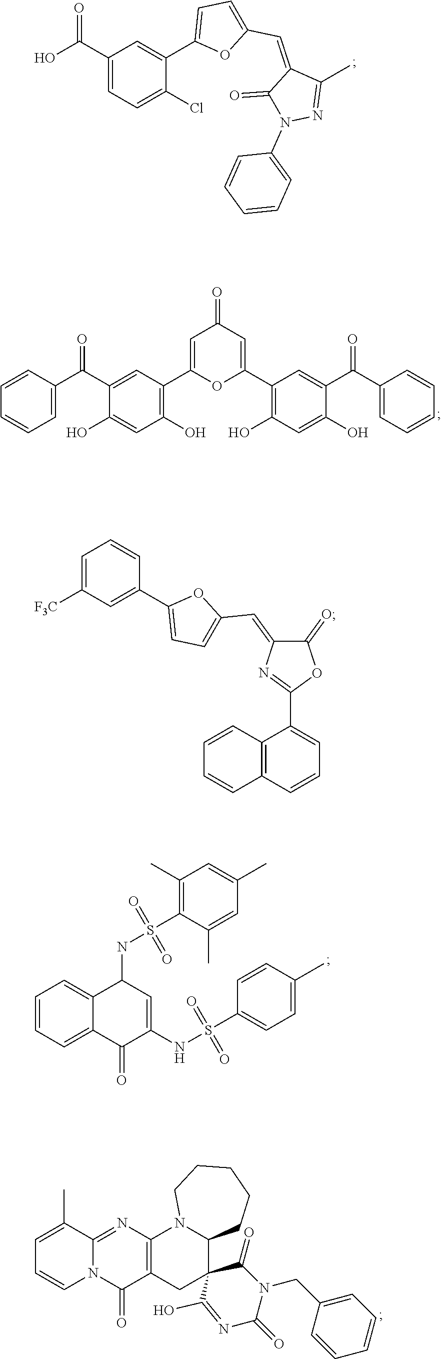 [SMILES: CC1=NN(c2ccccc2)C(=O)/C1=C\c1ccc(-c2cc(C(=O)O)ccc2Cl)o1 ; O=C(c1ccccc1)c1cc(-c2cc(=O)cc(-c3cc(C(=O)c4ccccc4)c(O)cc3O)o2)c(O)cc1O ; O=C1OC(c2cccc3ccccc23)=N/C1=C\c1ccc(-c2cccc(C(F)(F)F)c2)o1 ; Cc1ccc(S(=O)(=O)NC2=CC(NS(=O)(=O)c3c(C)cc(C)cc3C)c3ccccc3C2=O)cc1 ; Cc1cccn2c(=O)c3c(nc12)N1CCCCC[C@H]1[C@]1(C3)C(=O)N(Cc2ccccc2)C(=O)N=C1O]

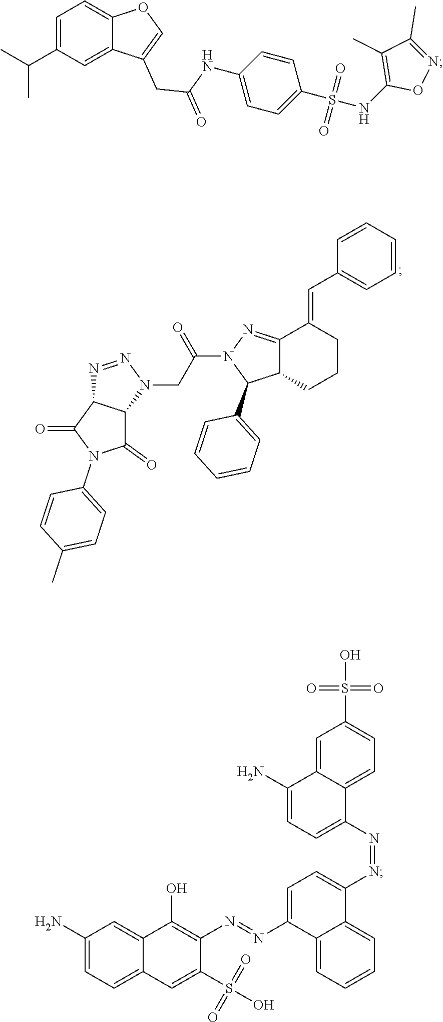 [SMILES: Cc1noc(NS(=O)(=O)c2ccc(NC(=O)Cc3coc4ccc(C(C)C)cc34)cc2)c1C ; Cc1ccc(N2C(=O)[C@@H]3[C@@H](N=NN3CC(=O)N3N=C4/C(=C/c5ccccc5)CCC[C@@H]4[C@H]3c3ccccc3)C2=O)cc1 ; Nc1ccc2cc(S(=O)(=O)O)c(/N=N/c3ccc(/N=N\c4ccc(N)c5cc(S(=O)(=O)O)ccc45)c4ccccc34)c(O)c2c1]

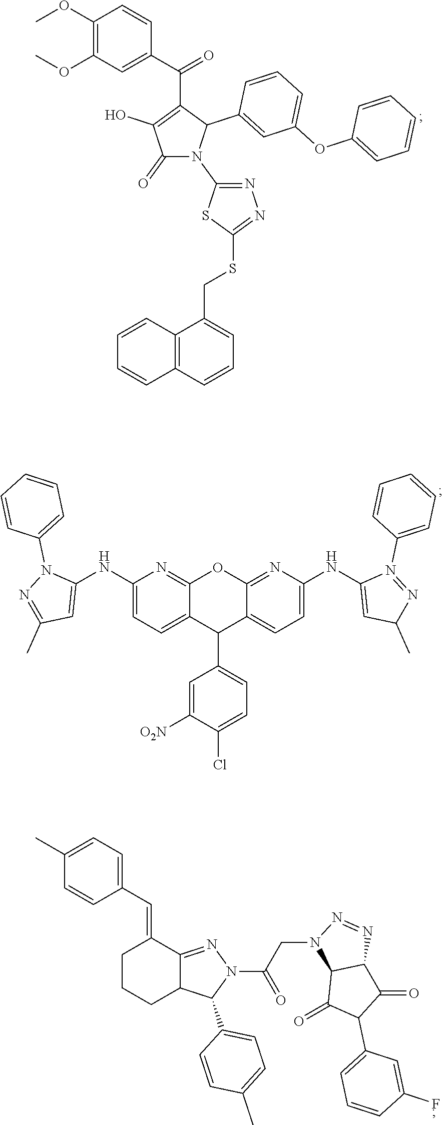

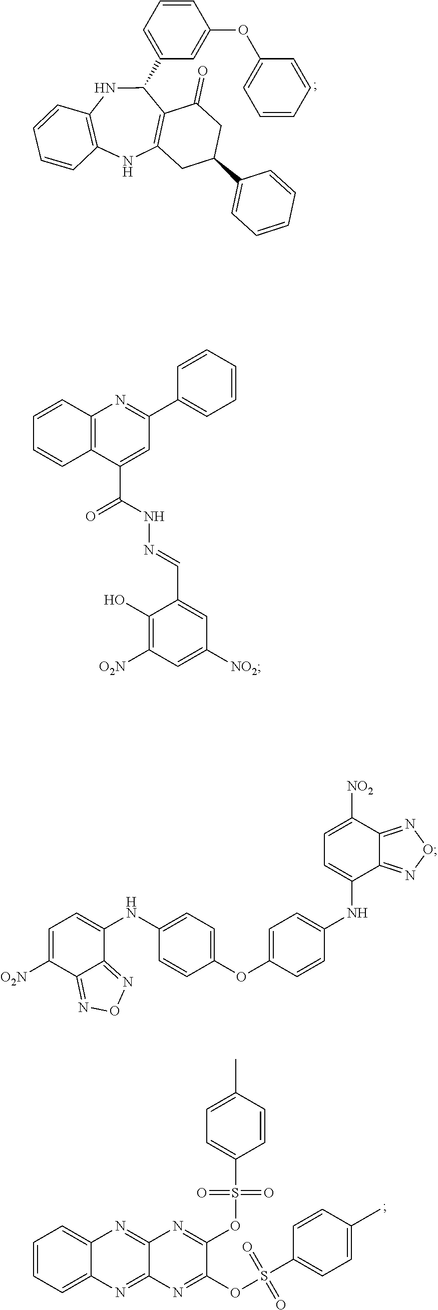 [SMILES: O=C1C[C@@H](c2ccccc2)CC2=C1[C@@H](c1cccc(Oc3ccccc3)c1)Nc1ccccc1N2 ; O=C(N/N=C/c1cc([N+](=O)[O-])cc([N+](=O)[O-])c1O)c1cc(-c2ccccc2)nc2ccccc12 ; O=[N+]([O-])c1ccc(Nc2ccc(Oc3ccc(Nc4ccc([N+](=O)[O-])c5nonc45)cc3)cc2)c2nonc12 ; Cc1ccc(S(=O)(=O)Oc2nc3nc4ccccc4nc3nc2OS(=O)(=O)c2ccc(C)cc2)cc1]

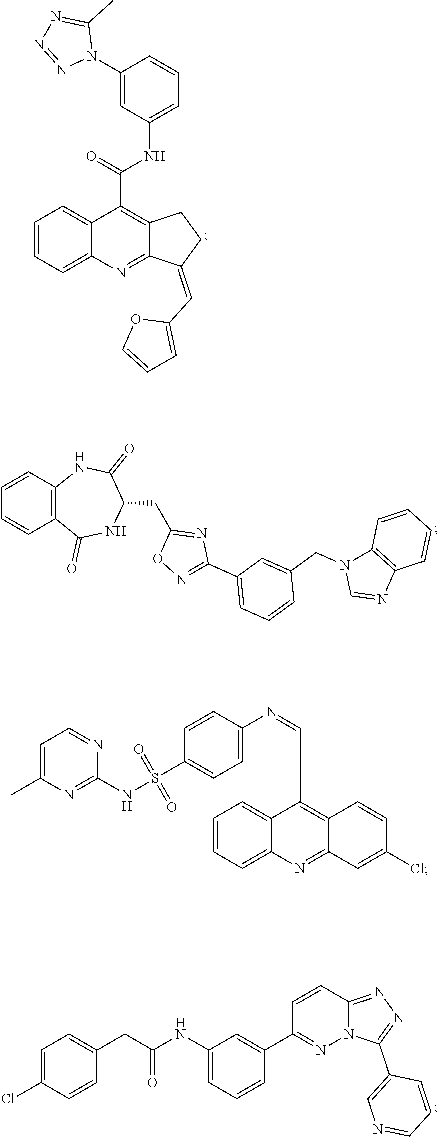 [SMILES: Cc1nnnn1-c1cccc(NC(=O)c2c3c(nc4ccccc24)/C(=C\c2ccco2)CC3)c1 ; O=C1N[C@@H](Cc2nc(-c3cccc(Cn4cnc5ccccc54)c3)no2)C(=O)Nc2ccccc21 ; Cc1ccnc(NS(=O)(=O)c2ccc(/N=C\c3c4ccccc4nc4cc(Cl)ccc34)cc2)n1 ; O=C(Cc1ccc(Cl)cc1)Nc1cccc(-c2ccc3nnc(-c4cccnc4)n3n2)c1]

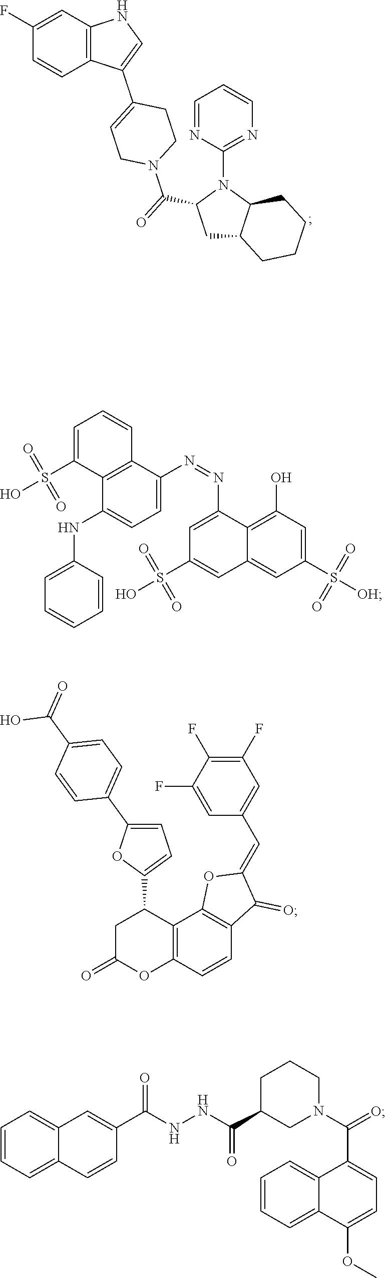 [SMILES: O=C([C@H]1C[C@H]2CCCC[C@@H]2N1c1ncccn1)N1CC=C(c2c[nH]c3cc(F)ccc23)CC1 ; O=S(=O)(O)c1cc(O)c2c(/N=N\c3ccc(Nc4ccccc4)c4c(S(=O)(=O)O)cccc34)cc(S(=O)(=O)O)cc2c1 ; O=C1C[C@H](c2ccc(-c3ccc(C(=O)O)cc3)o2)c2c(ccc3c2O/C(=C\c2cc(F)c(F)c(F)c2)C3=O)O1 ; COc1ccc(C(=O)N2CCC[C@H](C(=O)NNC(=O)c3ccc4ccccc4c3)C2)c2ccccc12]

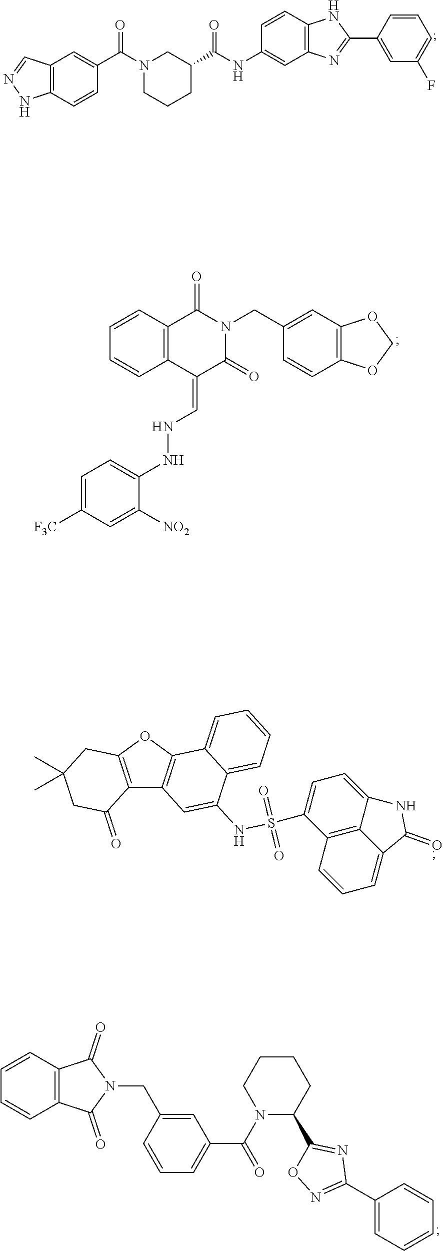 [SMILES: O=C(Nc1ccc2[nH]c(-c3cccc(F)c3)nc2c1)[C@@H]1CCCN(C(=O)c2ccc3[nH]ncc3c2)C1 ; O=C1/C(=C/NNc2ccc(C(F)(F)F)cc2[N+](=O)[O-])c2ccccc2C(=O)N1Cc1ccc2c(c1)OCO2 ; CC1(C)CC(=O)c2c(oc3c2cc(NS(=O)(=O)c2ccc4c5c(cccc25)C(=O)N4)c2ccccc23)C1 ; O=C1c2ccccc2C(=O)N1Cc1cccc(C(=O)N2CCCC[C@H]2c2nc(-c3ccccc3)no2)c1]

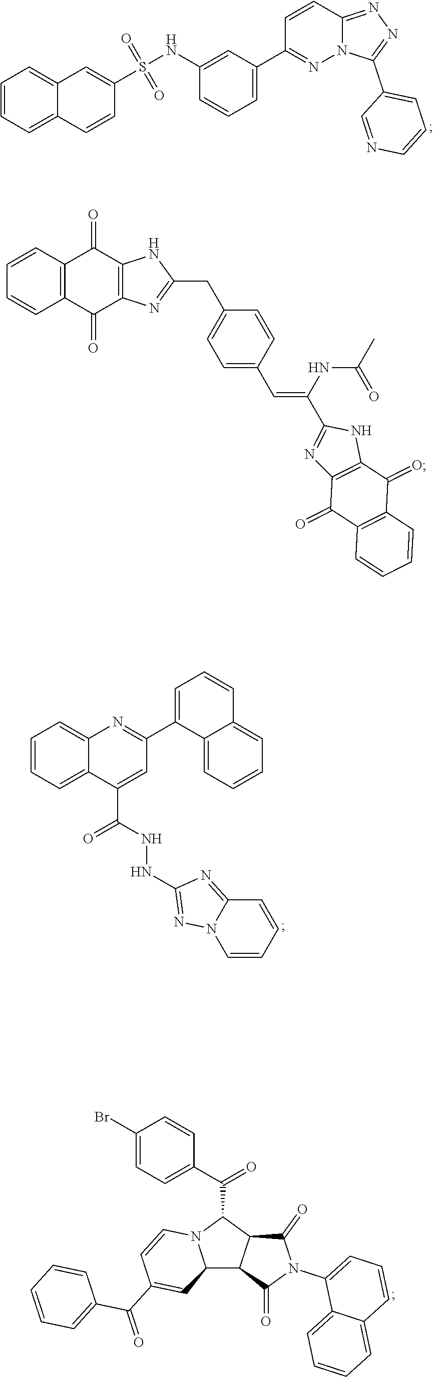 [SMILES: O=S(=O)(Nc1cccc(-c2ccc3nnc(-c4cccnc4)n3n2)c1)c1ccc2ccccc2c1 ; CC(=O)N/C(=C\c1ccc(Cc2nc3c([nH]2)C(=O)c2ccccc2C3=O)cc1)c1nc2c([nH]1)C(=O)c1ccccc1C2=O ; O=C(NNc1nc2ccccn2n1)c1cc(-c2cccc3ccccc23)nc2ccccc12 ; O=C(C1=C[C@@H]2[C@@H]3C(=O)N(c4cccc5ccccc45)C(=O)[C@@H]3[C@@H](C(=O)c3ccc(Br)cc3)N2C=C1)c1ccccc1]

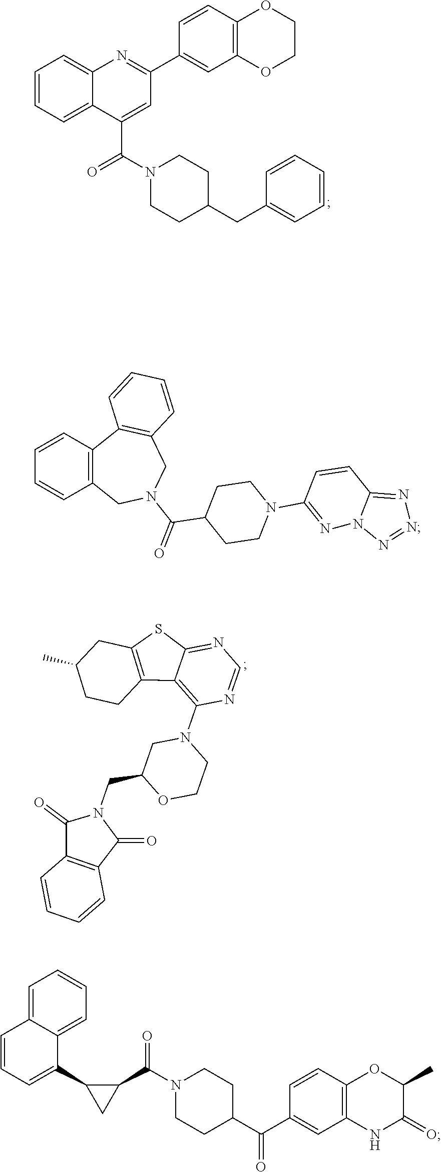 [SMILES: O=C(c1cc(-c2ccc3c(c2)OCCO3)nc2ccccc12)N1CCC(Cc2ccccc2)CC1 ; O=C(C1CCN(c2ccc3nnnn3n2)CC1)N1Cc2ccccc2-c2ccccc2C1 ; C[C@H]1CCc2c(sc3ncnc(N4CCO[C@@H](CN5C(=O)c6ccccc6C5=O)C4)c23)C1 ; C[C@@H]1Oc2ccc(C(=O)C3CCN(C(=O)[C@H]4C[C@H]4c4cccc5ccccc45)CC3)cc2NC1=O]

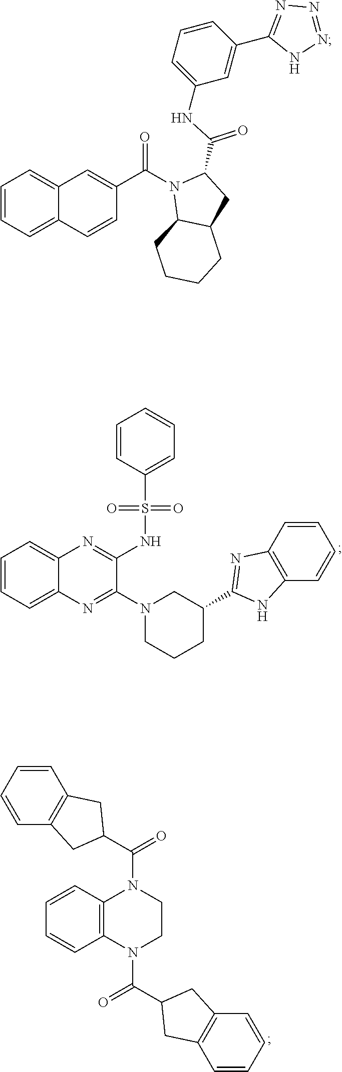 [SMILES: O=C(Nc1cccc(-c2nnn[nH]2)c1)[C@@H]1C[C@H]2CCCC[C@H]2N1C(=O)c1ccc2ccccc2c1 ; O=S(=O)(Nc1nc2ccccc2nc1N1CCC[C@@H](c2nc3ccccc3[nH]2)C1)c1ccccc1 ; O=C(C1Cc2ccccc2C1)N1CCN(C(=O)C2Cc3ccccc3C2)c2ccccc21]

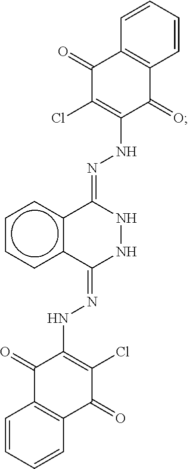

or a pharmaceutically acceptable salt, solvate, hydrate, polymorph, co-crystal, tautomer, stereoisomer, isotopically labeled derivative, or prodrug thereof.

Embodiment 3: The method of Embodiment 1 or 2, wherein the disease is associated with oxidative stress.

Embodiment 4: The method of any of Embodiments 1-3, wherein the disease is a metabolic disease, an inflammatory disease, an autoimmune disease, a lung disease, a cardiovascular disease, a liver disease, a kidney disease, an ophthalmological disease, a gastrointestinal tract disease, a neurological disease, a neurodegenerative disease, or cancer.

Embodiment 5: The method of any of Embodiments 1-4, wherein the disease is a metabolic disease.

Embodiment 6: The method of any of Embodiments 1-5, wherein the disease is metabolic syndrome, type 2 diabetes, diabetic nephropathy, diabetic cardiopathy, obesity, or insulin resistance.

Embodiment 7: The method of any of Embodiments 1-4, wherein the disease is a liver disease.

Embodiment 8: The method of any of Embodiments 1-4 or 7, wherein the disease is hepatic fibrosis, autosomal dominant polycystic liver disease, hepatic steatosis, non-alcoholic steatohepatitis (NASH), or non-alcoholic fatty liver disease (NAFLD).

Embodiment 9: The method of any of Embodiments 1-4, wherein the disease is an inflammatory disease.

Embodiment 10: The method of any of Embodiments 1-4 or 9, wherein the disease is asthma, chronic obstructive pulmonary disease (COPD), cystic fibrosis, or airway hyperresponsiveness.

Embodiment 11: The method of any of Embodiments 1-4, wherein the disease is an autoimmune disease.

Embodiment 12: The method of any of Embodiments 1-4 or 11, wherein the disease is multiple sclerosis, psoriasis, connective tissue disease, or pulmonary arterial hypertension associated with connective tissue disease.

Embodiment 13: The method of any of Embodiments 1-4, wherein the disease is a kidney disease.

Embodiment 14: The method of any of Embodiments 1-4 or 13, wherein the disease is renal fibrosis, Alport syndrome, autosomal dominant polycystic kidney disease, chronic kidney disease, IgA nephropathy, type 1 diabetes, type 2 diabetes mellitus, and focal segmental glomerulosclerosis, or nephropathy.

Embodiment 15: The method of any of Embodiments 1-4, wherein the disease is a lung disease.

Embodiment 16: The method of any of Embodiments 1-4 or 15, wherein the disease is pulmonary arterial hypertension, pulmonary hypertension-interstitial lung disease, pulmonary fibrosis, cystic fibrosis, emphysema, chronic obstructive pulmonary disease (COPD), or chronic bronchitis.

Embodiment 17: The method of any of Embodiments 1-4, wherein the disease is a cardiovascular disease.

Embodiment 18: The method of any of Embodiments 1-4 or 17, wherein the disease is atherosclerosis, heart failure, myocardial infarction, reperfusion injury, or stroke.

Embodiment 19: The method of any of Embodiments 1-4, wherein the disease is a neurological or neurodegenerative disease.

Embodiment 20: The method of any of Embodiments 1-4 or 19, wherein the disease is Friedreich's ataxia, subarachnoid hemorrhage, amyotrophic lateral sclerosis, Parkinson's disease, Parkinson's disease with dementia with Lewy body, Huntington's Disease, Batten Disease, multiple system atrophy (MSA), progressive supranuclear palsy (PSA), corticobasal degeneration (CBD), frontotemporal lobe degeneration, Alzheimer's disease, Fragile X syndrome, chronic fatigue syndrome, cerebral ischemia, neuronal cell death, Creutzfeldt-Jakob disease, Lewy body disease, Pick's disease, or neurofibromatosis.

Embodiment 21: The method of any of Embodiments 1-4, wherein the disease is an ophthalmological disease.

Embodiment 22: The method of any of Embodiments 1-4 or 21, wherein the disease is dry eye macular degeneration, retinovascular disease, or retinopathy.

Embodiment 23: The method of any of Embodiments 1-4, wherein the disease is cancer.

Embodiment 24: The method of any of Embodiments 1-4 or 23, wherein the disease is breast cancer, liver cancer, lung cancer, breast cancer, prostate cancer, colon cancer, neuroblastoma, or leukemia.

Embodiment 25: The method of any of Embodiments 1-24, wherein the subject is a human.

Embodiment 26: The method of any of Embodiments 1-25, wherein the compound is of Formula (I):

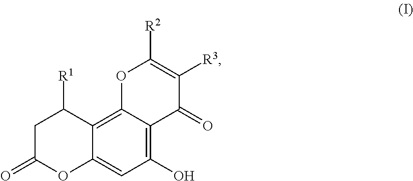

or a pharmaceutically acceptable salt, solvate, hydrate, polymorph, co-crystal, tautomer, stereoisomer, isotopically labeled derivative, or prodrug thereof.

Embodiment 27: The method of any of Embodiments 1-26, wherein the compound is of formula:

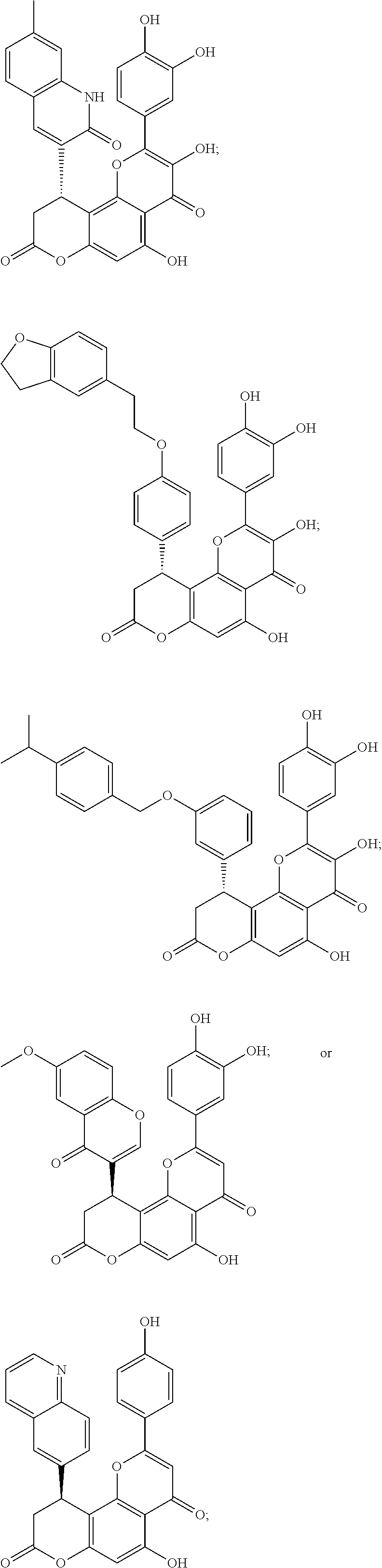 [SMILES: Cc1ccc2cc([C@H]3CC(=O)Oc4cc(O)c5c(=O)c(O)c(-c6ccc(O)c(O)c6)oc5c43)c(=O)[nH]c2c1 ; O=C1C[C@H](c2ccc(OCCc3ccc4c(c3)CCO4)cc2)c2c(cc(O)c3c(=O)c(O)c(-c4ccc(O)c(O)c4)oc23)O1 ; CC(C)c1ccc(COc2cccc([C@H]3CC(=O)Oc4cc(O)c5c(=O)c(O)c(-c6ccc(O)c(O)c6)oc5c43)c2)cc1 ; COc1ccc2occ([C@@H]3CC(=O)Oc4cc(O)c5c(=O)cc(-c6ccc(O)c(O)c6)oc5c43)c(=O)c2c1 ; O=C1C[C@@H](c2ccc3ncccc3c2)c2c(cc(O)c3c(=O)cc(-c4ccc(O)cc4)oc23)O1]

or a pharmaceutically acceptable salt, solvate, hydrate, polymorph, co-crystal, tautomer, stereoisomer, isotopically labeled derivative, or prodrug thereof.

Embodiment 28: The method of any of Embodiments 1-25, wherein the compound is of Formula (II):

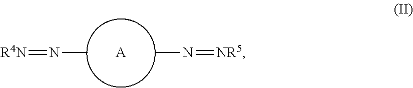

or a pharmaceutically acceptable salt, solvate, hydrate, polymorph, co-crystal, tautomer, stereoisomer, isotopically labeled derivative, or prodrug thereof.

Embodiment 29: The method of any of Embodiments 1-25 or 28, wherein the compound is selected from the following:

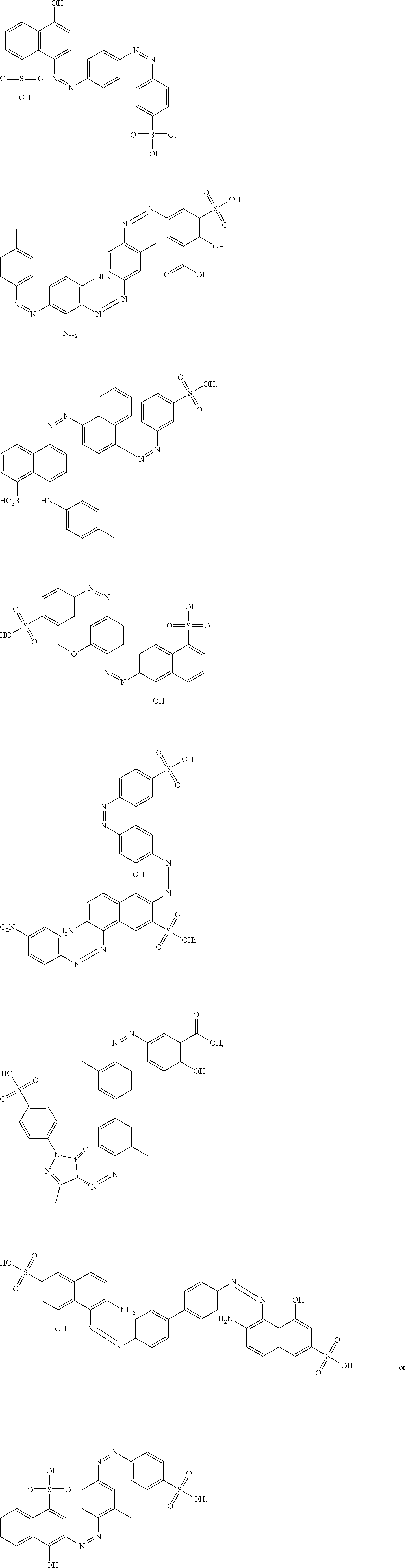

or a pharmaceutically acceptable salt, solvate, hydrate, polymorph, co-crystal, tautomer, stereoisomer, isotopically labeled derivative, or prodrug thereof.

Embodiment 30: The method of any of Embodiments 1-25, wherein the compound is of Formula (III):

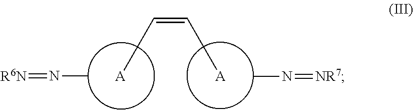

or a pharmaceutically acceptable salt, solvate, hydrate, polymorph, co-crystal, tautomer, stereoisomer, isotopically labeled derivative, or prodrug thereof.

Embodiment 31: The method of any of Embodiments 1-25 or 30, wherein the compound is of formula:

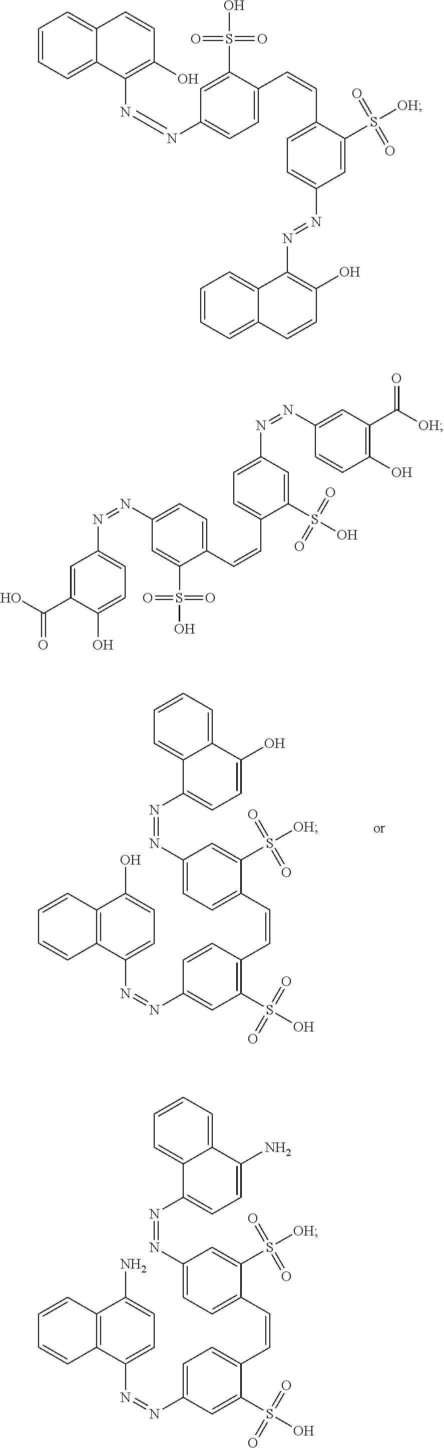 [SMILES: O=S(=O)(O)c1cc(/N=N\c2c(O)ccc3ccccc23)ccc1/C=C\c1ccc(/N=N/c2c(O)ccc3ccccc23)cc1S(=O)(=O)O ; O=C(O)c1cc(/N=N\c2ccc(/C=C\c3ccc(/N=N\c4ccc(O)c(C(=O)O)c4)cc3S(=O)(=O)O)c(S(=O)(=O)O)c2)ccc1O ; O=S(=O)(O)c1cc(/N=N\c2ccc(O)c3ccccc23)ccc1/C=C\c1ccc(/N=N\c2ccc(O)c3ccccc23)cc1S(=O)(=O)O ; Nc1ccc(/N=N\c2ccc(/C=C\c3ccc(/N=N\c4ccc(N)c5ccccc45)cc3S(=O)(=O)O)c(S(=O)(=O)O)c2)c2ccccc12]

or a pharmaceutically acceptable salt, solvate, hydrate, polymorph, co-crystal, tautomer, stereoisomer, isotopically labeled derivative, or prodrug thereof.

Embodiment 32: The method of any of Embodiments 1-25, wherein the compound is of formula:

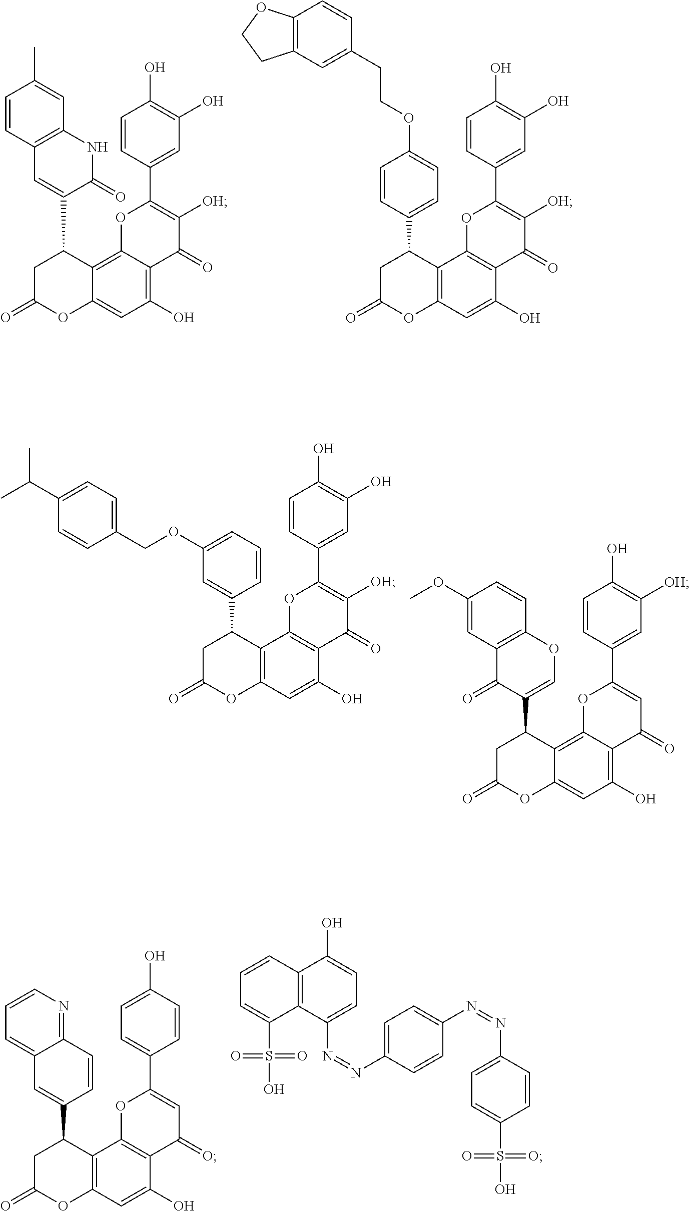

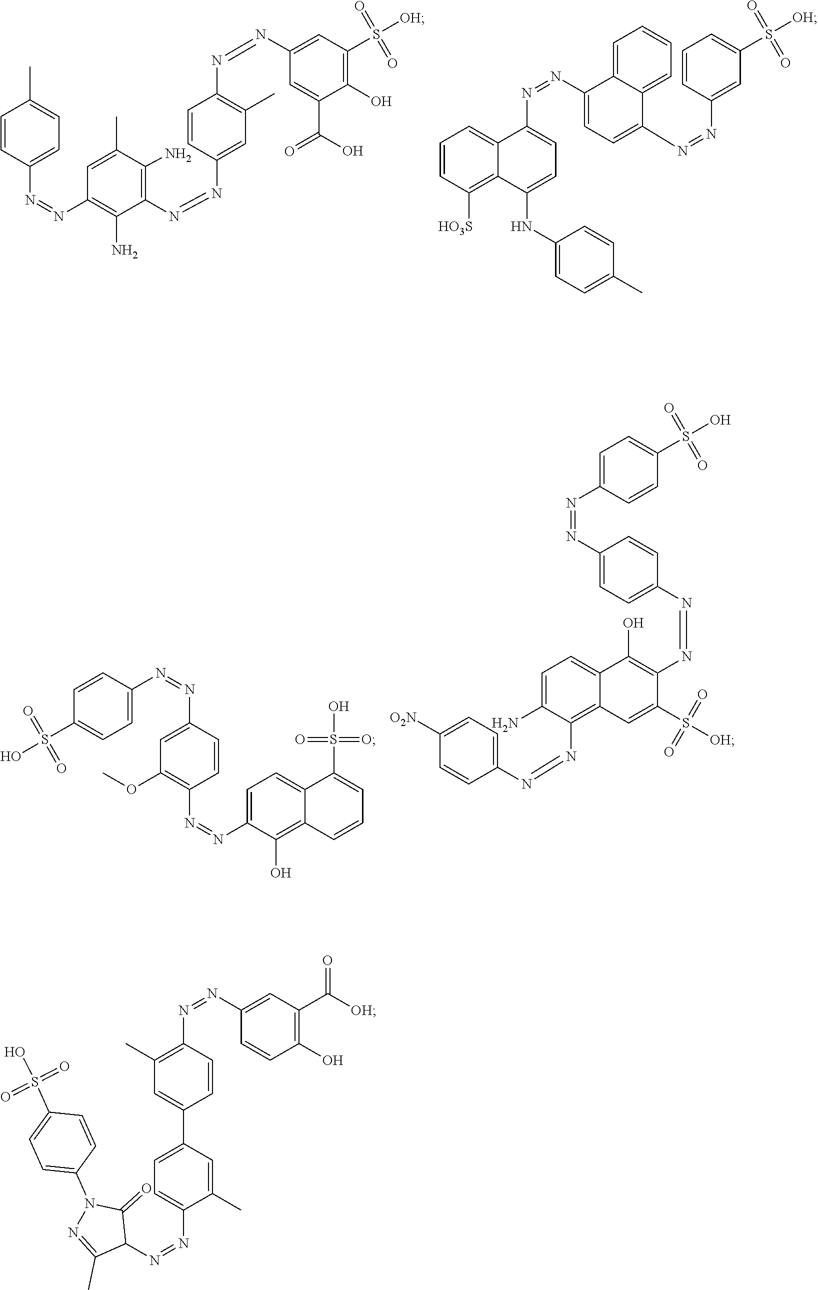 [SMILES: Cc1ccc(/N=N\c2cc(C)c(N)c(/N=N\c3ccc(/N=N\c4cc(C(=O)O)c(O)c(S(=O)(=O)O)c4)c(C)c3)c2N)cc1 ; Cc1ccc(Nc2ccc(/N=N\c3ccc(/N=N\c4cccc(S(=O)(=O)O)c4)c4ccccc34)c3cccc(S(=O)(=O)O)c23)cc1 ; COc1cc(/N=N\c2ccc(S(=O)(=O)O)cc2)ccc1/N=N\c1ccc2c(S(=O)(=O)O)cccc2c1O ; Nc1ccc2c(O)c(/N=N\c3ccc(/N=N\c4ccc(S(=O)(=O)O)cc4)cc3)c(S(=O)(=O)O)cc2c1/N=N\c1ccc([N+](=O)[O-])cc1 ; CC1=NN(c2ccc(S(=O)(=O)O)cc2)C(=O)C1/N=N\c1ccc(-c2ccc(/N=N\c3ccc(O)c(C(=O)O)c3)c(C)c2)cc1C]

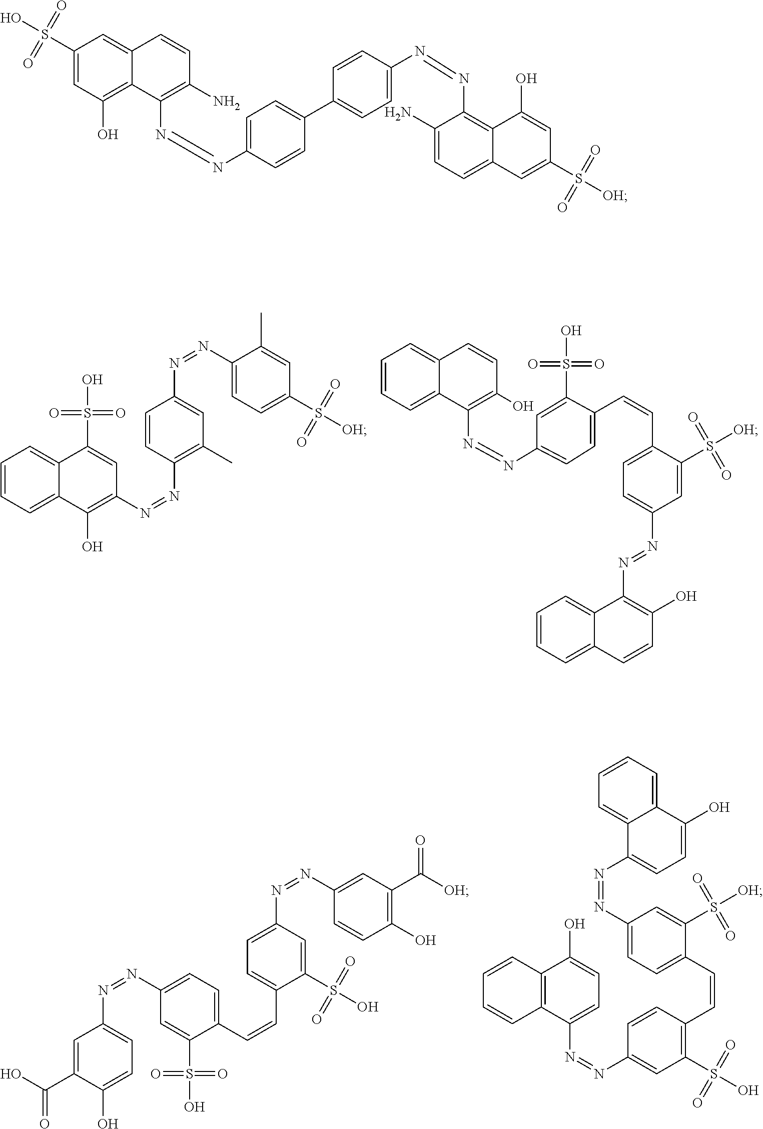 [SMILES: Nc1ccc2cc(S(=O)(=O)O)cc(O)c2c1/N=N\c1ccc(-c2ccc(/N=N\c3c(N)ccc4cc(S(=O)(=O)O)cc(O)c34)cc2)cc1 ; Cc1cc(S(=O)(=O)O)ccc1/N=N\c1ccc(/N=N\c2cc(S(=O)(=O)O)c3ccccc3c2O)c(C)c1 ; O=S(=O)(O)c1cc(/N=N\c2c(O)ccc3ccccc23)ccc1/C=C\c1ccc(/N=N/c2c(O)ccc3ccccc23)cc1S(=O)(=O)O ; O=C(O)c1cc(/N=N\c2ccc(/C=C\c3ccc(/N=N\c4ccc(O)c(C(=O)O)c4)cc3S(=O)(=O)O)c(S(=O)(=O)O)c2)ccc1O ; O=S(=O)(O)c1cc(/N=N\c2ccc(O)c3ccccc23)ccc1/C=C\c1ccc(/N=N\c2ccc(O)c3ccccc23)cc1S(=O)(=O)O]

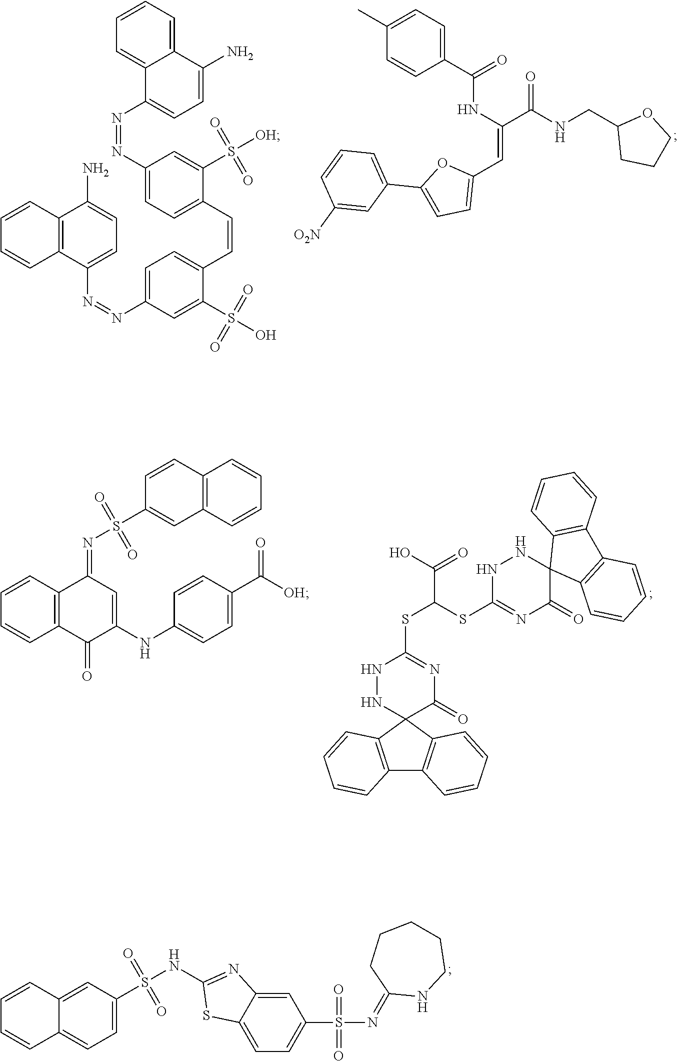 [SMILES: Nc1ccc(/N=N\c2ccc(/C=C\c3ccc(/N=N\c4ccc(N)c5ccccc45)cc3S(=O)(=O)O)c(S(=O)(=O)O)c2)c2ccccc12 ; Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1]

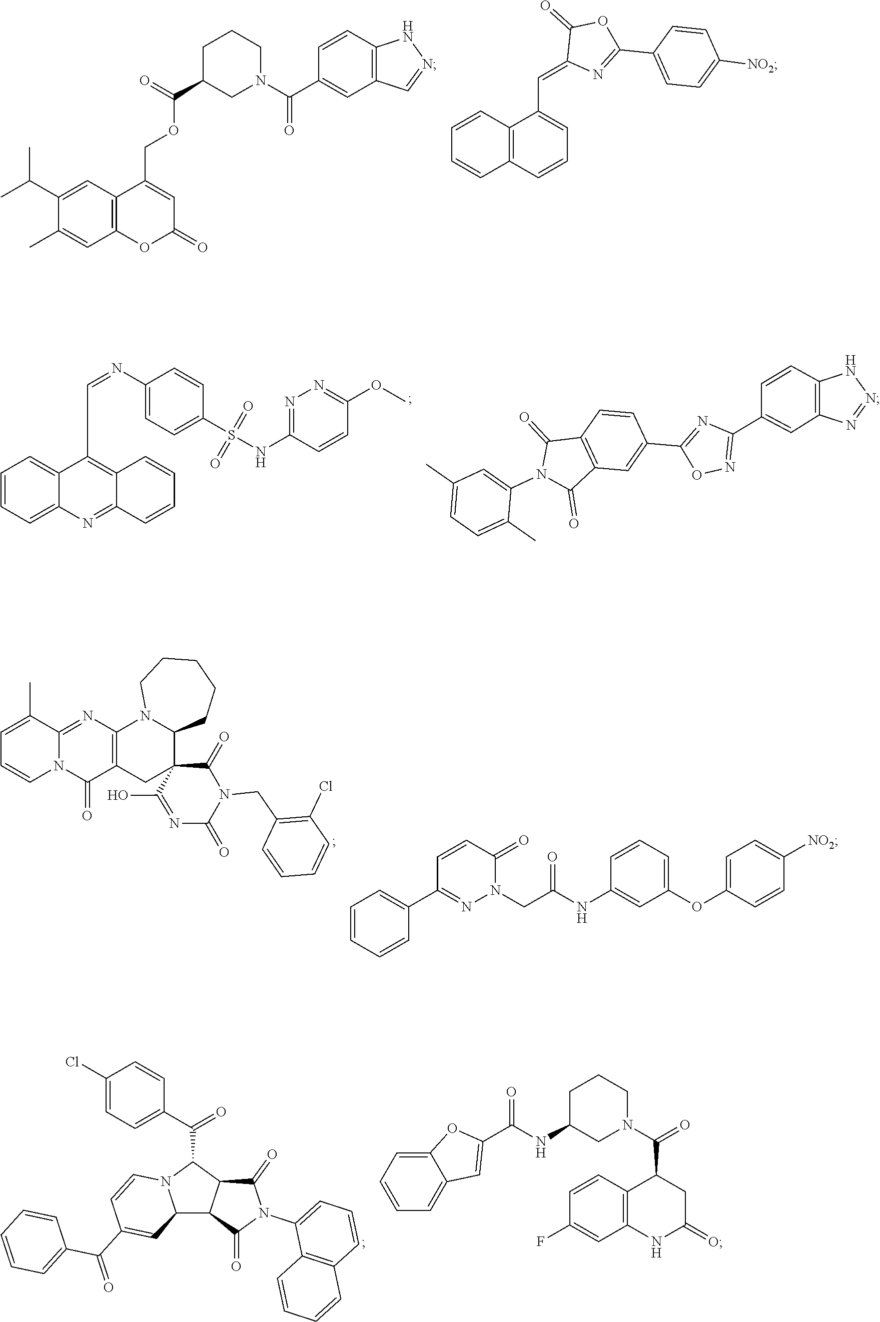

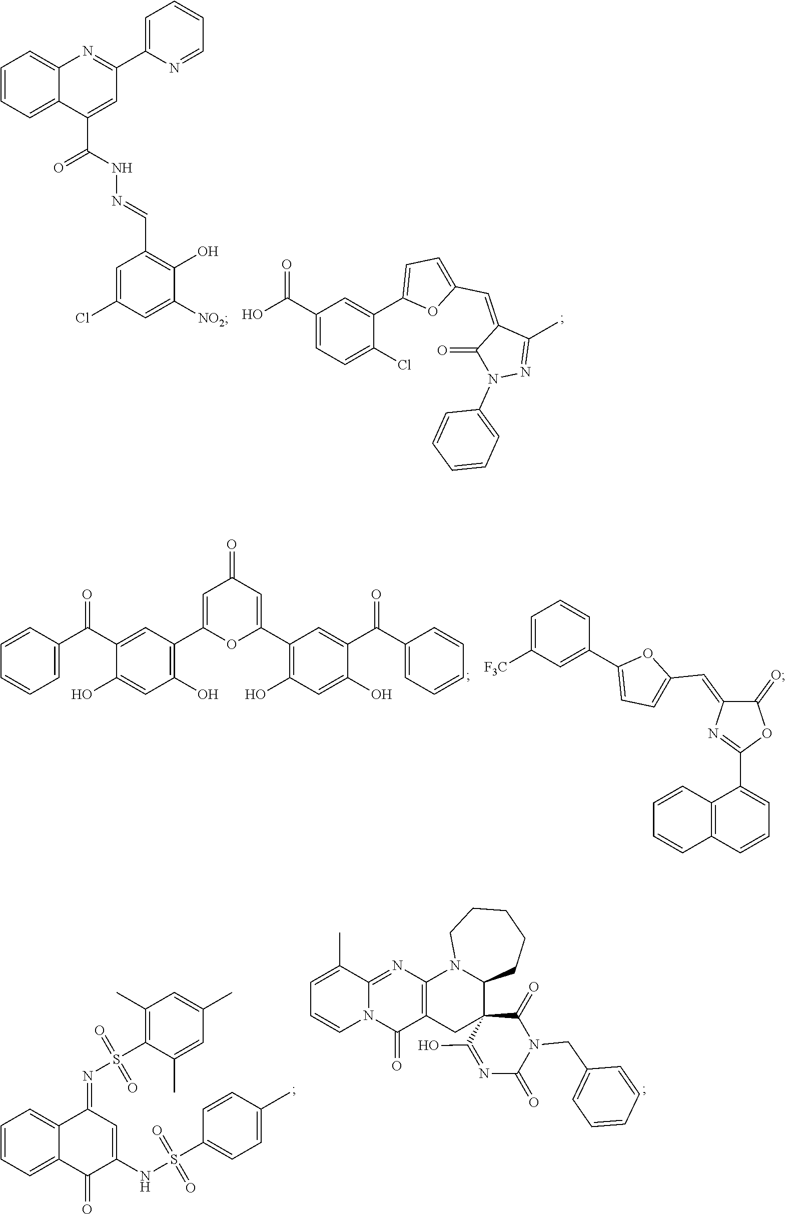

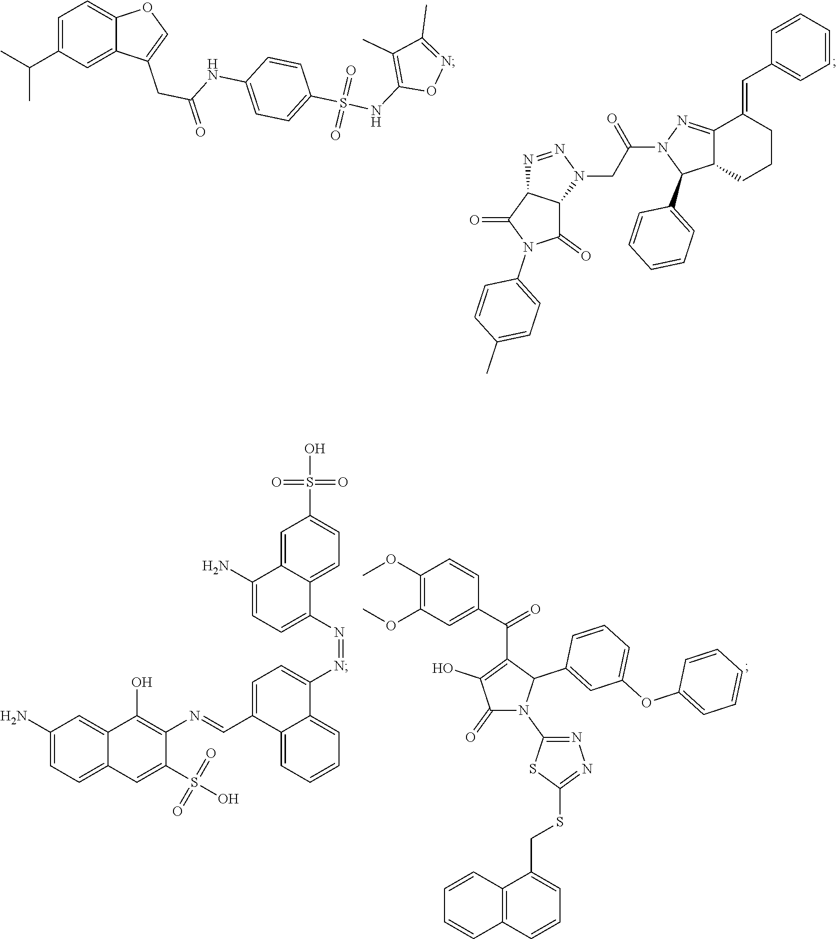

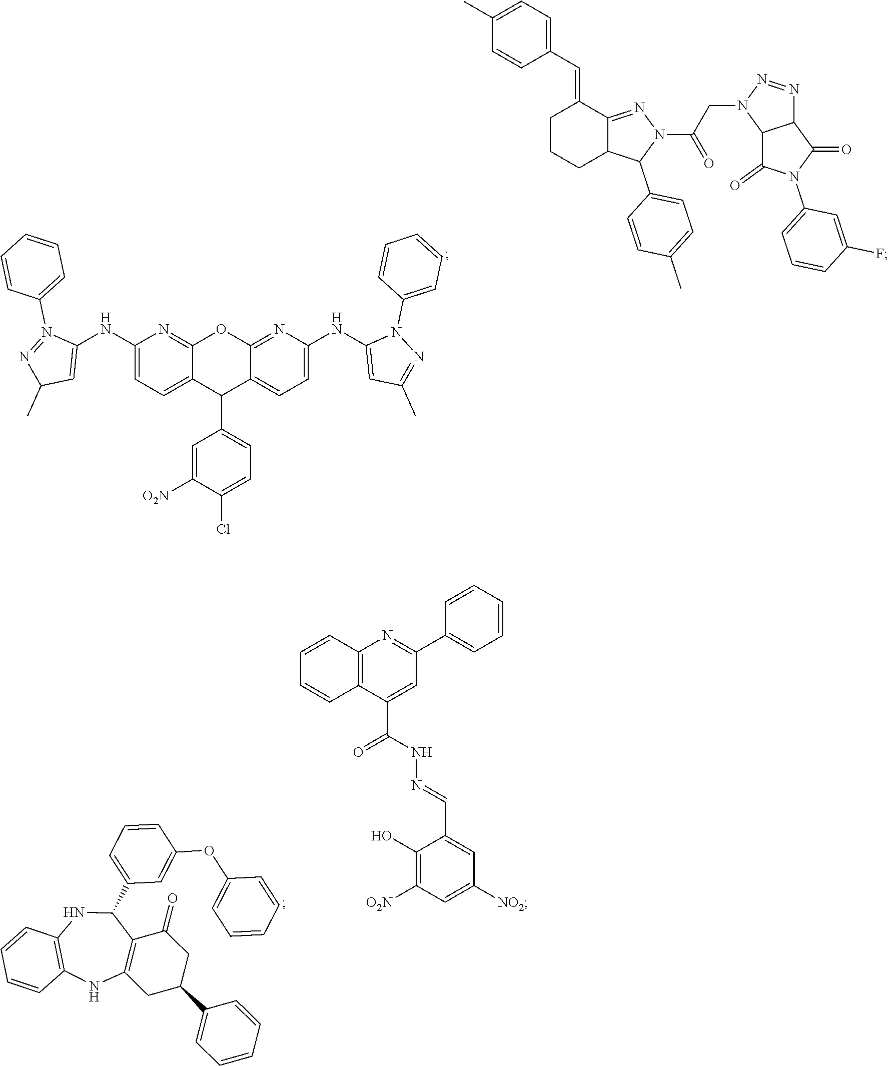

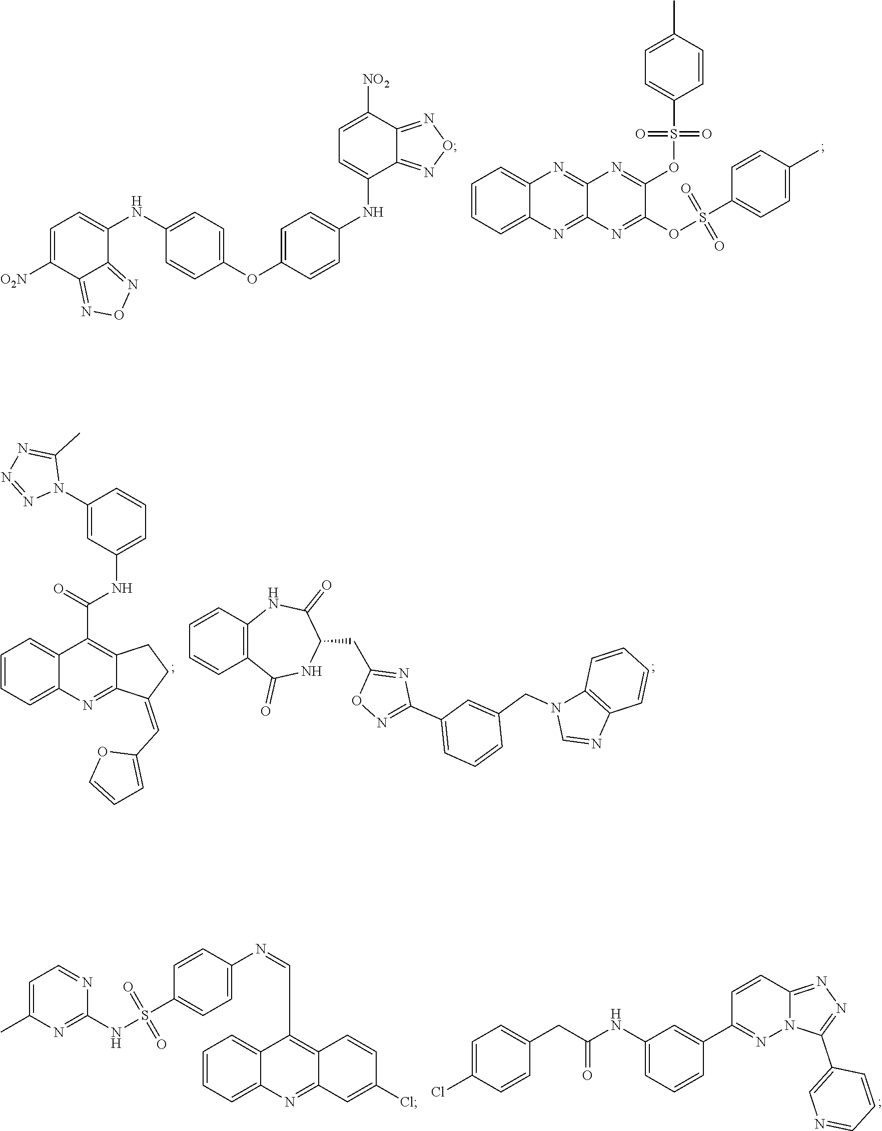

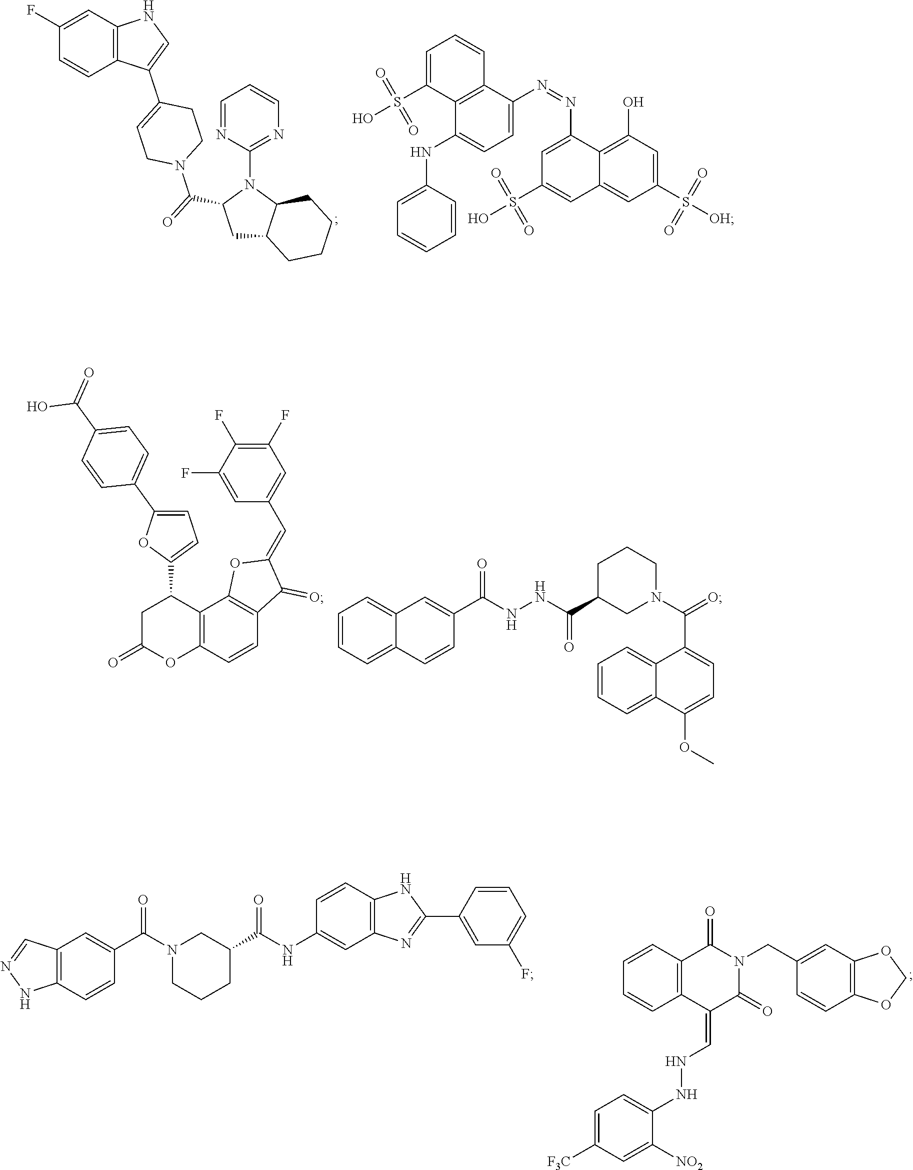

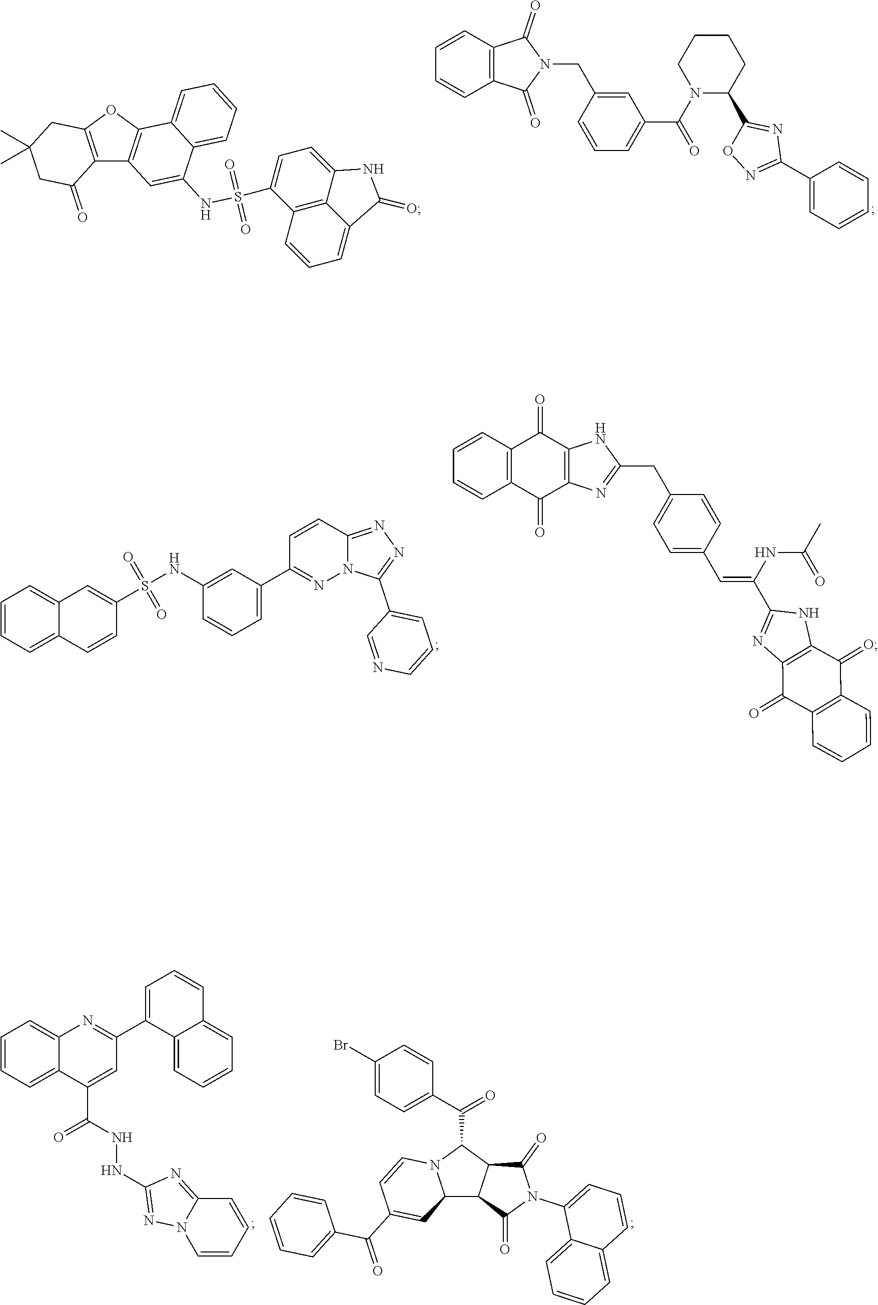

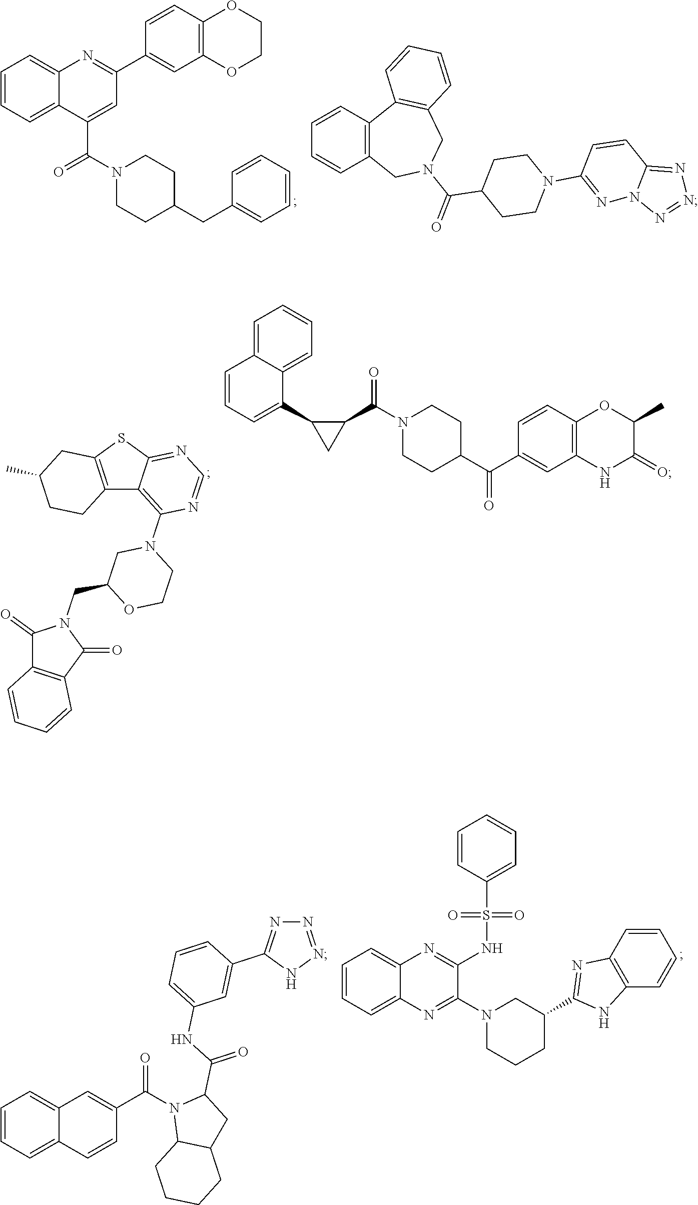

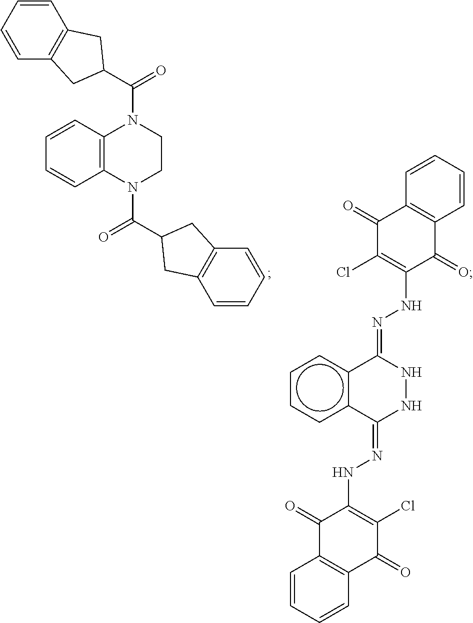

or a pharmaceutically acceptable salt, solvate, hydrate, polymorph, co-crystal, tautomer, stereoisomer, isotopically labeled derivative, or prodrug thereof.

Embodiment 33: The method of any of Embodiments 1-25, wherein the compound is of formula:

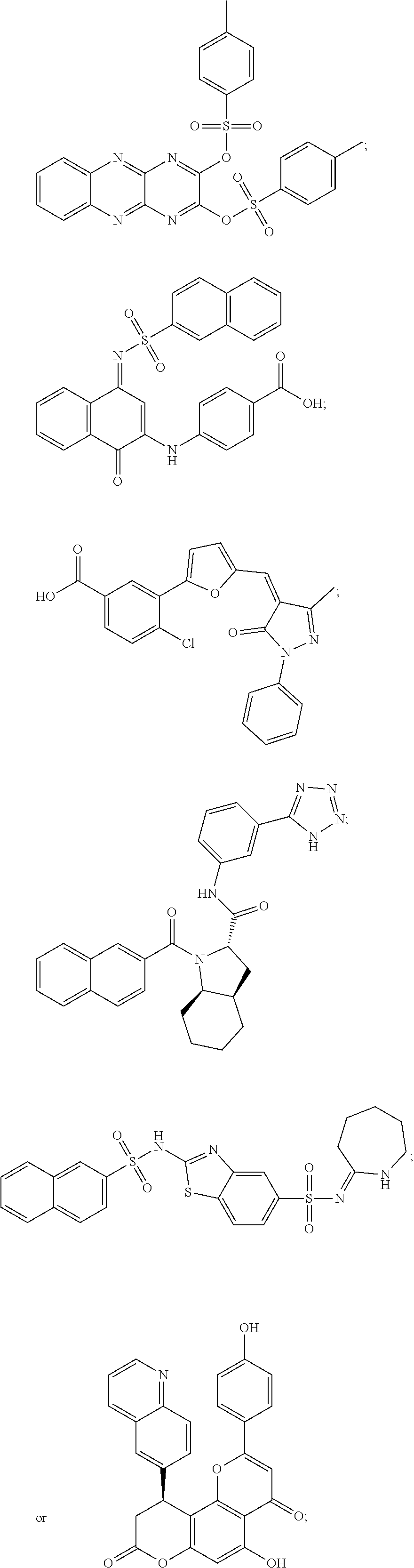 [SMILES: Cc1ccc(S(=O)(=O)Oc2nc3nc4ccccc4nc3nc2OS(=O)(=O)c2ccc(C)cc2)cc1 ; O=C(O)c1ccc(NC2=C/C(=N\S(=O)(=O)c3ccc4ccccc4c3)c3ccccc3C2=O)cc1 ; CC1=NN(c2ccccc2)C(=O)/C1=C\c1ccc(-c2cc(C(=O)O)ccc2Cl)o1 ; O=C(Nc1cccc(-c2nnn[nH]2)c1)[C@@H]1C[C@H]2CCCC[C@H]2N1C(=O)c1ccc2ccccc2c1 ; O=S(=O)(/N=C1\CCCCCN1)c1ccc2sc(NS(=O)(=O)c3ccc4ccccc4c3)nc2c1 ; O=C1C[C@@H](c2ccc3ncccc3c2)c2c(cc(O)c3c(=O)cc(-c4ccc(O)cc4)oc23)O1]

or a pharmaceutically acceptable salt, solvate, hydrate, polymorph, co-crystal, tautomer, stereoisomer, isotopically labeled derivative, or prodrug thereof.

Embodiment 34: A method of inhibiting KEAP1, the method comprising contacting KEAP1 with a compound of the formula:

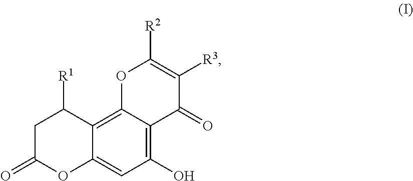

or a pharmaceutically acceptable salt, solvate, hydrate, polymorph, co-crystal, tautomer, stereoisomer, isotopically labeled derivative, or prodrug thereof, wherein:
R¹ is substituted or unsubstituted aryl, substituted or unsubstituted heteroaryl, or substituted or unsubstituted heterocyclyl;
R² is substituted or unsubstituted aryl, substituted or unsubstituted heteroaryl, or substituted or unsubstituted heterocyclyl; and
R³ is hydrogen, —OR^{A}, —SR^{A}, or —N(R^{A})₂;
each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl; or a compound of Formula (II):

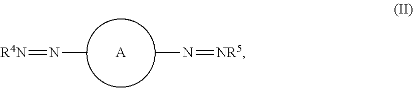

or a pharmaceutically acceptable salt, solvate, hydrate, polymorph, co-crystal, tautomer, stereoisomer, isotopically labeled derivative, or prodrug thereof, wherein:
A is substituted or unsubstituted aryl, substituted or unsubstituted biaryl, or substituted or unsubstituted heteroaryl;
R⁴ is substituted or unsubstituted aryl, substituted or unsubstituted heteroaryl, or substituted or unsubstituted heterocyclyl; and
R⁵ is substituted or unsubstituted aryl, substituted or unsubstituted heteroaryl, or substituted or unsubstituted heterocyclyl; or a compound of Formula (III):

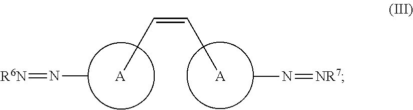

or a pharmaceutically acceptable salt, solvate, hydrate, polymorph, co-crystal, tautomer, stereoisomer, isotopically labeled derivative, or prodrug thereof, wherein:
each A is independently substituted or unsubstituted aryl, or substituted or unsubstituted heteroaryl;
R⁶ is substituted or unsubstituted aryl, substituted or unsubstituted heteroaryl, or substituted or unsubstituted heterocyclyl; and
R⁷ is substituted or unsubstituted aryl, substituted or unsubstituted heteroaryl, or substituted or unsubstituted heterocyclyl; or
   a compound selected from the following:

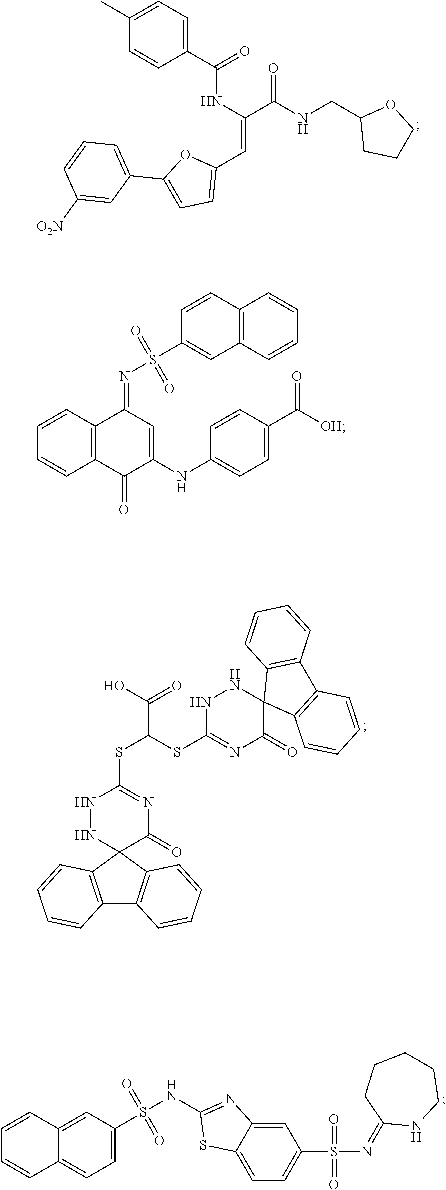 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1]

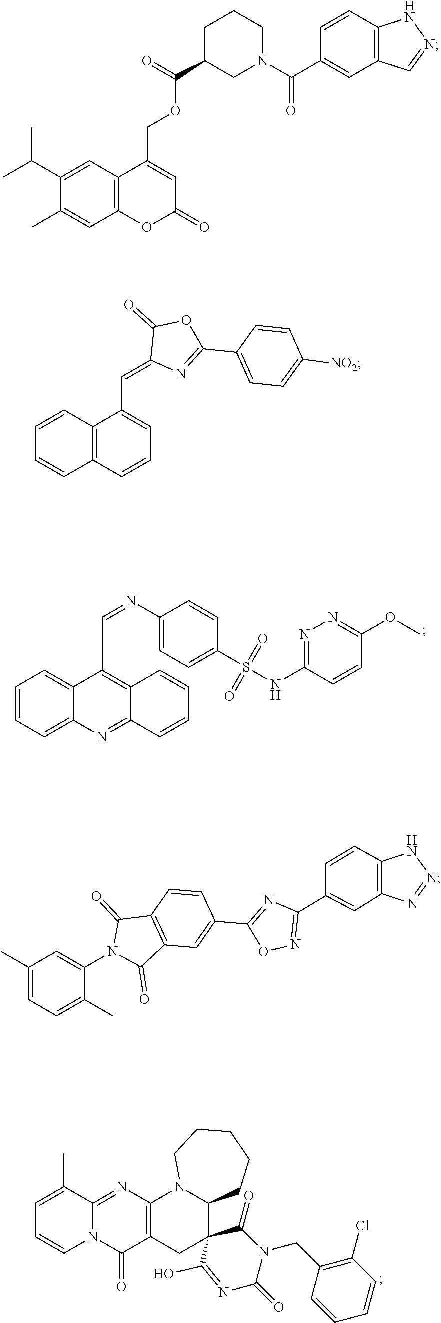

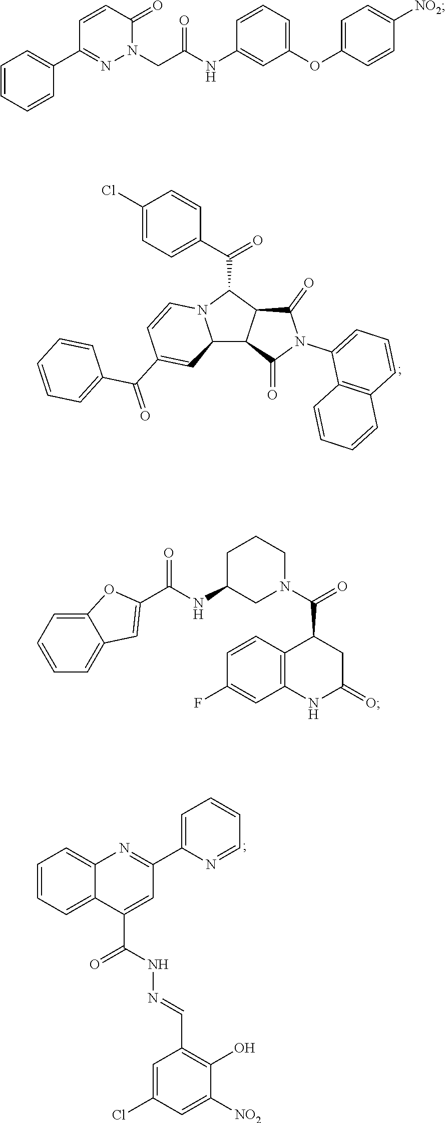 [SMILES: O=C(Cn1nc(-c2ccccc2)ccc1=O)Nc1cccc(Oc2ccc([N+](=O)[O-])cc2)c1 ; O=C(C1=C[C@@H]2[C@@H]3C(=O)N(c4cccc5ccccc45)C(=O)[C@@H]3[C@@H](C(=O)c3ccc(Cl)cc3)N2C=C1)c1ccccc1 ; O=C1C[C@H](C(=O)N2CCC[C@H](NC(=O)c3cc4ccccc4o3)C2)c2ccc(F)cc2N1 ; O=C(N/N=C/c1cc(Cl)cc([N+](=O)[O-])c1O)c1cc(-c2ccccn2)nc2ccccc12]

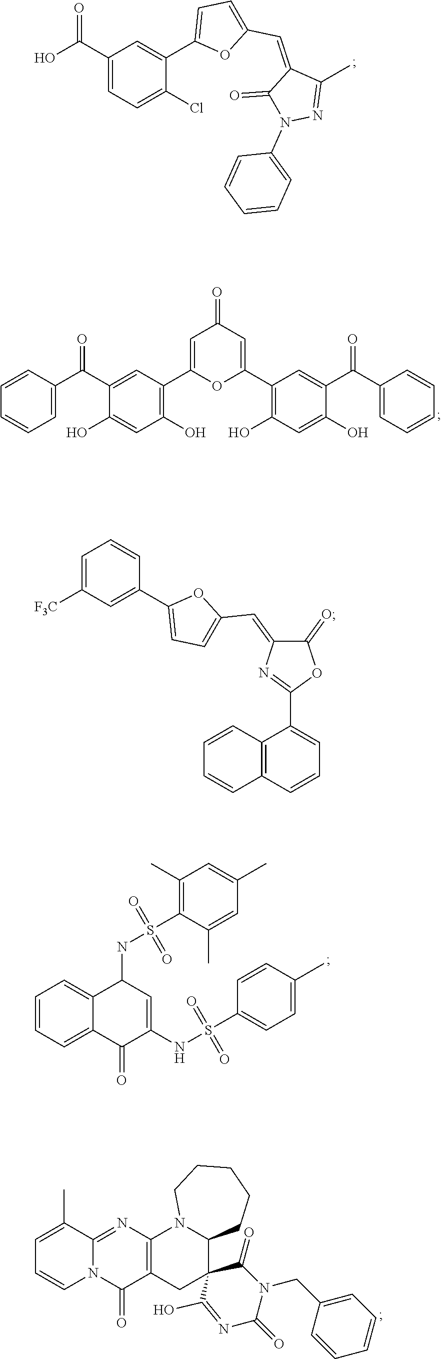 [SMILES: CC1=NN(c2ccccc2)C(=O)/C1=C\c1ccc(-c2cc(C(=O)O)ccc2Cl)o1 ; O=C(c1ccccc1)c1cc(-c2cc(=O)cc(-c3cc(C(=O)c4ccccc4)c(O)cc3O)o2)c(O)cc1O ; O=C1OC(c2cccc3ccccc23)=N/C1=C\c1ccc(-c2cccc(C(F)(F)F)c2)o1 ; Cc1ccc(S(=O)(=O)NC2=CC(NS(=O)(=O)c3c(C)cc(C)cc3C)c3ccccc3C2=O)cc1 ; Cc1cccn2c(=O)c3c(nc12)N1CCCCC[C@H]1[C@]1(C3)C(=O)N(Cc2ccccc2)C(=O)N=C1O]

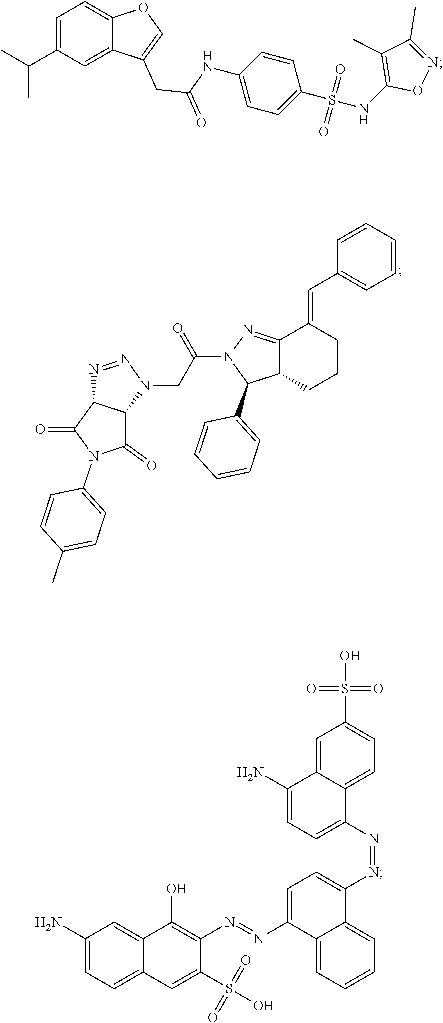 [SMILES: Cc1noc(NS(=O)(=O)c2ccc(NC(=O)Cc3coc4ccc(C(C)C)cc34)cc2)c1C ; Cc1ccc(N2C(=O)[C@@H]3[C@@H](N=NN3CC(=O)N3N=C4/C(=C/c5ccccc5)CCC[C@@H]4[C@H]3c3ccccc3)C2=O)cc1 ; Nc1ccc2cc(S(=O)(=O)O)c(/N=N/c3ccc(/N=N\c4ccc(N)c5cc(S(=O)(=O)O)ccc45)c4ccccc34)c(O)c2c1]

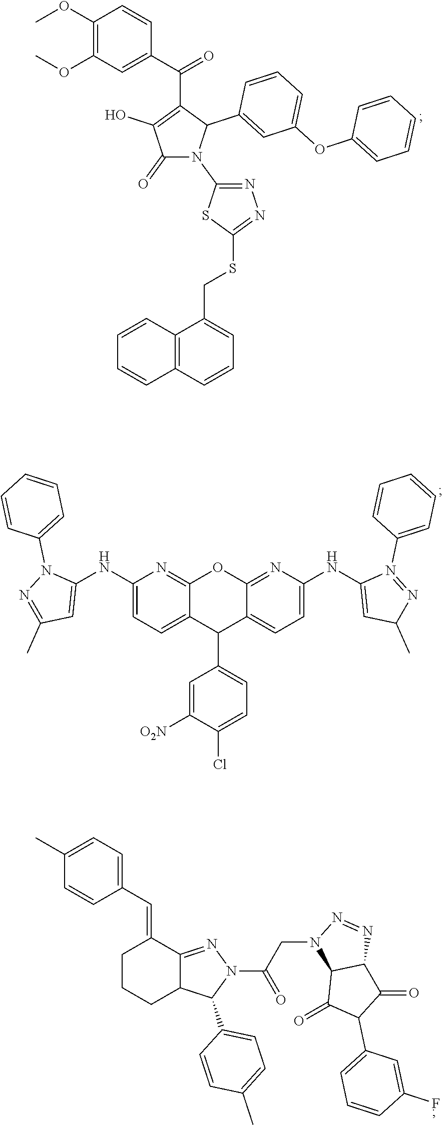

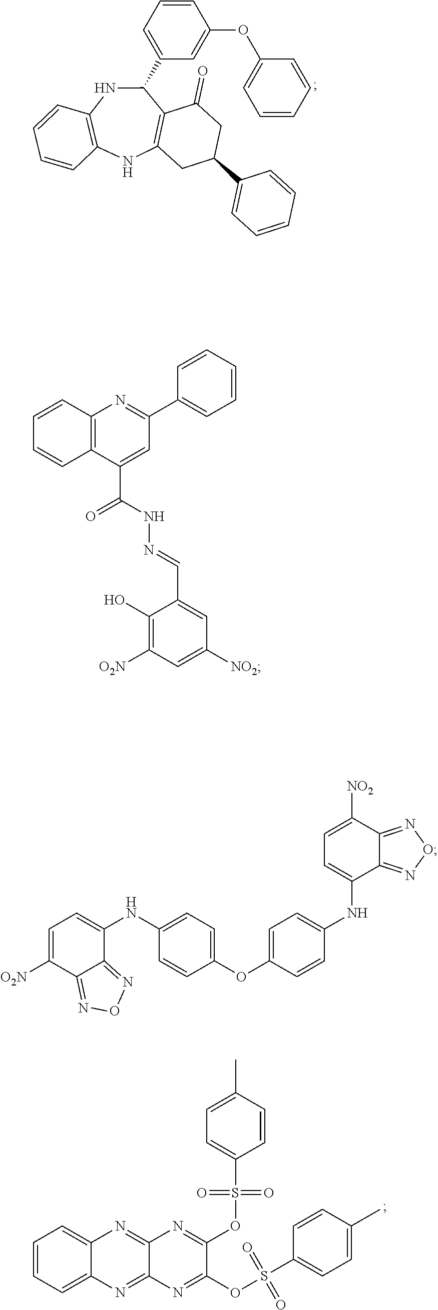 [SMILES: O=C1C[C@@H](c2ccccc2)CC2=C1[C@@H](c1cccc(Oc3ccccc3)c1)Nc1ccccc1N2 ; O=C(N/N=C/c1cc([N+](=O)[O-])cc([N+](=O)[O-])c1O)c1cc(-c2ccccc2)nc2ccccc12 ; O=[N+]([O-])c1ccc(Nc2ccc(Oc3ccc(Nc4ccc([N+](=O)[O-])c5nonc45)cc3)cc2)c2nonc12 ; Cc1ccc(S(=O)(=O)Oc2nc3nc4ccccc4nc3nc2OS(=O)(=O)c2ccc(C)cc2)cc1]

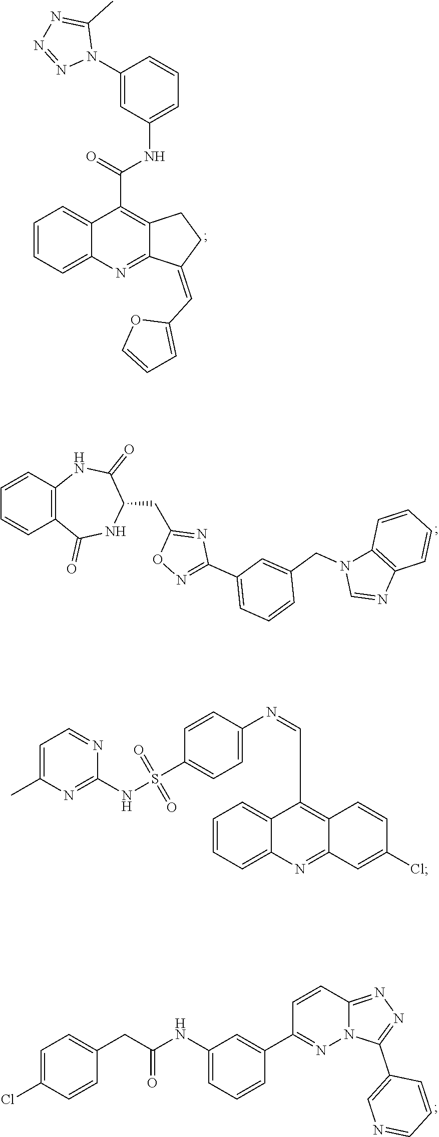 [SMILES: Cc1nnnn1-c1cccc(NC(=O)c2c3c(nc4ccccc24)/C(=C\c2ccco2)CC3)c1 ; O=C1N[C@@H](Cc2nc(-c3cccc(Cn4cnc5ccccc54)c3)no2)C(=O)Nc2ccccc21 ; Cc1ccnc(NS(=O)(=O)c2ccc(/N=C\c3c4ccccc4nc4cc(Cl)ccc34)cc2)n1 ; O=C(Cc1ccc(Cl)cc1)Nc1cccc(-c2ccc3nnc(-c4cccnc4)n3n2)c1]

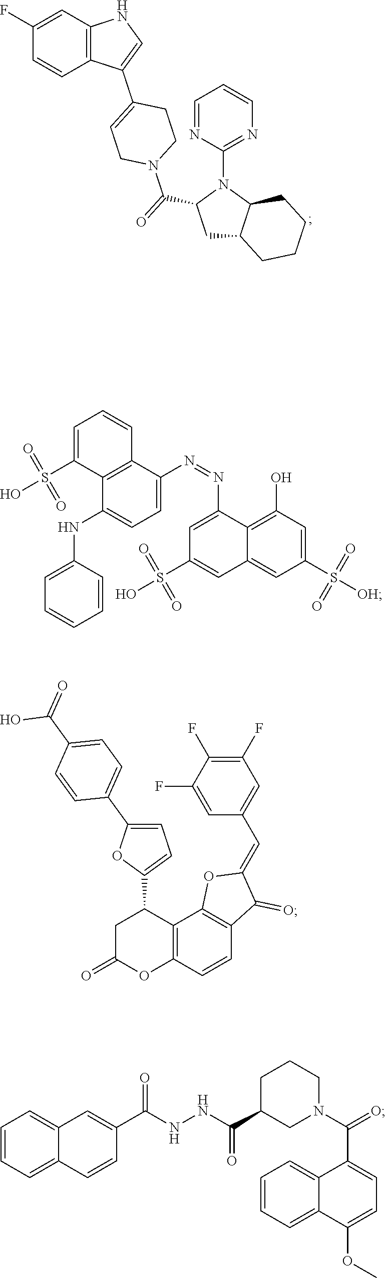 [SMILES: O=C([C@H]1C[C@H]2CCCC[C@@H]2N1c1ncccn1)N1CC=C(c2c[nH]c3cc(F)ccc23)CC1 ; O=S(=O)(O)c1cc(O)c2c(/N=N\c3ccc(Nc4ccccc4)c4c(S(=O)(=O)O)cccc34)cc(S(=O)(=O)O)cc2c1 ; O=C1C[C@H](c2ccc(-c3ccc(C(=O)O)cc3)o2)c2c(ccc3c2O/C(=C\c2cc(F)c(F)c(F)c2)C3=O)O1 ; COc1ccc(C(=O)N2CCC[C@H](C(=O)NNC(=O)c3ccc4ccccc4c3)C2)c2ccccc12]

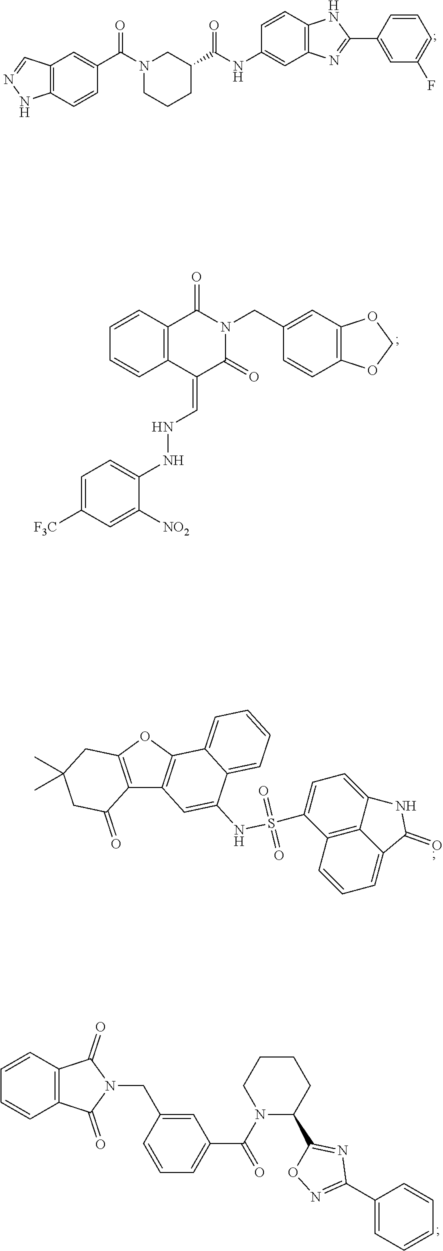 [SMILES: O=C(Nc1ccc2[nH]c(-c3cccc(F)c3)nc2c1)[C@@H]1CCCN(C(=O)c2ccc3[nH]ncc3c2)C1 ; O=C1/C(=C/NNc2ccc(C(F)(F)F)cc2[N+](=O)[O-])c2ccccc2C(=O)N1Cc1ccc2c(c1)OCO2 ; CC1(C)CC(=O)c2c(oc3c2cc(NS(=O)(=O)c2ccc4c5c(cccc25)C(=O)N4)c2ccccc23)C1 ; O=C1c2ccccc2C(=O)N1Cc1cccc(C(=O)N2CCCC[C@H]2c2nc(-c3ccccc3)no2)c1]

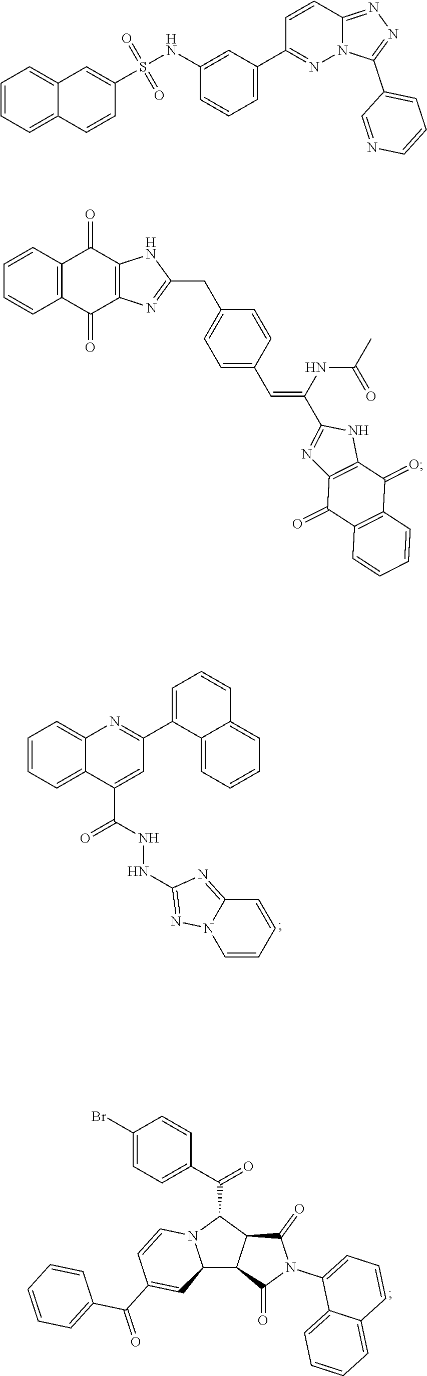 [SMILES: O=S(=O)(Nc1cccc(-c2ccc3nnc(-c4cccnc4)n3n2)c1)c1ccc2ccccc2c1 ; CC(=O)N/C(=C\c1ccc(Cc2nc3c([nH]2)C(=O)c2ccccc2C3=O)cc1)c1nc2c([nH]1)C(=O)c1ccccc1C2=O ; O=C(NNc1nc2ccccn2n1)c1cc(-c2cccc3ccccc23)nc2ccccc12 ; O=C(C1=C[C@@H]2[C@@H]3C(=O)N(c4cccc5ccccc45)C(=O)[C@@H]3[C@@H](C(=O)c3ccc(Br)cc3)N2C=C1)c1ccccc1]

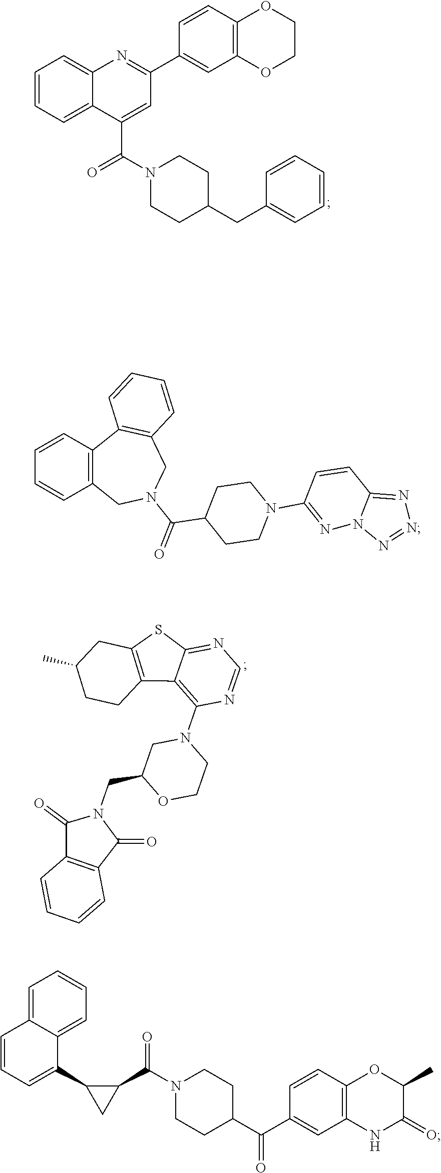 [SMILES: O=C(c1cc(-c2ccc3c(c2)OCCO3)nc2ccccc12)N1CCC(Cc2ccccc2)CC1 ; O=C(C1CCN(c2ccc3nnnn3n2)CC1)N1Cc2ccccc2-c2ccccc2C1 ; C[C@H]1CCc2c(sc3ncnc(N4CCO[C@@H](CN5C(=O)c6ccccc6C5=O)C4)c23)C1 ; C[C@@H]1Oc2ccc(C(=O)C3CCN(C(=O)[C@H]4C[C@H]4c4cccc5ccccc45)CC3)cc2NC1=O]

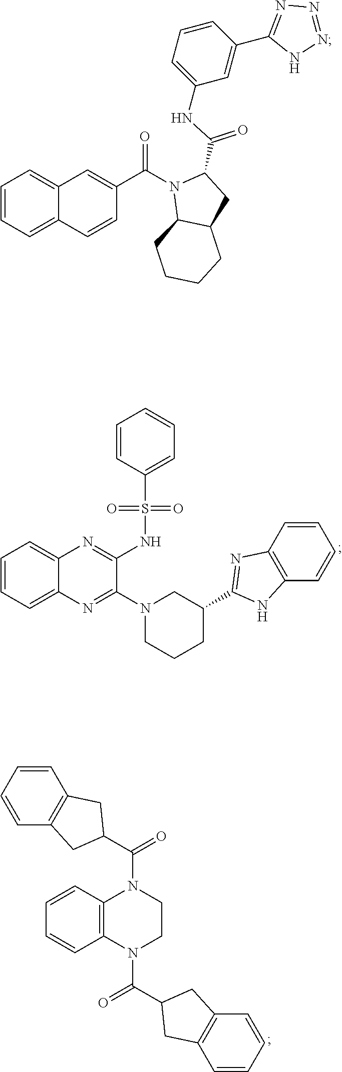 [SMILES: O=C(Nc1cccc(-c2nnn[nH]2)c1)[C@@H]1C[C@H]2CCCC[C@H]2N1C(=O)c1ccc2ccccc2c1 ; O=S(=O)(Nc1nc2ccccc2nc1N1CCC[C@@H](c2nc3ccccc3[nH]2)C1)c1ccccc1 ; O=C(C1Cc2ccccc2C1)N1CCN(C(=O)C2Cc3ccccc3C2)c2ccccc21]

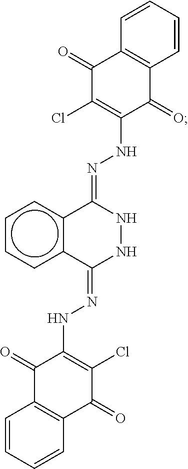

or a pharmaceutically acceptable salt, solvate, hydrate, polymorph, co-crystal, tautomer, stereoisomer, isotopically labeled derivative, or prodrug thereof.

Embodiment 35: A method of disrupting the interaction between KEAP1 and Nrf2, the method comprising contacting KEAP1 with a compound of Formula (I):

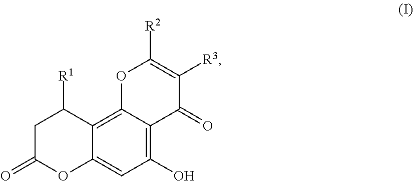

or a pharmaceutically acceptable salt, solvate, hydrate, polymorph, co-crystal, tautomer, stereoisomer, isotopically labeled derivative, or prodrug thereof, wherein:
R¹ is substituted or unsubstituted aryl, substituted or unsubstituted heteroaryl, or substituted or unsubstituted heterocyclyl;
R² is substituted or unsubstituted aryl, substituted or unsubstituted heteroaryl, or substituted or unsubstituted heterocyclyl; and
R³ is hydrogen, —OR^{A}, —SR^{A}, or —N(R^{A})₂;
each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl; or
   a compound of Formula (II):

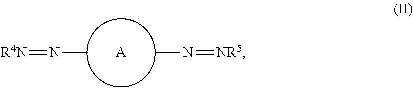

or a pharmaceutically acceptable salt, solvate, hydrate, polymorph, co-crystal, tautomer, stereoisomer, isotopically labeled derivative, or prodrug thereof, wherein:
A is substituted or unsubstituted aryl, substituted or unsubstituted biaryl, or substituted or unsubstituted heteroaryl;
R⁴ is substituted or unsubstituted aryl, substituted or unsubstituted heteroaryl, or substituted or unsubstituted heterocyclyl; and
R⁵ is substituted or unsubstituted aryl, substituted or unsubstituted heteroaryl, or substituted or unsubstituted heterocyclyl; or
   a compound of Formula (III)

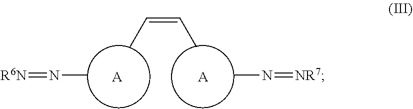

or a pharmaceutically acceptable salt, solvate, hydrate, polymorph, co-crystal, tautomer, stereoisomer, isotopically labeled derivative, or prodrug thereof, wherein:
each A is independently substituted or unsubstituted aryl, or substituted or unsubstituted heteroaryl;
R⁶ is substituted or unsubstituted aryl, substituted or unsubstituted heteroaryl, or substituted or unsubstituted heterocyclyl; and
R⁷ is substituted or unsubstituted aryl, substituted or unsubstituted heteroaryl, or substituted or unsubstituted heterocyclyl; or
   a compound selected from the following:

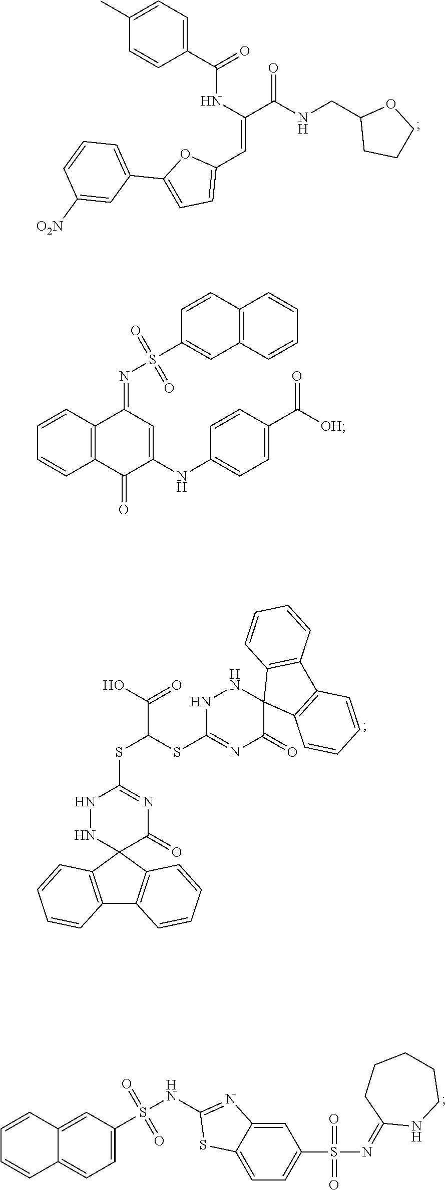 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1]

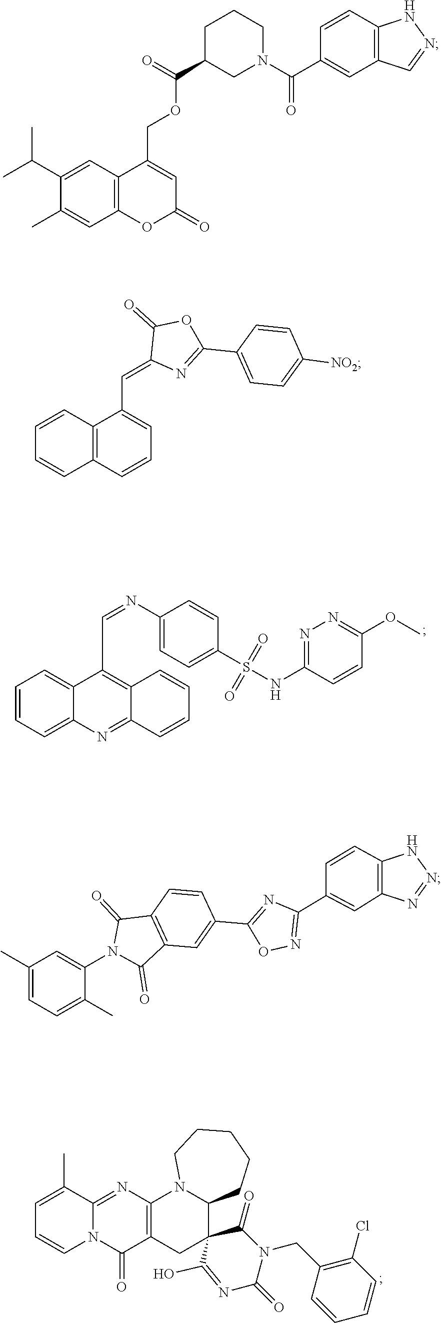

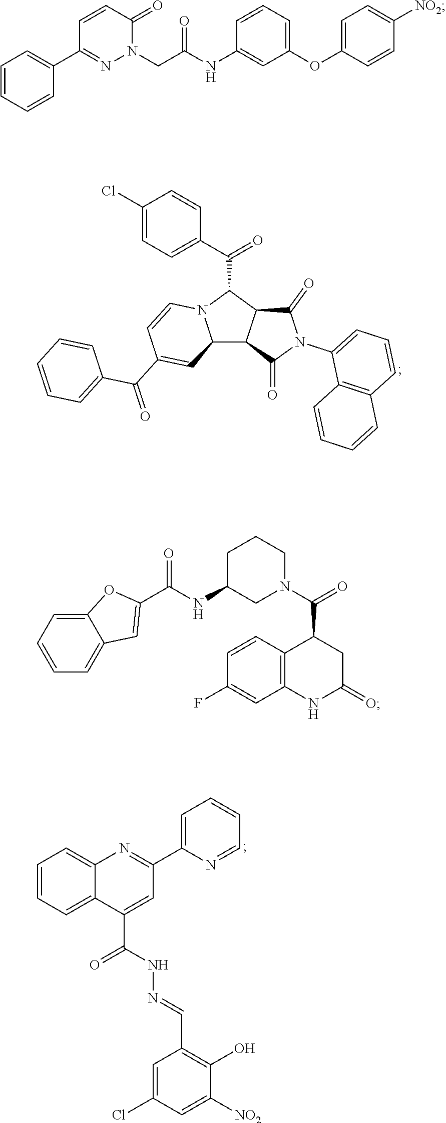 [SMILES: O=C(Cn1nc(-c2ccccc2)ccc1=O)Nc1cccc(Oc2ccc([N+](=O)[O-])cc2)c1 ; O=C(C1=C[C@@H]2[C@@H]3C(=O)N(c4cccc5ccccc45)C(=O)[C@@H]3[C@@H](C(=O)c3ccc(Cl)cc3)N2C=C1)c1ccccc1 ; O=C1C[C@H](C(=O)N2CCC[C@H](NC(=O)c3cc4ccccc4o3)C2)c2ccc(F)cc2N1 ; O=C(N/N=C/c1cc(Cl)cc([N+](=O)[O-])c1O)c1cc(-c2ccccn2)nc2ccccc12]

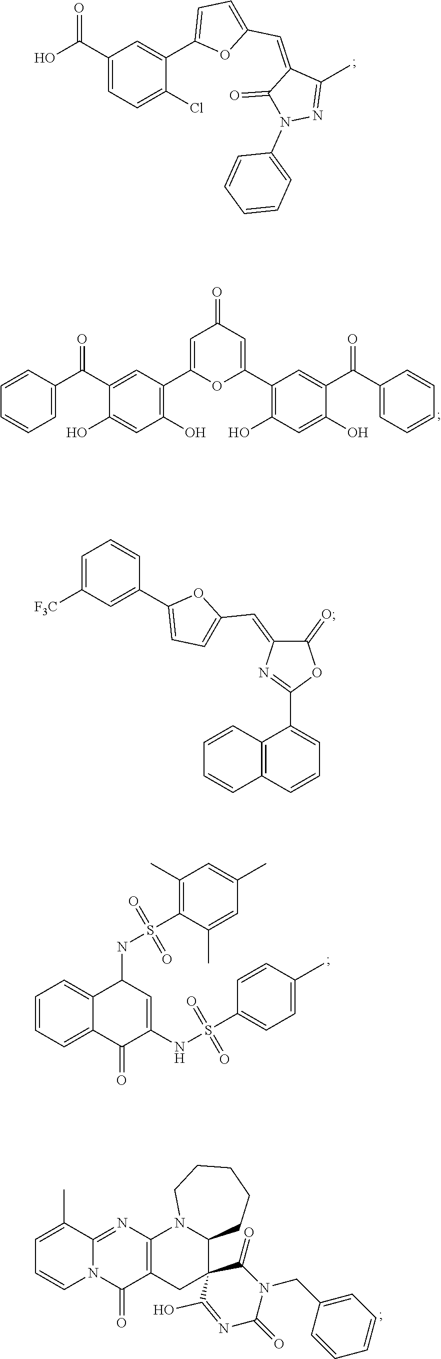 [SMILES: CC1=NN(c2ccccc2)C(=O)/C1=C\c1ccc(-c2cc(C(=O)O)ccc2Cl)o1 ; O=C(c1ccccc1)c1cc(-c2cc(=O)cc(-c3cc(C(=O)c4ccccc4)c(O)cc3O)o2)c(O)cc1O ; O=C1OC(c2cccc3ccccc23)=N/C1=C\c1ccc(-c2cccc(C(F)(F)F)c2)o1 ; Cc1ccc(S(=O)(=O)NC2=CC(NS(=O)(=O)c3c(C)cc(C)cc3C)c3ccccc3C2=O)cc1 ; Cc1cccn2c(=O)c3c(nc12)N1CCCCC[C@H]1[C@]1(C3)C(=O)N(Cc2ccccc2)C(=O)N=C1O]

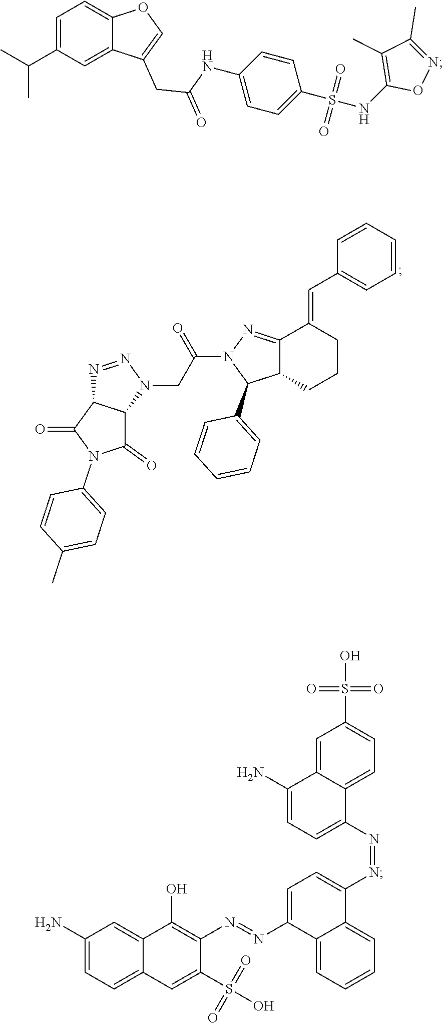 [SMILES: Cc1noc(NS(=O)(=O)c2ccc(NC(=O)Cc3coc4ccc(C(C)C)cc34)cc2)c1C ; Cc1ccc(N2C(=O)[C@@H]3[C@@H](N=NN3CC(=O)N3N=C4/C(=C/c5ccccc5)CCC[C@@H]4[C@H]3c3ccccc3)C2=O)cc1 ; Nc1ccc2cc(S(=O)(=O)O)c(/N=N/c3ccc(/N=N\c4ccc(N)c5cc(S(=O)(=O)O)ccc45)c4ccccc34)c(O)c2c1]

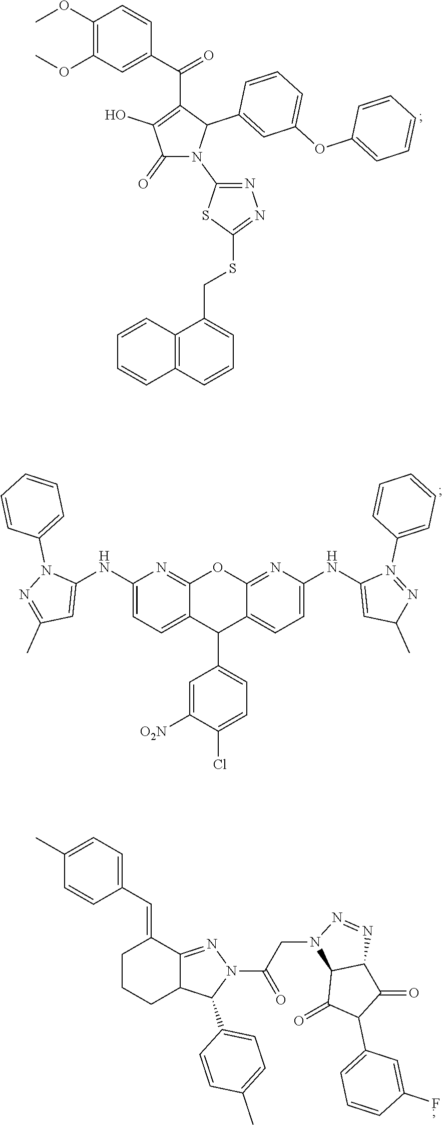

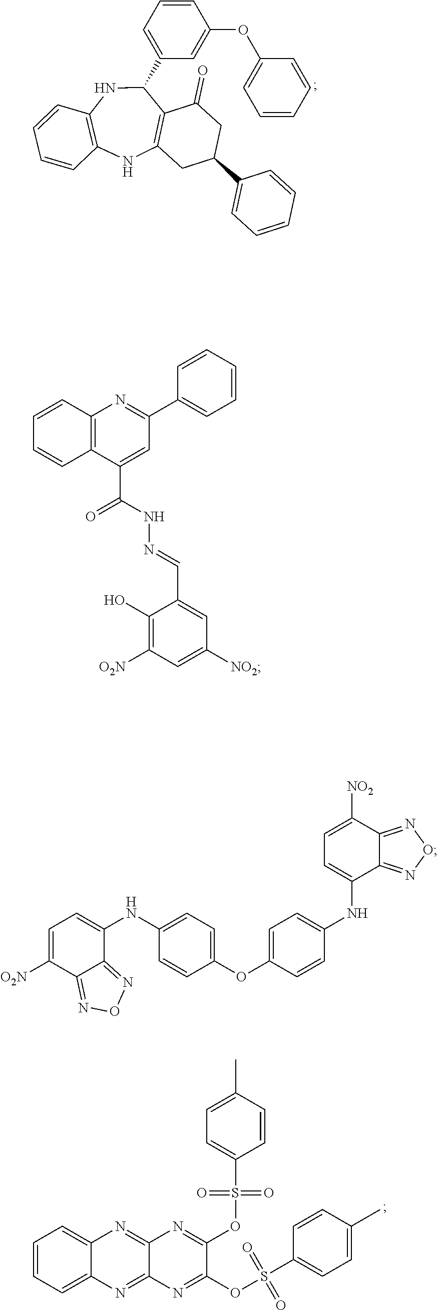 [SMILES: O=C1C[C@@H](c2ccccc2)CC2=C1[C@@H](c1cccc(Oc3ccccc3)c1)Nc1ccccc1N2 ; O=C(N/N=C/c1cc([N+](=O)[O-])cc([N+](=O)[O-])c1O)c1cc(-c2ccccc2)nc2ccccc12 ; O=[N+]([O-])c1ccc(Nc2ccc(Oc3ccc(Nc4ccc([N+](=O)[O-])c5nonc45)cc3)cc2)c2nonc12 ; Cc1ccc(S(=O)(=O)Oc2nc3nc4ccccc4nc3nc2OS(=O)(=O)c2ccc(C)cc2)cc1]

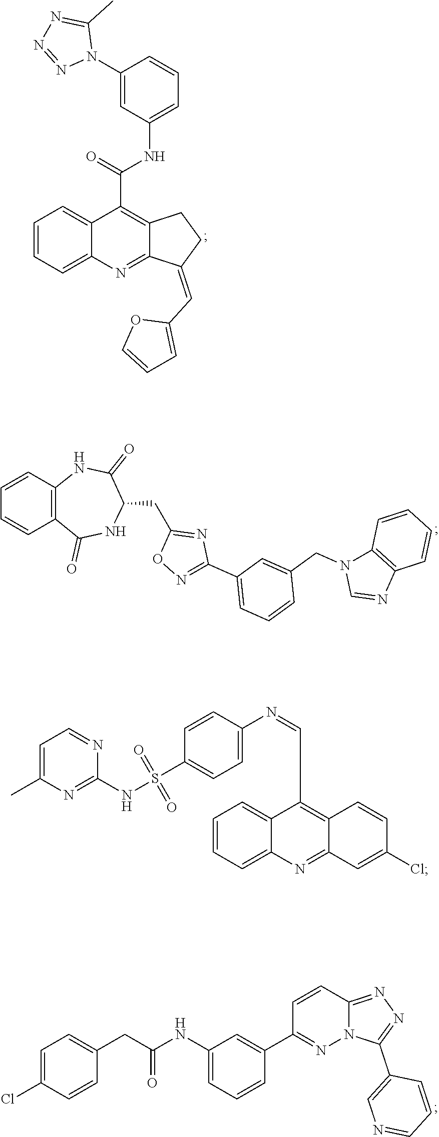 [SMILES: Cc1nnnn1-c1cccc(NC(=O)c2c3c(nc4ccccc24)/C(=C\c2ccco2)CC3)c1 ; O=C1N[C@@H](Cc2nc(-c3cccc(Cn4cnc5ccccc54)c3)no2)C(=O)Nc2ccccc21 ; Cc1ccnc(NS(=O)(=O)c2ccc(/N=C\c3c4ccccc4nc4cc(Cl)ccc34)cc2)n1 ; O=C(Cc1ccc(Cl)cc1)Nc1cccc(-c2ccc3nnc(-c4cccnc4)n3n2)c1]

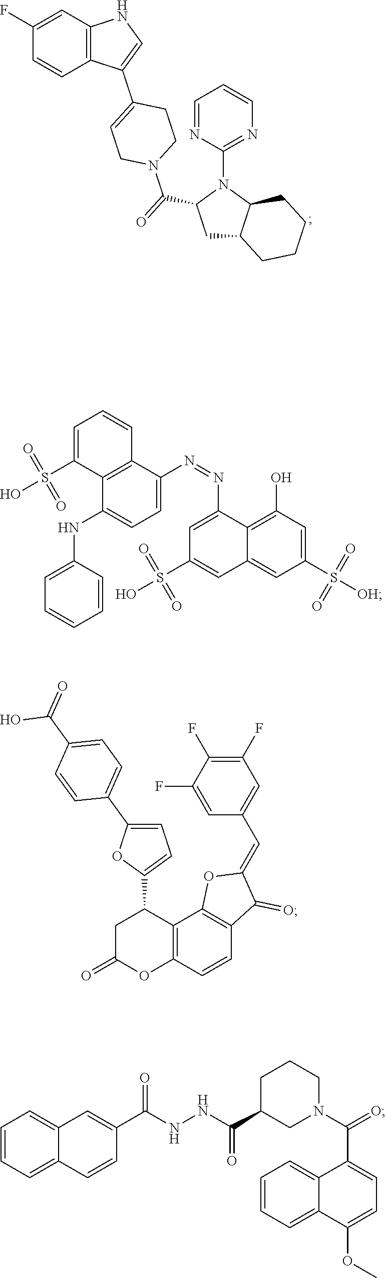 [SMILES: O=C([C@H]1C[C@H]2CCCC[C@@H]2N1c1ncccn1)N1CC=C(c2c[nH]c3cc(F)ccc23)CC1 ; O=S(=O)(O)c1cc(O)c2c(/N=N\c3ccc(Nc4ccccc4)c4c(S(=O)(=O)O)cccc34)cc(S(=O)(=O)O)cc2c1 ; O=C1C[C@H](c2ccc(-c3ccc(C(=O)O)cc3)o2)c2c(ccc3c2O/C(=C\c2cc(F)c(F)c(F)c2)C3=O)O1 ; COc1ccc(C(=O)N2CCC[C@H](C(=O)NNC(=O)c3ccc4ccccc4c3)C2)c2ccccc12]

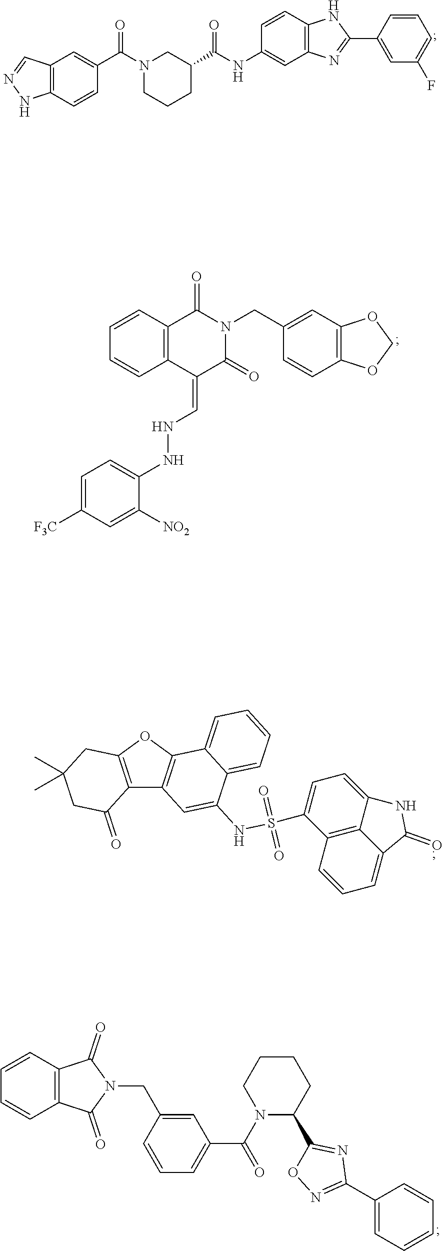 [SMILES: O=C(Nc1ccc2[nH]c(-c3cccc(F)c3)nc2c1)[C@@H]1CCCN(C(=O)c2ccc3[nH]ncc3c2)C1 ; O=C1/C(=C/NNc2ccc(C(F)(F)F)cc2[N+](=O)[O-])c2ccccc2C(=O)N1Cc1ccc2c(c1)OCO2 ; CC1(C)CC(=O)c2c(oc3c2cc(NS(=O)(=O)c2ccc4c5c(cccc25)C(=O)N4)c2ccccc23)C1 ; O=C1c2ccccc2C(=O)N1Cc1cccc(C(=O)N2CCCC[C@H]2c2nc(-c3ccccc3)no2)c1]

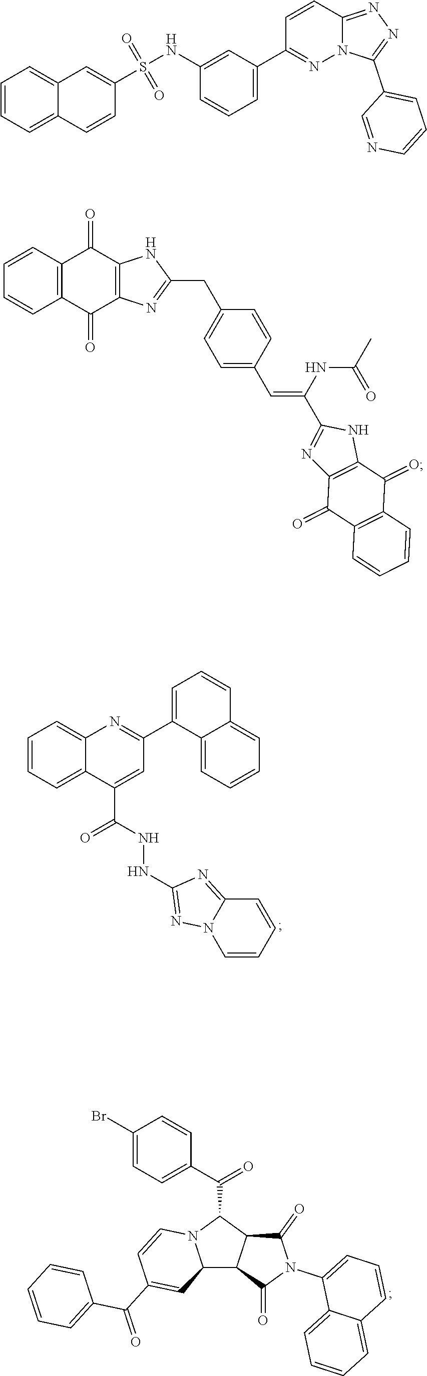 [SMILES: O=S(=O)(Nc1cccc(-c2ccc3nnc(-c4cccnc4)n3n2)c1)c1ccc2ccccc2c1 ; CC(=O)N/C(=C\c1ccc(Cc2nc3c([nH]2)C(=O)c2ccccc2C3=O)cc1)c1nc2c([nH]1)C(=O)c1ccccc1C2=O ; O=C(NNc1nc2ccccn2n1)c1cc(-c2cccc3ccccc23)nc2ccccc12 ; O=C(C1=C[C@@H]2[C@@H]3C(=O)N(c4cccc5ccccc45)C(=O)[C@@H]3[C@@H](C(=O)c3ccc(Br)cc3)N2C=C1)c1ccccc1]

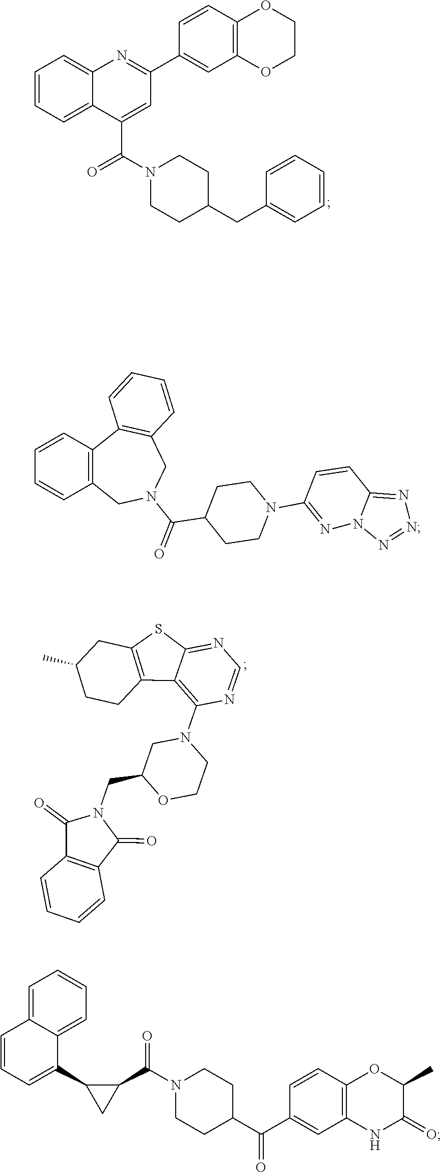 [SMILES: O=C(c1cc(-c2ccc3c(c2)OCCO3)nc2ccccc12)N1CCC(Cc2ccccc2)CC1 ; O=C(C1CCN(c2ccc3nnnn3n2)CC1)N1Cc2ccccc2-c2ccccc2C1 ; C[C@H]1CCc2c(sc3ncnc(N4CCO[C@@H](CN5C(=O)c6ccccc6C5=O)C4)c23)C1 ; C[C@@H]1Oc2ccc(C(=O)C3CCN(C(=O)[C@H]4C[C@H]4c4cccc5ccccc45)CC3)cc2NC1=O]

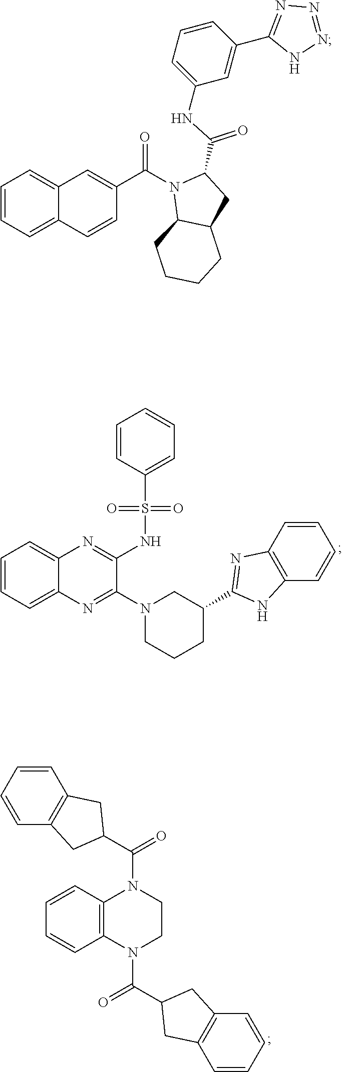 [SMILES: O=C(Nc1cccc(-c2nnn[nH]2)c1)[C@@H]1C[C@H]2CCCC[C@H]2N1C(=O)c1ccc2ccccc2c1 ; O=S(=O)(Nc1nc2ccccc2nc1N1CCC[C@@H](c2nc3ccccc3[nH]2)C1)c1ccccc1 ; O=C(C1Cc2ccccc2C1)N1CCN(C(=O)C2Cc3ccccc3C2)c2ccccc21]

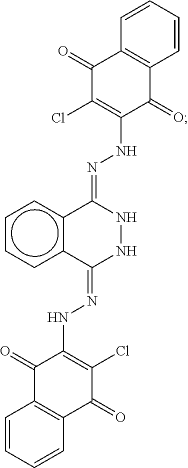

or a pharmaceutically acceptable salt, solvate, hydrate, polymorph, co-crystal, tautomer, stereoisomer, isotopically labeled derivative, or prodrug thereof.

Embodiment 36: A method of preventing or inhibiting the interaction between KEAP1 and Nrf2, the method comprising contacting KEAP1 with a compound of Formula (I):

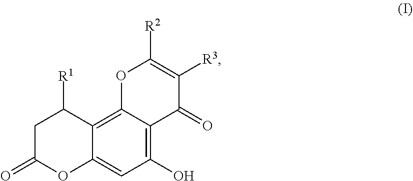

or a pharmaceutically acceptable salt, solvate, hydrate, polymorph, co-crystal, tautomer, stereoisomer, isotopically labeled derivative, or prodrug thereof, wherein:
R¹ is substituted or unsubstituted aryl, substituted or unsubstituted heteroaryl, or substituted or unsubstituted heterocyclyl;
R² is substituted or unsubstituted aryl, substituted or unsubstituted heteroaryl, or substituted or unsubstituted heterocyclyl; and
R³ is hydrogen, —OR^{A}, —SR, or —N(R^{A})₂;
each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl; or
   a compound of Formula (II):

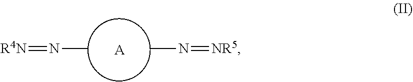

or a pharmaceutically acceptable salt, solvate, hydrate, polymorph, co-crystal, tautomer, stereoisomer, isotopically labeled derivative, or prodrug thereof, wherein:
A is substituted or unsubstituted aryl, substituted or unsubstituted biaryl, or substituted or unsubstituted heteroaryl;
R⁴ is substituted or unsubstituted aryl, substituted or unsubstituted heteroaryl, or substituted or unsubstituted heterocyclyl; and
R⁵ is substituted or unsubstituted aryl, substituted or unsubstituted heteroaryl, or substituted or unsubstituted heterocyclyl; or
   a compound of Formula (III):

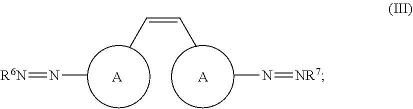

or a pharmaceutically acceptable salt, solvate, hydrate, polymorph, co-crystal, tautomer, stereoisomer, isotopically labeled derivative, or prodrug thereof, wherein:
each A is independently substituted or unsubstituted aryl, or substituted or unsubstituted heteroaryl;
R⁶ is substituted or unsubstituted aryl, substituted or unsubstituted heteroaryl, or substituted or unsubstituted heterocyclyl; and
R⁷ is substituted or unsubstituted aryl, substituted or unsubstituted heteroaryl, or substituted or unsubstituted heterocyclyl; or
   a compound selected from the following:

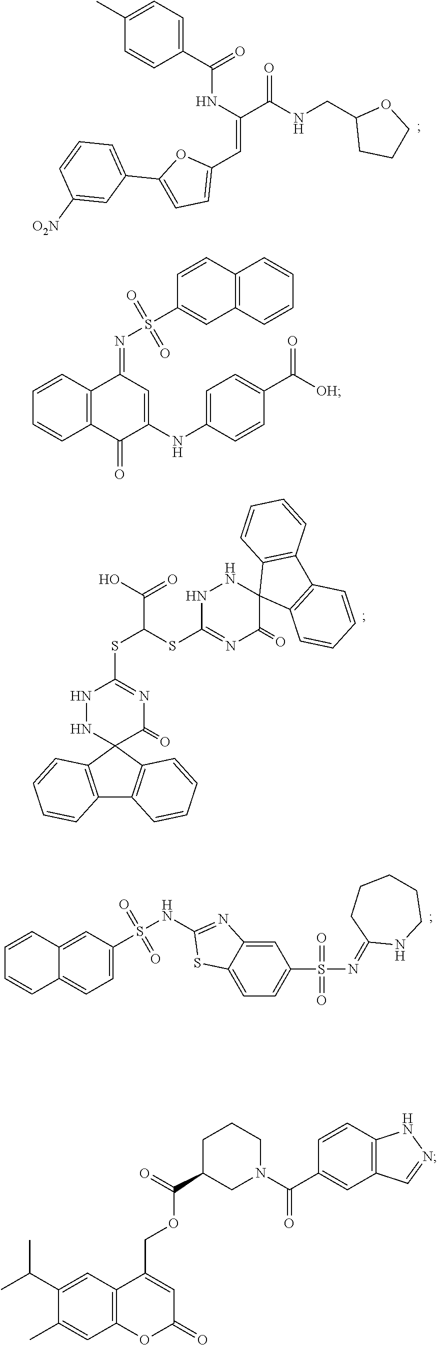 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1 ; Cc1cc2oc(=O)cc(COC(=O)[C@H]3CCCN(C(=O)c4ccc5[nH]ncc5c4)C3)c2cc1C(C)C]

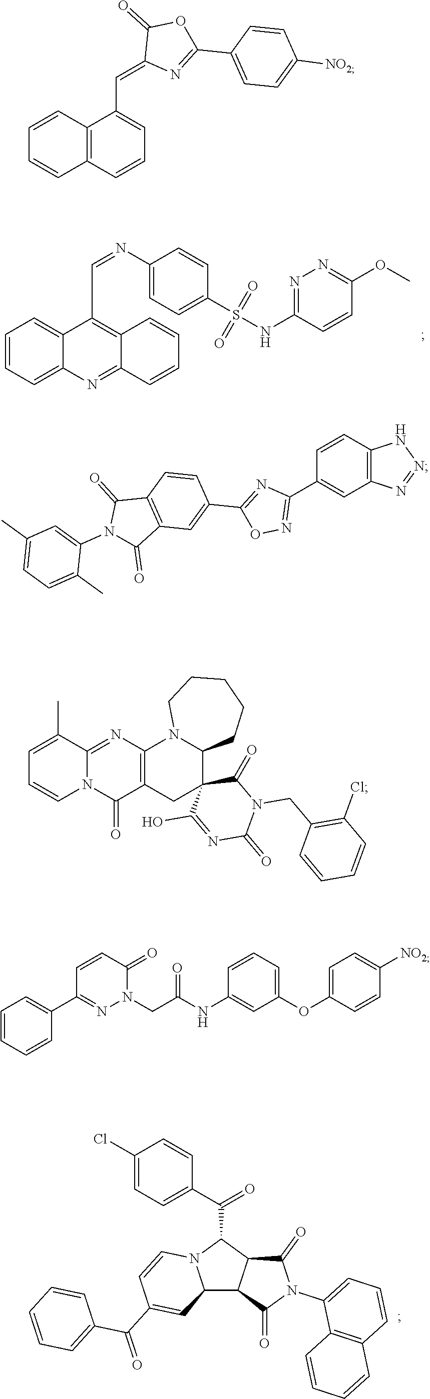

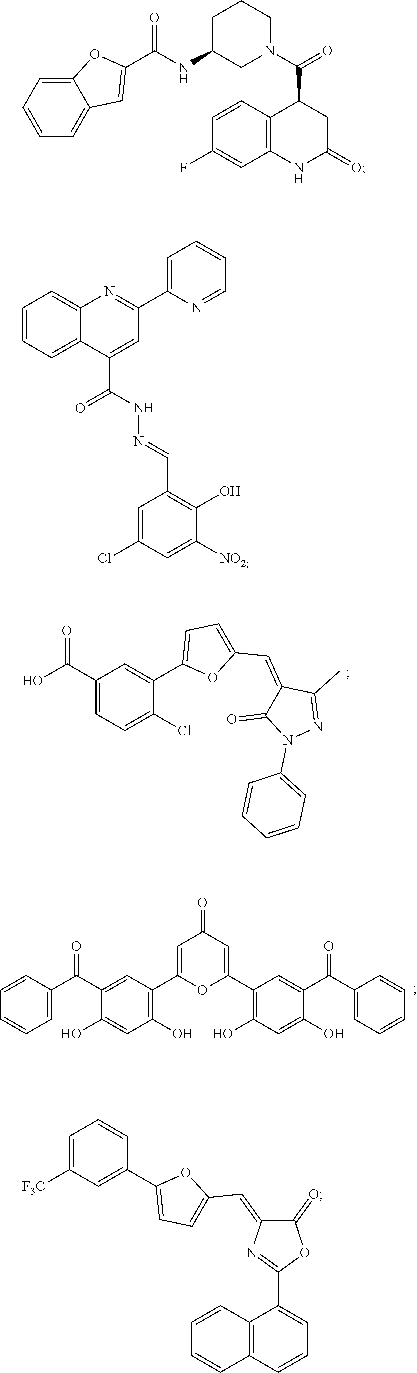 [SMILES: O=C1C[C@H](C(=O)N2CCC[C@H](NC(=O)c3cc4ccccc4o3)C2)c2ccc(F)cc2N1 ; O=C(N/N=C/c1cc(Cl)cc([N+](=O)[O-])c1O)c1cc(-c2ccccn2)nc2ccccc12 ; CC1=NN(c2ccccc2)C(=O)/C1=C\c1ccc(-c2cc(C(=O)O)ccc2Cl)o1 ; O=C(c1ccccc1)c1cc(-c2cc(=O)cc(-c3cc(C(=O)c4ccccc4)c(O)cc3O)o2)c(O)cc1O ; O=C1OC(c2cccc3ccccc23)=N/C1=C\c1ccc(-c2cccc(C(F)(F)F)c2)o1]

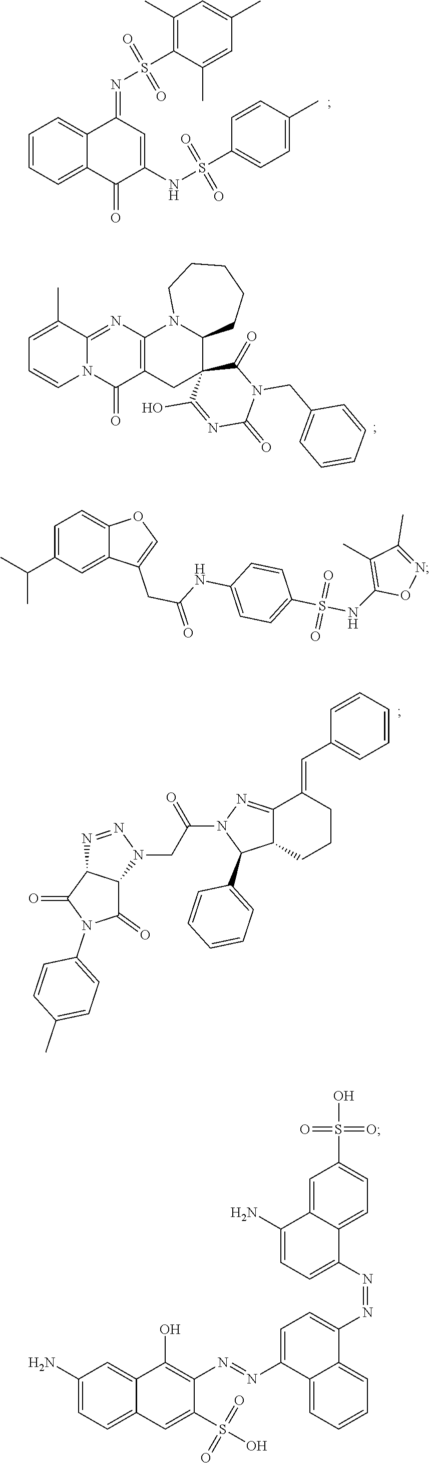

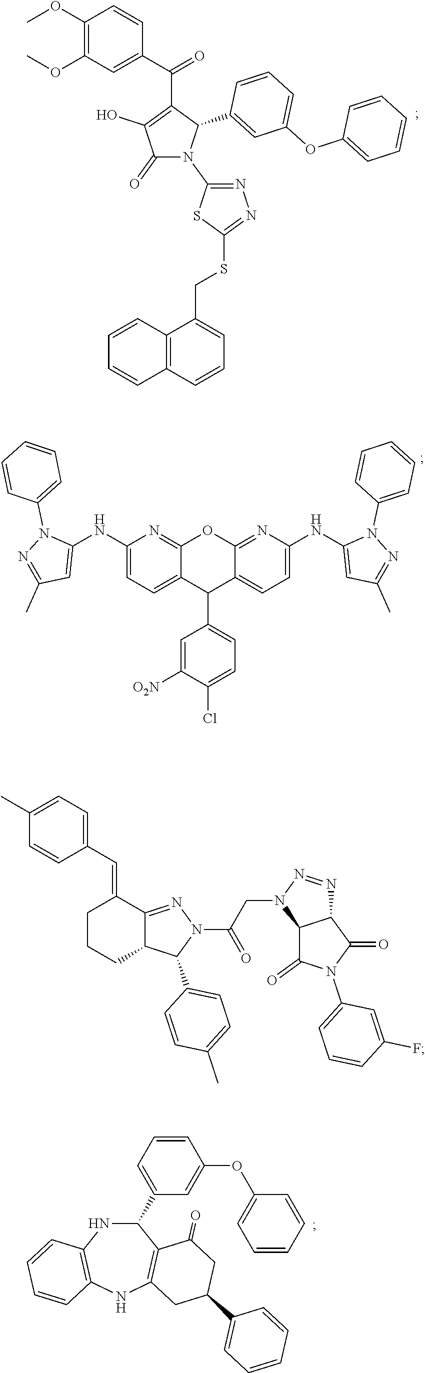 [SMILES: COc1ccc(C(=O)C2=C(O)C(=O)N(c3nnc(SCc4cccc5ccccc45)s3)[C@H]2c2cccc(Oc3ccccc3)c2)cc1OC ; Cc1cc(Nc2ccc3c(n2)Oc2nc(Nc4cc(C)nn4-c4ccccc4)ccc2C3c2ccc(Cl)c([N+](=O)[O-])c2)n(-c2ccccc2)n1 ; Cc1ccc(/C=C2\CCC[C@@H]3C2=NN(C(=O)CN2N=N[C@@H]4C(=O)N(c5cccc(F)c5)C(=O)[C@H]42)[C@@H]3c2ccc(C)cc2)cc1 ; O=C1C[C@@H](c2ccccc2)CC2=C1[C@@H](c1cccc(Oc3ccccc3)c1)Nc1ccccc1N2]

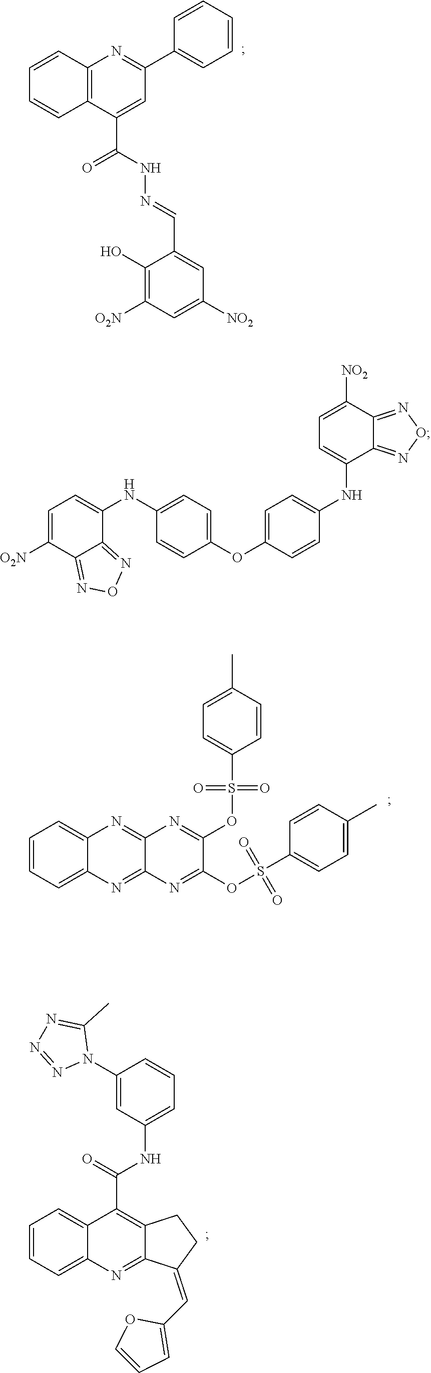 [SMILES: O=C(N/N=C/c1cc([N+](=O)[O-])cc([N+](=O)[O-])c1O)c1cc(-c2ccccc2)nc2ccccc12 ; O=[N+]([O-])c1ccc(Nc2ccc(Oc3ccc(Nc4ccc([N+](=O)[O-])c5nonc45)cc3)cc2)c2nonc12 ; Cc1ccc(S(=O)(=O)Oc2nc3nc4ccccc4nc3nc2OS(=O)(=O)c2ccc(C)cc2)cc1 ; Cc1nnnn1-c1cccc(NC(=O)c2c3c(nc4ccccc24)/C(=C\c2ccco2)CC3)c1]

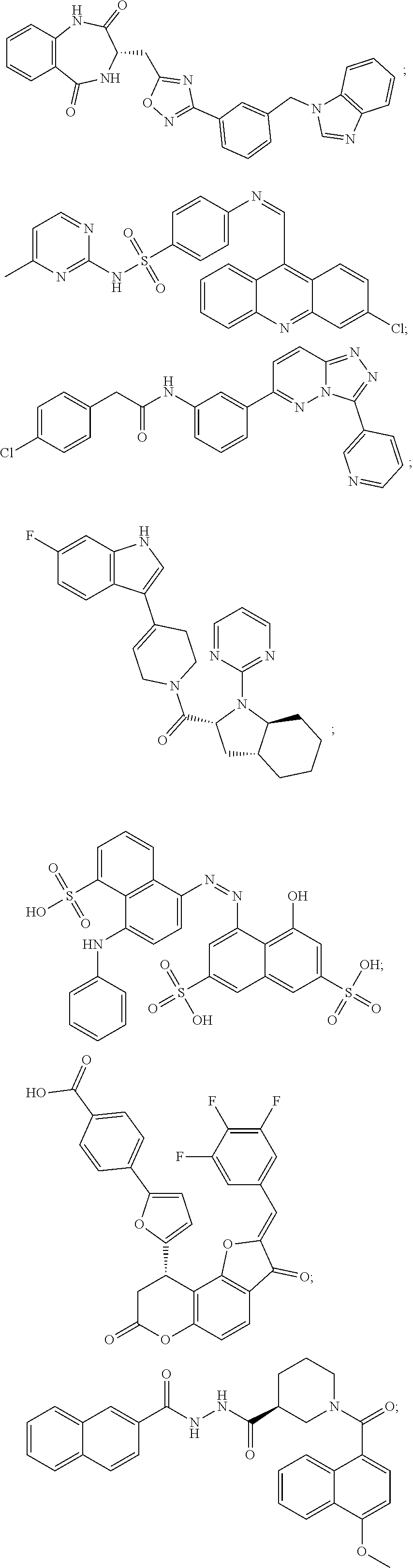

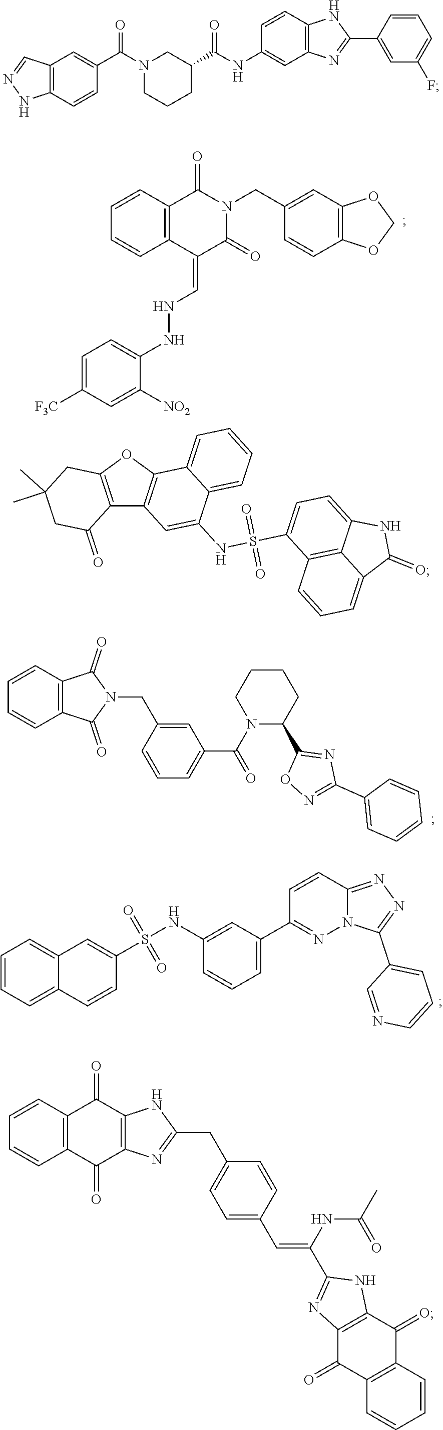

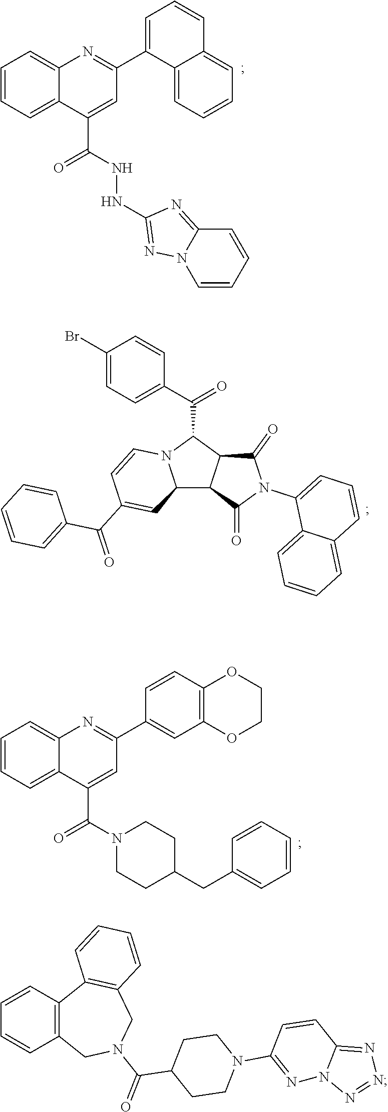 [SMILES: O=C(NNc1nc2ccccn2n1)c1cc(-c2cccc3ccccc23)nc2ccccc12 ; O=C(C1=C[C@@H]2[C@@H]3C(=O)N(c4cccc5ccccc45)C(=O)[C@@H]3[C@@H](C(=O)c3ccc(Br)cc3)N2C=C1)c1ccccc1 ; O=C(c1cc(-c2ccc3c(c2)OCCO3)nc2ccccc12)N1CCC(Cc2ccccc2)CC1 ; O=C(C1CCN(c2ccc3nnnn3n2)CC1)N1Cc2ccccc2-c2ccccc2C1]

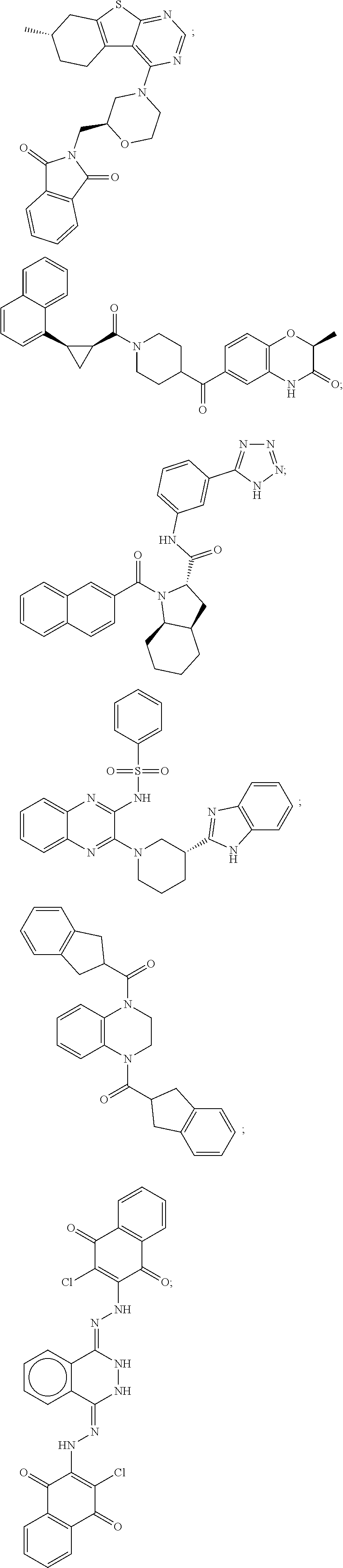

or a pharmaceutically acceptable salt, solvate, hydrate, polymorph, co-crystal, tautomer, stereoisomer, isotopically labeled derivative, or prodrug thereof.

Embodiment 37: A method of activating Nrf2, the method comprising contacting KEAP1 with a compound of Formula (I):

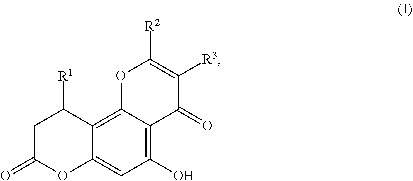

or a pharmaceutically acceptable salt, solvate, hydrate, polymorph, co-crystal, tautomer, stereoisomer, isotopically labeled derivative, or prodrug thereof, wherein:
R¹ is substituted or unsubstituted aryl, substituted or unsubstituted heteroaryl, or substituted or unsubstituted heterocyclyl;
R² is substituted or unsubstituted aryl, substituted or unsubstituted heteroaryl, or substituted or unsubstituted heterocyclyl; and
R³ is hydrogen, —OR^{A}, —SR^{A}, or —N(R^{A})₂;
each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl; or
   a compound of Formula (II):

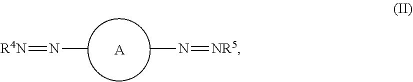

or a pharmaceutically acceptable salt, solvate, hydrate, polymorph, co-crystal, tautomer, stereoisomer, isotopically labeled derivative, or prodrug thereof, wherein:
A is substituted or unsubstituted aryl, substituted or unsubstituted biaryl, or substituted or unsubstituted heteroaryl;
R⁴ is substituted or unsubstituted aryl, substituted or unsubstituted heteroaryl, or substituted or unsubstituted heterocyclyl; and
R⁵ is substituted or unsubstituted aryl, substituted or unsubstituted heteroaryl, or substituted or unsubstituted heterocyclyl; or
   a compound of Formula (III):

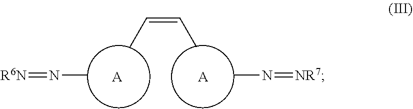

or a pharmaceutically acceptable salt, solvate, hydrate, polymorph, co-crystal, tautomer, stereoisomer, isotopically labeled derivative, or prodrug thereof, wherein:
each A is independently substituted or unsubstituted aryl, or substituted or unsubstituted heteroaryl;
R⁶ is substituted or unsubstituted aryl, substituted or unsubstituted heteroaryl, or substituted or unsubstituted heterocyclyl; and
R⁷ is substituted or unsubstituted aryl, substituted or unsubstituted heteroaryl, or substituted or unsubstituted heterocyclyl; or
   a compound selected from the following:

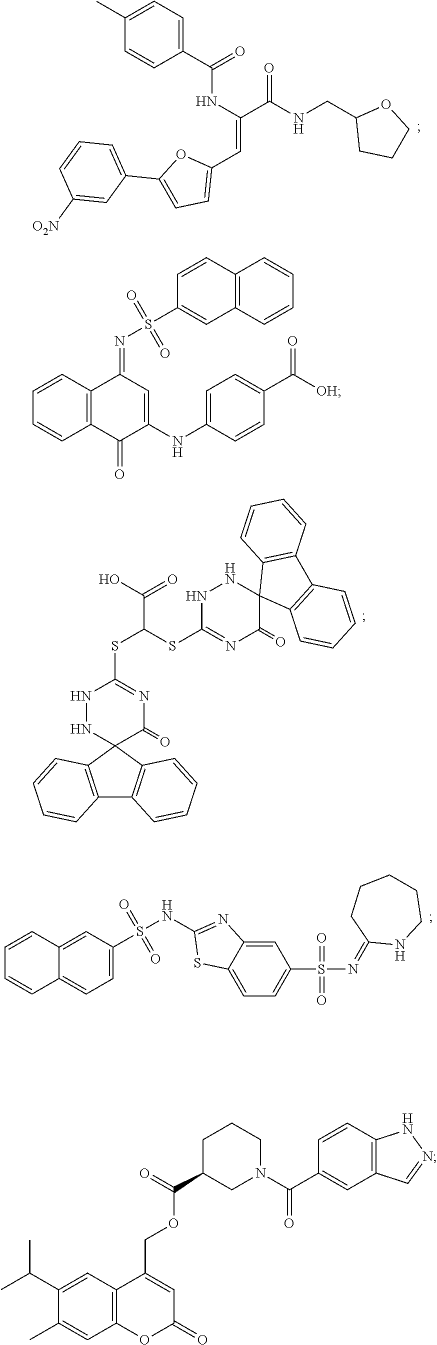 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1 ; Cc1cc2oc(=O)cc(COC(=O)[C@H]3CCCN(C(=O)c4ccc5[nH]ncc5c4)C3)c2cc1C(C)C]

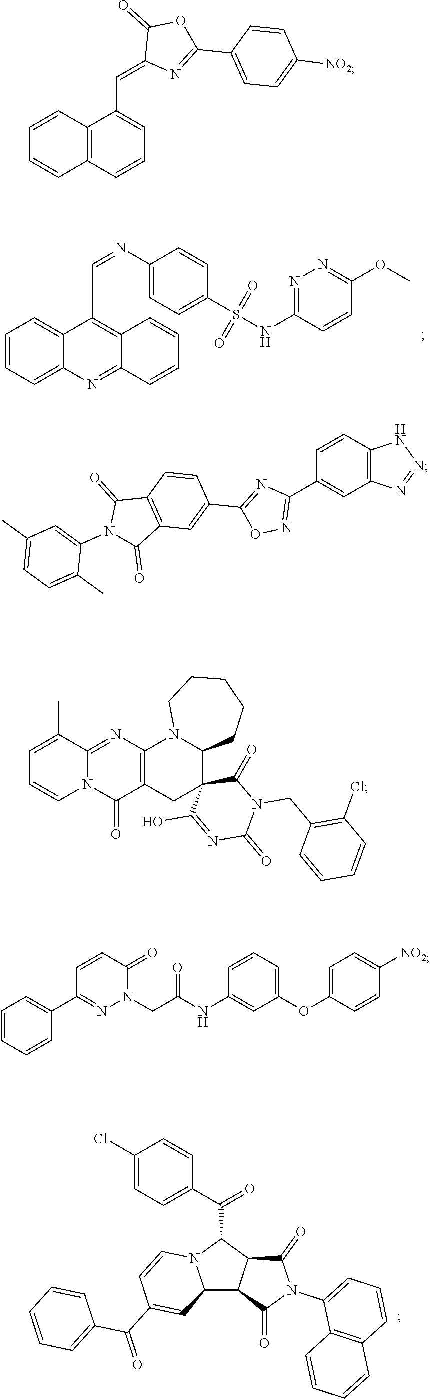

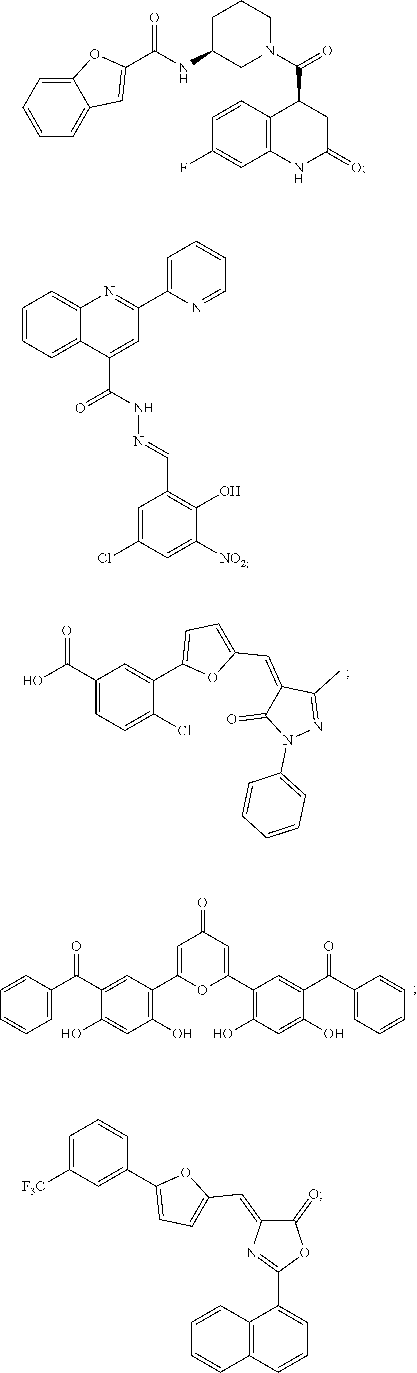 [SMILES: O=C1C[C@H](C(=O)N2CCC[C@H](NC(=O)c3cc4ccccc4o3)C2)c2ccc(F)cc2N1 ; O=C(N/N=C/c1cc(Cl)cc([N+](=O)[O-])c1O)c1cc(-c2ccccn2)nc2ccccc12 ; CC1=NN(c2ccccc2)C(=O)/C1=C\c1ccc(-c2cc(C(=O)O)ccc2Cl)o1 ; O=C(c1ccccc1)c1cc(-c2cc(=O)cc(-c3cc(C(=O)c4ccccc4)c(O)cc3O)o2)c(O)cc1O ; O=C1OC(c2cccc3ccccc23)=N/C1=C\c1ccc(-c2cccc(C(F)(F)F)c2)o1]

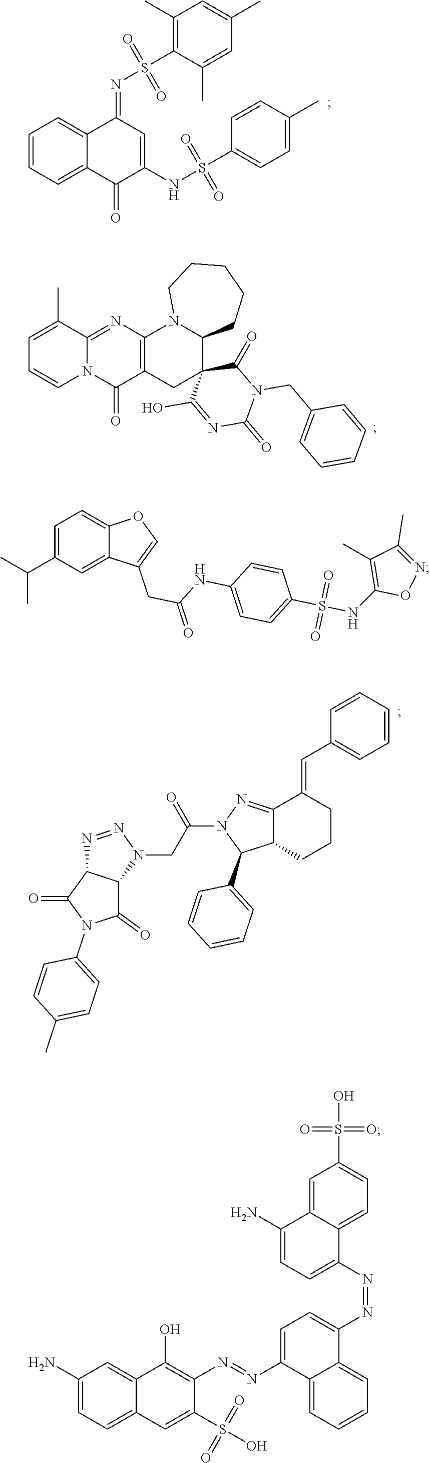

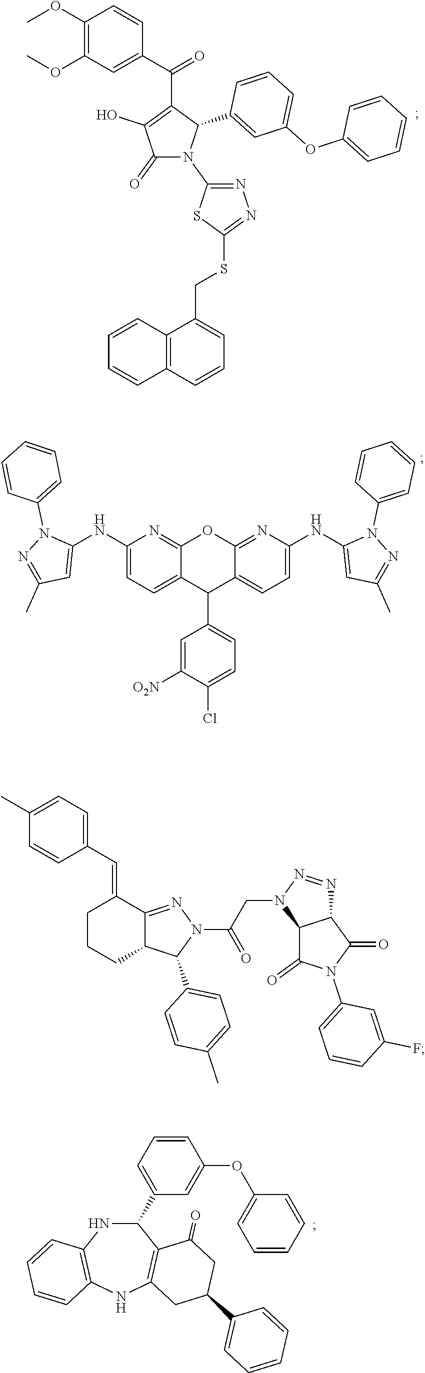 [SMILES: COc1ccc(C(=O)C2=C(O)C(=O)N(c3nnc(SCc4cccc5ccccc45)s3)[C@H]2c2cccc(Oc3ccccc3)c2)cc1OC ; Cc1cc(Nc2ccc3c(n2)Oc2nc(Nc4cc(C)nn4-c4ccccc4)ccc2C3c2ccc(Cl)c([N+](=O)[O-])c2)n(-c2ccccc2)n1 ; Cc1ccc(/C=C2\CCC[C@@H]3C2=NN(C(=O)CN2N=N[C@@H]4C(=O)N(c5cccc(F)c5)C(=O)[C@H]42)[C@@H]3c2ccc(C)cc2)cc1 ; O=C1C[C@@H](c2ccccc2)CC2=C1[C@@H](c1cccc(Oc3ccccc3)c1)Nc1ccccc1N2]

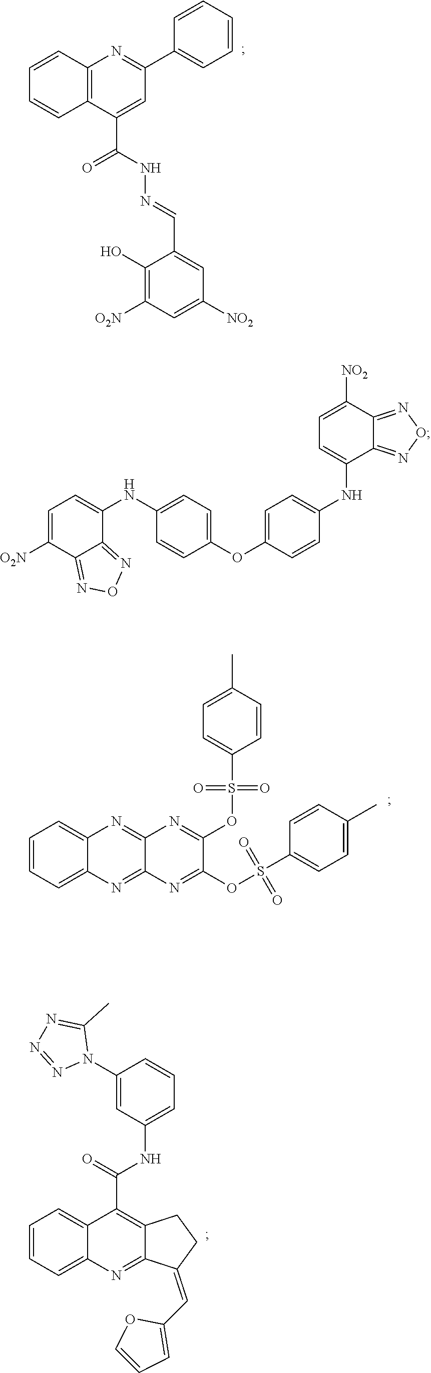 [SMILES: O=C(N/N=C/c1cc([N+](=O)[O-])cc([N+](=O)[O-])c1O)c1cc(-c2ccccc2)nc2ccccc12 ; O=[N+]([O-])c1ccc(Nc2ccc(Oc3ccc(Nc4ccc([N+](=O)[O-])c5nonc45)cc3)cc2)c2nonc12 ; Cc1ccc(S(=O)(=O)Oc2nc3nc4ccccc4nc3nc2OS(=O)(=O)c2ccc(C)cc2)cc1 ; Cc1nnnn1-c1cccc(NC(=O)c2c3c(nc4ccccc24)/C(=C\c2ccco2)CC3)c1]

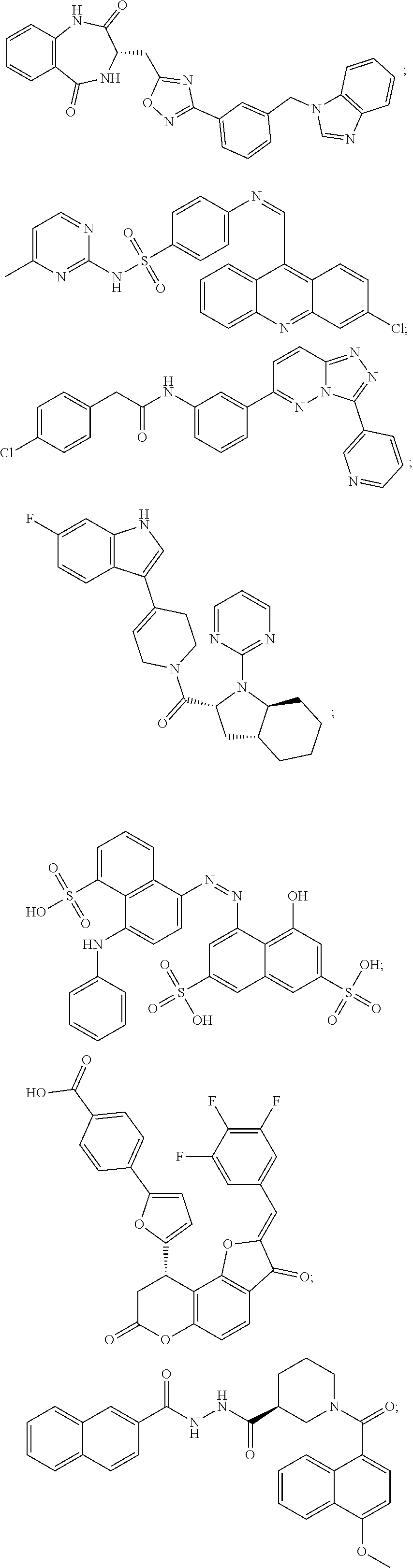

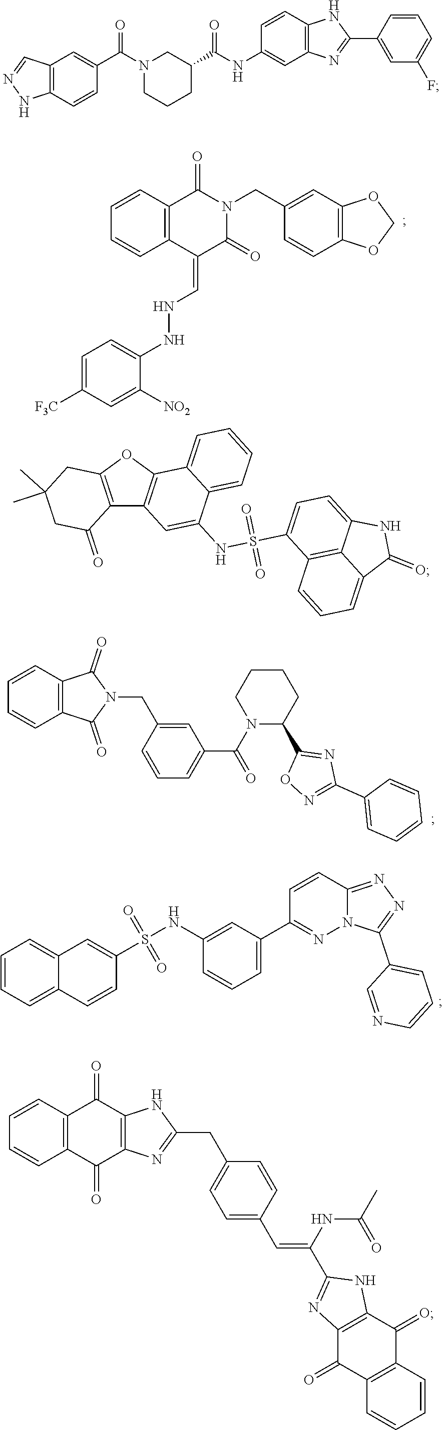

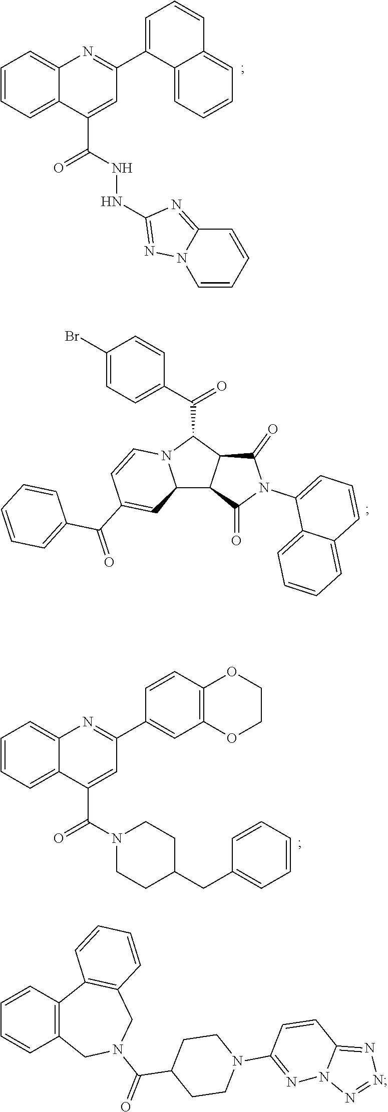 [SMILES: O=C(NNc1nc2ccccn2n1)c1cc(-c2cccc3ccccc23)nc2ccccc12 ; O=C(C1=C[C@@H]2[C@@H]3C(=O)N(c4cccc5ccccc45)C(=O)[C@@H]3[C@@H](C(=O)c3ccc(Br)cc3)N2C=C1)c1ccccc1 ; O=C(c1cc(-c2ccc3c(c2)OCCO3)nc2ccccc12)N1CCC(Cc2ccccc2)CC1 ; O=C(C1CCN(c2ccc3nnnn3n2)CC1)N1Cc2ccccc2-c2ccccc2C1]

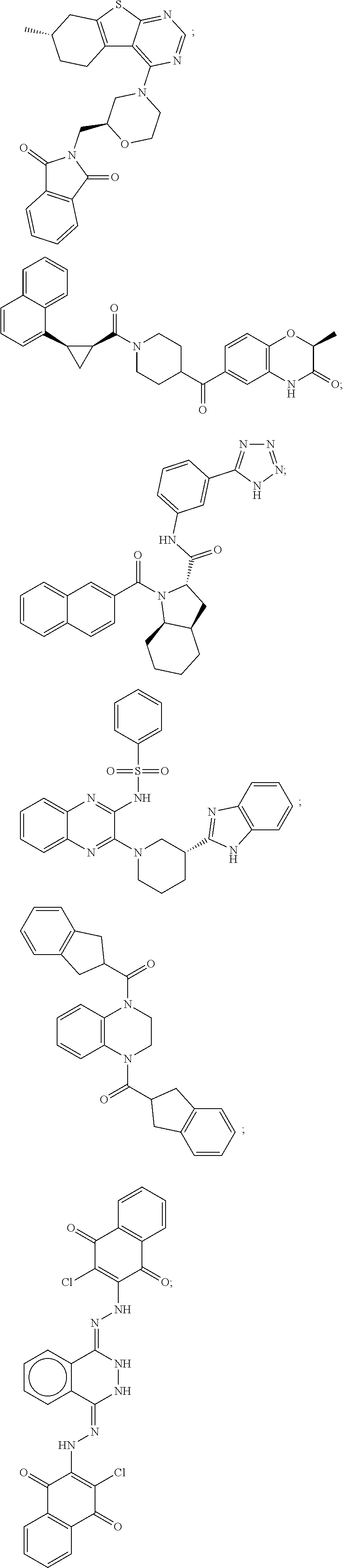

or a pharmaceutically acceptable salt, solvate, hydrate, polymorph, co-crystal, tautomer, stereoisomer, isotopically labeled derivative, or prodrug thereof.

Embodiment 38: A pharmaceutical composition comprising a compound of the formula:

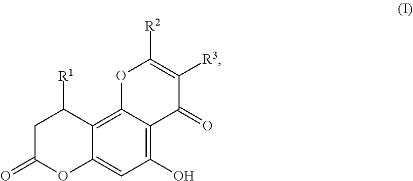

or a pharmaceutically acceptable salt, solvate, hydrate, polymorph, co-crystal, tautomer, stereoisomer, isotopically labeled derivative, or prodrug thereof, wherein:
R¹ is substituted or unsubstituted aryl, substituted or unsubstituted heteroaryl, or substituted or unsubstituted heterocyclyl;
R² is substituted or unsubstituted aryl, substituted or unsubstituted heteroaryl, or substituted or unsubstituted heterocyclyl; and
R³ is hydrogen, —OR^{A}, —SR^{A}, or —N(R^{A})₂;
each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl; or
   a compound of Formula (II):

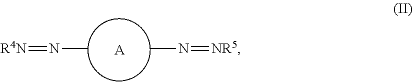

or a pharmaceutically acceptable salt, solvate, hydrate, polymorph, co-crystal, tautomer, stereoisomer, isotopically labeled derivative, or prodrug thereof, wherein:
A is substituted or unsubstituted aryl, substituted or unsubstituted biaryl, or substituted or unsubstituted heteroaryl;
R⁴ is substituted or unsubstituted aryl, substituted or unsubstituted heteroaryl, or substituted or unsubstituted heterocyclyl; and
R⁵ is substituted or unsubstituted aryl, substituted or unsubstituted heteroaryl, or substituted or unsubstituted heterocyclyl; or
   a compound of Formula (III):

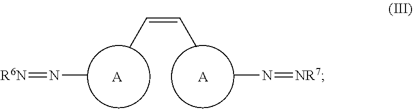

or a pharmaceutically acceptable salt, solvate, hydrate, polymorph, co-crystal, tautomer, stereoisomer, isotopically labeled derivative, or prodrug thereof, wherein:
each A is independently substituted or unsubstituted aryl, or substituted or unsubstituted heteroaryl;
R⁶ is substituted or unsubstituted aryl, substituted or unsubstituted heteroaryl, or substituted or unsubstituted heterocyclyl; and
R⁷ is substituted or unsubstituted aryl, substituted or unsubstituted heteroaryl, or substituted or unsubstituted heterocyclyl; or
   a compound selected from the following:

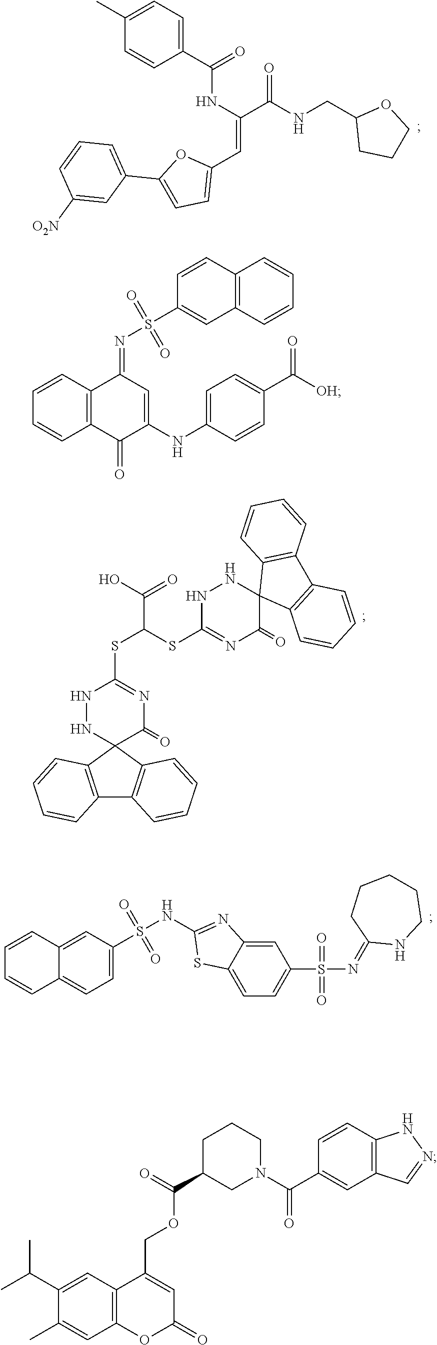 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1 ; Cc1cc2oc(=O)cc(COC(=O)[C@H]3CCCN(C(=O)c4ccc5[nH]ncc5c4)C3)c2cc1C(C)C]

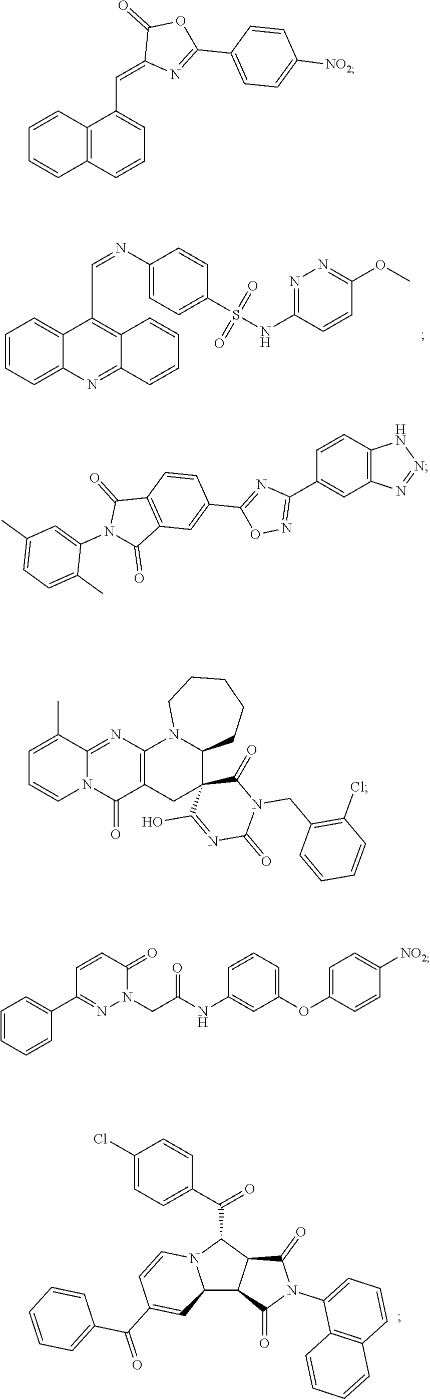

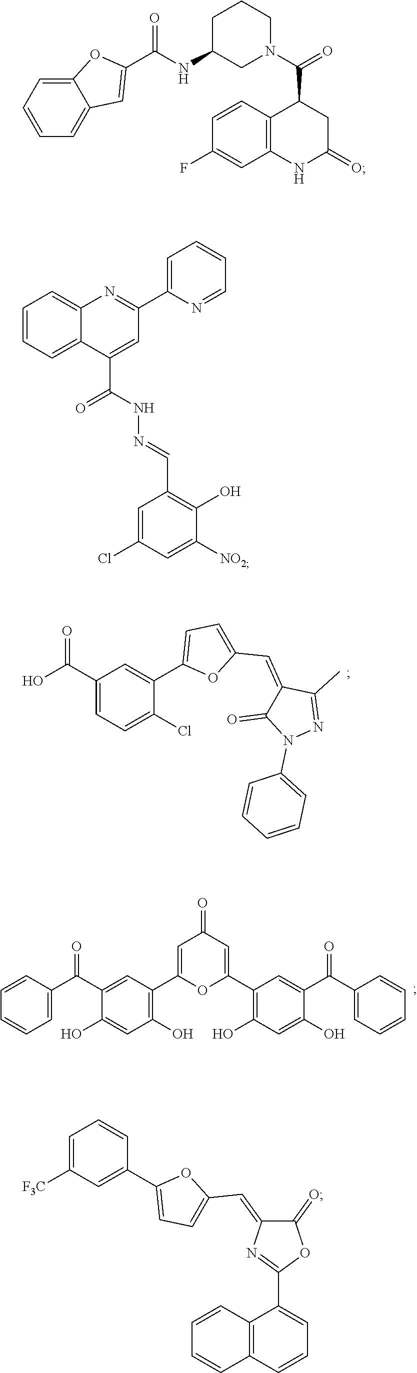 [SMILES: O=C1C[C@H](C(=O)N2CCC[C@H](NC(=O)c3cc4ccccc4o3)C2)c2ccc(F)cc2N1 ; O=C(N/N=C/c1cc(Cl)cc([N+](=O)[O-])c1O)c1cc(-c2ccccn2)nc2ccccc12 ; CC1=NN(c2ccccc2)C(=O)/C1=C\c1ccc(-c2cc(C(=O)O)ccc2Cl)o1 ; O=C(c1ccccc1)c1cc(-c2cc(=O)cc(-c3cc(C(=O)c4ccccc4)c(O)cc3O)o2)c(O)cc1O ; O=C1OC(c2cccc3ccccc23)=N/C1=C\c1ccc(-c2cccc(C(F)(F)F)c2)o1]

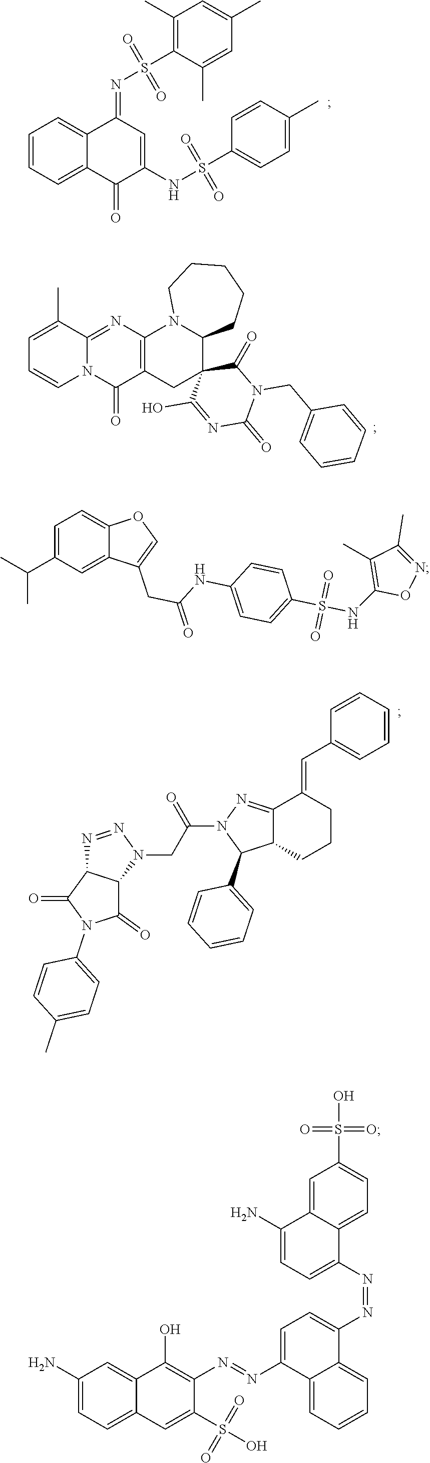

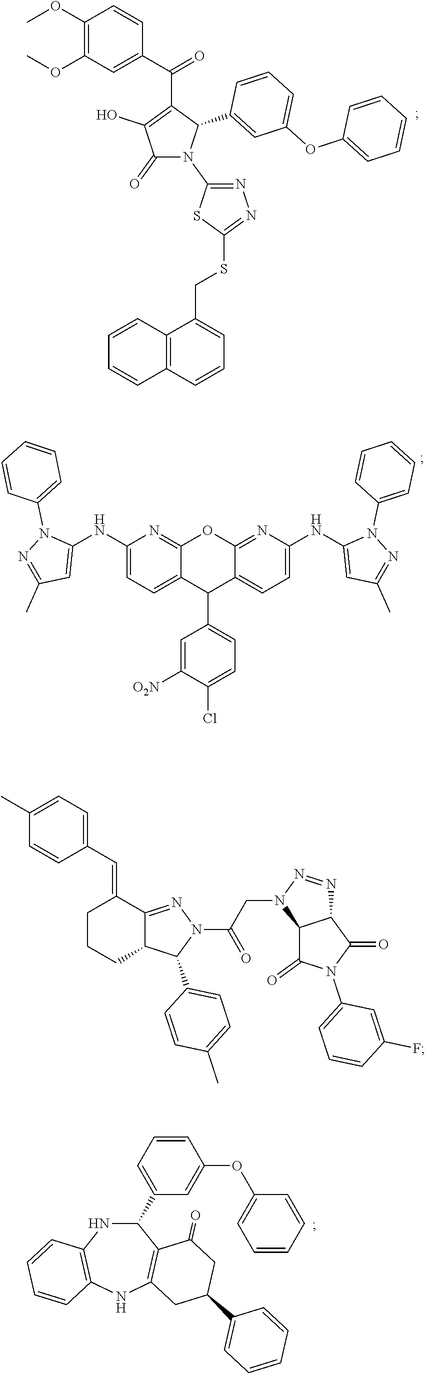 [SMILES: COc1ccc(C(=O)C2=C(O)C(=O)N(c3nnc(SCc4cccc5ccccc45)s3)[C@H]2c2cccc(Oc3ccccc3)c2)cc1OC ; Cc1cc(Nc2ccc3c(n2)Oc2nc(Nc4cc(C)nn4-c4ccccc4)ccc2C3c2ccc(Cl)c([N+](=O)[O-])c2)n(-c2ccccc2)n1 ; Cc1ccc(/C=C2\CCC[C@@H]3C2=NN(C(=O)CN2N=N[C@@H]4C(=O)N(c5cccc(F)c5)C(=O)[C@H]42)[C@@H]3c2ccc(C)cc2)cc1 ; O=C1C[C@@H](c2ccccc2)CC2=C1[C@@H](c1cccc(Oc3ccccc3)c1)Nc1ccccc1N2]

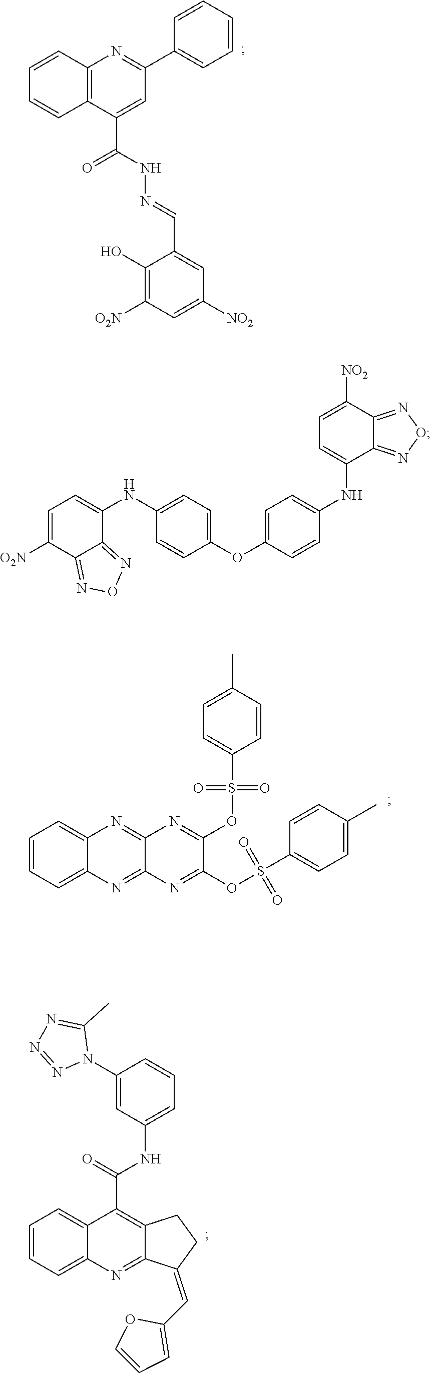 [SMILES: O=C(N/N=C/c1cc([N+](=O)[O-])cc([N+](=O)[O-])c1O)c1cc(-c2ccccc2)nc2ccccc12 ; O=[N+]([O-])c1ccc(Nc2ccc(Oc3ccc(Nc4ccc([N+](=O)[O-])c5nonc45)cc3)cc2)c2nonc12 ; Cc1ccc(S(=O)(=O)Oc2nc3nc4ccccc4nc3nc2OS(=O)(=O)c2ccc(C)cc2)cc1 ; Cc1nnnn1-c1cccc(NC(=O)c2c3c(nc4ccccc24)/C(=C\c2ccco2)CC3)c1]

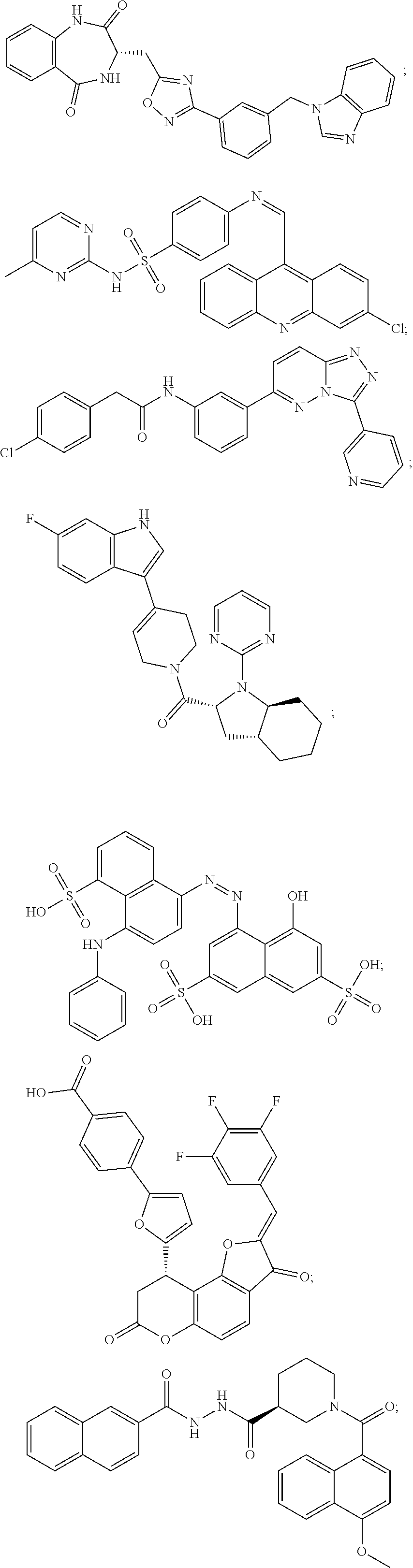

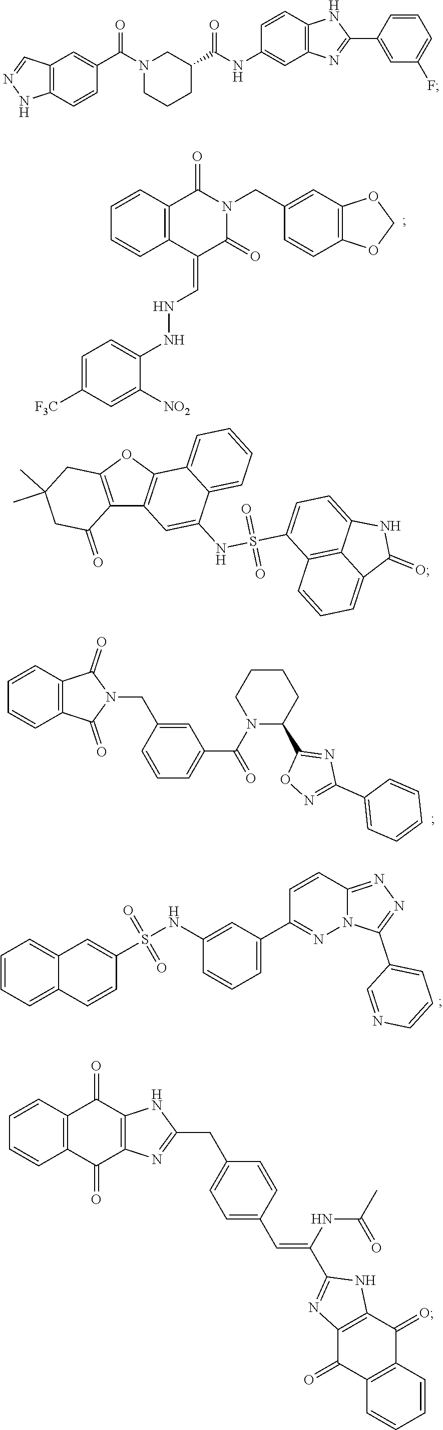

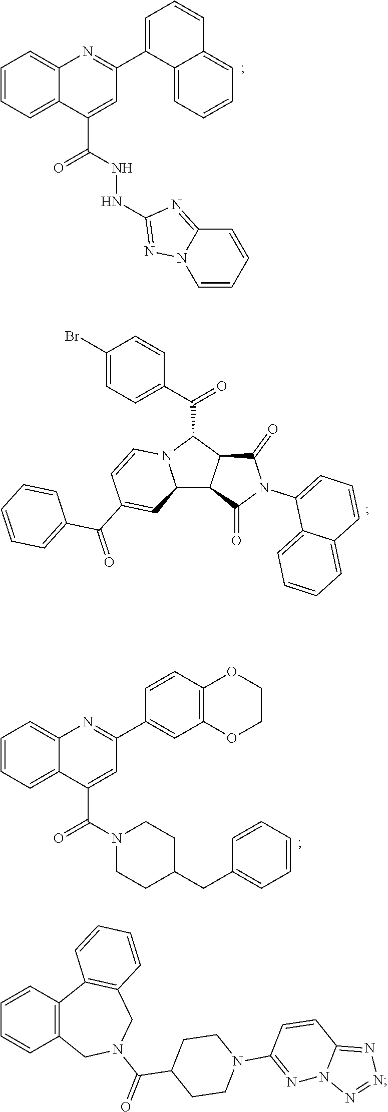 [SMILES: O=C(NNc1nc2ccccn2n1)c1cc(-c2cccc3ccccc23)nc2ccccc12 ; O=C(C1=C[C@@H]2[C@@H]3C(=O)N(c4cccc5ccccc45)C(=O)[C@@H]3[C@@H](C(=O)c3ccc(Br)cc3)N2C=C1)c1ccccc1 ; O=C(c1cc(-c2ccc3c(c2)OCCO3)nc2ccccc12)N1CCC(Cc2ccccc2)CC1 ; O=C(C1CCN(c2ccc3nnnn3n2)CC1)N1Cc2ccccc2-c2ccccc2C1]

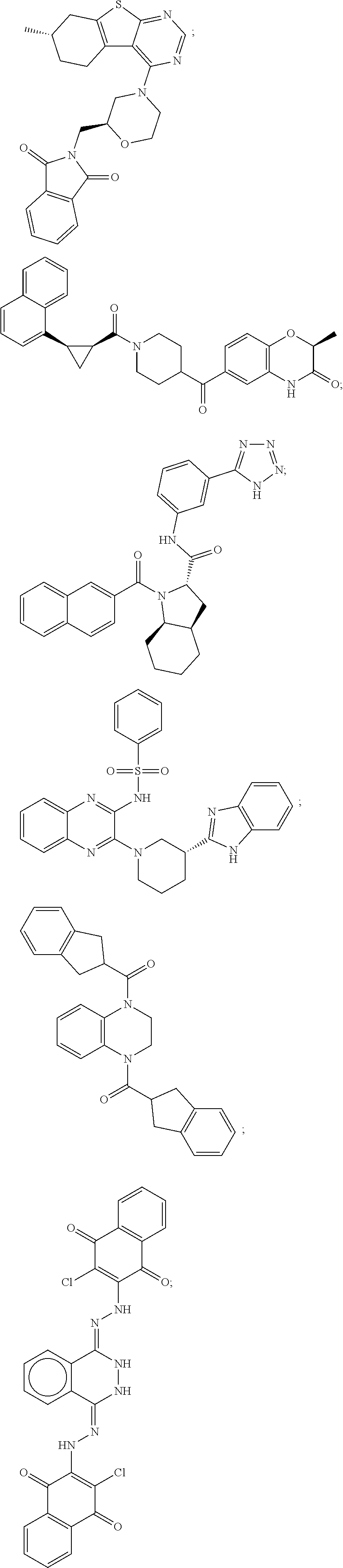

or a pharmaceutically acceptable salt, solvate, hydrate, polymorph, co-crystal, tautomer, stereoisomer, isotopically labeled derivative, or prodrug thereof.

### Some Selected Definitions

For convenience, certain terms employed herein, in the specification, examples and appended claims are collected herein. Unless stated otherwise, or implicit from context, the following terms and phrases include the meanings provided below Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%, ±1.5%, ±2%, ±2.5%, ±3%, ±3.5%, ±4%, ±4.5%, or ±5%.

The singular terms “a,” “an,” and “the” include plural referents unless context clearly indicates otherwise. Similarly, the word “or” is intended to include “and” unless the context clearly indicates otherwise.

As used herein the terms “comprising” or “comprises” means “including” or “includes” and are used in reference to compositions, methods, systems, and respective component(s) thereof, that are useful to the invention, yet open to the inclusion of unspecified elements, whether useful or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, systems, and respective components thereof as described herein, which are exclusive of any element not recited in that description of the embodiment.

The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

As used herein, the terms “treat,” “treatment,” “treating,” or “amelioration” are used herein to characterize a method or process that is aimed at (1) delaying or preventing the onset of a disease or condition; (2) slowing down or stopping the progression, aggravation, or deterioration of the symptoms of the disease or condition; (3) bringing about ameliorations of the symptoms of the disease or condition; or (4) curing the disease or condition. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased morbidity or mortality. The term “treatment” of a disease also includes providing relief from the symptoms or side-effects of the disease (including palliative treatment). A treatment can be administered prior to the onset of the disease, for a prophylactic or preventive action. Alternatively, or additionally, the treatment can be administered after initiation of the disease or condition, for a therapeutic action.

In some embodiments, treatment is therapeutic and does not include prophylactic treatment.

The terms “co-administration” or the like, as used herein, are meant to encompass administration of the selected therapeutic agents to a single patient and are intended to include treatment regimens in which the agents are administered by the same or different route of administration or at the same or different time. The particular combination of therapies (therapeutics or procedures) to employ in such a combination regimen will take into account compatibility of the desired therapeutics and/or procedures and the desired therapeutic effect to be achieved.

As used herein, the term “subject” refers to any living organism which can be administered compound and/or pharmaceutical compositions of the present invention. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult, child and newborn subjects, whether male or female, are intended to be covered. The term “subject” is also intended to include living organisms susceptible to conditions or disease states as generally disclosed, but not limited to, throughout this specification. Examples of subjects include humans, dogs, cats, cows, goats, and mice. The term subject is further intended to include transgenic species. The term “subject” and “individual” are used interchangeably herein, and refer to an animal, for example a human or non-human mammals/animals, to whom treatment, including prophylactic treatment, with the compounds and compositions according to the present invention, is provided. The term “non-human animals” and “non-human mammals” are used interchangeably herein and include all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc.

In some embodiments, the subject is a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of diseases or disorders associated with dysfunction of Nrf2/KEAP1 axis.

It is noted that a human subject can be of any age, gender, race or ethnic group, e.g., Caucasian (white), Asian, African, black, African American, African European, Hispanic, Middle eastern, etc.

In addition, the methods described herein can be used to treat domesticated animals and/or pets. A subject can be male or female. A subject can be one who has been previously diagnosed with or identified as suffering from or having a disease or disorder associated with dysfunction of Nrf2/KEAP1 axis, but need not have already undergone treatment.

In some embodiments of any one of the aspects, the subject is human.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxy, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxy, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, —NHCH₂CH₂CH₃, and —NHCH(CH₃)₂. Exemplary dialkylamino includes, but is not limited to, —N(C₁-C₁₀alkyl)₂, such as N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxy” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O-alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl- group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or wherein any alkyl, cycloalkyl or heterocyclyl is optionally substituted with oxo; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1: New Inhibitors for the KEAP1-Nrf2 Protein-Protein Interaction

On average, an approved drug today costs $2-3 billion and takes over ten years to develop¹. In part, this is due to expensive and time-consuming wet-lab experiments, poor initial hit compounds, and the high attrition rates in the (pre-)clinical phases. Structure-based virtual screening (SBVS) has the potential to mitigate these problems. With SBVS, the quality of the hits improves with the number of compounds screened². However, despite the fact that large compound databases exist, the ability to carry out large-scale SBVSs on computer clusters in an accessible, efficient, and flexible manner has remained elusive. Here we designed VirtualFlow, a highly automated and versatile open-source platform with perfect scaling behaviour that is able to prepare and efficiently screen ultra-large ligand libraries of compounds. VirtualFlow is able to use a variety of the most powerful docking programs. Using VirtualFlow, we have prepared the largest and freely available ready-to-dock ligand library available, with over 1.4 billion commercially available molecules. To demonstrate the power of VirtualFlow, we screened over 1 billion compounds and discovered a small molecule inhibitor (iKeap1) that engages KEAP1 with nanomolar affinity (K_{d}=114 nM) and disrupts the interaction between KEAP1 and the transcription factor NRF2. We also identified a set of structurally diverse molecules that bind to KEAP1 with submicromolar affinity. This illustrates the potential of VirtualFlow to access vast regions of the chemical space and identify binders with high affinity for target proteins.

Repeated optimization of lead compounds and late-stage failure of drug candidates are the primary causes of longer development times and increased costs in drug development. Improving the quality of the initial lead compounds would minimize these lead optimization cycles and result in drug candidates entering (pre-)clinical phases with greater specificity and higher affinity. Virtual screening to identify molecules that bind to a specified site on a receptor protein has become an important part of the drug discovery pipeline²⁻⁵.

Current virtual screening paradigms routinely sample only a tiny fraction, on the order of 10⁶-10⁷ molecules, of the total chemical space of small organic compounds suitable for drug discovery, estimated to encompass more than 10⁶⁰ molecules⁶.

However, the scale of a virtual screen is of central importance because the more compounds that are screened, (a) the lower the rate of false positives, and (b) the more favourable the quality of the lead compounds (e.g. higher-affinity binders). It was recently shown experimentally that ultra-large scale screening improves the rate of true positives². Here we derived a probabilistic model of the true-positive rate as a function of the number of compounds screened, and analysis of our ultra-large screen confirms that the docking score of the highest-scoring compounds improve with the scale. Increasing the scale of a virtual screen can improve the quality of initial hits in two distinct ways: (1) by identifying hits with tighter binding affinity, which can result in lowered dosages and fewer off-target effects, and (2) by discovering compounds with more favourable pharmacokinetic and/or less inherent cytotoxic properties.

To increase the number of compounds evaluated in a virtual screen by orders of magnitude and make it accessible to any researcher, there is a dire need for a platform that can integrate all the tasks in the virtual screening process. Such a platform should ideally (1) scale linearly with the number of CPUs, (2) efficiently handle billions of files, (3) minimize input and output (I/O) load, (4) run robustly (e.g. skip incorrectly encoded ligands, resist temporary I/O problems, and resume following unexpected termination), (5) run on any type of computing cluster (including cloud platforms), and (6) be user-friendly and easy to use for non-computational scientists. Furthermore, to provide flexibility, a SBVS platform should be able to interface with a variety of docking programs, support both rigid and flexible receptor docking, test multiple docking scenarios in a single workflow, allow for consensus and ensemble docking, and carry out multiple replicas of the same docking scenario. Lastly, to democratize access, facilitate widespread usage, and catalyse further development, such a platform would need to be open source.

With these requirements in mind, we designed VirtualFlow, an open-source platform that is able to screen chemical space on an unprecedented scale. Screening one billion compounds on a single processor core, with an average docking time of 15 seconds per ligand, would take 475 years. By contrast, VirtualFlow can dock one billion compounds in approximately two weeks by leveraging 10,000 CPU cores simultaneously. Such high performance computing facilities are available to researchers through several potential sources, including local institute computer clusters, national super-computing centres, or cloud computing platforms.

### Targeting KEAP1 Using VirtualFlow

To test the advantages of ultra-large-scale in silico screening and the performance of the VirtualFlow platform we decided to target the challenging and therapeutically relevant protein-protein interaction (PPI) between nuclear factor erythroid-derived 2-related factor 2 (NRF2) and Kelch-like ECH-associated protein 1 (KEAP1). NRF2 is a master regulator of cellular resistance to oxidative stress and cellular repair⁷. Under unstressed conditions, NRF2 is sequestered by KEAP1, an E3 ubiquitin ligase substrate adaptor, and targeted for degradation⁸. However, upon oxidative stress, reactive oxidants dissociate NRF2 from KEAP1 and NRF2 translocates to the nucleus to activate its transcriptional program of approximately 250 genes⁹. The NRF2-KEAP1 pathway is critical in protecting the cell under oxidative stress and inflammation and is implicated in a number of diseases¹⁰. There are ten drugs targeting KEAP1 that are in clinical trials and nine more that are at the preclinical stage¹⁰. Using VirtualFlow, we screened ˜1.3 billion compounds (˜1 billion compounds from the Enamine REAL Library and ˜330 million compounds from the ZINC library) against the NRF2 interaction interface on KEAP1. First, we would like to describe the salient features of VirtualFlow and its scalability.

### Characteristic Features of VirtualFlow

One of the key features of VirtualFlow is its linear scaling behaviour (O(N)) with respect to the number of CPUs and nodes utilized. VirtualFlow can run on computer clusters operated with any of the major resource managers (SLURM¹¹, Moab/TORQUE¹², PBS¹³, LSF¹⁴ and SGE¹⁵), and compatibility with additional job schedulers can be easily added. Thus VirtualFlow is also ideally configured for cloud computing platforms like Amazon's Web Services (AWS), Microsoft's Azure and Google's Cloud Platform (GCP). VirtualFlow is able to run autonomously from the first to the last ligand in the screening pipeline, a feature facilitated by automatic submission of new batch system jobs. The workflow can be monitored and controlled in real time. The VirtualFlow package consists of two applications that work seamlessly together: The VFLP (VirtuaFlow for Ligand Preparation) module, which prepares small molecules for screening; and the VFVS (VirtualFlow for Virtual Screening) module, which executes the virtual screening procedures. The separation of ligand preparation and virtual screening is desirable because the same ready-to-dock ligand library can be used in any number of VFVS virtual screens.

### VirtualFlow for Ligand Preparation (VFLP)

VFLP (VirtualFlow for Ligand Preparation) prepares ligand databases by converting them from the SMILES format into any desired target format (e.g. the PDBQT format, which is required by many of the AutoDock-based docking programs). VFLP uses ChemAxon's JChem package as well as Open Babel to desalt ligands, neutralize them, generate (even multiple) tautomeric states, compute protonation states at specific pH values, calculate 3D coordinates, and convert the molecules into desired target formats.

### Preparation of the Enamine REAL Library

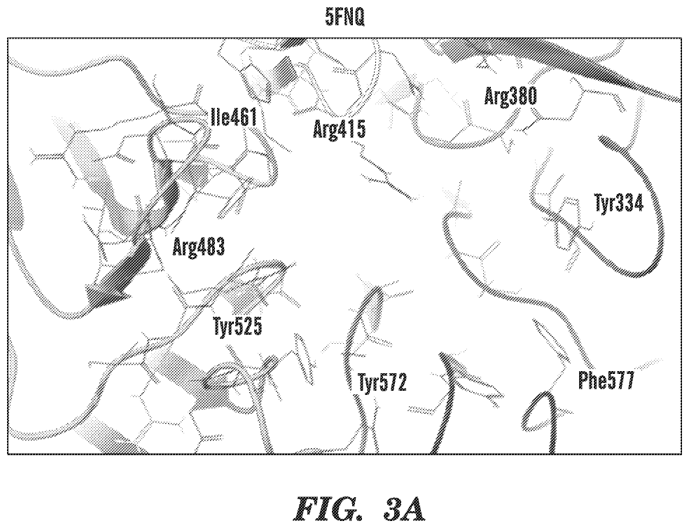

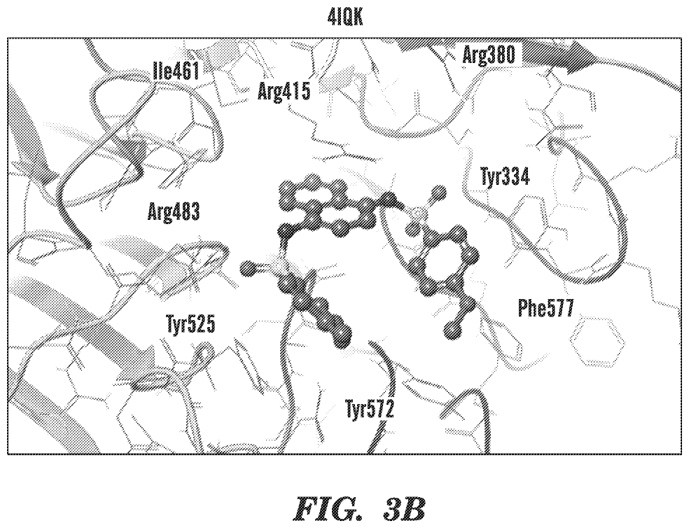

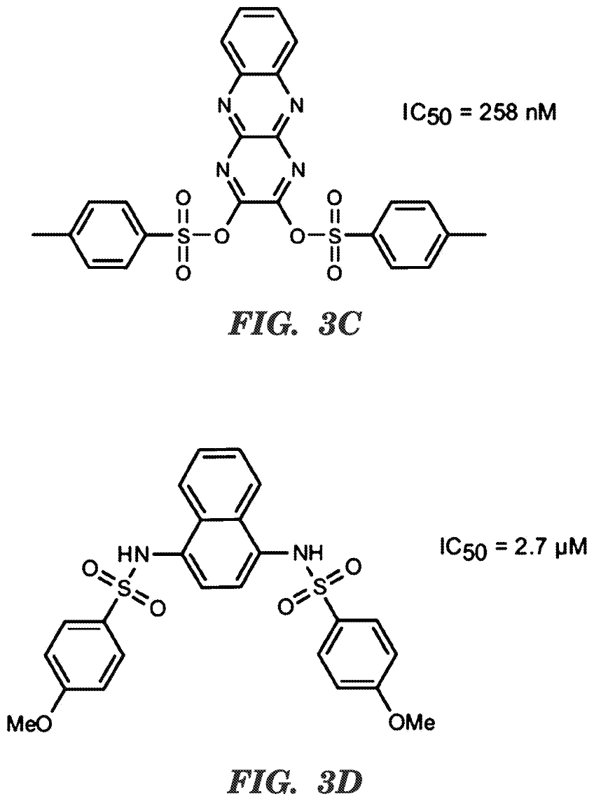

Commercially available compounds constitute the most interesting subset of the chemical space, since these compounds can be readily purchased. The largest vendor library available today is the REAL library of Enamine, containing approximately 1.4 billion make-on-demand compounds (as of October 2019 the ZINC 15 database contained 1.46 billion compounds, but only provided 630 million molecules in a ready-to-dock format). We have used VFLP to convert the ˜1.4 billion compounds of the REAL library into PDBQT format (see Methods), and have made it freely available on the VirtualFlow homepage, accessible via a graphical interface (FIGS. 3A-3D). The entire database has a six-dimensional lattice architecture, the general concept of which was modelled after the ZINC 15 database¹⁶, where each dimension corresponds to a physico-chemical property of the compounds (molecular weight, partition coefficient, number of hydrogen bond donors and acceptors, number of rotatable bonds, and the topological polar surface area). The preparation of ligands using VFLP is a one-time effort.

### VirtualFlow for Virtual Screening (VFVS)

To set up a virtual screen with VFVS, a set of docking scenarios is specified by the user. Docking scenarios are defined by the choice of the external docking program, the receptor structure, and the docking parameters (which include the pre-defined docking surface on the receptor, residues on the receptor that are allowed to be flexible during docking, and the rigor of the docking routine). VirtualFlow currently supports the following docking programs: AutoDock Vina¹⁷, QuickVina 2^{[18]}, Smina (which includes the Vinardo and AutoDock 4 scoring functions)¹⁹, AutoDockFR (ADFR)²⁰, QuickVina-W⁵, VinaXB²¹, and VinaCarb²². By supporting an array of different docking programs, VFVS can be used in a variety of cases by leveraging the unique advantages of each program. VFVS allows the specification of multiple docking scenarios to be carried out for each ligand, enabling consensus docking procedures, as well as ensemble docking procedures^{23,24}. VirtualFlow is also amenable to the integration of other docking programs that are not currently a part of this platform.

### Scaling Behaviour of VFVS

In order to measure the scaling behaviour of VFVS, we measured the performance on two local clusters, LC1 and LC2. On LC1, we used 18,000 CPU cores of heterogeneous composition (different models of Intel Xeon and AMD Opteron processors), whereas on LC2 we employed up to 30,000 Intel Xeon 8268 CPUs. The scaling behaviour was effectively linear in both cases (i.e., O(N), where N is the number of cores). These results meet theoretical expectations since there is no direct communication between the processes running in parallel, which is key to perfect scaling behaviour without bounds. The independence of its parallel processes means that VirtualFlow is expected to scale linearly even if millions of cores are used. We also tested the performance of the platform on cloud-based computing systems including GCP and AWS. On the GCP we carried out large-scale benchmarks with up to 160,000 CPUs, and despite this massive scaling in CPU volume, VirtualFlow still exhibited linear scaling. A typical high-throughput screen, such as the one described in this study, of 1 billion compounds will take ˜15 hours on the GCP with 160,000 CPUs, making VirtualFlow suitable for the highly anticipated exascale computing age.

Multistaged Virtual Screens with VFVS

VFVS can also be used to organize virtual screens with multiple stages to substantially increase the quality of the results. In the multi-staging approach, several virtual screens are executed in succession. The number of top-scoring compounds that advance from one stage to the next is successively reduced, with concomitant increases in docking accuracy and computational cost.

### Using VFVS to Screen 1.3 Billion Ligands

In order to validate the performance of VFVS we screened a virtual library of 1.3 billion commercially available compounds (˜330 million compounds from the ZINC 15 database¹⁶, and ˜1 billion compounds from the Enamine REAL library) against KEAP1. % It should be noted that there is some overlap of compounds between the two libraries.

This effort was completed in around four weeks, using on average approximately 8,000 cores on a heterogeneous Linux cluster.

To illustrate the benefit of an ultra-large-scale screen, we chose a subsets of the ligands (0.1, 1, 10, and 100 million compounds) randomly from the ˜1 billion compound screen of the REAL library and considered the scores of the top 50 compounds. As the scale of the screen increased, the average docking score increased thus improving the chances of identifying tighter binders. This in turn leads to higher true hit rates and tighter experimental binding affinities, as predicted by a probabilistic model experimentally demonstrated previously².

To demonstrate VirtualFlow in a multi-staging context we subjected the top ˜3 million ranking compounds from the primary virtual screen to a rescoring procedure. In stage-2, the 13 residues of KEAP1 at the NFR2 interaction interface were allowed to be flexible. This flexibility accounted for the movement/dynamics of the amino acids at the binding interface, not captured by a static structure. In the re-scoring procedure we utilized two different docking programs (Smina Vinardo and AutoDock Vina), and two replicas of each docking scenario were carried out to further increase the conformational space sampled during the docking runs. The necessity of multi-stage screening depends on the target of choice and the computational resources available, but this type of virtual screen is particularly useful in cases where dynamics at the docking interface is expected to play a significant role.

### Experimental Validation

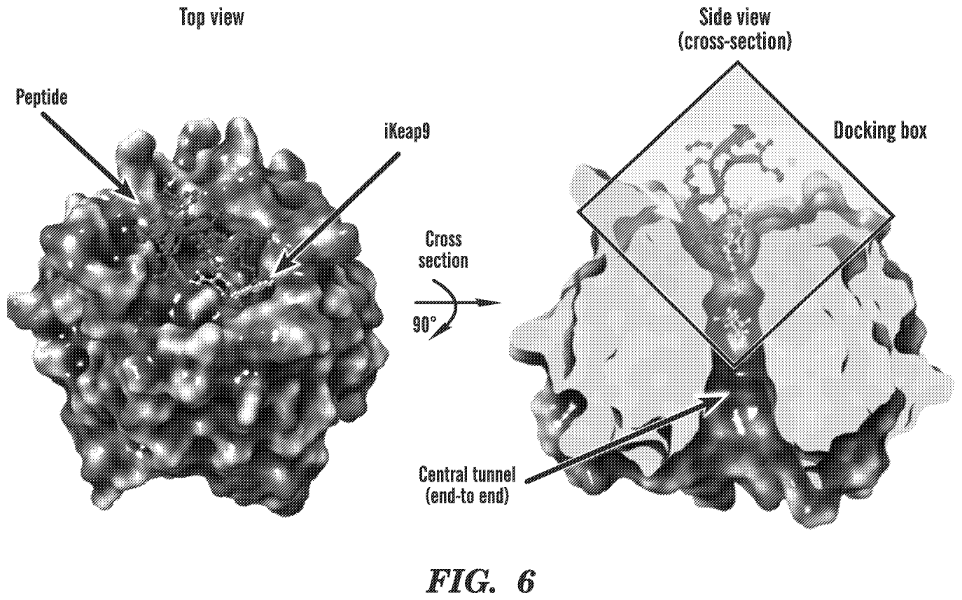

From the in silico screen described above, we chose 590 hits for experimental validation. Of these, 492 compounds were from the top 0.03% of stage-2 screen and 98 compounds were from the top 0.0001% of stage-1. Hits from stage-1 were ordered to compare the true hit rate between stage-1 and stage-2 hits, in a multistage setting. In addition to the ranking by docking score, the choice of these compounds was based on factors like drug-likeness, availability for procurement, ligand efficiency and chemical diversity. We used four established biophysical methods: fluorescence polarization (FP), surface plasmon resonance (SPR), nuclear magnetic resonance (NMR), and bio-layer interferometry (BLI) to experimentally validate the binding of the VirtualFlow-derived hits to KEAP1. FP and SPR were initially used in a high-throughput fashion (Level-1) to detect binding and the compounds identified here were subsequently validated with more scrutiny in a detailed and low-throughput fashion (Level-2). We used a recombinantly expressed and purified Kelch domain of mouse KEAP1, henceforth referred to as KEAP1. A detailed description of the experimental procedure is provided in the methods section. Of these four biophysical methods, FP and BLI detect the ability of the hits to displace the NRF2 peptide from KEAP1, identifying hits we refer to as displacers. SPR and NMR directly detect binding of hits to KEAP1, identifying hits referred to as binders. VirtualFlow identifies molecules that potentially bind to the NRF2-interacting interface on KEAP1, but the in silico screen is performed using KEAP1 alone, in the absence of NRF2. The NRF2 binding surface on KEAP1 is part of the deep pocket/tunnel of the KEAP1 β-barrel with NRF2 binding to the entrance of this tunnel. However, some compounds could bind more tightly by inserting deep into this central tunnel of KEAP1 rather than embracing the surface like the NRF2 peptide, and/or bind to parts of KEAP1 not engaged by NRF2. Such binders might not effectively disrupt the interaction with NRF2, while still engaging KEAP1 with high affinity (FIG. 6). In our experimental validation we identified both displacers and binders.

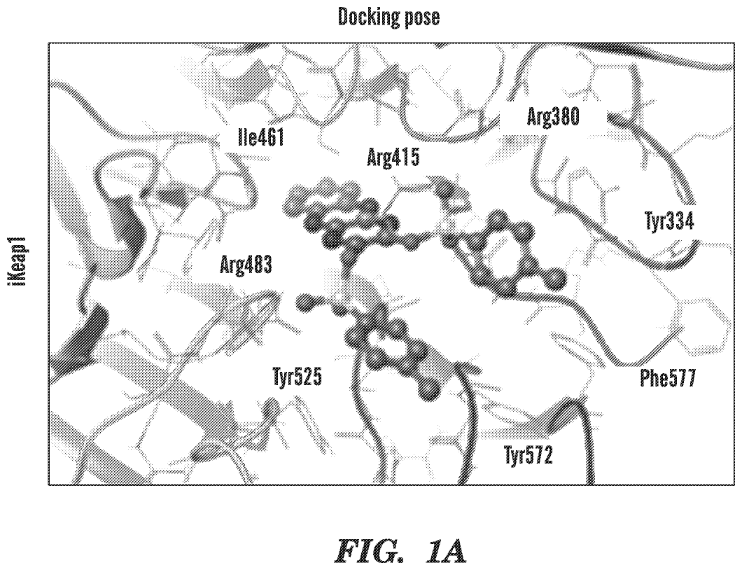

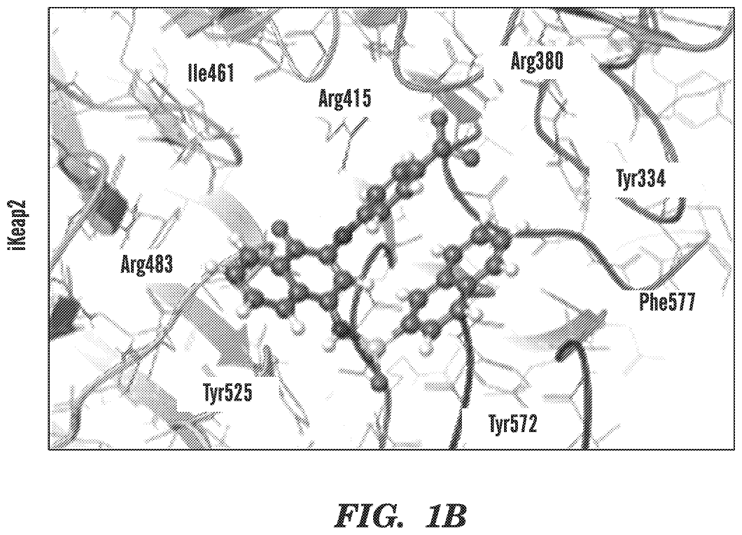

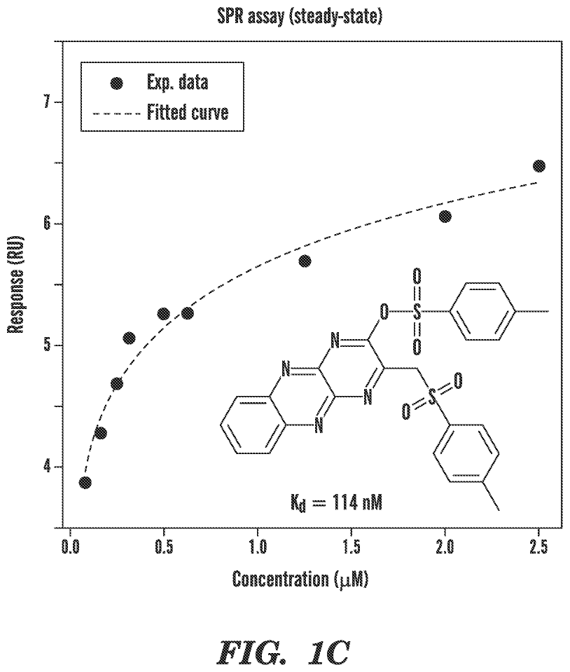

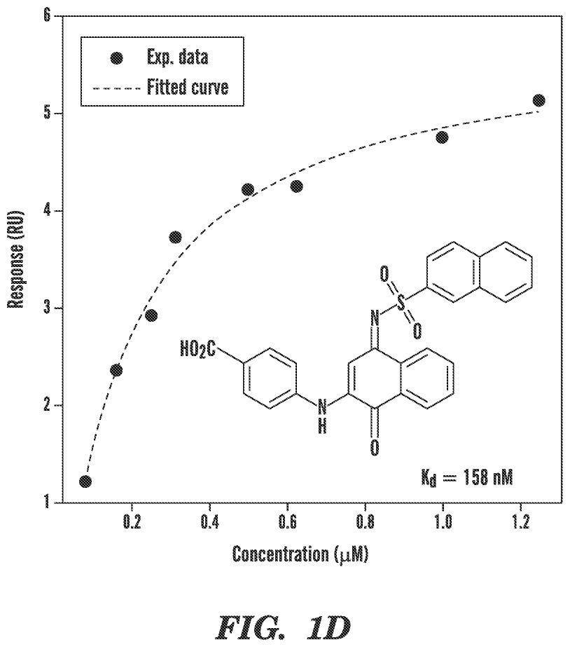

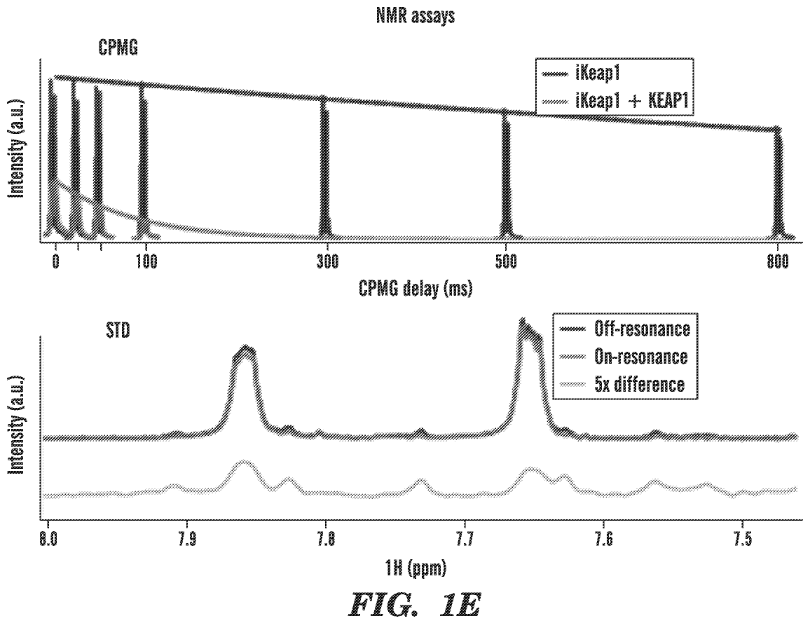

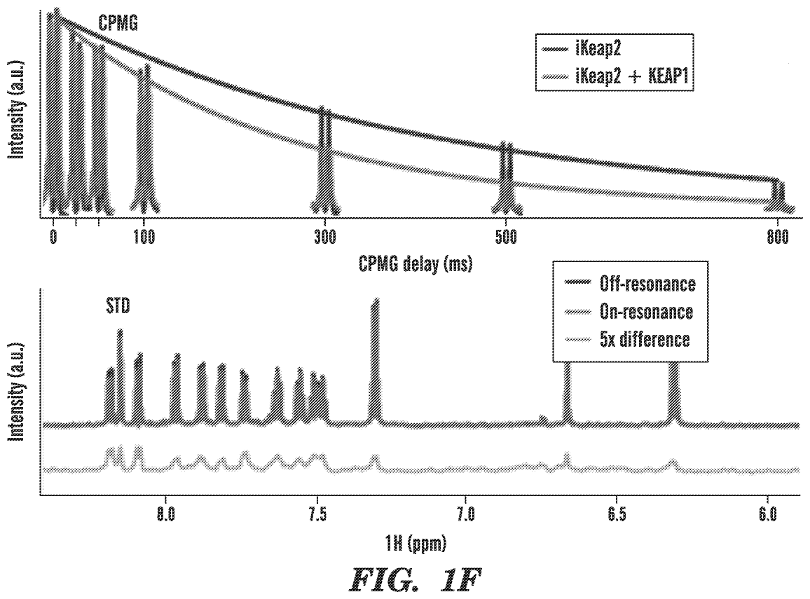

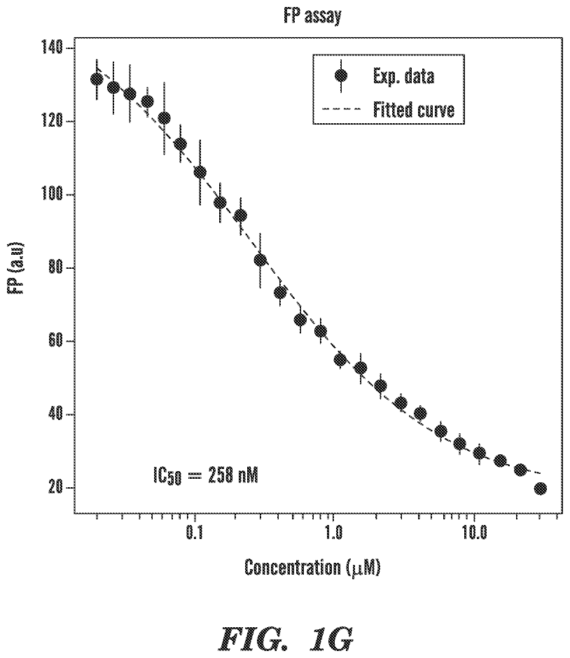

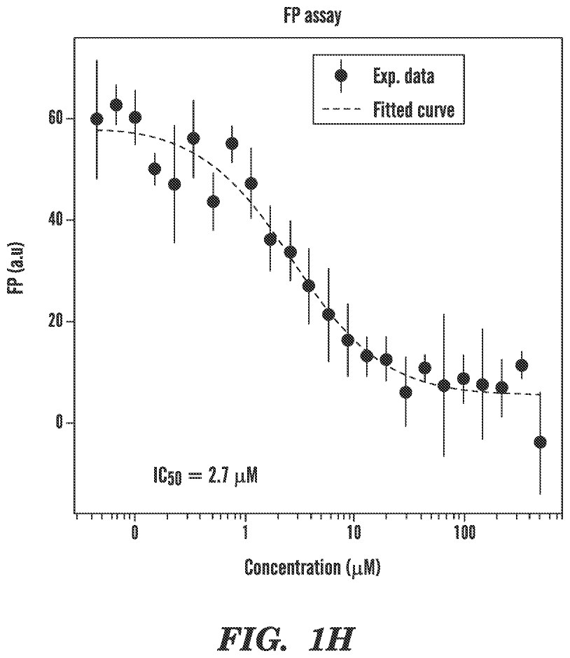

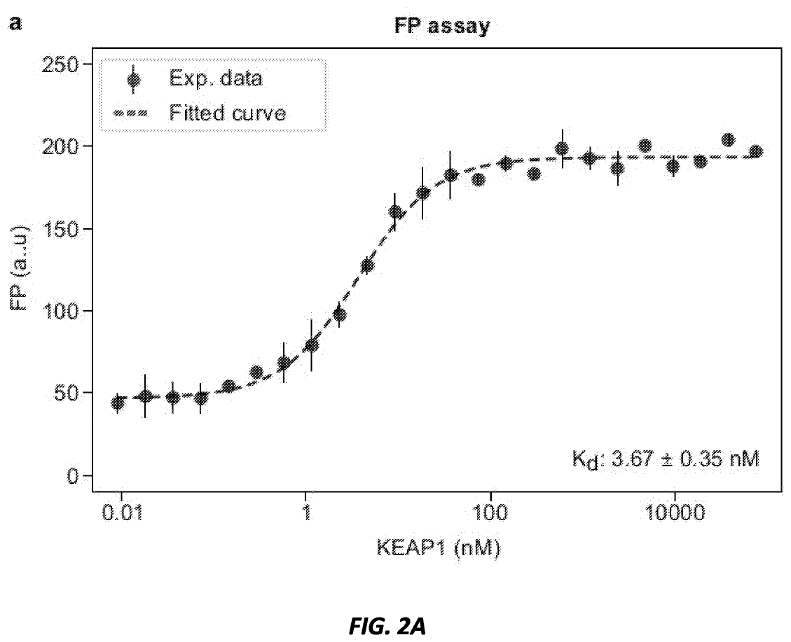

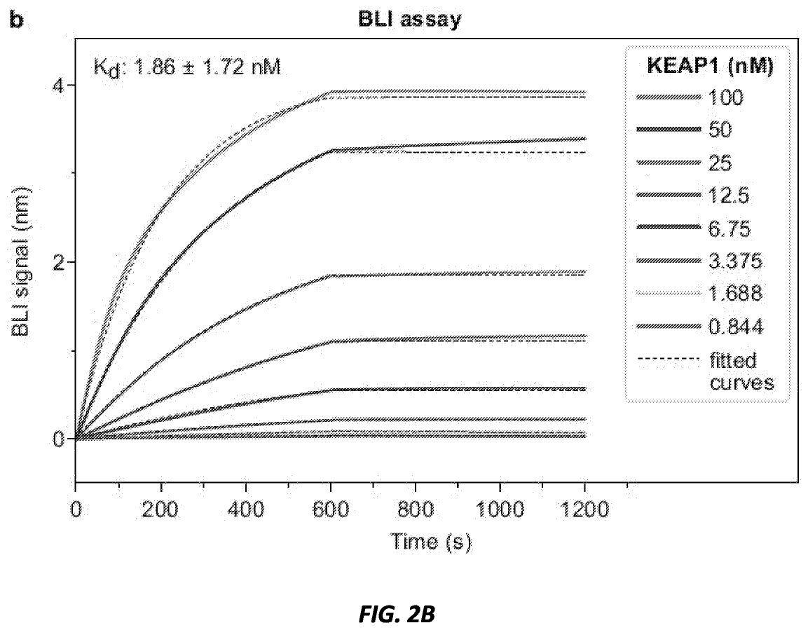

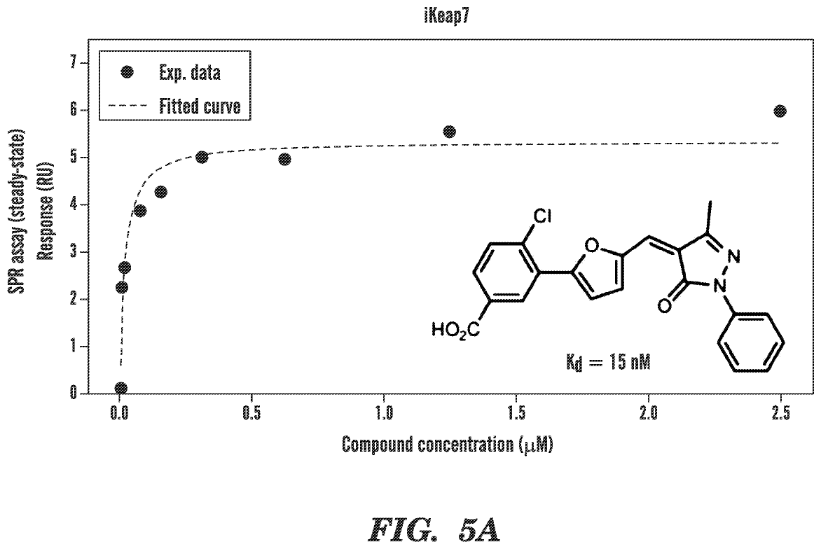

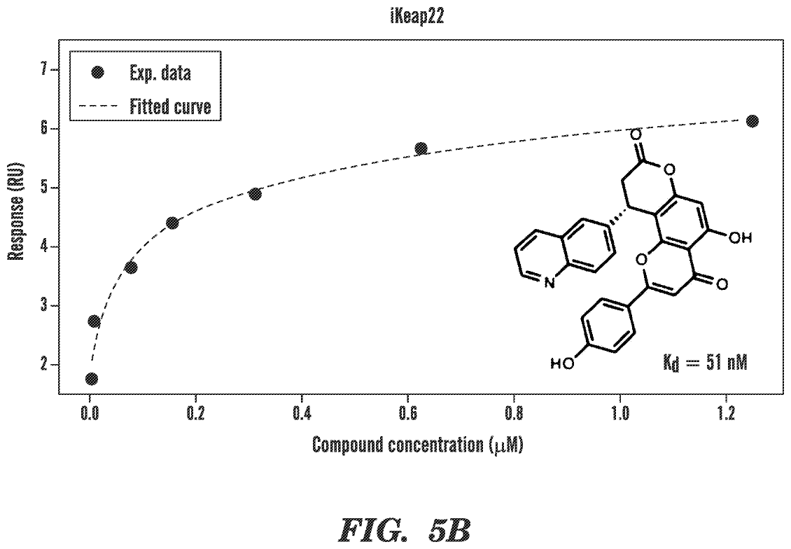

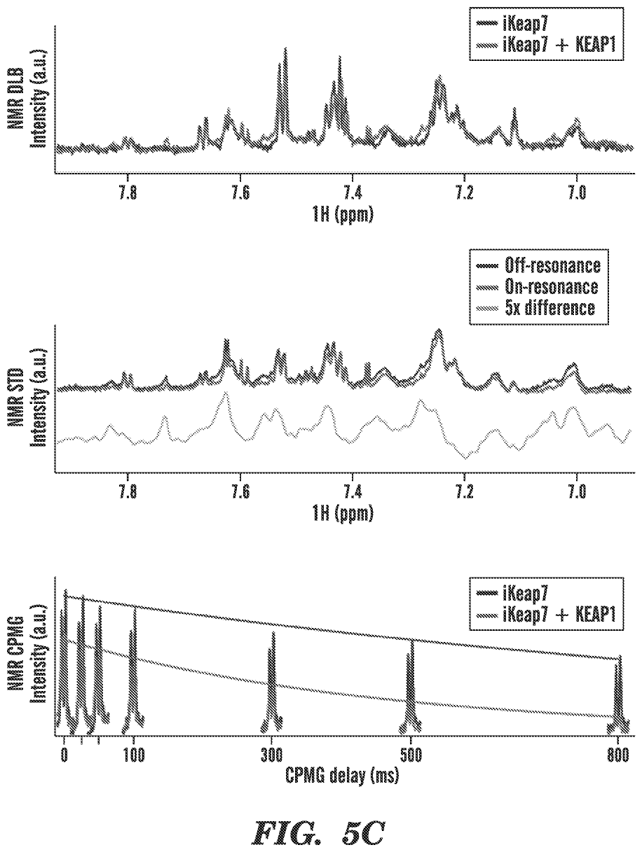

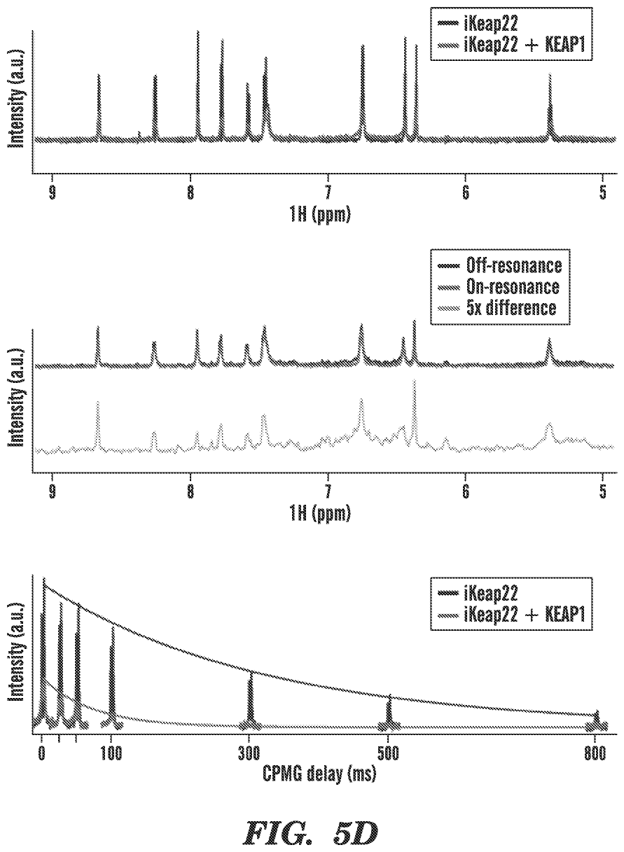

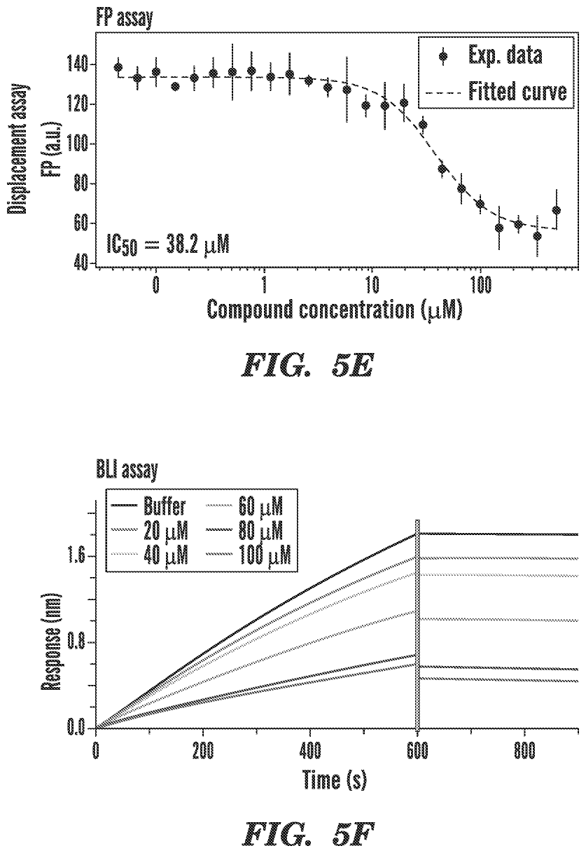

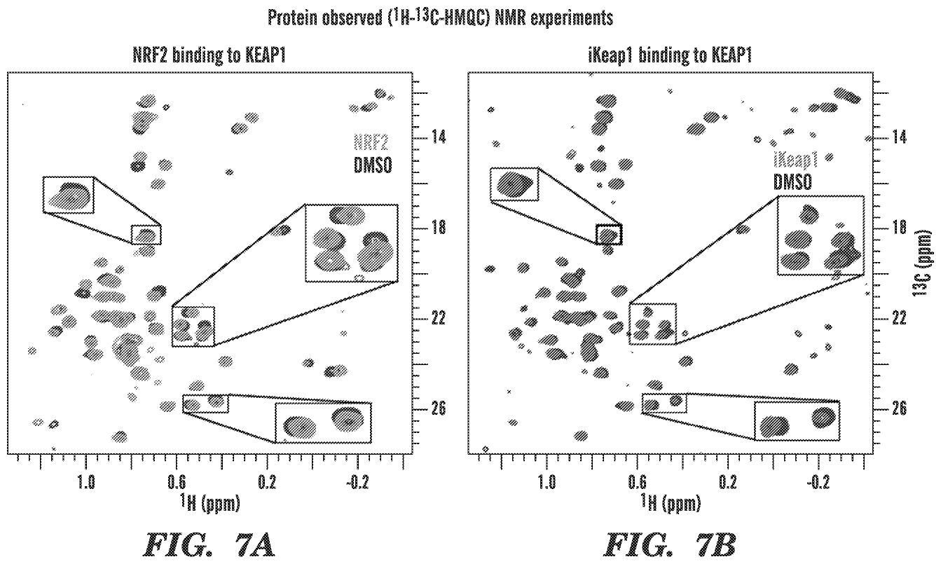

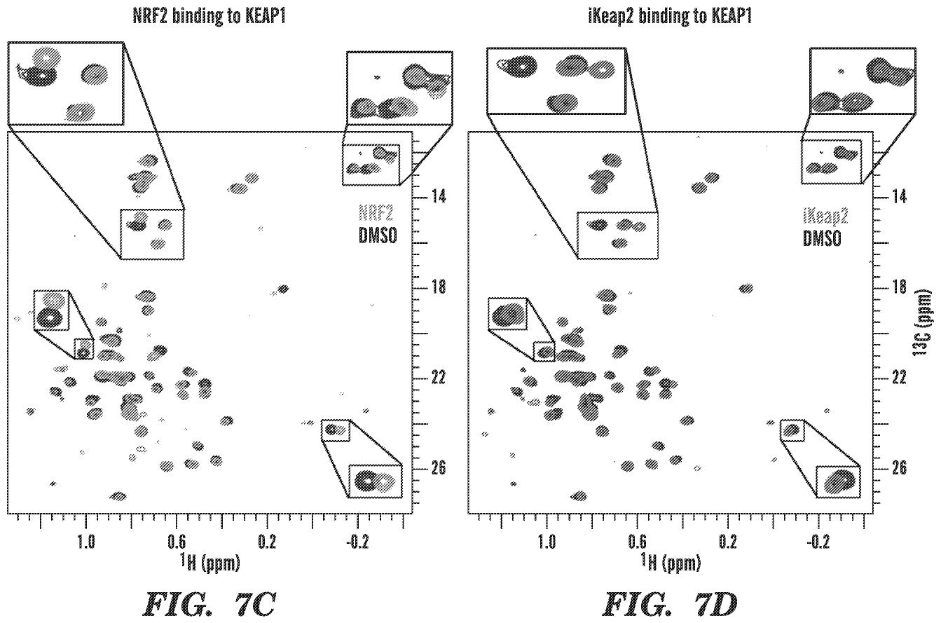

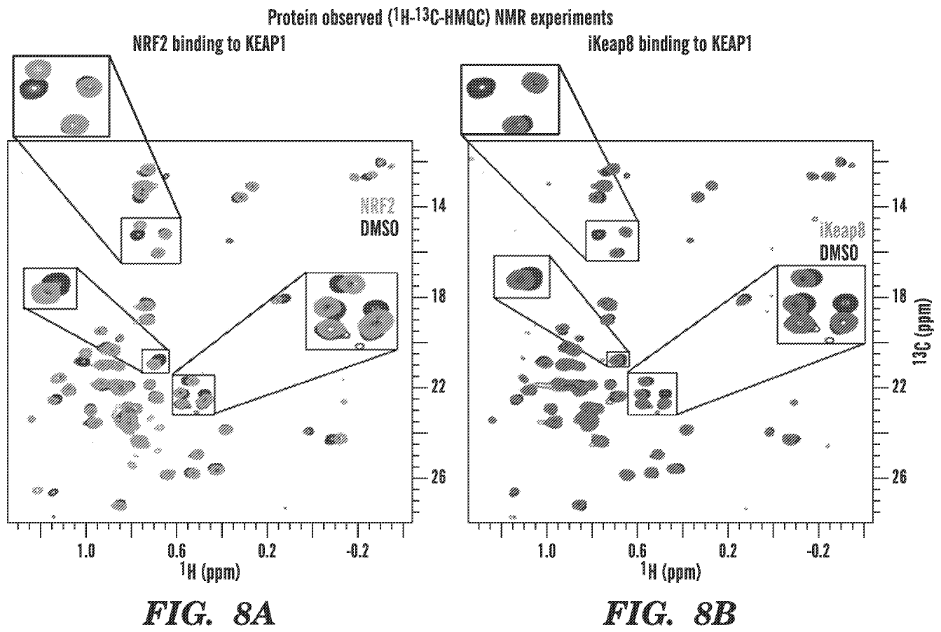

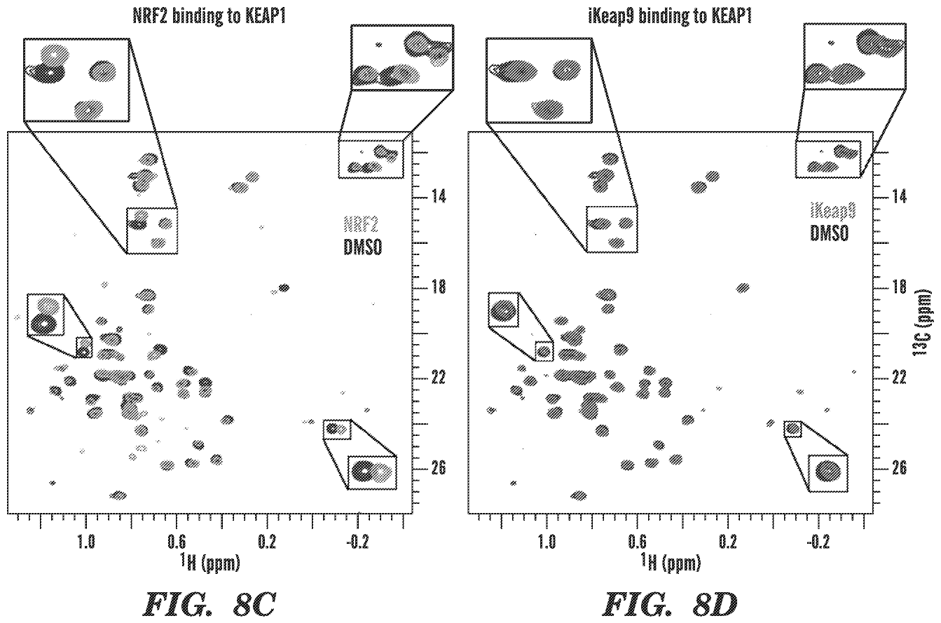

Out of the cherry-picked 590 compounds, 69 were confirmed to bind to KEAP1 by Level-2 SPR. To assess the ability of the compounds to displace the NRF2 peptide we used the FP assay. Ten compounds were confirmed to be displacers with an IC₅₀<60 μM by FP and all of them were identified as a binder by Level-2 SPR. Interference by autofluorescence from the compounds themselves prevented the analysis of some of the compounds by FP. Thus, we used BLI as an orthogonal assay to assess the ability of the compounds to displace NRF2. The binding affinity of the NRF2 peptide to KEAP1 as measured by BLI was 1.86 nM which is similar to that measured by FP, 3.67 nM (Extended Data FIGS. 2A and 2B). 40 compounds of the 69 SPR Level-2 active compounds were able to disrupt the NRF2-KEAP1 interaction as observed by BLI. Of these 40 compounds, 16 were able to displace NRF2 from KEAP1 at a compound concentration of 20 μM, while all 40 compounds could do so at 100 μM. Using BLI, we were able to identify displacers that were missed by FP due to autofluorescence (an example is shown in Extended Data FIGS. 5A-5F). We tested all the SPR Level-2 active compounds for potential aggregation by Dynamic Light Scattering (DLS). We identified seven compounds that aggregated in the DLS assay and hence were not considered for further evaluation (Table 6). Based on the SPR Level-2 and the FP Level-2 binding data, we selected 23 compounds for SPR Level-3 experiments to determine the binding affinity. All 23 compounds had affinities in the low micromolar to nanomolar range, and 12 compounds had submicromolar K_{d} values. From these 23 compounds, we tested the binding of six compounds (iKeap1, 2, 7, 8, 9 and 22) to KEAP1 by a suite of NMR-based ligand-detected experiments. Out of these six compounds, five are displacers and one of them (iKeap9) is a binder. These six compounds were selected on the basis of the solubility constraints of the NMR experiments, the SPR K_{d} value, and/or their ability to displace the peptide. We used differential line broadening (DLB), saturation transfer difference (STD), Car-Purcell-Meiboom-Gill (CPMG)-based transverse relaxation time experiments, and protein-observed ¹H—¹³C heteronuclear multiple-quantum correlation (HMQC) experiments to confirm binding of the compounds to KEAP1. The ligand-detected NMR experiments confirmed that all six of the tested SPR Level-3 active compounds bind to KEAP1 (FIGS. 1A-1H, 4A-4H and 5A-5F). Protein-detected ¹H—¹³C HMQC experiments show that the compounds engage KEAP1 in a specific manner, at the targeted NRF2 binding site. In the absence of resonance assignments, we use the fact that the compounds perturb a subset of KEAP1 resonances affected by the addition of the NRF2 peptide as evidence for competitive binding. These compounds are shown in FIGS. 7A-9D. Details about the other active compounds is provided in Supplementary Information Section B.

Two of our top hits, iKeap1 and iKeap2 are able to displace the NFR2 peptide from KEAP1. Both of the compounds are predicted to engage the NRF2 binding pocket on KEAP1, located at the entrance to the tunnel formed by the β-barrel. (FIGS. 1A and 1B). In comparison to iKeap2, iKeap1 descends deeper into this central tunnel of KEAP1. SPR results showed that iKeap1 and iKeap2 bind to KEAP1 with a binding affinity of 114 nM and 158 nM, respectively (FIGS. 1C and 1D). NMR-based ligand-detected experiments confirmed that both iKeap1 and iKeap2 directly bind to KEAP1 (FIGS. 1E and 1F). FP assays showed that iKeap1 is able to displace NRF2 peptide with an IC₅₀ of 258 nM and iKeap2 displaces the NRF2 peptide with an IC₅₀ of 2.7 μM (FIGS. 1G and 1H). BLI measurements additionally confirmed that both iKeap1 and iKeap2 are able to displace the NRF2 peptide from KEAP1. iKeap1 exhibits similarity to a previously reported naphthalene-based compound with a lower IC₅₀ (IC₅₀=2.7 μM; compound C17 in Table 2 and FIGS. 3A-3D)²⁵. C17 was identified as the best hit in a high-throughput screen (HTS) of 270,000 compounds²⁵.

We would also like to highlight iKeap7, which has the highest affinity as assayed by SPR (K_{d}=15 nM) and displaces the NRF2 peptide with an IC₅₀ of 38.2 μM (FIGS. 5A-5F). It should be noted that of the 14 hits described in the manuscript, only two hits, namely iKeap2 and iKeap7, contain pan-assay interference compound (PAINS) sub-structures. However, we performed a series of orthogonal binding assays, which confirmed that iKeap2 and iKeap7 are not experimental false positives. For details and discussion on how we verified that our experimental results were not affected by PAINS see Supplementary Section B.

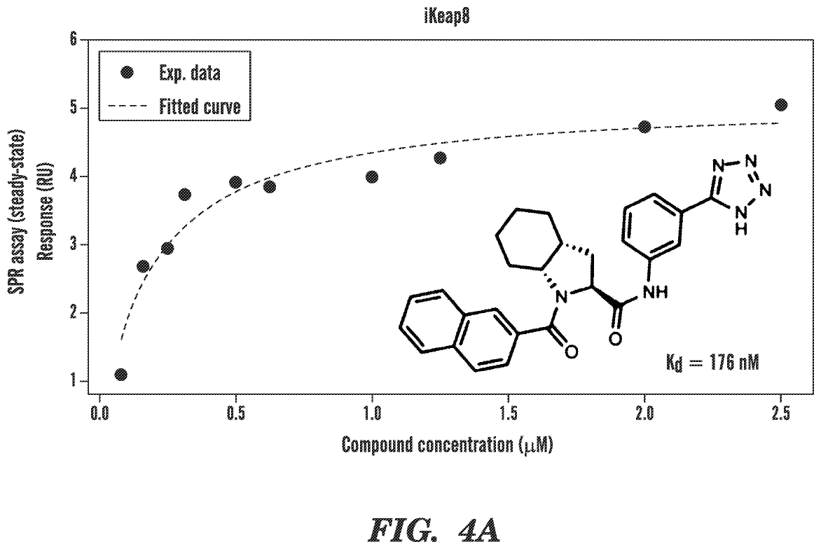

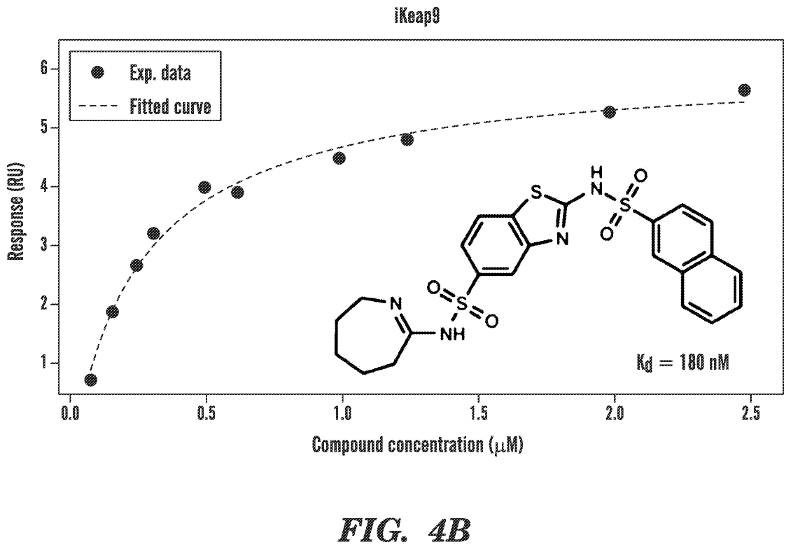

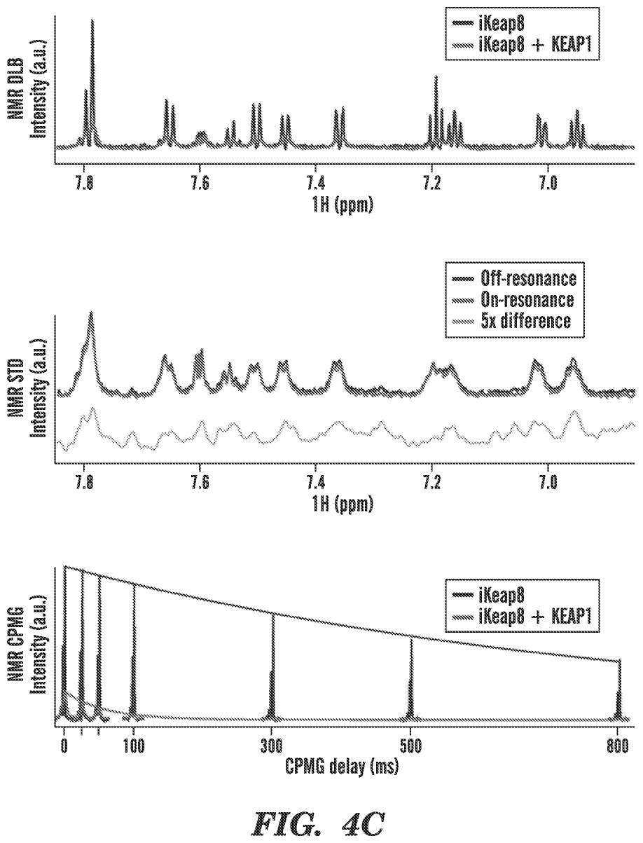

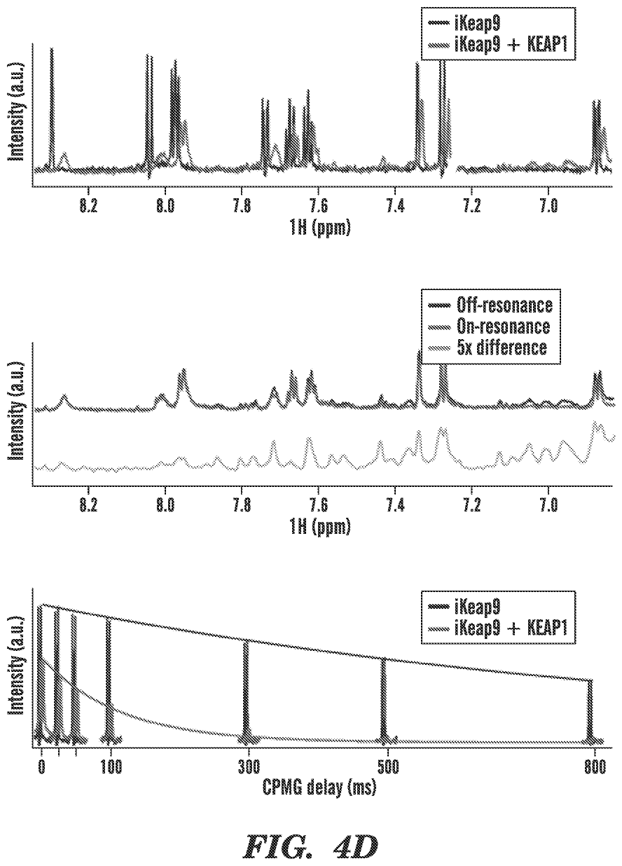

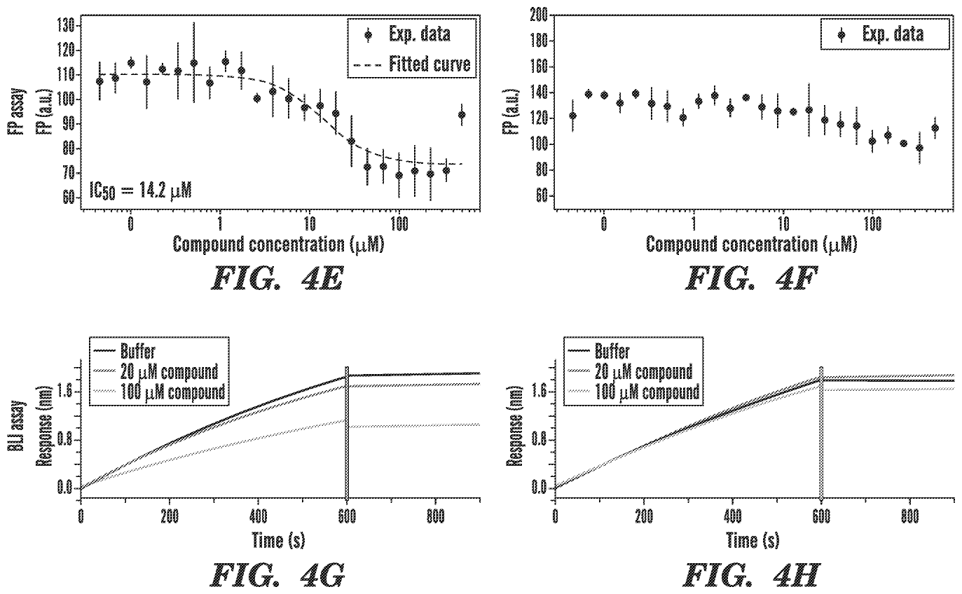

Typically PPIs have a larger interaction interface as compared to that of the active site of an enzyme. Hence the in silico screen can identify binders that either partially overlap with the binding site of the interacting protein, such as iKeap9 (FIGS. 4A-4H), or those that bind in a manner which energetically favours the formation of the protein-protein complex. Examples of the latter, referred to as glues, have been previously described in the literature²⁶.

### An Open Source Platform

To allow VirtualFlow to be used widely and develop dynamically, it is set up as a free and open source (FOSS) project. GPU support is planned for the future and will be incorporated into VirtualFlow both natively and via external docking programs such as Gnina²⁷. We encourage scientists to join the project and contribute to improving existing features, adding new features and functionality. The primary homepage of VirtualFlow, which provides additional resources, can be accessed at https.//www.virtual-flow.org.

### Outlook

VFVS can be used to search extremely large regions of the chemical space, which is the key to identifying promising small-molecule binders. VFVS is able to accomplish this by efficiently utilizing high-performance computing resources, which will continue to increase in availability and power in the years to come, and novel virtual screening databases such as the Chemical Universe Databases (GDBs), which contain billions to trillions of compounds, are still waiting to be explored²⁸.

### Methods

Parallelization of the Virtual Screen with VirtualFlow

VirtualFlow employs four levels of parallelization in a hierarchical manner to permit it to run on batch system-managed Linux clusters of any configuration while allowing for perfect scaling behaviour. Each instance of VirtualFlow can submit multiple jobs, each job may use several job steps (currently only supported when using SLURM and Moab/TORQUE/PBS as the resource manager, while for SGE and LSF only single job steps per job are possible), one job step is able to execute an arbitrary number of queues, and each queue executes the external programs which are processing the ligands. These programs may be additionally parallelized internally, for instance via multithreading.

### Workload Balancing

When processing ligands in parallel, there needs to be a mechanism which makes sure that each ligand is treated only once. However, one main problem with parallelization is that most cluster file systems are too slow to work off a single simple task list. This is because when different processes access the file at the same time, clashes can occur, as it may take up to several seconds until one job sees the changes made to a file by another job. These latency problems also mean that file locking mechanisms do not prevent these clashes. The standard solution for solving this kind of problem is to let different processes communicate directly with each other or via a central master process. However, in most cases, this results in sub-linear scaling behaviour, which normally worsens as more and more parallel running processes become involved. Moreover, many advanced parallelization methods such as MPI or OpenMP, do not allow for inter-job communication, while in many cases multiple simultaneously running jobs are needed. Therefore, in order to maintain perfect scaling behaviour which allows multiple jobs and a virtually unrestricted number of CPUs, we have developed an advanced task-list mechanism. The key is to minimize the number of instances that the parallel processes need to access the task list. The mechanism we have implemented requires only a single access per batch system job, each of which can contain a large number of parallel running processes. For this purpose, we have implemented a workload balancer, which distributes the tasks from the central task list at the beginning of each job to all the queues belonging to it. The central task list contains collections of ligands as elementary components (rather than individual ligands), and the workload balancer takes into account the length of each collection when distributing them among the queues. This approach dramatically reduces the number of times the central task list has to be accessed. For example, if the workflow employs 10 jobs in parallel, and each job runs on 100 nodes with a wall time (real run time) of one week and 24 CPU cores per node, and one ligand requires approximately 30 seconds to be docked, then the central task list needs to be accessed only 10 times per week to feed a total of 24,000 parallel running queues (assuming each queue runs on one CPU core). In this case, approximately 483,840,000 ligands are processed in one week, which means that the advanced task list approach reduced the number of accesses to the central task list by a factor of 48,384,000 in comparison to the number needed by a trivial task list approach (one access per ligand processed). This factor can be improved even further depending on the job size and the cluster wall time. In case two parallel processes want to access the central task list simultaneously, two backup mechanisms were implemented. The first mechanism is a time-dispersion mechanism, which spreads out simultaneously arriving jobs in time, and further stalls subsequent jobs until the workload balancer of the current job is finished. If this mechanism should fail to prevent a simultaneous access event, which could result in a damaged or empty task file, a second mechanism restores the task list using an automatically backed-up copy of a previous version of the central task list.

### Reduction of I/O Load

One of the potential bottlenecks of computer clusters is the I/O load they can handle, even when they utilize shared cluster file systems with high bandwidth. The limit of the I/O capacities of a cluster can be easily reached if many small processes that individually handle their I/O and use the shared file system are running in parallel. This circumstance can pose a serious problem when running large-scale workflows with thousands of queues working in parallel, and can easily lead to crashing the cluster file system. To address this problem and dramatically minimize the load on the shared file system, VirtualFlow is able to perform most I/O operations on the local temporary file systems of the computing nodes, which are normally fast RAM-based (virtual) drives readily available on any Linux system (usually/dev/shm). The final output files are then stored in batches at large time intervals on the permanent cluster file system.

### Preparation of the Ligand Databases

One of the ligand databases which was screened originates from the state of the ZINC 15 database in the autumn of 2016. Approximately 330 million compounds were downloaded in the SMILES format and converted into three-dimensional PDBQT files with VFLP because, at the time, the ZINC 15 database only provided a fraction of the compounds in a ready-to-dock format. During the conversion, the molecules were protonated with ChemAxon's cxcalc and the three-dimensional structure of the ligand was computed by ChemAxon's molconvert tool²⁹. If protonation or the generation of the three-dimensional structure failed, Open Babel³⁰ was employed as a fallback option. Other preparation steps, such as desalting, were not carried out on these compounds as they had already undergone these basic preparation steps for the ZINC 15 database.

We also prepared the compounds in the REAL database provided by Enamine³¹. Approximately 700 million partially-stereospecific SMILES were expanded into fully stereospecific SMILES, resulting in around 1.4 billion molecules. These were then prepared with VFLP into a ready-to-dock format. Specifically, the compounds were desalted and neutralized with ChemAxon's cxcalc, major tautomers were computed with cxcalc, and then protonated with cxcalc (using Open Babel as fallback), the 3D coordinates were computed with ChemAxon's molconvert²⁹ (using Open Babel as a fallback), and finally converted into the PDBQT format with Open Babel. This library has been made available via an interactive web interface (Supplementary Section C). The scaling behaviour of VFLP was measured on the GCP up to 20,000 CPU cores (data not shown).

### Computation Time of VFVS

The total computation time (T) is directly proportional to the number of ligands screened (N) and the processing time per ligand (P), and inversely proportional to the number of CPUs used (C):

T≈(P*N)/C

The processing time per ligand (P) depends mainly on the specific docking scenario (which includes the receptor and all the possible docking options/parameters) and the speed of CPUs used, and can be approximated by the equation

P≈(E*Θ+ζ)/η

where η is a factor representing the CPU speed relative to a reference CPU, E is the docking exhaustiveness parameter (elaborated in the next paragraph below), Θ is the docking time per unit exhaustiveness on the reference CPU, and ζ is the initial setup time required by the docking program on the reference CPU. For a typical case of a large-scale first-stage virtual screen on one of the newer Intel CPUs, the average processing time per ligand (P) is roughly 5 seconds using the fastest docking settings. It follows that when 5,000 CPUs are used, the total screening time for 100 million compounds will be roughly 30 hours.

### Relationship Between the Exhaustiveness Parameter and the Docking Time

The time to dock a single molecule depends on the number of conformations that are sampled, and this number is largely independent of the size of the docking box or surface area. The number of conformations sampled can be controlled by the exhaustiveness parameter of the docking programs. Docking time has a linear dependency on the exhaustiveness parameter. The inset in the graph shows the slope for each of the docking programs, providing an estimate of the degree of dependency between the computational time and the exhaustiveness parameter for individual docking programs.

Since most docking programs utilize a probabilistic search algorithm, the results of separate iterations with the same starting set-up can differ. This circumstance can be beneficial as it can be more efficient to carry out multiple less exhaustive docking iterations than to run one highly exhaustive iteration. The exhaustiveness here is a measure of the extent to which the conformational space of the ligand, and potentially the protein side chains, is explored by the search algorithm during the docking procedure. In light of this, VFVS can be configured to carry out multiple replicas per docking scenario, thus improving the overall efficiency.

### Lead Optimization Using VFVS

The operational flexibility enables VFVS to also be used during lead. In this context, a library of analogues of a chosen lead compound can be prepared with VFLP and screened by VFVS with high docking accuracy (e.g. setting the exhaustive parameter to a high value, allowing specific amino acids in the binding interface to be flexible, using multiple docking programs, and/or multiple receptor (backbone) conformations), which can considerably accelerate the lead optimization process.

### Parameters of the Virtual Screen Against the KEAP1 Target

For the virtual screening validation test, the crystal structure of the KEAP1 Kelch domain (PDB-ID 5FNQ⁹) was used. The protein was stripped of all small molecules present (including water), was protonated at physiological pH, and then converted into PDBQT format using AutoDockTools³².

The NRF2 binding interface on KEAP1 was chosen as the target of the screening, and the exact location determined by previously published co-crystal structures of KEAP1 and the NRF2 peptide (such as PDB ID: 4IFL). The in silico screen was carried out as follows: VFVS used the docking program QuickVina 2 in an initial (primary) virtual screen with the mouse KEAP1 as a rigid receptor structure.

In this primary virtual screening, the docking search space was a rectangular parallelepiped (i.e. a cuboid) of size 15.0×16.5×14.275 Å. The exhaustiveness parameter was set to 1, which favours fast computational times. The quality of individual docking results, and therefore the ranking, depends largely on the external docking program chosen (which is independent of VirtualFlow).

In the rescoring procedure, the following amino acid side chains at the binding interface were allowed to be flexible: Tyr334, Arg380, Asn382, Arg415, Cys434, His436, Ile461, Phe478, Arg483, Ser508, Tyr525, Tyr572 and Phe577. AutoDockTools was used to generate the rigid and flexible receptor structures in PDBQT format. The exhaustiveness was set to 1, and two replicas (iterations) were carried out of each docking scenario (with Smina Vinardo and AutoDock Vina as the docking programs). The size of the docking box was set to 27.0×27.0×24.0 Å.

### Expression and Purification of GST-KEAP1:

A codon optimized vector of the mouse KEAP1 Kelch domain (residues 322-624) cloned into a pGEX-6P-3 vector with BamHI and XhoI cloning sites, and an NRF2 peptide (AFFAQLQLDEETGEFL (SEQ ID NO: 1) with an N-terminal tetramethylrhodamine (TAMRA) fluorophore were purchased from GenScript USA Inc. (NJ, USA). The pGEX-6P-3 vector contains an N-terminal glutathione S-transferase (GST) tag which is expressed as a fusion with the target sequence, resulting in a gene product that will henceforth be referred to as GST-KEAP1. The vector carrying GST-KEAP1 was transformed into BL21(DE3) E. coli. The transformed cells were grown at 37° C. to an optical density of 0.6 at a measurement wavelength of 600 nm and protein expression was induced with 0.5 mM isopropyl β-D-1-thiogalactopyranoside (IPTG). The cells were allowed to grow for 12-16 h at 18° C. and subsequently harvested by centrifugation at 4,200 rpm for 20 min at 4° C.

To purify GST-KEAP1, cell pellets from 2 L of culture were resuspended in 40 mL of GST binding buffer (25 mM Tris-HCl, pH 8.0, 150 mM NaCl, 1 mM EDTA) supplemented with 3.5 mM β-mercaptoethanol and protease inhibitors (Roche). Cells were lysed by sonication, the insoluble fraction was removed by centrifugation at 16,000 rpm and the soluble fraction was applied to 10 mL of GST slurry (GoldBio, MO). The suspension was mutated for 4 hours at 4° C., and the unbound fraction was removed by gravity-flow chromatography. The slurry was washed twice with GST binding buffer supplemented with 3.5 mM β-mercaptoethanol. The bound fraction was eluted from the slurry with 20 mM reduced glutathione in GST binding buffer. The resulting eluate was loaded on a Superdex 200 size exclusion column (SEC) pre-equilibrated in SEC buffer (20 mM Tris-HCl, pH 8.0, 50 mM NaCl, 10 mM dithiothreitol).

### Fluorescence Polarization (FP) Assays:

Dissociation constant of the NRF2-KEAP1 interaction: We prepared 2 nM TAMRA-NRF2 peptide in FP buffer (20 mM Tris-HCl pH: 8.0, 50 mM NaCl, 10 mM DTT, 2 mM 3-[(3-Cholamidopropyl)-dimethylammonio]-1-propanesulfonate (CHAPS), 0.005% BSA, 1% DMSO) in Corning 3575 384-well plates, to establish the dissociation constant (K_{d}) of the TAMRA-NRF2-GST-KEAP1 interaction. GST-KEAP1 was titrated into the TAMRA-NRF2 peptide starting at a concentration of 76 NM GST-KEAP1 followed by two-fold dilutions for a total of 24 points. A K_{d} of 3.67±0.35 nM was determined for the interaction (FIGS. 2A and 2B).

FP Level-1 (high-throughput screening of compounds): All 590 compounds ordered for testing were dissolved in DMSO-d₆ to a final concentration of 10 mM. Two AB1056 (Abgene, NH, USA) plates were prepared as source plates for screening. The first source plate contained 11 μL of each of the 10 mM compounds. The second source plate was filled with 9 NL DMSO and 1 μL from the first source plate was transferred into the second via pin transfer with a Vprep liquid handling pipetting station (Agilent, CA), resulting in a final concentration of 1 mM for each compound in the second source plate. 384-well Corning 3575 (Corning, N.Y., USA) assay plates were pre-loaded with 7 nM GST-KEAP1 in FP buffer (30 μL/well). 300 nL, 100 nL, and 33 nL volumes were transferred from each source plate (the 10 mM and 1 mM plates) to pre-loaded 384-well assay plates. The assay plates were incubated for 1 hour at room temperature before 2 nM TAMRA-NRF2 peptide was added to each well with an HP D300 (Hewlett-Packard, CA). After 3 hours of incubation at room temperature, fluorescence polarization (excitation 485 nm/emission 520 nm) was measured using an EnVision plate reader (PerkinElmer, MA, USA). This assay resulted in six-point titrations, which are not sufficient to calculate accurate IC₅₀ values, but allow for the selection of top binders.

FP Level-2 (screening of top hits): The 27 compounds which were active in the FP Level-1 assay were subjected to a second 24-point FP screen (Level-2), starting from 500 μM compound followed by 1.5-fold serial dilution. For the best compound, iKeap1, the starting concentration was lowered to 30 μM and the following concentrations were used in the titration: 30.00 μM, 21.60 μM, 15.50 μM, 11.10 μM, 8.00 μM, 5.76 μM, 4.14 μM, 2.98 μM, 2.15 μM, 1.54 μM, 1.11 μM, 0.80 μM, 0.576 μM, 0.414 μM, 0.298 μM, 0.215 μM, 0.154 μM, 0.111 μM, 0.080 μM, 0.0606 μM, 0.0459 μM, 0.0348 μM, 0.0264 μM, 0.02 μM. The measurements were carried out in triplicate, and the three data points for each concentration averaged. IC₅₀ values were determined by fitting the averaged data points to a four parameter logistic curve using the non-linear least squares method provided by the SciPy library for Python³³. The standard error (see Table 5) on the IC₅₀ was computed by taking the square root of the diagonal of the parameter covariant matrix.

### Bio-Layer Interferometry (BLI) Assays

Bio-layer interferometry binding and displacement assays: NRF2-KEAP1 binding BLI experiments were performed on an Octet RED384 (ForteBio, Menlo Park, Calif., USA) using streptavidin-coated Dip and Read Biosensors (ForteBio) and 384 well plates with 120 μL volume. The sensors were incubated for 5 minutes in 500 nM biotinylated NRF2 peptide in binding buffer (10 mM HEPES, pH 7.5, 50 mM NaCl, 0.1% (v/v) Tween20 with 0.5 mM TCEP and 1% DMSO). To test for nonspecific binding of GST-KEAP1 protein, reference tips were incubated in buffer alone. The tips were washed with buffer for 2 minutes to obtain a baseline reading and then transferred to wells in various concentrations of GST-KEAP1 protein (100 nM, 50 nM, 25 nM, 12.5 nM, 6.75 nM, 3.375 nM, 1.679 nM, 0.844 nM) for 10 minutes. After measuring association, tips were moved to wells containing buffer, and dissociation was measured for 5 minutes. The data were processed and analysed using the Octet data analysis software version 11.0 (ForteBio, Inc., Menlo Park, Calif., USA). The association-dissociation curve for each concentration was fitted using a 1:1 model given by the equations

$${R}_{t}^{on} = \frac{{k}_{on} \cdot C}{{k}_{on} \cdot C + {k}_{on}} {R}_{max} ( 1 - {e}^{- ( {k}_{on} \cdot C + {k}_{off} ) \cdot t} ) \qquad ( 3 )$$

$${R}_{t}^{off} = {R}_{eq} \cdot {e}^{- {k}_{off} \cdot t} \qquad ( 4 )$$

where Rₜ^{on} and Rₜ^{off} are the BLI signals at time t, R_{eq} is the equilibrium response, kₒₙ is the association rate constant, k_{off} is the dissociation rate constant, C is the analyte (protein) concentration, and R_{eq} is the signal level at the equilibrium of association which depends on the analyte (protein) concentration and the maximal capacity (Rₘₐₓ) of the sensor surface. By computing the ratio k_{off}, kₒₙ the apparent equilibrium constant K_{d} is obtained. The resulting apparent K_{d} values were averaged.

Compound screening by BLI displacement assay: The BLI displacement assays were setup as described above. The biotinylated NRF2 peptide was used at a concentration of 500 nM and GST-KEAP1 protein was used at a concentration of 25 nM. The compounds were used at concentrations of 20 and 100 NM, and pre-incubated with GST-KEAP1 protein. The association phase was measured in the well containing compound with GST-KEAP1 protein for 10 minutes, and followed by a dissociation phase in buffer for 5 minutes. The inhibition percentage was the average BLI signal in the last 50 seconds of the dissociation phase, normalized against the condition of GST-KEAP1 protein in the absence of compound. The dose-dependent experiment with iKeap22 was carried out at 10 μM, 20 μM, 40 μM, 80 μM and 100 μM compound concentration and pre-incubated with 25 nM GST-KEAP1 protein.

To test for nonspecific binding of the compounds, the sensor was coupled with biotinylated NRF2 peptide and the compounds were used at 20 μM concentration without protein.

### Surface Plasmon Resonance (SPR) Binding Assays

All SPR binding experiments were performed on a BiacoreT200 (GE Healthcare, Sweden) instrument at 25° C. in running buffer (10 mM HEPES pH 7.5, 50 mM NaCl, 0.1% (v/v) Tween20 with or without 0.5 mM TCEP, 1% DMSO). The running buffer was prepared freshly on each day of use, filtered and degassed prior to the SPR experiments. The target protein (GST-KEAP1) was anchored on a CM5 chip via a GST labelling kit (GE Healthcare, Sweden)³⁴, where a polyclonal goat anti-GST antibody was immobilized on a CM5 sensor chip by the amine coupling method 1.

SPR Level-1 (1-point HTS): The SPR Level-1 screening was carried out as previously reported³⁵. First, we prepared 10 mM d6-DMSO stock solutions of the 590 compounds which were procured in powder form. 20 μM samples of the compounds were made by diluting the stock compounds in running buffer with 0.5 mM TCEP and 1% DMSO. The anti-GST immobilizing chip was saturated with GST at the reference channel and GST-KEAP1 at the target channel with resonance unit (RU) values of 750-800 for the GST and 2,000-3,000 for GST-KEAP1. Binding of compounds to the immobilized protein was monitored for 60 seconds in both the association and dissociation phase. Additional injection of the running buffer was performed after every compound binding. All binding signals (RUₘₐₓ=16-29 RU, 1:1 stoichiometry) were corrected for the signals from the reference channel and buffer blank. Compounds were classified as an SPR Level-1 hit if the condition RU>4 was satisfied. This criterion was based on the positive control (iKeap1, RU=4.65±0.74).

SPR Level-2 (5-point HTS of the SPR Level-1 hits): The hits from the SPR Level-1 assay were re-screened at five different compound concentrations (0.5, 1, 5, 10 and 20 μM), in running buffer with 0.5 mM TCEP and 1% DMSO, at a rate of 30 μL/min. The hits were classified as hits if they produced a concentration dependent SPR response and an RU value >4 at a compound concentration of 20 μM.

SPR Level-3 (SPR experiments of selected SPR Level-2 hits): 23 out of the 69 SPR Level-2 hits were chosen for Level-3 analysis. Given the low-throughput of the Level-3 SPR assay, we chose a subset of the SPR Level-2 hits, which included the displacers from the Level-3 FP assay, the compounds that were tested by NMR, and select SPR Level-2 hits. SPR experiments were carried out in which the target protein (GST-KEAP1) was captured and regenerated in each compound cycle. All SPR data processing and analyses were performed using the BiaEvaluation software (version 3.0). For steady-state binding, the R_{eq} signal was plotted against the analyte concentration and fitted to the one-site or the biphasic binding model (see Table 4) via the Levenberg-Marquardt algorithm used by the BiaEvaluation software. The one-site binding model is given by the equation

R_{eq}=(Rₘₐₓ*C)/(K_{d}+C)+b, (1)

where R_{eq} is the SPR signal at equilibrium, Rₘₐₓ is the SPR signal at saturation of the binding mode, K_{d} is the dissociation constant of the compound, b is the offset, and C is the concentration of the compound. The biphasic binding model is given by the equation

R_{eq}=(R_{max,1}*C)/(K_{d,1}+C)+R_{max,2}*C/(K_{d,2}+C)+b, (2)

where R_{eq} the SPR signal at equilibrium, R_{max,1} and R_{max,2} are the SPR signals at saturation of the two binding modes, K_{d,1} and K_{d,2} are the dissociation constants of the compound corresponding to the two binding modes, b is the offset, and C is the concentration of the compound.

Standard errors of the estimated K_{d} values were computed with the BiaEvaluation software, which computes them via the diagonal elements of the covariance matrix and the residual. The software operates based on the equations found on page 378 in the book ‘Receptor-Ligand Interactions: A Practical Approach’³⁶.

### Ligand-Detected NMR Experiments

The differential line broadening (DLB) experiments serve as simple one-dimensional experiments, where the proton signal of the ligand is monitored. The ligand concentration exceeds the receptor concentration (e.g. 10-20-fold) in this experiment and broadening of the resonance frequencies in presence of the receptor is a consequence of ligand molecules shuttling between free and bound states. DLB manifests as a broadening of the ligand resonance due to binding a protein. The ligand is in equilibrium between the free and protein-bound states dictated by the equilibrium constant. DLB is the result of the change in relaxation rate and the difference in chemical shift of the bound ligand. In the STD experiments, a region of the spectral space (−1 to 0.5 ppm) that has resonances from the receptor but not the ligand is selectively saturated. Resonances from methyl bearing amino acids (Ile, Leu, Val) often populate this region of the spectral space. This saturation is transferred to the rest of the protein and eventually to the bound ligand by spin diffusion (NOE). In the implementation of STD, two one-dimensional spectra are recorded in an interleaved fashion. In the first experiment neither the receptor nor the ligand is saturated (off-resonance) and in the second the receptor is selectively saturated (on-resonance). Spectra of free ligand are observed in both experiments. However, if the ligand transiently binds to the receptor then the saturated receptor will transfer magnetization to the ligand. This transfer will be reflected as reduced intensity in the on-resonance saturated spectrum compared to the off-resonance saturation. The results are often presented as a difference spectrum between the on and off-resonance saturation experiments. The appearance of ligand resonances in the difference spectrum is indicative of ligand binding. Measurement of the transverse relaxation rate of the ligand is another complementary strategy to detect ligand binding to a receptor. The free ligand behaves like a small molecule and experiences slow transverse relaxation, however transient binding to the receptor enhances the transverse relaxation rate of the ligand. Thus, an increased transverse relaxation rate in presence of a receptor directly indicates binding to the receptor. In the experimental setup a series of 1D experiments where the coherences of ligand spends increasing amounts of time in the transverse plane is recorded. Ligands that engage the protein will relax faster than unbound ligand. We refer to these experiments as hear as CPMG-R2 or CPMG experiments. While any of these experiments are in principle sufficient to demonstrate ligand binding, false positives for either of these experiments have been reported. However, a detection of a false positive hit is highly unlikely if all three experiments indicate binding, which is the case with all the hits reported here.

All the ligand-detected experiments were performed with 50 μM compound alone or in presence of 5 μM KEAP1 (without the GST tag) in NMR buffer (phosphate saline buffer supplemented with 5% DMSO-d₆ and 4 mM deuterated DTT at pH 7.4) unless otherwise noted. For iKeap1 and iKeap2, the protein concentration was kept at 2.5 μM due to tight binding. ¹H 1D spectra of the compounds were recorded in the absence and in the presence of KEAP1 to assess line broadening. Saturation transfer difference spectra of the compounds in presence of KEAP1 were recorded with 3 second saturation time on (0 ppm) and off (−20 ppm) resonance, respectively. The relaxation rate of the compounds was measured in the absence and the presence of KEAP1 with a series of ¹H 1D experiments with CPMG-based transverse relaxation time filters of various lengths: 1 ms, 25 ms, 50 ms, 100 ms, 300 ms, 500 ms and 800 ms. Data were analysed and visualized in Matlab (MathWorks, MA).

### Protein-Detected NMR Experiments

The cleaved mouse KEAP1 Kelch domain (residues 322-624) consists of 308 amino acids with close to 300 detectable amide resonances. Therefore, correlating chemical shift perturbations of small molecule inhibitors to perturbations introduced by NRF2 would have been prohibitively difficult in ¹H-¹⁵N HSQC spectra without full backbone assignment. Our aim here was to rely on methodology that can be quickly and easily implemented even for very large proteins for which backbone assignment might not be feasible. Indeed, with a molecular weight of 33.7 kDa, the mouse KEAP1 Kelch domain (322-624) is already on the larger side for NMR backbone assignment. To overcome the spectral crowding in a ¹H—¹⁵N HSQC spectrum and minimize problems due to low ligand solubility, we implemented a ¹H—¹³C TROSY-HMQC experiment coupled with fast data acquisition. For the protein detected ¹H—¹³C HMQC experiments a sample of KEAP1 which is selectively ¹H and ¹³C labelled at the methyl groups of isoleucine, leucine, and valine residues, in an otherwise deuterated background was used. This labelling strategy is referred to as ILV labelling. ILV-labelled samples of KEAP1 were prepared by culturing BL21(DE3) cells containing a plasmid for GST-KEAP1, in perdeuterated M9 medium with 1 g ¹⁵N—NH₄Cl and 2 g ²H-culturing-¹²C-glucose in ²H₂O. One hour before induction with IPTG, 330 mg/L 2-(¹³C)methyl-4-(²H₃)-acetolactate (precursor for leucine and valine) was added. Prior to that the acetolactate was activated as previously described³⁷. 20 min before induction 75 mg/L ¹³C/¹H methyl—and otherwise deuterated—ketobutyrate sodium (precursor for isoleucine) was added. The use of acetolactate resulted in stereospecific ¹H—¹³C labelling of only one of the leucine (λ2) and valine (γ2) methyl groups as previously described³⁷. The protein was purified as described above. The GST-tag was cleaved by preScission protease cleavage and the free mouse KEAP1 Kelch domain was eluted in NMR buffer from a size exclusion column. All NMR measurements for the ILV-labelled KEAP1 were performed at a protein concentration of 5 μM. The protein concentration was kept low to account for poor solubility (for NMR) of some of the compounds. The concentrations of the compounds were 50 μM, except for iKeap1 and iKeap2, where the concentrations were 25 μM, due to poor solubility.

Given the low concentration of the protein, we used the methyl SOFAST methyl TROSY with 46 ms and 18 ms acquisition times in the direct and indirect dimensions, respectively³⁸. The spectral width was set to 14 ppm (¹H) and 20 ppm (¹³C) in the direct and indirect dimensions, respectively and the spectrum was recorded at 298 K on an 800 MHz Bruker spectrometer equipped with an AVANCE III console and a cryogenically cooled probe. A 4.5 ms Pc9_4_90.1000 pulse was used for selective excitation of the methyl ¹H resonances and a 1.2 ms Rsnob. 1000 pulse was used to selectively refocus proton chemical shift evolution and ¹H—¹³C J-coupling during ¹³C chemical shift evolution. Proper choice and calibration of the excitation and refocusing pulses is crucial to avoid perturbing the water signal, which can significantly lower the achievable signal to noise. Fast data acquisition was achieved with a 150 ms recycling delay, which allowed for the recording of experiments with 512 scans in 5 hours.

### Detecting Aggregation Using Dynamic Light Scattering (DLS)

To test the potential aggregation of hits we used DLS experiments. The experiments were performed on a ZS90 Zetasizer instrument (Malvern Panalytical, UK). Measurements were done in triplicate with 10 scans per run (100 s). The compounds were used at 20 μM concentration in running buffer (10 mM HEPES pH 7.5, 50 mM NaCl, 0.1% (v/v) Tween20 with or without 0.5 mM TCEP, 2% DMSO) which was filtered before usage. The 20 μM working solution was made from a 1 mM stock of the compound in DMSO. The data was analysed by the built-in software. Compounds were classified as aggregated when the radius of the measured particles was above the minimum colloidal aggregate size (for small molecules) of 50 nm³⁹.

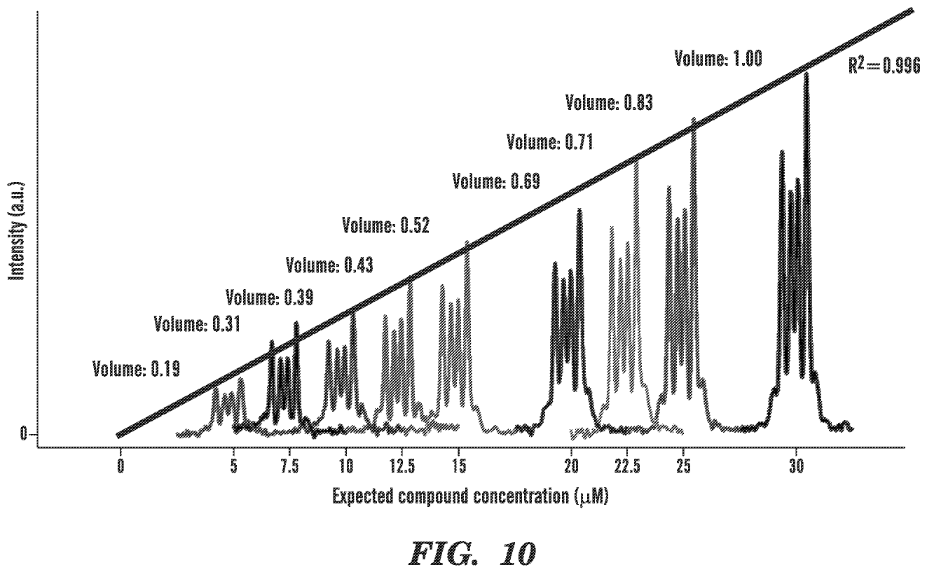

In addition, the solubility of iKeap1, our most potent displacer, was analysed with an NMR solubility assay based on a technique described previously⁴⁰. We made individual samples of iKeap1 at various concentrations (in PBS buffer, pH 7.4) ranging from 5 μM to 30 μM and measured the 1D NMR spectrum of each sample with identical experimental conditions. The resonances of iKeap1 were then integrated and plotted as a function of concentration. The plot shows a linear trend (R²=0.996) indicating that iKeap1 does not aggregate in this concentration range (FIG. 10).

Excluding Interference from PAINS

PAINS comprise 480 markers initially identified as moieties postulated to cause interference in experimental high-throughput screens⁴¹. PAINS compounds are often found in the databases commonly used for in silico screens, and the user should be cognizant of the fact that a potential hit could harbour a PAINS sub-structure. However, it should also be noted that certain PAINS-like aspects can be mitigated by judicious use of medicinal chemistry, and some aspects of PAINS could have no effect, depending on the target of choice and/or the experimental assays used^{42,43}. Attention should be paid to identifying and rigorously characterizing any PAINS compounds amongst the hits identified in an in silico screen.

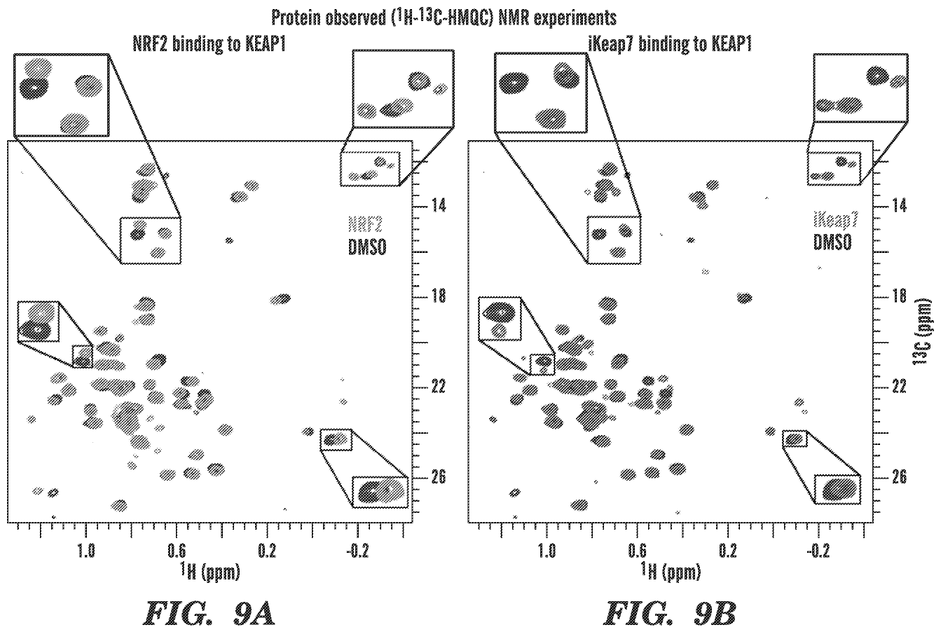

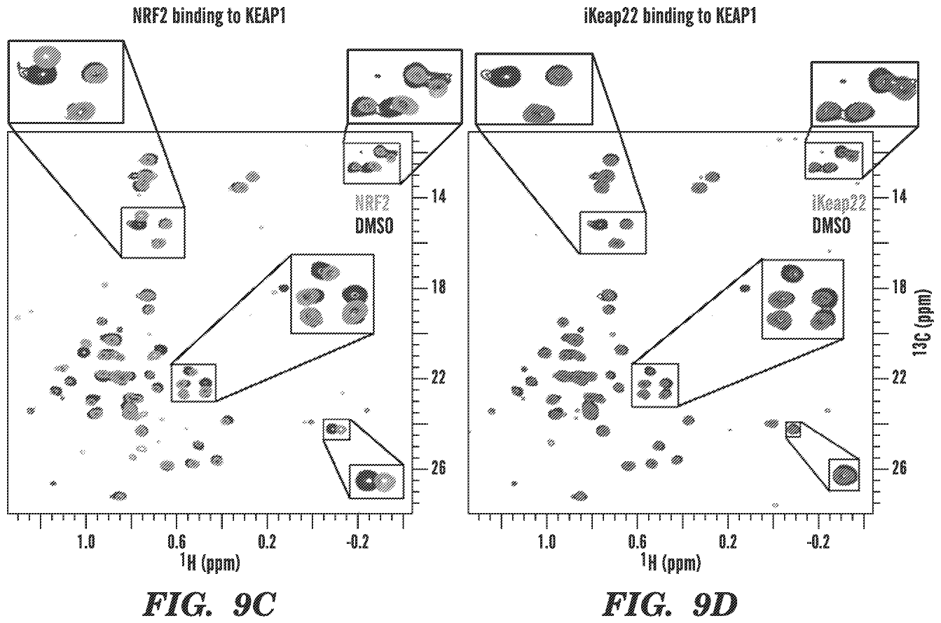

Two of the hit compounds (iKeap2 and iKeap7) reported in this manuscript harbour PAINS substructures. We performed additional experiments to confirm that iKeap2 and iKeap7 are not false positives due to assay interference. Primarily, we used 1) DLS to confirm that all the compounds shown here do not aggregate at the concentrations used in the various experiments (Table 6), 2) ligand-detected NMR experiments, STD and CPMG, performed with a 10-fold excess of the compound to show that iKeap2 and iKeap7 bind KEAP1 in a reversible manner (FIGS. 1A-1H and 5A-5F), and 3) protein-observed ¹H—¹³C HMQC experiments to show that both iKeap2 and iKeap7 engage KEAP1 in a specific manner at the NRF2 binding site and do not aggregate the protein (FIGS. 7A-7H and 9A-9H). In the event these compounds caused the KEAP1 to aggregate, all the resonances will be broadened, which was not the case here.

### Statistics and Reproducibility

Screening size 100K: minimum: −10.3; maximum: −11.6; median: −10.4; Q₁: −10.4, Q₃: −10.6. Screening size 1M: minimum: −10.9; maximum: −12; median: −11; Q₁: −11.1, Q₃: −11.3. Screening size 10M: minimum: −11.675; maximum: −12.3; median: −11.5; Q₁: −11.4, Q₃: −11.5. Screening size 100M: minimum: −11.8; maximum: −12.6; median: −11.9; Q₁: −11.8, Q₃: −12.1. Screening size 1B: minimum: −12.3; maximum: −13.4; median: −12.4; Q₁: −12.3, Q₃: −12.6.

### Data Availability

The ready-to-dock library from Enamine is freely available online on the homepage of VirtualFlow at http://virtual-flow.org/real-library. Source data of FIGS. 3A-3H, 4A-4H and 5A-5F is available online at https://doi.org.

### Code Availability

VirtualFlow is mainly written in Bash (a Turing complete command language), which not only makes it simple for anyone to modify and extend the code, but also has essentially no computational overhead and is readily available in any major Linux distribution. The code for VirtualFlow is freely available on https://github.com/VirtualFlow, distributed under the GNU GPL open-source license. The primary homepage for end users where additional resources including documentation, ligand libraries, tutorials and video demonstrations is available at https://www.virtual-flow.org. The external docking programs discussed here are available as follows: AutoDock Vina is available at http://vina.scripps.edu, QuickVina 2 and QuickVina-W at https://qvina.github.io, Vina-Carb at http://glycam.org/docs/othertoolsservice/download-docs/publication-materials/vina-carb, Smina at https://sourceforge.net/projects/smina, AutoDockFR at http://adfr.scripps.edu and VinaXB at https://github.com/ssirimulla/vinaXB.

### Supplementary Information

A. KEAP1 Binders from the Literature

Table 2 lists the experimentally verified binders to the NRF2-binding domain of KEAP1 found in [19, 15, 29, 11], which were added to the primary virtual screening for the purpose of further validation of VirtualFlow. The threshold docking score (resembling the free energy of binding ΔG) for the top 10% of the compounds is −8.6 kcal/mol. All 17 of the compounds have a predicted docking score above that threshold, indicating that the docking procedure in the primary virtual screening has worked well, despite the use of the lowest possible docking accuracy.

TABLE 2

| | | | | Docking |
|---|---|---|---|---|
| | | IC₅₀ | K_{d} | Score |
| Compound | SMILES | [μM] | [μM] | [kcal/mol] |
| C1 | O═C(O)[C@@H]1CCCC[C@@H]1C(═O) | 3 | | −8.7 |
| | N3CCe2ccccc2C3Cn5c(═O)c4ccccc4c5═O | | | |
| C2 | O═C(O)[C@H]1CCCC[C@H]1C(═O)] | 2.3 | | −9.0 |
| | N1CCc2ccccc2[C@@H]1CN1C(═O) | | | |
| | c2ccccc3c(═O) | | | |
| C3 | O═C(O)[C@H]1CCC[C@H]1C(═O) | 2.2 | | −9.8 |
| | N3CCc2ccccc2[C@H]3Cn5e(═O) | | | |
| | c4ccccc4c5═O | | | |
| C4 | O═C(O)[C@H]1CC[C@H]3C(═O) | 8.0 | | −9.3 |
| | N3CCc2cccccc2[C@H]3Cn5c(═O) | | | |
| | c4ccccc4c5═O | | | |
| C5 | O═C(O)[C@H]1C[C@H]1C(═O) | 20.8 | | −9.6 |
| | N3CCe2cccc2[C@H]3Cn5c(═O) | | | |
| | c4cccc4c5═O | | | |
| C6 | O═C(O)[C@H]1CCNC[C@H]1C(═O) | 69.7 | | −8.7 |
| | N3CCe2ccccc2[C@H]3Cn5c(═O) | | | |
| | c4ccccc4c5═O | | | |
| C7 | O═C(O)[C@H]1CCCC[C@H]1C(═O) | 1.1 | | −9.5 |
| | N1CCc2ccccc2[C@H]1CN1Ce2ccccc2C1═O | | | |
| C8 | O═C(O)[C@H]1CCCC[C@H]1C(═O) | 1.2 | | −9.1 |
| | N3CCc2ccccc2[C@H]3CN4C(═O)CCC4═O | | | |
| C9 | O═C1c2ccccc2C(═O) | 7.4 | | −10.8 |
| | N1C[C@@H]1c2ccccc2CCN1C | | | |
| | (═O)[C@@H]1CCCC[C@@H]1c1nno[ [symbol] | | | |
| C10 | Cc1cc(C)c(C)c(S(═O)(═O)Nc2ccc | 0.14 | | −10.5 |
| | (N3CC[C@@H]1C(═O)O)C3)c3cccc23c1C | | | |
| C11 | CC(C)c1ccc(S(═O)(═O)Nc2cc(Sc3ncn(nH)3)c(O) | | 3.9 | −9.4 |
| | c3cccc23jcc1 | | | |
| C12 | O═C(O)c4cccc3ccc(C═c2c(═O)(cH)n(c1cccc(C) | | 15.2 | −8.8 |
| | cc1)c2═O)c3)c4C1 | | | |
| C13 | Cc4cccc1NC(═O)Cn4c(═O)sc | | 10.4 | −16.0 |
| | (═cc2cccn2C3CCCC(C(═O)O)C3)C4═O | | | |
| C14 | CN(Cc1cc[C@H](CC(═O)O)c2ccc3c(c2) | 3.4 | | −8.7 |
| | non3Cccc)ClS(C)(═O)═O | | | |
| C15 | CN(Cc1cc[C@H](OC(═O)O)c2cc3c(c2)mm3C) | 0.27 | | −9.7 |
| | ccc(C)S(═O)(═O)c1cccc1 | | | |
| C16 | Cc3ccc([C@H](CC(═O)O)c1ccc2c(c1nnn3C) | 0.015 | | −10.8 |
| | nn3CN3C[C@@H](CcOc4ccccc4S5(═O)═O | | | |
| C17 | COc1ccccS(═O)(═O)Nc3ccc(NS(═O)(═O) | 2.7 | | −9.9 |
| | c3ccc(OC)cc3)c3ccccc23)cc1 | | | |

[symbol]

indicates data missing or illegible when filed

Previously identified binders to the NRF2-binding domain of KEAP1. Shown are the SMILES-formatted chemical structures, the reported IC₅₀ values, and the predicted docking scores from the primary virtual screen. The goal of the primary virtual screen was to distinguish binders from non-binders for demonstration purposes, and the stringency was set to the lowest possible level.

In FIG. 3A the crystal structure which was used for the virtual screening procedure is shown, as well as the structure of iKeap1 (FIG. 3C). The structure of iKeap1 is similar to a previously published inhibitor of KEAP1, shown as compound C17 (N,N′-Naphthalene-1,4-Diylbis(4-Methoxybenzenesulfonamide) in Table 2 and FIG. 3D [19]. The predicted docking position for iKeap1 (FIG. 1A) is very similar to the co-crystal structure of compound C17 (FIG. 3D 5d). These similarities further substantiate that iKeap1 binds to the KEAP-1-NRF2 interface.

### B. Experimental Validation Experiments

The SPR Level-2 compounds can be filtered using several characteristics, such as their suitability for medicinal chemistry, predicted pan assay inference (PAINS), or ability to displace the NRF2 peptide. In Table 3 we are listing the hit compounds which are presented in the manuscript. Also included in the table are all the other hits which are able to displace the NRF2 peptide, which do not harbour any PAINS substructures, and which do not contain other problematic substructures (such as azo-dye compounds or compounds which are unsuitable for medicinal chemistry). It should be noted that, in addition to the compounds listed here, our experimental validation identified other potent displacers and binders which were filtered out due PAINS and/or non-drug like properties.

TABLE 3

| ID | SMILES | FP Assay | BLI Assay |
|---|---|---|---|
| iKeap1 | Cc3ccc(S(═O)(═O)Cn2nc3nc4ccccc4cc3ns2OS | active | active |
| | (═O)(═O)c2ccc(O)cc2cc1 | | |
| iKeap2 | O═C(O)c4ccc(NC2═C/C(═N/S(═O)(═O) | active | active |
| | c3ccc4ccccc4e3nc3ccccc3C2═Occ) | | |
| iKeap7 | OC3═NN(c2ccccc2)C(═O)/C1═/s4ccc | active | active |
| | (-c2cc(C(═O)(═O)ccc2C [symbol] | | |
| iKeap8 | O═C(Nc1cccc | active | active |
| | (-c2ccn[nH]3 [symbol] [C@@H]3C[C@H]2CCCC[C@H]2N1C | | |
| | (═O)c1ccc2ccccc2c1 | | |
| iKeap9 | O═S(═O)c(N═C1CCCCCN1)C1═CC═C25C | not active | not active |
| | (NS(═O)(═O) | | |
| | C3═CC═C4C═CC═CC4═C3)═NC2═C1 | | |
| iKeap12 | O═C(c3ccccc [symbol] C1═C[C@@H]2[C@@H]3C(═O)N | active | active |
| | (c4ccccSccccc54)C(═O)[C@@H]3[C@@H](C4═O) | | |
| | c3cccCl)cc3)N2C═O1 | | |
| iKeap22 | O═c1cc(-c2cge(O)cc2nc2c1c(O)cc1c2[C@H] | not active | active |
| | (c2ccc3ncccc3c2)CC(═O)O1 | | |
| iKeap27 | Cc1ccc(C)c(N2C(═O)c3ccc(-c4nc(-c5ccc[nH]nnc6c5) | not active | active |
| | nn4)cc3C2═Ojc1 | | |
| iKeap33 | O═S(═O)Nc [symbol] 2cccc2nc1N1CCC[C@@H] | not active | active |
| | (c2nc3ccccc3[nH]3C1)c1ccccc1 | | |
| iKeap41 | O═C(c1cc(nc2ccc3 [symbol] 2)OCCO3)MC2CCCCC21) | not active | active |
| | N1CCC(CC2CCCCC2)CC1 | | |
| iKeap48 | CC(═O)S/C(nC)c1ccc(Cc2nc3c [symbol] C(═O)c2ccccc3C | not active | active |
| | (═O)cc4c32sc2c[nH]C(═O)c3ccccc1C3═O | | |
| iKeap73 | C[C@H]1CC2cnc3nccs(N4CCO[C@@H](CN5C(═O) | not active | active |
| | c5ccccc6C5═O)C4)c23)C1 | | |
| iKeap74 | C[C@@H}1Oc2ccc(C(═O)C3CCN(C(═O) | not active | active |
| | [C@H]4C[C@H]4c4ccc5cccc45)CC3)cc2NC3═O | | |
| iKeap75 | Cc3cccn2c(═O)c3c(nc32)N1CCCCC[C@H]3[C@H] | not active | active |
| | (C3)C(═O)N(Cc2ccccc2C)C(═O)[N═C]O | | |

[symbol]

indicates data missing or illegible when filed

SPR Level-2 hit compounds, which were also tested experimentally via a fluorescence polarization (FP) assay and BLI experiments for their ability to displace the NRF2-peptide. All compounds shown in this table have also been verified by protein detected NMR experiments (STD, CPMG, DLB, ¹H—¹³C HMQC), or have passed additional filters to remove compounds with problematic substructures (regarding pan assay interference or suitability for medicinal chemistry).

Two of the hit compounds (iKeap2 and iKeap7) contain PAINS alerts. We have carried a suite of orthogonal experiments to show that the compounds are indeed true and specific binders, and are not artifacts due to pan assay interference. DLS and 1D NMR show the compounds are not aggregating, and they are not similar to any known aggregators as assessed by the Tanimoto similarity measure [IDT⁺ 15]. STD-NMR and R2 measurements show the compounds are not covalently binding. The FP assay shows that the compounds bind at the targeted peptide-binding site by displacing it. The FP assay is done in the presence of BSA and to account for non-specific binding. Protein-observed NMR experiments (¹H—¹³C-HMQC) clearly shown the compounds site-specifically engage KEAP1 in a manner similar to NRF2. The protein-detected NMR experiments show that both iKeap2 and iKeap7 do not aggregate the protein (KEAP1).

TABLE 4

| | K_{d, 1} | K_{d, 2} | K_{d, 3} | | | | K_{d}^{average} | SD |
|---|---|---|---|---|---|---|---|---|
| Compound | (nM) | (nM) | (nM) | χ₁² | χ₂² | χ₃² | (nM) | (nM) |
| ikeap1* | 114 | 178 | 85 | 0.026 | 0.404 | 0.033 | 125.667 | 47.585 |
| iKeap2 | 158 | 263 | 334 | 0.031 | 0.333 | 0.265 | 251.667 | 88.546 |
| iKeap7 | 23 | 15 | 16 | 0.0305 | 0.306 | 0.019 | 18.000 | 4.359 |
| iKeap8* | 176 | 115 | 187 | 0.118 | 0.130 | 0.397 | 159.333 | 38.786 |
| iKeap9* | 180 | 187 | 190 | 0.278 | 0.242 | 0.158 | 185.667 | 5.132 |
| iKeap22* | 62 | 51 | 52 | 0.082 | 0.109 | 0.051 | 55.000 | 6.083 |

Kd values which were determined by SPR Level-3 experiments of the compounds shown in more detail above (FIGS. 1A-1H, 4A-4H and 5A-5F). For each compound three independent experiments were carried out, giving rise to three K_{d,i} and χi² values, where i indicates the independent experiment. Compounds marked with an asterisk were determined by fitting the biphasic binding model given by equation (6) to the experimental data,

TABLE 5

| Compound | IC₅₀ (μM) | Standard Error [μM] |
|---|---|---|
| iKeap1 | 0.258 | 0.037 |
| iKeap2 | 2.7 | 0.6 |
| iKeap7 | 38.2 | 3.9 |
| iKeap8 | 14.2 | 4.0 |

IC₅₀ values and the associated errors which were determined by the Level-2 FP experiments are shown here for the compounds highlighted in the manuscript. Representative curves for these FP hits are shown in more detail in the manuscript (FIGS. 1A-1H, 4A-4H and 5A-5F)

TABLE 6

| | Radius | Standard |
|---|---|---|
| Compound | [nm] | Error [nm] |
| buffer | 8.781 | 6.782 |
| iKeap1 | 2.306 | 2.534 |
| iKeap2 | 12.492 | 2.008 |
| iKeap7 | 3.663 | 3.449 |
| iKeap8 | 3.340 | 1.117 |
| iKeap9 | 1.862 | 0.603 |
| iKeap12 | 44.780 | 31.646 |
| iKeap22 | 2.547 | 1.890 |
| iKeap27 | 2.643 | 3.017 |
| iKcap33 | 1.585 | 1.779 |
| iKeap41 | 4.446 | 0.887 |
| iKeap48 | 25.579 | 31.497 |
| iKeap73 | 2.510 | 1.583 |
| iKeap74 | 3.034 | 1.972 |
| iKeap75 | 3.612 | 1.527 |

DLS data for the hit compounds listed in Table 3 showing that these compounds are not aggregating. According to [IDT+15] colloidal aggregates of compounds have radii in the range of 50-800 nm. All measurements were done in triplicates and the average and standard error is shown here.

### REFERENCES

1. DiMasi, J. A., Grabowski, H. G. & Hansen, R. W Innovation in the pharmaceutical industry: New estimates of R&D costs. J. Health Econ. 47, 20-33 (2016).
2. Lyu, J. et al. Ultra-large library docking for discovering new chemotypes. Nature 566, 224-229 (2019).
3. Zhang, S., Kumar, K., Jiang, X., Wallqvist, A. & Reifman, J. DOVIS: an implementation for high-throughput virtual screening using AutoDock. BMC Bioinformatics 9, 126 (2008).
4. Jiang, X., Kumar, K., Hu, X., Wallqvist, A. & Reifman, J. DOVIS 2.0: an efficient and easy to use parallel virtual screening tool based on AutoDock 4.0. Chem. Cent. J 2, 18 (2008).
5. Hassan, N. M., Alhossary, A. A., Mu, Y & Kwoh, C.-K. Protein-Ligand Blind Docking Using QuickVina-W With Inter-Process Spatio-Temporal Integration. Sci. Rep. 7, 15451 (2017).
6. Bohacek, R. S., McMartin, C. & Guida, W. C. The art and practice of structure-based drug design: A molecular modeling perspective. Med. Res. Rev. 16, 3-50 (1996).
7. Yonchuk, J. G. et al. Characterization of the Potent, Selective Nrf2 Activator, 3-(Pyridin-3-Ylsulfonyl)-5-(Trifluoromethyl)-2 H-Chromen-2-One, in Cellular and In Vivo Models of Pulmonary Oxidative Stress. J Pharmacol. Exp. Ther 363, 114-125 (2017).
8. Pallesen, J. S., Tran, K. T. & Bach, A. Non-covalent Small-Molecule Kelch-like ECH-Associated Protein 1-Nuclear Factor Erythroid 2-Related Factor 2 (Keap1-Nrf2) Inhibitors and Their Potential for Targeting Central Nervous System Diseases. J. Med. Chem. 61, 8088-8103 (2018).
9. Davies, T. G. et al. Monoacidic Inhibitors of the Kelch-like ECH-Associated Protein 1: Nuclear Factor Erythroid 2-Related Factor 2 (KEAP1:NRF2) Protein-Protein Interaction with High Cell Potency Identified by Fragment-Based Discovery. J Med. Chem. 59, 3991-4006 (2016).
10. Cuadrado, A. et al. Therapeutic targeting of the NRF2 and KEAP1 partnership in chronic diseases. Nat. Rev. Drug Discov. 18, 295-317 (2019).
11. SLURM. https://slurm.schedmd.com. Accessed: 2018-Dec.-22.
12. Moab/TORQUE. http://www.adaptivecomputing.com. Accessed: 2018-Dec.-22.
13. PBS Professional. http://www.pbspro.org. Accessed: 2018-Dec.-22.
14. LFS. https://www.ibm.com/us-en/marketplace/hpc-workload-management. Accessed: 2018-Dec.-22.
15. SGE. http://gridscheduler.sourceforge.net. Accessed: 2018-Dec.-22.
16. Sterling, T. & Irwin, J. J. ZINC 15—Ligand Discovery for Everyone. J Chem. Inf Model. 55, 2324-2337 (2015).
17. Trott, O. & Olson, A. J. AutoDock Vina: improving the speed and accuracy of docking with a new scoring function, efficient optimization, and multithreading. J. Comput. Chem. 31, 455-61(2010).
18. Alhossary, A., Handoko, S. D., Mu, Y & Kwoh, C.-K. Fast, accurate, and reliable molecular docking with QuickVina 2. Bioinformatics 31, 2214-2216 (2015).
19. Koes, D. R., Baumgartner, M. P. & Camacho, C. J. Lessons Learned in Empirical Scoring with smina from the CSAR 2011 Benchmarking Exercise. J Chem. Inf Model. 53, 1893-1904 (2013).
20. Ravindranath, P. A., Forli, S., Goodsell, D. S., Olson, A. J. & Sanner, M. F. AutoDockFR: Advances in Protein-Ligand Docking with Explicitly Specified Binding Site Flexibility. PLOS Comput. Biol. 11, e1004586 (2015).
21. Koebel, M. R., Schmadeke, G., Posner, R. G. & Sirimulla, S. AutoDock VinaXB: implementation of XBSF, new empirical halogen bond scoring function, into AutoDock Vina. J. Cheminformatics 8, 27 (2016).
22. Nivedha, A. K., Thieker, D. F., Makeneni, S., Hu, H. & Woods, R. J. Vina-Carb: Improving Glycosidic Angles during Carbohydrate Docking. J Chem. Theory Comput. 12, 892-901 (2016).
23. Amaro, R. E. et al. Ensemble Docking in Drug Discovery. Biophys. J. 114, 2271-2278 (2018).
24. Houston, D. R. & Walkinshaw, M. D. Consensus Docking: Improving the Reliability of Docking in a Virtual Screening Context. J. Chem. Inf Model. 53, 384-390 (2013).
25. Marcotte, D. et al. Small molecules inhibit the interaction of Nrf2 and the Keap1 Kelch domain through a non-covalent mechanism. Bioorg. Med. Chem. 21, 4011-4019 (2013).
26. Andrei, S. A. et al. Stabilization of protein-protein interactions in drug discovery. Expert Opin. Drug Discov. 12, 925-940 (2017).
27. Ragoza, M., Hochuli, J., Idrobo, E., Sunseri, J. & Koes, D. R. Protein-Ligand Scoring with Convolutional Neural Networks. J Chem. Inf Model. 57, 942-957 (2017).
28. Reymond, J. The Chemical Space Project. Acc. Chem. Res. 48, 722-730 (2015).
29. ChemAxon. JChem Suite. https://chemaxon.com/products/jchem-engines. Accessed: 2020-Jan.-10.
30. O'Boyle, N. M. et al. Open Babel: An open chemical toolbox. J Cheminformatics 3, 1-14 (2011).
31. Enamine. REAL Library. https://enamine.net/library-synthesis real-compounds. Accessed: 2020-Jan.-10.
32. Morris, G. M. et al. AutoDock4 and AutoDockTools4: Automated docking with selective receptor flexibility. J Comput. Chem. 30, 2785-2791 (2009).
33. SciPy developers. SciPy. https://www.scipy.org. Accessed: 2020-Jan.-10.
34. Surface Plasmon Resonance. vol. 627 (Humana Press, 2010).
35. Hämäläinen, M. D. et al. Label-free primary screening and affinity ranking of fragment libraries using parallel analysis of protein panels. J Biomol. Screen. 13, 202-209 (2008).
36. Receptor-Ligand Interactions A Practical Approach. (Oxford University Press, 1992).
37. Gans, P. et al. Stereospecific Isotopic Labeling of Methyl Groups for NMR Spectroscopic Studies of High-Molecular-Weight Proteins. Angew. Chem. Int. Ed. 49, 1958-1962 (2010).
38. Lu, M. et al. Discovery of a Keap1-dependent peptide PROTAC to knockdown Tau by ubiquitination-proteasome degradation pathway. Eur J Med. Chem. 146, 251-259 (2018).
39. Irwin, J. J. et al. An Aggregation Advisor for Ligand Discovery. J. Med. Chem. 58, 7076-7087 (2015).
40. Laplante, S. R. et al. Compound Aggregation in Drug Discovery: Implementing a Practical NMR Assay for Medicinal Chemists. J Med. Chem. 56, 5142-5150 (2013).
41. Baell, J. B. & Holloway, G. A. New substructure filters for removal of pan assay interference compounds (PAINS) from screening libraries and for their exclusion in bioassays. J. Med. Chem. 53, 2719-2740 (2010).
42. Baell, J. B. & Nissink, J. W. M. Seven Year Itch: Pan-Assay Interference Compounds (PAINS) in 2017—Utility and Limitations. ACS Chem. Biol. 13, 36-44 (2018).
43. Capuzzi, S. J., Muratov, E. N. & Tropsha, A. Phantom PAINS: Problems with the Utility of Alerts for Pan-Assay INterference CompoundS. J Chem. Inf Model. 57, 417-427 (2017).

### Example 2: NQO1 Assay

NQO1 (NAD(P)H Quinone Dehydrogenase is a target gene of NRF2 and has been used to monitor the activity of the NRF2 pathway. See, for example, A. T. Dinkova-Kostova and P. Talalay, NAD(P)H:quinone acceptor oxidoreductase 1 (NQO1), a multifunctional antioxidant enzyme and exceptionally versatile cytoprotector, Arch. Biochem. Biophys. (2010), vol. 501, pp. 116-123. NQO1 is a ubiquitous flavoenzyme that catalyzes the two-electron reduction of quinones to hydroquinones. NQO1 gene expression and enzymatic activity is known to increase proportionately with Nrf2 activation, and this enzyme is routinely used by the field as a NRF2 biomarker for in vitro cell-based assays of compound activity.

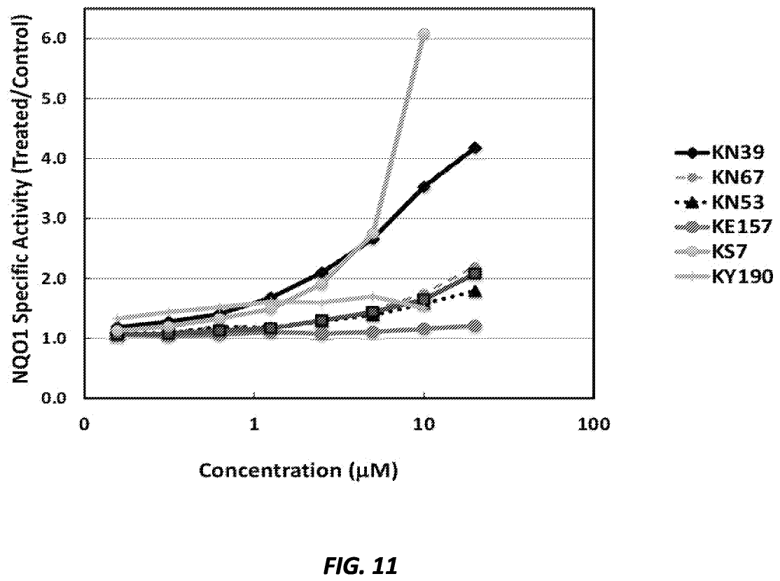

In this study, the inventors used a calorimetric assay to quantitate the NQO1 levels. See, for example, H. J. Prochaska and A. B. Santamaria, Direct measurement of NAD(P)H:quinone reductase from cells cultured in microtiter wells: a screening assay for anticarcinogenic enzyme inducers, Anal. Biochem. (1988), vol. 169, pp. 328-336, and J. W. Fahey et al., A. T. Dinkova-Kostova, K. K. Stephenson, P. Talalay, The “Prochaska” microtiter plate bioassay for inducers of NQO1, Methods Enzymol. (2004), vol. 382, pp. 243-258. Specifically, Hepa1c1c7 cells were exposed in 8 replicates to a vehicle (0.200 DMSO) or 8 serial dilutions of each compound for 48 h. Cells were then lysed and the specific activity of NQO1 was determined using menadione as a substrate. Results are shown in FIG. 11 and summarized in Table 7.

TABLE 7

| Publication IDs: | | | | | | |
|---|---|---|---|---|---|---|
| iKeap1 | iKeap4 | iKeap18 | iKeap8 | iKeap7 | iKeap2 | iKeap17 |

HMS IDs:

| uM | KN39 | KN67 | KN53 | KE157 | KX29 | KS7 | KY190 |
|---|---|---|---|---|---|---|---|
| 0 | 1 | 1 | 1 | 1 | 1 | 1 | 1 |
| 0.156 | 1.19 | 1.06 | 1.09 | 1.07 | 1.06 | 1.13 | 1.33 |
| 0.313 | 1.28 | 1.1 | 1.09 | 1.04 | 1.08 | 1.2 | 1.44 |
| 0.625 | 1.41 | 1.21 | 1.19 | 1.06 | 1.13 | 1.33 | 1.52 |
| 1.25 | 1.68 | 1.18 | 1.18 | 1.11 | 1.17 | 1.49 | 1.61 |
| 2.5 | 2.1 | 1.27 | 1.29 | 1.08 | 1.3 | 1.91 | 1.6 |
| 5 | 2.66 | 1.47 | 1.39 | 1.11 | 1.44 | 2.75 | 1.7 |
| 10 | 3.53 | 1.75 | 1.58 | 1.16 | 1.65 | 6.08 | 1.51 |
| 20 | 4.18 | 2.2 | 1.79 | 1.21 | 2.08 | | 1.43 |
| CD (uM) | 2 | 15 | 40 (extp) | | 20 | 2.7 | |

Note:
Compound KS7 was toxic at concentrations higher than 2.5 uM

All patents and other publications identified herein are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

What is claimed is:

1. A method of inhibiting Kelch-like ECH-associated protein 1 (KEAP1), the method comprising contacting KEAP1 with a compound selected from the following:
(i) compounds of Formula (I): 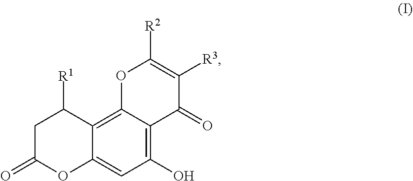
wherein:
R¹ is substituted or unsubstituted aryl, substituted or unsubstituted heteroaryl, or substituted or unsubstituted heterocyclyl;
R² is substituted or unsubstituted aryl, substituted or unsubstituted heteroaryl, or substituted or unsubstituted heterocyclyl;
R³ is hydrogen, —OR^{A}, —SR^{A}, or —N(R^{A})₂; and
each R^{A} independently is H, substituted or unsubstituted alkyl, substituted or unsubstituted alkenyl, substituted or unsubstituted aryl, substituted or unsubstituted heteroaryl, substituted or unsubstituted heterocyclyl, or substituted or unsubstituted cyclyl,
or a pharmaceutically acceptable salt, solvate, hydrate, polymorph, co-crystal, tautomer, stereoisomer, isotopically labeled derivative, or prodrug thereof;
(ii) compounds of Formula (II): 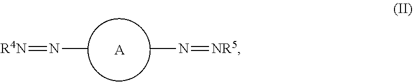
wherein:
A is substituted or unsubstituted arylene, substituted or unsubstituted biarylene, or substituted or unsubstituted heteroarylene;
R⁴ is substituted or unsubstituted aryl, substituted or unsubstituted heteroaryl, or substituted or unsubstituted heterocyclyl; and
R⁵ is substituted or unsubstituted aryl, substituted or unsubstituted heteroaryl, or substituted or unsubstituted heterocyclyl;
or a pharmaceutically acceptable salt, solvate, hydrate, polymorph, co-crystal, tautomer, stereoisomer, isotopically labeled derivative, or prodrug thereof;
(iii) compounds of Formula (III): 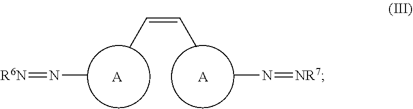
wherein:
each A is independently substituted or unsubstituted aryl, or substituted or unsubstituted heteroaryl;
R⁶ is substituted or unsubstituted aryl, substituted or unsubstituted heteroaryl, or substituted or unsubstituted heterocyclyl; and
R⁷ is substituted or unsubstituted aryl, substituted or unsubstituted heteroaryl, or substituted or unsubstituted heterocyclyl;
or a pharmaceutically acceptable salt, solvate, hydrate, polymorph, co-crystal, tautomer, stereoisomer, isotopically labeled derivative, or prodrug thereof;
or
(iv) a compound selected from Group A, wherein the Group A comprises the compounds: 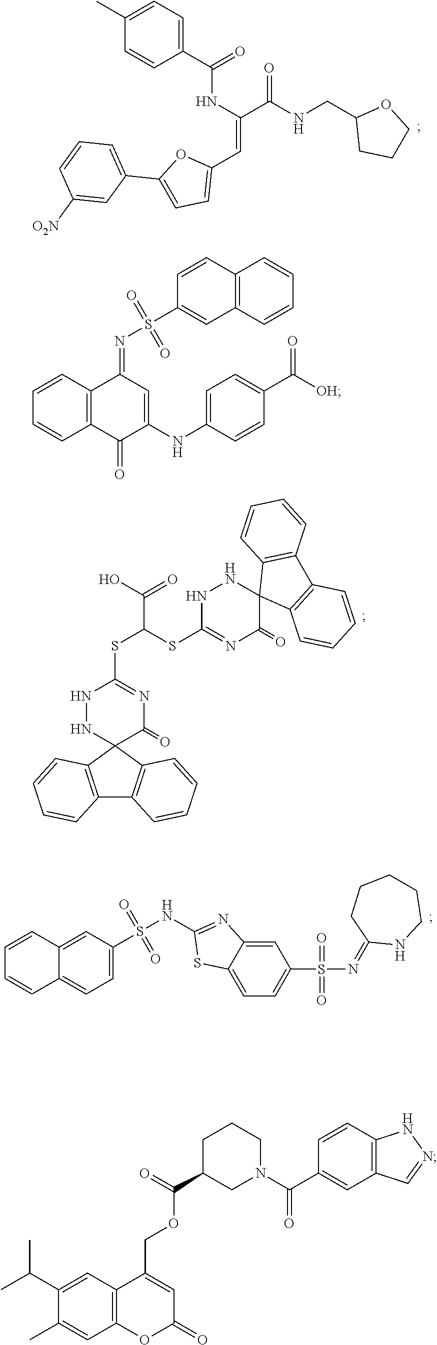 [SMILES: Cc1ccc(C(=O)N/C(=C\c2ccc(-c3cccc([N+](=O)[O-])c3)o2)C(=O)NCC2CCCO2)cc1 ; O=C(O)c1ccc(NC2=C/C(=N\S(=O)(=O)c3ccc4ccccc4c3)c3ccccc3C2=O)cc1 ; O=C(O)C(SC1=NC(=O)C2(NN1)c1ccccc1-c1ccccc12)SC1=NC(=O)C2(NN1)c1ccccc1-c1ccccc12 ; O=S(=O)(/N=C1\CCCCCN1)c1ccc2sc(NS(=O)(=O)c3ccc4ccccc4c3)nc2c1 ; Cc1cc2oc(=O)cc(COC(=O)[C@H]3CCCN(C(=O)c4ccc5[nH]ncc5c4)C3)c2cc1C(C)C] 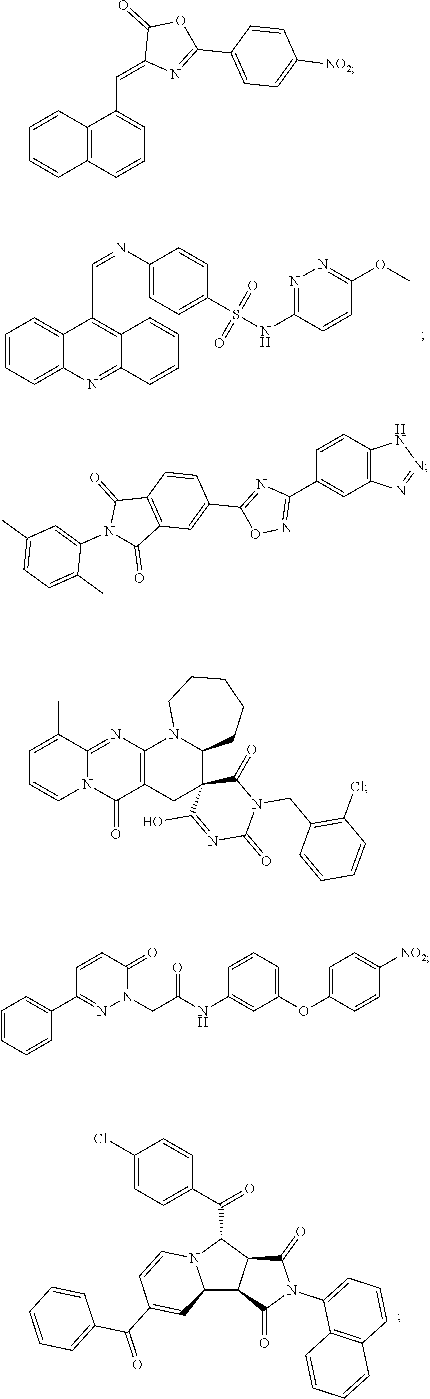 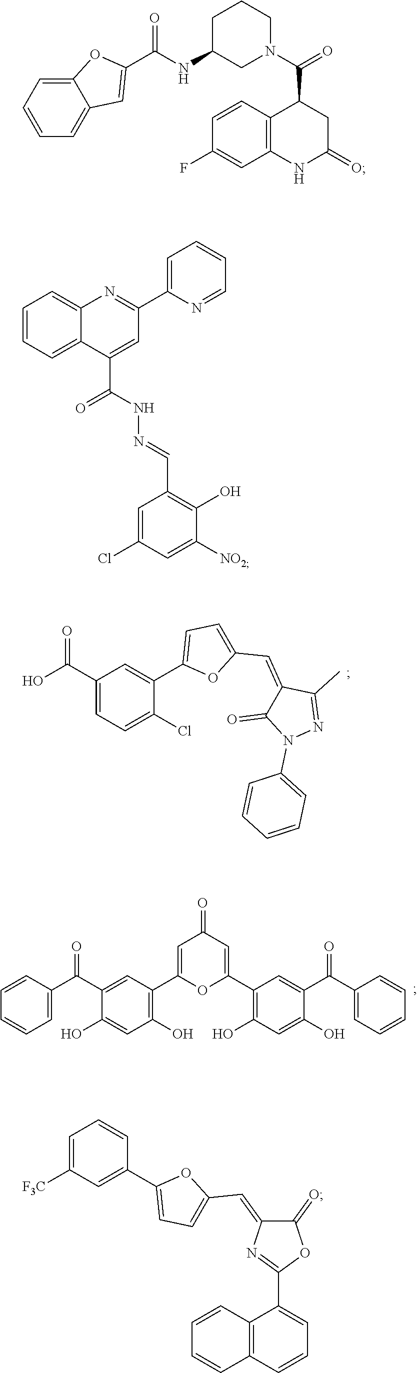 [SMILES: O=C1C[C@H](C(=O)N2CCC[C@H](NC(=O)c3cc4ccccc4o3)C2)c2ccc(F)cc2N1 ; O=C(N/N=C/c1cc(Cl)cc([N+](=O)[O-])c1O)c1cc(-c2ccccn2)nc2ccccc12 ; CC1=NN(c2ccccc2)C(=O)/C1=C\c1ccc(-c2cc(C(=O)O)ccc2Cl)o1 ; O=C(c1ccccc1)c1cc(-c2cc(=O)cc(-c3cc(C(=O)c4ccccc4)c(O)cc3O)o2)c(O)cc1O ; O=C1OC(c2cccc3ccccc23)=N/C1=C\c1ccc(-c2cccc(C(F)(F)F)c2)o1] 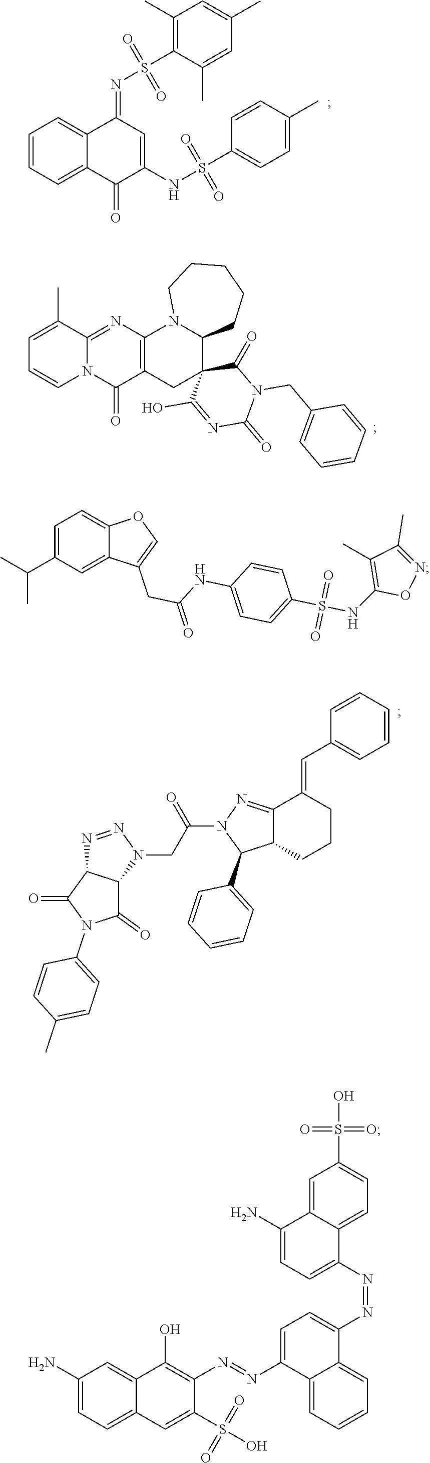 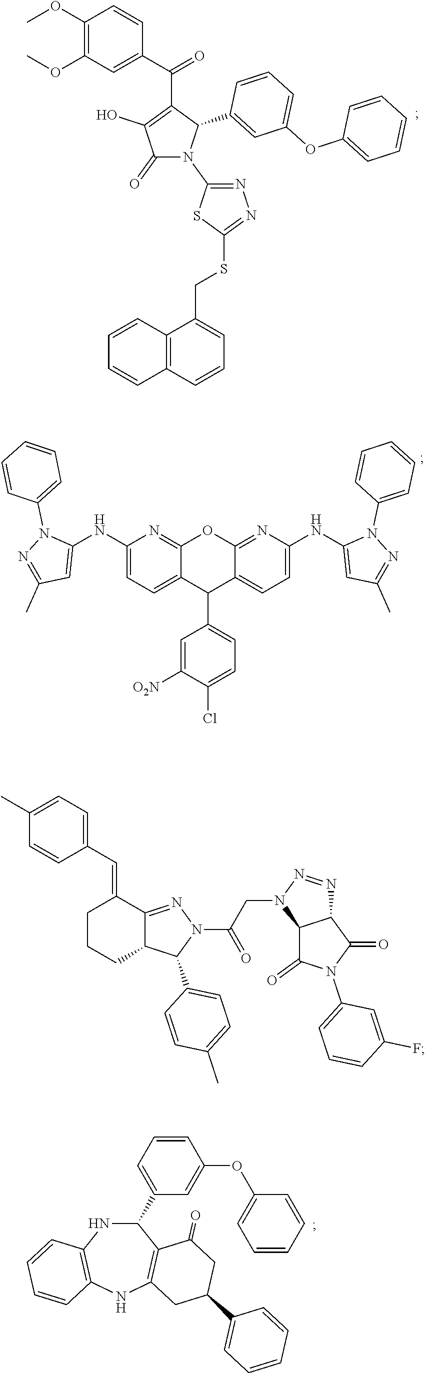 [SMILES: COc1ccc(C(=O)C2=C(O)C(=O)N(c3nnc(SCc4cccc5ccccc45)s3)[C@H]2c2cccc(Oc3ccccc3)c2)cc1OC ; Cc1cc(Nc2ccc3c(n2)Oc2nc(Nc4cc(C)nn4-c4ccccc4)ccc2C3c2ccc(Cl)c([N+](=O)[O-])c2)n(-c2ccccc2)n1 ; Cc1ccc(/C=C2\CCC[C@@H]3C2=NN(C(=O)CN2N=N[C@@H]4C(=O)N(c5cccc(F)c5)C(=O)[C@H]42)[C@@H]3c2ccc(C)cc2)cc1 ; O=C1C[C@@H](c2ccccc2)CC2=C1[C@@H](c1cccc(Oc3ccccc3)c1)Nc1ccccc1N2] 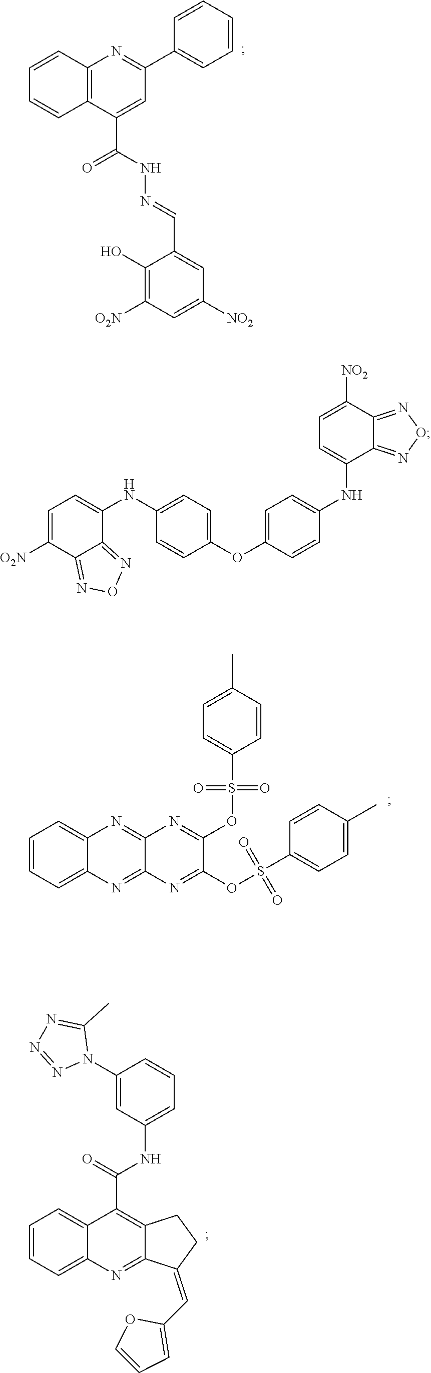 [SMILES: O=C(N/N=C/c1cc([N+](=O)[O-])cc([N+](=O)[O-])c1O)c1cc(-c2ccccc2)nc2ccccc12 ; O=[N+]([O-])c1ccc(Nc2ccc(Oc3ccc(Nc4ccc([N+](=O)[O-])c5nonc45)cc3)cc2)c2nonc12 ; Cc1ccc(S(=O)(=O)Oc2nc3nc4ccccc4nc3nc2OS(=O)(=O)c2ccc(C)cc2)cc1 ; Cc1nnnn1-c1cccc(NC(=O)c2c3c(nc4ccccc24)/C(=C\c2ccco2)CC3)c1] 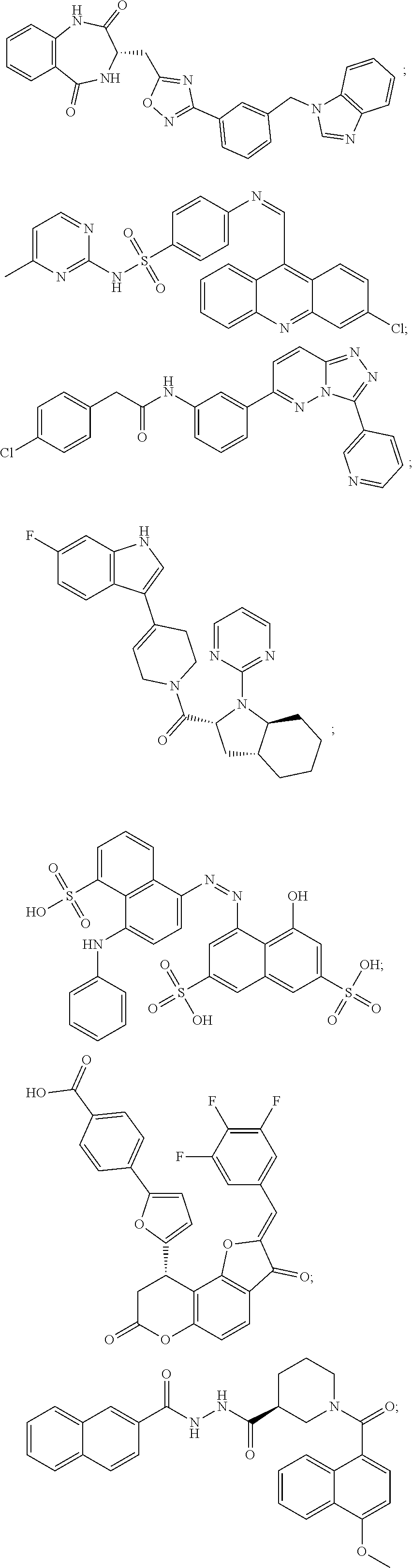 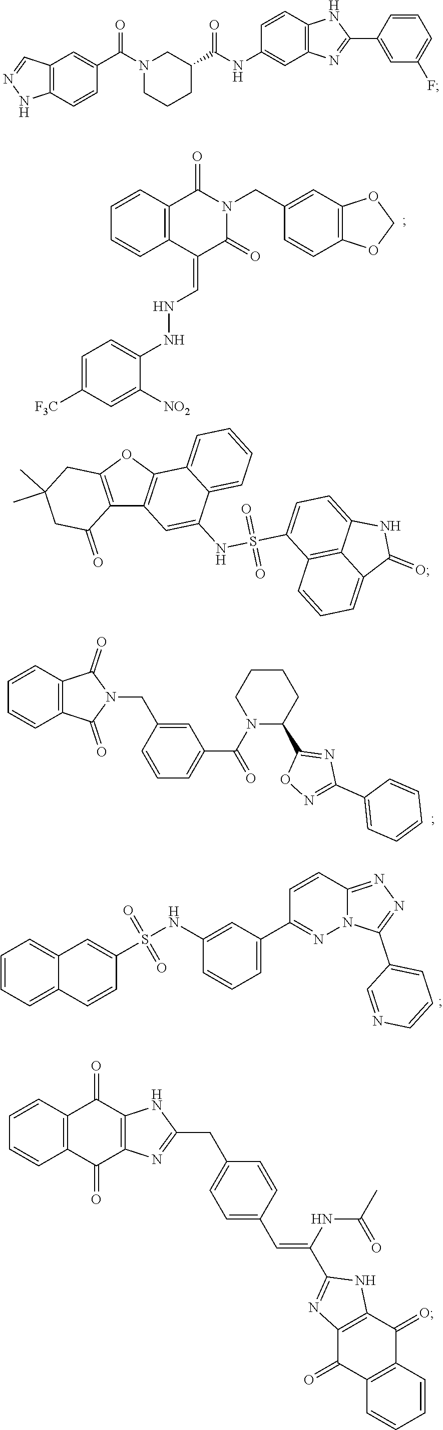 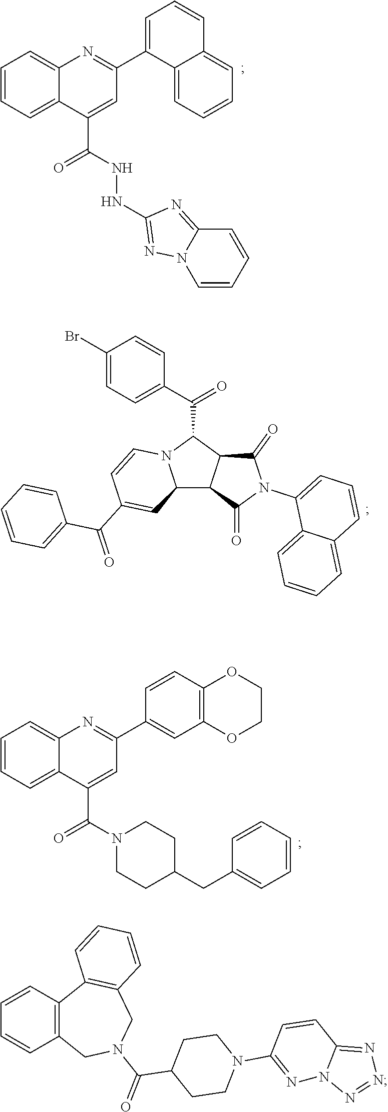 [SMILES: O=C(NNc1nc2ccccn2n1)c1cc(-c2cccc3ccccc23)nc2ccccc12 ; O=C(C1=C[C@@H]2[C@@H]3C(=O)N(c4cccc5ccccc45)C(=O)[C@@H]3[C@@H](C(=O)c3ccc(Br)cc3)N2C=C1)c1ccccc1 ; O=C(c1cc(-c2ccc3c(c2)OCCO3)nc2ccccc12)N1CCC(Cc2ccccc2)CC1 ; O=C(C1CCN(c2ccc3nnnn3n2)CC1)N1Cc2ccccc2-c2ccccc2C1] 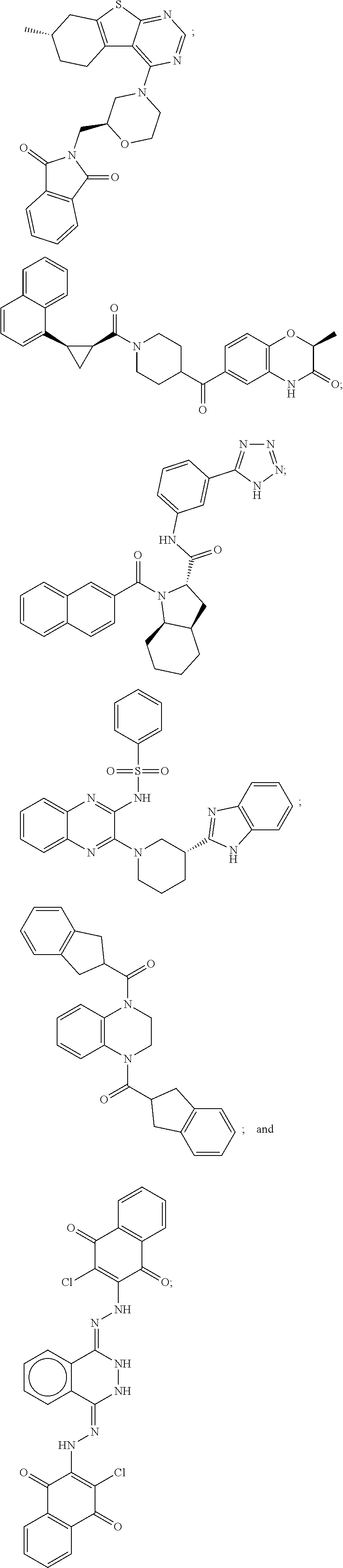
or a pharmaceutically acceptable salt, solvate, hydrate, polymorph, co-crystal, tautomer, stereoisomer, isotopically labeled derivative, or prodrug thereof.

2. (canceled)

3. (canceled)

4. The method of claim 1, wherein the KEAP1 is in a cell and the method comprised administering the compound to the cell.

5. The method of claim 4, wherein said administering to the cell is in vitro.

6. The method of claim 4, wherein said administering to the cell is in vivo.

7. The method of claim 6, wherein said administering to the cell is in a subject having or diagnosed with a disease associated with dysfunction of Nrf2-KEAP1 axis or a disease associated with Nrf2-KEAP1 interaction.

8. (canceled)

9. A method treating a disease associated with dysfunction of the Nrf2-KEAP1 axis or a disease associated with Nrf2-KEAP1 interaction in a subject in need thereof, the method comprising administering to a subject in need thereof a therapeutically effective amount a compound selected from of the group consisting of compounds of Formula (I), compounds of Formula (II), compounds of Formula (III), compounds of Group A, or a pharmaceutically acceptable salt, solvate, hydrate, polymorph, co-crystal, tautomer, stereoisomer, isotopically labeled derivative, or prodrug thereof.

10. (canceled)

11. The method of claim 9, wherein the disease is associated with oxidative stress.

12. The method of claim 11, wherein the disease is selected from the group consisting abdominal aortic aneurysm, acute kidney injury, adult brain glioblastoma, advanced solid tumors lymphoid malignancies, aging, alcohol sensitivity, allergic, Alport syndrome, Alzheimer's disease, asthma, atopic asthmatics, autism spectrum disorder, autosomal dominant polycystic kidney, Barrett esophagus, low-grade dysplasia, brain ischemia, breast cancer or breast neoplasm, cardiovascular risk, cataract surgery, cholelithiasis, cholestasis, chronic hepatitis c, chronic kidney disease, chronic lymphocytic leukemia, chronic renal insufficiency, chronic schizophrenia, chronic subclinical inflammation, CKD associated with type 1 diabetes, cognition, colon cancer, COPD, corneal endothelial cell loss, crohn's disease, cutaneous t cell lymphoma, diabetes mellitus, diabetic nephropathy, diarrhea, endometriosis, environmental carcinogenesis, focal segmental glomerulosclerosis, Friedreich's ataxia, healthy, Helicobacter pylori infection, hepatic impairment, healthy, huntington disease, IgA nephropathy, inflammation and pain following ocular surgery, insulin resistance, liver disease, lung cancer, major depression, melanoma, metabolic syndrome x, mild cognitive impairment, mitochondrial myopathy, multiple sclerosis, neoplasms, nonalcoholic fatty liver or nonalcoholic steatohepatitis, noninsulin-dependent, nonischemic cardiomyopathy, obstructive sleep apnea, ocular inflammation, ocular pain, polymorphism, prediabetes, primary biliary cirrhosis, primary focal segmental glomerulosclerosis (FSGS), prostate cancer, psoriasis, psychosis, pulmonary arterial hypertension (pah), pulmonary hypertension, redox status, rheumatoid arthritis, rhinitis, schistosomiasis, schizophrenia, small lymphocytic lymphoma, subarachnoid haemorrhage, and type 2 (type 2 diabetes).

13. The method of claim 9, wherein the subject is a mammal.

14. The method of claim 9, wherein the subject is human.

15. The method of any claim 9, wherein the compound is of Formula (I).

16. The method of claim 9, wherein the compound is 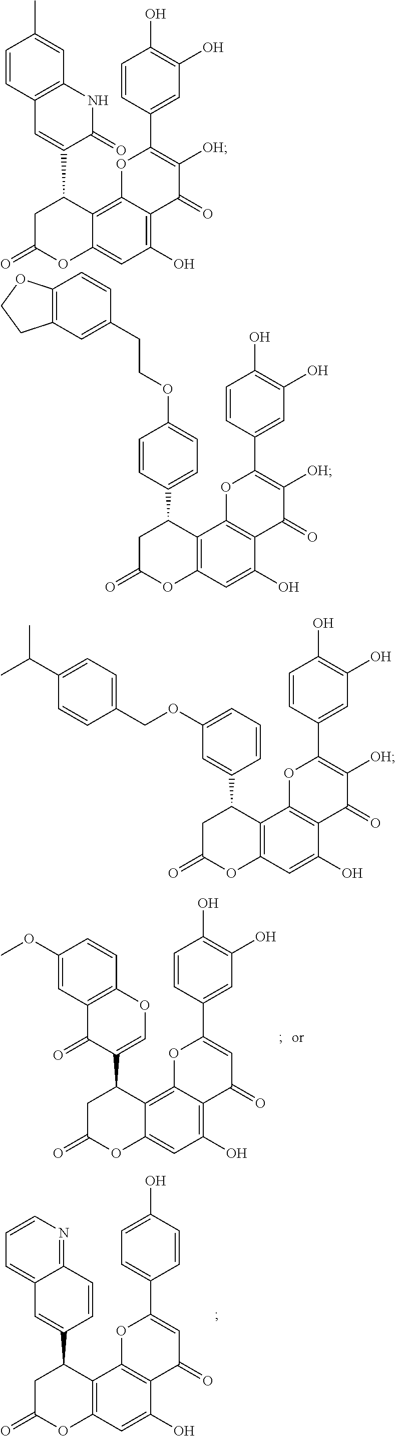
or a pharmaceutically acceptable salt, solvate, hydrate, polymorph, co-crystal, tautomer, stereoisomer, isotopically labeled derivative, or prodrug thereof

17. The method of claim 9, wherein the compound is of Formula (II).

18. The method of claim 9, wherein the compound is 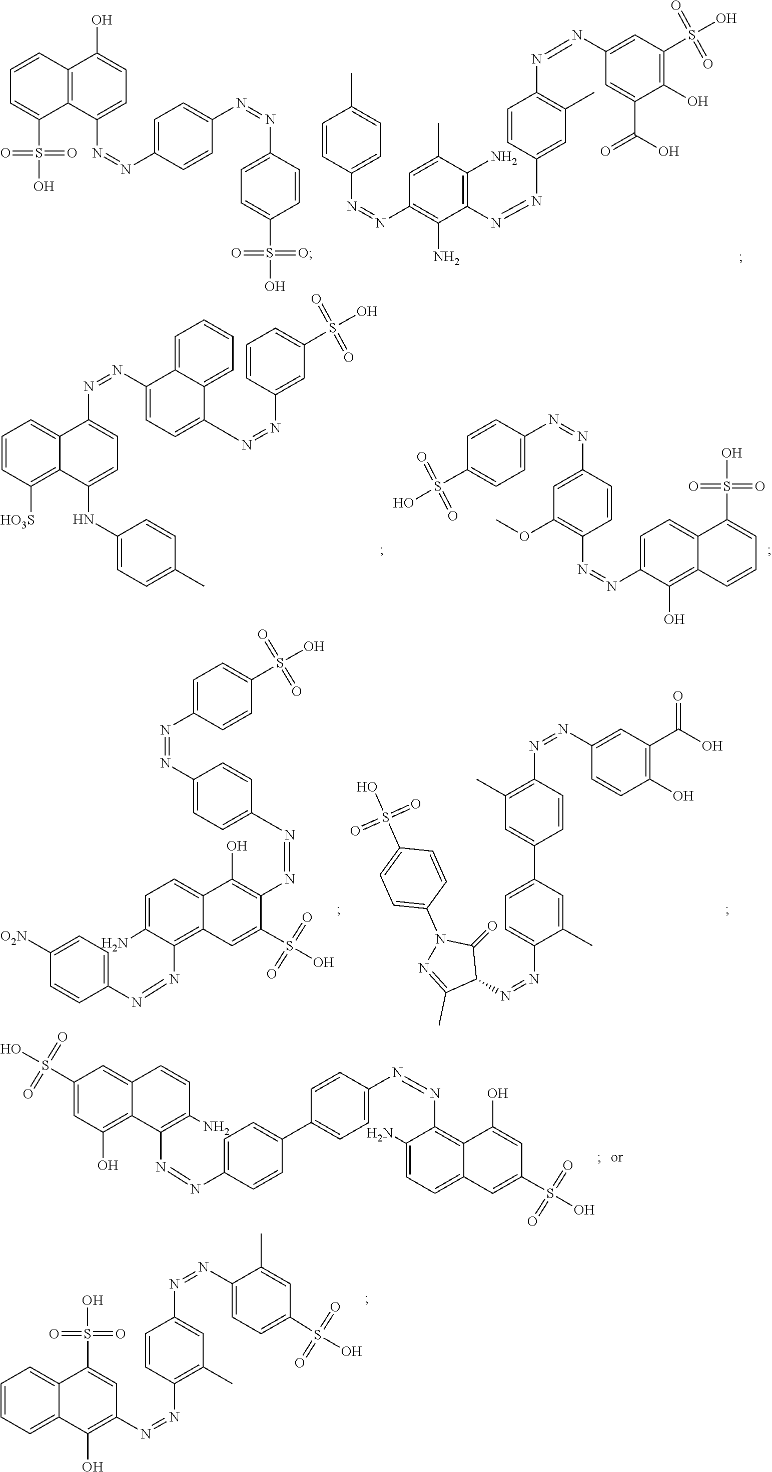
or a pharmaceutically acceptable salt, solvate, hydrate, polymorph, co-crystal, tautomer, stereoisomer, isotopically labeled derivative, or prodrug thereof.

19. The method of claim 9, wherein the compound is of Formula (III).

20. The method of claim 9, wherein the compound is 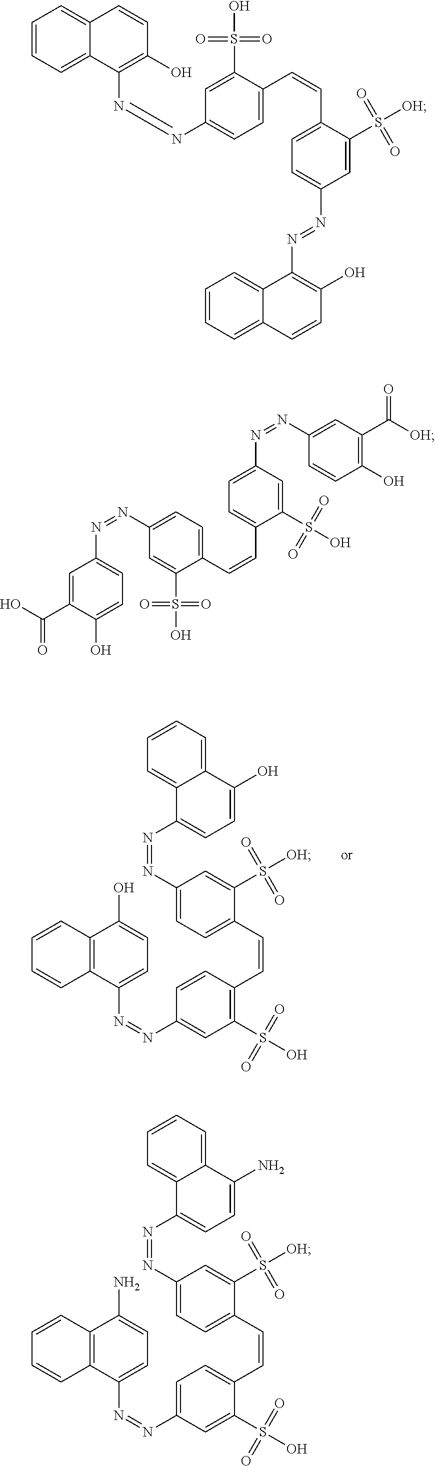 [SMILES: O=S(=O)(O)c1cc(/N=N\c2c(O)ccc3ccccc23)ccc1/C=C\c1ccc(/N=N/c2c(O)ccc3ccccc23)cc1S(=O)(=O)O ; O=C(O)c1cc(/N=N\c2ccc(/C=C\c3ccc(/N=N\c4ccc(O)c(C(=O)O)c4)cc3S(=O)(=O)O)c(S(=O)(=O)O)c2)ccc1O ; O=S(=O)(O)c1cc(/N=N\c2ccc(O)c3ccccc23)ccc1/C=C\c1ccc(/N=N\c2ccc(O)c3ccccc23)cc1S(=O)(=O)O ; Nc1ccc(/N=N\c2ccc(/C=C\c3ccc(/N=N\c4ccc(N)c5ccccc45)cc3S(=O)(=O)O)c(S(=O)(=O)O)c2)c2ccccc12]
or a pharmaceutically acceptable salt, solvate, hydrate, polymorph, co-crystal, tautomer, stereoisomer, isotopically labeled derivative, or prodrug thereof

21. The method of claim 9, wherein the compound is a compound selected from Group A.

22. The method of claim 9, wherein the compound has a structure defined by a Simplified Molecular Input Line Entry System (SMILES) selected from the group consisting of:
CC1=CC═C(C(═O)NC(═CC2=CC═C(C3=CC═CC([N+](═O)[O—])═C3)O2)C(═O)NCC2CCCO2)C═C1 (iKeap 28),
O═C(O)c1ccc(NC2=C/C(═N\S(═O)(═O)c3ccc4ccccc4c3)c3ccccc3C2=O)cc1 (iKeap2),
O═C(O)C(SC1=NC(═O)C2(NN1)c1ccccc1-c1ccccc12)SC1=NC(═O)C2(NN1)c1ccccc1-c1ccccc12 (iKeap 24),
O═S(═O)(N═C1CCCCCN1)C1=CC═C2SC(NS(═O)(═O)C3=CC═C4C═CC═CC4=C3)=NC2=C1 (iKeap9),
Cc1cc2oc(=O)cc(COC(═O)[C@H]3CCCN(C(═O)c4ccc5[nH]ncc5c4)C3)c2cc1C(C)C (iKeap20), O═C1OC(c2ccc([N+](═O)[O-])cc2)=N/C1=C\c1cccc2ccccc12 (iKeap4),
COc1ccc(NS(═O)(═O)c2ccc(/N=C\c3c4ccccc4nc4ccccc43)cc2)nn1 (iKeap29),
Cc1ccc(C)c(N2C(═O)c3ccc(-c4nc(-c5ccc6[nH]nnc6c5)no4)cc3C2=O)c1 (iKeap27),
Cc1cccn2c(=O)c3c(nc12)N1CCCCC[C@H]1[C@]1(C3)C(═O)N(Cc2ccccc2C1)C(═O)N═C1 O (iKeap75), O═C(Cn1nc(-c2ccccc2)ccc1=O)Nc1cccc(Oc2ccc([N+](═O)[O-])cc2)c1 (iKeap51),
O═C(c1ccccc1)C1=C[C@@H]2[C@@H]3C(═O)N(c4cccc5ccccc54)C(═O)[C@@H]3[C@@H](C(═O)c3ccc(C1)cc3)N2C═C1 (iKeap12),
O═S(═O)(O)c1ccc(/N=N\c2ccc(/N=N\c3ccc(O)c4cccc(S(═O)(═O)O)c34)cc2)cc1 (iKeap18),
O═C1C[C@H](C(═O)N2CCC[C@H](NC(═O)c3cc4ccccc4o3)C2)c2ccc(F)cc2N1 (iKeap26),
O═C(NN═CC1=C(O)C([N+](═O)[O—])═CC(C1)=C1)C1=CC(C2=CC═CC═N2)=NC2=CC═CC═C12 (iKeap36),
Cc1ccc2cc([C@H]3CC(═O)Oc4cc(O)c5c(=O)c(O)c(-c6ccc(O)c(O)c6)oc5c43)c(=O)[nH]c2c1 (iKeap52),
CC1=NN(c2ccccc2)C(═O)/C1=Cc1ccc(-c2cc(C(═O)O)ccc2C1)o1 (iKeap7),
O═C1C[C@H](c2ccc(OCCc3ccc4c(c3)CCO4)cc2)c2c(cc(O)c3c(=O)c(O)c(-c4ccc(O)c(O)c4)oc32)O1 (iKeap31), O═C(c1ccccc1)c1cc(-c2cc(=O)cc(-c3cc(C(═O)c4ccccc4)c(O)cc3O)o2)c(O)cc1O (iKeap16),
CC(C)c1ccc(COc2cccc([C@H]3CC(═O)Oc4cc(O)c5c(=O)c(O)c(-c6ccc(O)c(O)c6)oc5c43)c2)cc1 (Ikeap34), O═C1OC(c2cccc3ccccc32)=N/C1=Cc1ccc(-c2cccc(C(F)(F)F)c2)o1 (Ikeap13), COc1ccc2occ([C@@H]3CC(═O)Oc4cc(O)c5c(=O)cc(-c6ccc(O)c(O)c6)oc5c43)c(=O)c2c1 (IKeap 62),
CC1=CC═C(S(═O)(═O)NC2=CC(═NS(═O)(═O)C3=C(C)C═C(C)C═C3C)C3=CC═CC═C3C 2=O)C═C1 (iKeap69),
Cc1cccn2c(=O)c3c(nc12)N1CCCCC[C@H]1[C@]1(C3)C(═O)N(Cc2ccccc2)C(═O)N═C1O (iKeap 39),
CC1=NOC(NS(═O)(═O)C2=CC═C(NC(═O)CC3=COC4=CC═C(C(C)C)C═C34)C═C2)=C1C (Ikeap68),
Cc1ccc(N2C(═O)[C@@H]3N═NN(CC(═O)N4N═C5/C(=C/c6ccccc6)CCC[C@@H]5[C@H]4c4ccccc4)[C@@H]3C2=O)cc1 (iKeap40),
Nc1ccc2cc(S(═O)(═O)O)c(/N═N/c3ccc(/N═N\c4ccc(N)c5cc(S(═O)(═O)O)ccc45)c4ccccc34) c(O)c2c1 (iKeap5),
Cc1ccc(/N═N\c2cc(C)c(N)c(/N═N\c3ccc(/N═N\c4cc(C(═O)O)c(O)c(S(═O)(═O)O)c4)c(C)c3)c2N)cc1 (iKeap30),
COc1ccc(C(═O)C2=C(O)C(═O)N(c3nnc(SCc4cccc5ccccc54)s3)[C@H]2c2cccc(Oc3ccccc3) c2)cc1OC (iKeap56),
CC1=NN(C2=CC═CC═C2)C(NC2=CC═C3C(═N2)OC2=NC(NC4=CC(C)═NN4C4=CC═C C═C4)=CC═C2C3C2=CC═C(C1)C([N+](═O)[O-])═C2)=C1 (iKeap35),
Cc1ccc(/C═C2\CCC[C@@H]3C2=NN(C(═O)CN2N═N[C@@H]4C(═O)N(c5cccc(F)c5)C(═O)[C@H]42)[C@@H]3c2ccc(C)cc2)cc1 (iKeap57),
Cc1ccc(Nc2ccc(/N═N\c3ccc(/N=N\c4cccc(S(═O)(═O)O)c4)c4ccccc34)c3cccc(S(═O)(═O)O) c23)cc1 (iKeap6),
COc1cc(/N═N\c2ccc(S(═O)(═O)O)cc2)ccc1/N=N\c1ccc2c(cccc2S(═O)(═O)O)c1O (iKeap 11),
O═C1C[C@@H](c2ccccc2)CC2=C1[C@@H](c1cccc(Oc3ccccc3)c1)Nc1ccccc1N2 (iKeap77),
O═S(═O)(O)c1cc(/N=N/c2c(O)ccc3ccccc32)ccc1/C═C\c1ccc(/N═N\c2c(O)ccc3ccccc32)cc1 S(═O)(═O)O (iKeap38),
O═C(O)c1cc(/N═N\c2ccc(/C═C\c3ccc(/N═N\c4ccc(O)c(C(═O)O)c4)cc3S(═O)(═O)O)c(S(═O)(═O)O)c2)ccc1O (iKeap23), O═C(N/N═C/c1cc([N+](═O)[O-])cc([N+](═O)[O-])c1O)c1cc(-c2ccccc2)nc2ccccc21 (iKeap54), O═[N+]([O-])c1ccc(Nc2ccc(Oc3ccc(Nc4ccc([N+](═O)[O—])c5nonc54)cc3)cc2)c2nonc21 (iKeap14),
Cc1ccc(S(═O)(═O)Oc2nc3nc4ccccc4nc3nc2OS(═O)(═O)c2ccc(C)cc2)cc1 (ikeap1),
Cc1nnnn1-c1cccc(NC(═O)c2c3c(nc4ccccc24)/C(═C\c2ccco2)CC3)c1 (iKeap46),
O═C1N[C@@H](Cc2nc(-c3cccc(Cn4cnc5ccccc54)c3)no2)C(═O)Nc2ccccc21 (iKeap60),
Cc1ccnc(NS(═O)(═O)c2ccc(/N═C\c3c4ccccc4nc4cc(C1)ccc34)cc2)n1 (iKeap50),
O═C(Cc1ccc(C1)cc1)Nc1cccc(-c2ccc3nnc(-c4cccnc4)n3n2)c1 (iKeap32),
O═C([C@H]1C[C@H]2CCCC[C@@H]2N1c1ncccn1)N1CC═C(c2c[nH]c3cc(F)ccc23)CC1 (iKeap67),
Nc1ccc2c(O)c(/N═N\c3ccc(/N═N\c4ccc(S(═O)(═O)O)cc4)cc3)c(S(═O)(═O)O)cc2c1/N═N\c1 ccc([N+](═O)[O-])cc1 (iKeap55),
Cc1cc(/N═N\c2ccc(S(═O)(═O)O)cc2C)ccc1/N═N\c1cc(S(═O)(═O)O)c2ccccc2c1O (iKeap3),
O═S(═O)(O)c1cc(O)c2c(c1)cc(S(═O)(═O)O)cc2/N═N\c1ccc(Nc2ccccc2)c2c1cccc2S(═O)(═O)O (iKeap25),
O═S(═O)(O)c1cc(/N═N\c2ccc(O)c3ccccc23)ccc1/C═C\c1ccc(/N═N\c2ccc(O)c3ccccc23)cc1 S(═O)(═O)O (iKeap59), CC1=NN(c2ccc(S(═O)(═O)O)cc2)C(═O)[C@@H]1/N═N\c1ccc(-c2ccc(/N═N\c3ccc(O)c(C(═O)O)c3)c(C)c2)cc1C (iKeap58),
Nc1ccc2cc(S(═O)(═O)O)cc(O)c2c1/N═N\c1ccc(-c2ccc(/N═N\c3c(N)ccc4cc(S(═O)(═O)O)cc(O)c43)cc2)cc1 (iKeap10), O═C(O)c1ccc(-c2ccc([C@H]3CC(═O)Oc4ccc5c(c43)O/C(═C\c3cc(F)c(F)c(F)c3)C5=O)o2)cc1 (iKeap71),
Nc1 ccc(/N═N\c2ccc(/C═C\c3ccc(/N═N\c4ccc(N)c5ccccc45)cc3S(═O)(═O)O)c(S(═O)(═O)O) c2)c2ccccc12 (iKeap45),
COc1ccc(C(═O)N2CCC[C@H](C(═O)NNC(═O)c3ccc4ccccc4c3)C2)c2ccccc12 (iKeap70),
O═C(Nc1ccc2[nH]c(-c3cccc(F)c3)nc2c1)[C@@H]1CCCN(C(═O)c2ccc3[nH]ncc3c2)C1 (iKeap64), O═C1c2ccccc2/C(═C\NNc2ccc(C(F)(F)F)cc2[N+](═O)[O—])C(═O)N1Cc1ccc2c(c1)OCO2 (iKeap43),
CC1(C)Cc2oc3c(cc(NS(═O)(═O)c4ccc5c6c(cccc64)C(═O)N5)c4ccccc34)c2C(═O)C1 (iKeap65), O═C1c2ccccc2C(═O)N1Cc1cccc(C(═O)N2CCCC[C@H]2c2nc(-c3ccccc3)no2)c1 (iKeap44), O═S(═O)(Nc1cccc(-c2ccc3nnc(-c4cccnc4)n3n2)c1)c1ccc2ccccc2c1 (iKeap66),
CC(═O)N/C(=Cc1ccc(Cc2nc3c([nH]2)C(═O)c2ccccc2C3=O)cc1)c1nc2c([nH]1)C(═O)c1ccc cc1C2=O (iKeap48), O═C(NNc1nc2ccccn2n1)c1cc(-c2cccc3ccccc23)nc2ccccc12 (iKeap21),
O═C(c1ccccc1)C1=C[C@@H]2[C@@H]3C(═O)N(c4cccc5ccccc54)C(═O)[C@@H]3[C@@H](C(═O)c3ccc(Br)cc3)N2C═C1 (iKeap15), O═C(c1cc(-c2ccc3c(c2)OCCO3)nc2ccccc21)N1CCC(Cc2ccccc2)CC1 (iKeap41),
O═C(C1CCN(c2ccc3nnnn3n2)CC1)N1Cc2ccccc2-c2ccccc2C1 (iKeap41), O═c1cc(-c2ccc(O)cc2)oc2c1c(O)cc1c2[C@H](c2ccc3ncccc3c2)CC(═O)O1 (iKeap22),
C[C@H]1CCc2c(sc3ncnc(N4CCO[C@@H](CN5C(═O)c6ccccc6C5=O)C4)c23)C1 (iKeap73),
C[C@@H]1Oc2ccc(C(═O)C3CCN(C(═O)[C@H]4C[C@H]4c4cccc5ccccc45)CC3)cc2NC1=O (iKeap74), O═C(Nc1cccc(-c2nnn[nH]2)c1)[C@@H]1C[C@H]2CCCC[C@H]2N1C(═O)c1ccc2ccccc2c1 (iKeap8),
O═S(═O)(Nc1nc2ccccc2nc1N1CCC[C@@H](c2nc3ccccc3[nH]2)C1)c1ccccc1 (iKeap33),
O═C(C1Cc2ccccc2C1)N1CCN(C(═O)C2Cc3ccccc3C2)c2ccccc21 (iKeap61),
O═C1c2ccccc2C(═O)C(N/N═c2/[nH][nH]/c(=N\NC3=C(C1)C(═O)c4ccccc4C3=O)c3ccccc32)=C1C1 (iKeap49),
CC(═O)N5CCCC4=CC(NC(═O)c3cccc(NC(═O)C2Cc1ccccc1O2)c3)=CCC45 (iKeap19),
O═C(OCc2cc([N+](═O)O)cc1COCOc12)c6c5CCC/C(═C/c4ccc3OCOc3c4)c5nc7ccccc67 (iKeap47), and
O═c3[nH]c2ccc(c1ccccc1)cc2c3=NNc6nc(c4ccccn4)nc5CCCc56 (iKeap72),
or a pharmaceutically acceptable salt, solvate, hydrate, polymorph, co-crystal, tautomer, stereoisomer, isotopically labeled derivative, or prodrug thereof.

23. The method claim 22, wherein the compound is selected from the group consisting of:
iKeap1, iKeap2, iKeap3, iKeap4, iKeap5, iKeap6, iKeap7, iKeap8, iKeap10, iKeap11, iKeap12, iKeap13, iKeap14, iKeap15, iKeap18, iKeap22, iKeap23, iKeap24, iKeap25, iKeap27, iKeap29, iKeap30, iKeap31, iKeap32, iKeap33, iKeap36, iKeap38, iKeap41, iKeap43, iKeap45, iKeap46, iKeap48, iKeap49, iKeap50, iKeap52, iKeap55, iKeap56, iKeap58, iKeap59, iKeap69, iKeap71, iKeap73, iKeap74, and iKeap75, or a pharmaceutically acceptable salt, solvate, hydrate, polymorph, co-crystal, tautomer, stereoisomer, isotopically labeled derivative, or prodrug thereof.

24. A compound having a structure defined by a Simplified Molecular Input Line Entry System (SMILES) selected from the group consisting of:
Cc1cc2oc(═O)cc(COC(═O)[C@H]3CCCN(C(═O)c4ccc5[nH]ncc5c4)C3)c2cc1C(C)C (iKeap20),
Cc1ccc(C)c(N2C(═O)c3ccc(-c4nc(-c5ccc6[nH]nnc6c5)no4)cc3C2=O)c1 (iKeap27),
O═C(Cn1nc(-c2ccccc2)ccc1=O)Nc1cccc(Oc2ccc([N+](═O)[O-])cc2)c1 (iKeap51),
O═C1C[C@H](C(═O)N2CCC[C@H](NC(═O)c3cc4ccccc4o3)C2)c2ccc(F)cc2N1 (iKeap26),
Cc1nnnn1-c1cccc(NC(═O)c2c3c(nc4ccccc24)/C(═C\c2ccco2)CC3)c1 (iKeap46),
O═C1N[C@@H](Cc2nc(-c3cccc(Cn4cnc5ccccc54)c3)no2)C(═O)Nc2ccccc21 (iKeap60),
O═C([C@H]1C[C@H]2CCCC[C@@H]2N1c1ncccn1)N1CC═C(c2c[nH]c3cc(F)ccc23)CC1 (iKeap67),
COc1ccc(C(═O)N2CCC[C@H](C(═O)NNC(═O)c3ccc4ccccc4c3)C2)c2ccccc12 (iKeap70),
O═C(Nc1ccc2[nH]c(-c3cccc(F)c3)nc2c1)[C@@H]1CCCN(C(═O)c2ccc3[nH]ncc3c2)C1 (iKeap64),
O═C1c2ccccc2/C(═C\NNc2ccc(C(F)(F)F)cc2[N+](═O)[O-])C(═O)N1Cc1ccc2c(c1)OCO2 (iKeap43),
O═C1c2ccccc2C(═O)N1Cc1cccc(C(═O)N2CCCC[C@H]2c2nc(-c3ccccc3)no2)c1 (iKeap44),
O═C(NNc1nc2ccccn2n1)c1cc(-c2cccc3ccccc23)nc2ccccc12 (iKeap21),
O═C(C1CCN(c2ccc3nnnn3n2)CC1)N1Cc2ccccc2-c2ccccc2C1 (iKeap76),
C[C@H]1CCc2c(sc3ncnc(N4CCO[C@@H](CN5C(═O)c6ccccc6C5=O)C4)c23)C1 (iKeap73),
C[C@@H]1Oc2ccc(C(═O)C3CCN(C(═O)[C@H]4C[C@H]4c4cccc5ccccc45)CC3)cc2NC1=O (iKeap74),
O═C(Nc1cccc(-c2nnn[nH]2)c1)[C@@H]1C[C@H]2CCCC[C@H]2N1C(═O)c1ccc2ccccc2c1 (iKeap8),
O═S(═O)(Nc1nc2ccccc2nc1N1CCC[C@@H](c2nc3ccccc3[nH]2)C1)c1ccccc1 (iKeap33), and
O═C(C1Cc2ccccc2C1)N1CCN(C(═O)C2Cc3ccccc3C2)c2ccccc21 (iKeap61)
Cc1cc2oc(═O)cc(COC(═O)[C@H]3CCCN(C(═O)c4ccc5[nH]ncc5c4)C3)c2cc1C(C)C (iKeap20),
Cc1ccc(C)c(N2C(═O)c3ccc(-c4nc(-c5ccc6[nH]nnc6c5)no4)cc3C2=O)c1 (iKeap27),
O═C(Cn1nc(-c2ccccc2)ccc1=O)Nc1cccc(Oc2ccc([N+](═O)[O-])cc2)c1 (iKeap51),
O═C1C[C@H](C(═O)N2CCC[C@H](NC(═O)c3cc4ccccc4o3)C2)c2ccc(F)cc2N1 (iKeap26),
Cc1nnnn1-c1cccc(NC(═O)c2c3c(nc4ccccc24)/C(═C\c2ccco2)CC3)c1 (iKeap46),
O═C1N[C@@H](Cc2nc(-c3cccc(Cn4cnc5ccccc54)c3)no2)C(═O)Nc2ccccc21 (iKeap60),
O═C([C@H]1C[C@H]2CCCC[C@@H]2N1c1ncccn1)N1CC═C(c2c[nH]c3cc(F)ccc23)CC1 (iKeap67), COc1ccc(C(═O)N2CCC[C@H](C(═O)NNC(═O)c3ccc4ccccc4c3)C2)c2ccccc12 (iKeap70), O═C(Nc1ccc2[nH]c(-c3cccc(F)c3)nc2c1)[C@@H]1CCCN(C(═O)c2ccc3[nH]ncc3c2)C1 (iKeap64),
O═C1c2ccccc2/C(═C\NNc2ccc(C(F)(F)F)cc2[N+](═O)[O-])C(═O)N1Cc1ccc2c(c1)OCO2 (iKeap43), O═C1c2ccccc2C(═O)N1Cc1cccc(C(═O)N2CCCC[C@H]2c2nc(-c3ccccc3)no2)c1 (iKeap44), O═C(NNc1nc2ccccn2n1)c1cc(-c2cccc3ccccc23)nc2ccccc12 (iKeap21),
O═C(C1CCN(c2ccc3nnnn3n2)CC1)N1Cc2ccccc2-c2ccccc2C1 (iKeap76),
C[C@H]1CCc2c(sc3ncnc(N4CCO[C@@H](CN5C(═O)c6ccccc6C5=O)C4)c23)C1 (iKeap73),
C[C@@H]1Oc2ccc(C(═O)C3CCN(C(═O)[C@H]4C[C@H]4c4cccc5ccccc45)CC3)cc2NC1=O (iKeap74), O═C(Nc1cccc(-c2nnn[nH]2)c1)[C@@H]1C[C@H]2CCCC[C@H]2N1C(═O)c1ccc2ccccc2c1 (iKeap8),
O═S(═O)(Nc1nc2ccccc2nc1N1CCC[C@@H](c2nc3ccccc3[nH]2)C1)c1ccccc1 (iKeap33),
O═C(C1Cc2ccccc2C1)N1CCN(C(═O)C2Cc3ccccc3C2)c2ccccc21 (iKeap61),
CC(═O)N5CCCC4=CC(NC(═O)c3cccc(NC(═O)C2Cc1ccccc1O2)c3)=CCC45 (iKeap19),
O═C(OCc2cc([N+](═O)O)cc1COCOc12)c6c5CCC/C(=C/c4ccc3OCOc3c4)c5nc7ccccc67 (iKeap47), and
O═c3[nH]c2ccc(c1ccccc1)cc2c3=NNc6nc(c4ccccn4)nc5CCCc56 (iKeap72),
or a pharmaceutically acceptable salt, solvate, hydrate, polymorph, co-crystal, tautomer, stereoisomer, isotopically labeled derivative, or prodrug thereof.

25. (canceled)

26. (canceled)
